SEQUENCE LISTING (84 sequences)

SEQ ID NO: 1 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 4 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 5 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKFHYTQKSLSLSPG

SEQ ID NO: 6 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPG

SEQ ID NO: 7 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKYHYTQKSLSLSPG

SEQ ID NO: 8 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALKFHYTQKSLSLSPG

SEQ ID NO: 9 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALKFHYTQKSLSLSPG

SEQ ID NO: 10 - protein, 585 aa, synthetic construct [sequence omitted]

SEQ ID NO: 11 - protein, 115 aa, synthetic construct [sequence omitted]

SEQ ID NO: 12 - protein, 115 aa, synthetic construct [sequence omitted]

SEQ ID NO: 13 - protein, 5 aa, synthetic construct
GGGGS

SEQ ID NO: 14 - protein, 35 aa, synthetic construct
GGGGSGGGGSGGGGSGGGGSGGGGSGGGGSGGGGS

SEQ ID NO: 15 - protein, 15 aa, synthetic construct
GGGGSGGGGSGGGGS

SEQ ID NO: 16 - protein, 816 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 17 - protein, 816 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 18 - protein, 846 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 846 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 20 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 21 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 22 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 23 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 24 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 25 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 26 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 27 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 28 - protein, 816 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 29 - protein, 816 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPGGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPGGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 31 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 32 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPGGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 33 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 34 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 35 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 36 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 37 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 38 - protein, 346 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 39 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 40 - protein, 356 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 41 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 42 - protein, 346 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPG

SEQ ID NO: 43 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPG

SEQ ID NO: 44 - protein, 356 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPG

SEQ ID NO: 45 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPG

SEQ ID NO: 46 - protein, 816 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 47 - protein, 816 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPGGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 48 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALKFHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 49 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALKFHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 50 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 51 - protein, 346 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALKFHYTQKSLSLSPG

SEQ ID NO: 52 - protein, 346 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPGGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 53 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPGGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 54 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 55 - protein, 356 aa, synthetic construct
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSGGGGSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALKFHYTQKSLSLSPG

SEQ ID NO: 56 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKYHYTQKSLSLSPG

SEQ ID NO: 58 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHFHYTQKSLSLSPG

SEQ ID NO: 59 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALRYHYTQKSLSLSPG

SEQ ID NO: 60 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALRFHYTQKSLSLSPG

SEQ ID NO: 61 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALRFHYTQKSLSLSPG

SEQ ID NO: 62 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 63 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 64 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 65 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKYHYTQKSLSLSPG

SEQ ID NO: 66 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHKDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 67 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHKDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 68 - protein, 371 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 69 - protein, 381 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSGGGGSGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 70 - protein, 356 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 71 - protein, 371 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 72 - protein, 376 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSGGGGSGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 73 - protein, 381 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKYHYTQKSLSLSPGGGGGSGGGGSGGGGSGGGGSGGGGSGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 74 - protein, 30 aa, synthetic construct
GGGGSGGGGSGGGGSGGGGSGGGGSGGGGS

SEQ ID NO: 75 - protein, 40 aa, synthetic construct
GGGGSGGGGSGGGGSGGGGSGGGGSGGGGSGGGGSGGGGS

SEQ ID NO: 76 - protein, 115 aa, synthetic construct [sequence omitted]

SEQ ID NO: 77 - protein, 5 aa, synthetic construct
GGGGG

SEQ ID NO: 78 - protein, 6 aa, synthetic construct
GGSGGD

SEQ ID NO: 79 - protein, 6 aa, synthetic construct
GGSGGE

SEQ ID NO: 80 - protein, 11 aa, synthetic construct
GGGSGSGGGGS

SEQ ID NO: 81 - protein, 11 aa, synthetic construct
GGGGGPGGGGP

SEQ ID NO: 82 - protein, 6 aa, synthetic construct
GGGGGG

SEQ ID NO: 83 - protein, 8 aa, synthetic construct
GGGGGGGG

SEQ ID NO: 84 - protein, 5 aa, synthetic construct
GGGGX

(19) United States
(12) Patent Application Publication
(10) Pub. No.: US 2025/0152704 A1
(43) Pub. Date: May 15, 2025

(54) FCRN ANTAGONISTS WITH IMPROVED HALF-LIFE AND METHODS OF USE

(71) Applicants: Paragon Therapeutics, Inc., Waltham, MA (US); Viridian Therapeutics, Inc., Waltham, MA (US)

(72) Inventors: Yuqi Liu, Belmont, MA (US); Jason Z. Oh, Shrewsbury, MA (US); Hussam Hisham Shaheen, Auburn, NH (US); Rita L. Vaz, Lisbon (PT); Jordan Tsai, Waltham, MA (US)

(21) Appl. No.: 18/917,838

(22) Filed: Oct. 16, 2024

Related U.S. Application Data
(60) Provisional application No. 63/590,691, filed on Oct. 16, 2023.
(60) Provisional application No. 63/702,533, filed on Oct. 2, 2024.

(51) Int. Cl. A61K 39/395; A61K 38/38; C07K 14/765

(52) U.S. Cl. CPC A61K 39/39541; A61K 38/385; C07K 14/765; C07K 2319/035; C07K 2319/30

(57) ABSTRACT

Described herein are Fc fusion molecules that bind FcRn (FcRn) with improved half-life and methods of use thereof.

### CROSS-REFERENCE TO RELATED APPLICATIONS

This application claims priority to, and the benefit of, U.S. provisional application Nos. 63/702,533, filed Oct. 2, 2024, and 63/590,691, filed Oct. 16, 2023, the disclosure of each of which is hereby incorporated by reference in its entirety.

### INCORPORATION-BY-REFERENCE OF SEQUENCE LISTING

The present specification refers to and includes a Sequence Listing (submitted electronically as an XML file entitled VRD-017US1_SL.xml. The .xml file was generated on Jan. 16, 2025, and is 98,985 bytes in size. The entire contents of the Sequence Listing are herein incorporated by reference in its entirety.

### BACKGROUND

Immunoglobulin gamma (IgG) antibodies play a key role in the pathology of many disorders, such as autoimmune diseases and inflammatory diseases. IgGs generally have a longer half-life than other plasma proteins, in part, due to the binding of the Fc region of IgG to the Fc receptor, FcRn. Although FcRn was originally characterized as a neonatal transport receptor for maternal IgG, it also functions in adults to protect IgG from degradation. FcRn binds to pinocytosed IgG and protects the IgG from transport to degradative lysosomes by recycling it back to the extracellular compartment where it is released from FcRn and can resume its biological function.

Therapeutics targeting FcRn generally function to inhibit recycling of IgGs by binding to domains responsible for recycling these molecules. As a result, IgG based FcRn antagonists have a significantly shorter half-life than other therapeutics due to the lack of recycling mechanism. As such, there is a need for improved FcRn antagonists with longer half-life.

### SUMMARY OF THE DISCLOSURE

The present invention provides, among other things, an Fc fusion molecule that binds to neonatal Fc receptor (FcRn) comprising a modified Fc fragment fused to a half-life extension domain. The present invention is based, in part, on the unexpected discovery that an Fc fusion molecule described herein decreases the concentration of IgG by at least 75% within 5 days.

In one aspect, the present invention provides, among other things, an Fc fusion molecule that binds to neonatal Fc receptor (FcRn) comprising, from N-terminus to C-terminus, an Fc fragment comprises amino acid substitutions (i) N434Y and (ii) H433R or H433K as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from C-terminus to N-terminus, an Fc fragment comprises amino acid substitutions (i) N434Y and (ii) H433R or H433K as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from N-terminus to C-terminus an Fc fragment comprises amino acid substitutions N434Y and H433R as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from C-terminus to N-terminus, an Fc fragment comprises amino acid substitutions N434Y and H433R as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from N-terminus to C-terminus, an Fc fragment comprises amino acid substitutions N434Y and H433K as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from N C-terminus to N-terminus, an Fc fragment comprises amino acid substitutions N434Y and H433K as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from N-terminus to C-terminus an Fc fragment comprises amino acid substitutions M428L and N434F as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from C-terminus to N-terminus, an Fc fragment comprises amino acid substitutions M428L and N434F as compared to an amino acid sequence set forth in SEQ ID NO: 56; a linker; and an albumin or an albumin binding domain.

In some embodiments, the Fc fragment further comprises amino acid substitutions of M252Y, S254T, and T256E. In some embodiments, the Fc fragment further comprises the amino acid substitution of H433R or H433K. In some embodiments, the Fc fragment comprises amino acid substitutions of M428L, N434F, M252Y, S254T, T256E, and H433R. In some embodiments, the Fc fragment comprises amino acid substitutions of M428L, N434F, M252Y, S254T, T256E, and H433K.

In one aspect, the present invention provides, among other things, an Fc fusion molecule that binds to neonatal Fc receptor (FcRn) comprising, from N-terminus to C-terminus, an Fc fragment comprises amino acid substitutions H433R and N434F as compared to an amino acid sequence set forth in SEQ ID NO: 56; a linker; and an albumin or an albumin binding domain.

In one aspect, the present invention provides, among other things, an Fc fusion molecule that binds to neonatal Fc receptor (FcRn) comprising, from C-terminus to N-terminus, an Fc fragment comprises amino acid substitutions H433R and N434F as compared to an amino acid sequence set forth in SEQ ID NO: 56; a linker; and an albumin or an albumin binding domain.

In some embodiments, the Fc fragment further comprises amino acid substitutions of M252Y, S254T, and T256E.

In some embodiments, the Fc fragment further comprises the amino acid substitution of M428L.

In some embodiments, the Fc fragment comprises an amino acid sequence at least 80%, at least 85%, at least 90%, at least 95%, at least 96%, at least 97%, at least 98%, or at least 99% identical to any one of SEQ ID NOs: 1-9 or 57-67. In some embodiments, the Fc fragment comprises an amino acid sequence identical to any one of SEQ ID NOs: 1-9 or 57-67.

In some embodiments, the Fc fragment comprises an amino acid sequence at least 80%, at least 85%, at least 90%, at least 95%, at least 96%, at least 97%, at least 98%, or at least 99% identical to SEQ ID NO: 1. In some embodiments, the Fc fragment comprises an amino acid sequence identical to SEQ ID NO: 1. In some embodiments, the Fc fragment comprises an amino acid sequence at least 80% identical to SEQ ID NO: 1. In some embodiments, the Fc fragment comprises an amino acid sequence at least 85% identical to SEQ ID NO: 1. In some embodiments, the Fc fragment comprises an amino acid sequence at least 90% identical to SEQ ID NO: 1. In some embodiments, the Fc fragment comprises an amino acid sequence at least 95% identical to SEQ ID NO: 1. In some embodiments, the Fc fragment comprises an amino acid sequence at least 96% identical to SEQ ID NO: 1. In some embodiments, the Fc fragment comprises an amino acid sequence at least 97% identical to SEQ ID NO: 1. In some embodiments, the Fc fragment comprises an amino acid sequence at least 98% identical to SEQ ID NO: 1. In some embodiments, the Fc fragment comprises an amino acid sequence at least 99% identical to SEQ ID NO: 1.

In some embodiments, the Fc fragment comprises an amino acid sequence at least 80%, at least 85%, at least 90%, at least 95%, at least 96%, at least 97%, at least 98%, or at least 99% identical to SEQ ID NO: 2. In some embodiments, the Fc fragment comprises an amino acid sequence identical to SEQ ID NO: 2. In some embodiments, the Fc fragment comprises an amino acid sequence at least 80% identical to SEQ ID NO: 2. In some embodiments, the Fc fragment comprises an amino acid sequence at least 85% identical to SEQ ID NO: 2. In some embodiments, the Fc fragment comprises an amino acid sequence at least 90% identical to SEQ ID NO: 2. In some embodiments, the Fc fragment comprises an amino acid sequence at least 95% identical to SEQ ID NO: 2. In some embodiments, the Fc fragment comprises an amino acid sequence at least 96% identical to SEQ ID NO: 2. In some embodiments, the Fc fragment comprises an amino acid sequence at least 97% identical to SEQ ID NO: 2. In some embodiments, the Fc fragment comprises an amino acid sequence at least 98% identical to SEQ ID NO: 2. In some embodiments, the Fc fragment comprises an amino acid sequence at least 99% identical to SEQ ID NO: 2.

In some embodiments, the Fc fragment comprises an amino acid sequence at least 80%, at least 85%, at least 90%, at least 95%, at least 96%, at least 97%, at least 98%, or at least 99% identical to SEQ ID NO: 3. In some embodiments, the Fc fragment comprises an amino acid sequence identical to SEQ ID NO: 3. In some embodiments, the Fc fragment comprises an amino acid sequence at least 80% identical to SEQ ID NO: 3. In some embodiments, the Fc fragment comprises an amino acid sequence at least 85% identical to SEQ ID NO: 3. In some embodiments, the Fc fragment comprises an amino acid sequence at least 90% identical to SEQ ID NO: 3. In some embodiments, the Fc fragment comprises an amino acid sequence at least 95% identical to SEQ ID NO: 3. In some embodiments, the Fc fragment comprises an amino acid sequence at least 96% identical to SEQ ID NO: 3. In some embodiments, the Fc fragment comprises an amino acid sequence at least 97% identical to SEQ ID NO: 3. In some embodiments, the Fc fragment comprises an amino acid sequence at least 98% identical to SEQ ID NO: 3. In some embodiments, the Fc fragment comprises an amino acid sequence at least 99% identical to SEQ ID NO: 3.

In some embodiments, the Fc fragment comprises an amino acid sequence at least 80%, at least 85%, at least 90%, at least 95%, at least 96%, at least 97%, at least 98%, or at least 99% identical to SEQ ID NO: 4. In some embodiments, the Fc fragment comprises an amino acid sequence identical to SEQ ID NO: 4. In some embodiments, the Fc fragment comprises an amino acid sequence at least 80% identical to SEQ ID NO: 4. In some embodiments, the Fc fragment comprises an amino acid sequence at least 85% identical to SEQ ID NO: 4. In some embodiments, the Fc fragment comprises an amino acid sequence at least 90% identical to SEQ ID NO: 4. In some embodiments, the Fc fragment comprises an amino acid sequence at least 95% identical to SEQ ID NO: 4. In some embodiments, the Fc fragment comprises an amino acid sequence at least 96% identical to SEQ ID NO: 4. In some embodiments, the Fc fragment comprises an amino acid sequence at least 97% identical to SEQ ID NO: 4. In some embodiments, the Fc fragment comprises an amino acid sequence at least 98% identical to SEQ ID NO: 4. In some embodiments, the Fc fragment comprises an amino acid sequence at least 99% identical to SEQ ID NO: 4.

In some embodiments, the Fc fragment comprises an amino acid sequence at least 80%, at least 85%, at least 90%, at least 95%, at least 96%, at least 97%, at least 98%, or at least 99% identical to SEQ ID NO: 5. In some embodiments, the Fc fragment comprises an amino acid sequence identical to SEQ ID NO: 5. In some embodiments, the Fc fragment comprises an amino acid sequence at least 80% identical to SEQ ID NO: 5. In some embodiments, the Fc fragment comprises an amino acid sequence at least 85% identical to SEQ ID NO: 5. In some embodiments, the Fc fragment comprises an amino acid sequence at least 90% identical to SEQ ID NO: 5. In some embodiments, the Fc fragment comprises an amino acid sequence at least 95% identical to SEQ ID NO: 5. In some embodiments, the Fc fragment comprises an amino acid sequence at least 96% identical to SEQ ID NO: 5. In some embodiments, the Fc fragment comprises an amino acid sequence at least 97% identical to SEQ ID NO: 5. In some embodiments, the Fc fragment comprises an amino acid sequence at least 98% identical to SEQ ID NO: 5. In some embodiments, the Fc fragment comprises an amino acid sequence at least 99% identical to SEQ ID NO: 5.

In some embodiments, the Fc fragment comprises an amino acid sequence at least 80%, at least 85%, at least 90%, at least 95%, at least 96%, at least 97%, at least 98%, or at least 99% identical to SEQ ID NO: 6. In some embodiments, the Fc fragment comprises an amino acid sequence identical to SEQ ID NO: 6. In some embodiments, the Fc fragment comprises an amino acid sequence at least 80% identical to SEQ ID NO: 6. In some embodiments, the Fc fragment comprises an amino acid sequence at least 85% identical to SEQ ID NO: 6. In some embodiments, the Fc fragment comprises an amino acid sequence at least 90% identical to SEQ ID NO: 6. In some embodiments, the Fc fragment comprises an amino acid sequence at least 95% identical to SEQ ID NO: 6. In some embodiments, the Fc fragment comprises an amino acid sequence at least 96% identical to SEQ ID NO: 6. In some embodiments, the Fc fragment comprises an amino acid sequence at least 97% identical to SEQ ID NO: 6. In some embodiments, the Fc fragment comprises an amino acid sequence at least 98% identical to SEQ ID NO: 6. In some embodiments, the Fc fragment comprises an amino acid sequence at least 99% identical to SEQ ID NO: 6.

In some embodiments, the Fc fragment comprises an amino acid sequence at least 80%, at least 85%, at least 90%, at least 95%, at least 96%, at least 97%, at least 98%, or at least 99% identical to SEQ ID NO: 7. In some embodiments, the Fc fragment comprises an amino acid sequence identical to SEQ ID NO: 7. In some embodiments, the Fc fragment comprises an amino acid sequence at least 80% identical to SEQ ID NO: 7. In some embodiments, the Fc fragment comprises an amino acid sequence at least 85% identical to SEQ ID NO: 7. In some embodiments, the Fc fragment comprises an amino acid sequence at least 90% identical to SEQ ID NO: 7. In some embodiments, the Fc fragment comprises an amino acid sequence at least 95% identical to SEQ ID NO: 7. In some embodiments, the Fc fragment comprises an amino acid sequence at least 96% identical to SEQ ID NO: 7. In some embodiments, the Fc fragment comprises an amino acid sequence at least 97% identical to SEQ ID NO: 7. In some embodiments, the Fc fragment comprises an amino acid sequence at least 98% identical to SEQ ID NO: 7. In some embodiments, the Fc fragment comprises an amino acid sequence at least 99% identical to SEQ ID NO: 7.

In some embodiments, the Fc fragment comprises an amino acid sequence at least 80%, at least 85%, at least 90%, at least 95%, at least 96%, at least 97%, at least 98%, or at least 99% identical to SEQ ID NO: 8. In some embodiments, the Fc fragment comprises an amino acid sequence identical to SEQ ID NO: 8. In some embodiments, the Fc fragment comprises an amino acid sequence at least 80% identical to SEQ ID NO: 8. In some embodiments, the Fc fragment comprises an amino acid sequence at least 85% identical to SEQ ID NO: 8. In some embodiments, the Fc fragment comprises an amino acid sequence at least 90% identical to SEQ ID NO: 8. In some embodiments, the Fc fragment comprises an amino acid sequence at least 95% identical to SEQ ID NO: 8. In some embodiments, the Fc fragment comprises an amino acid sequence at least 96% identical to SEQ ID NO: 8. In some embodiments, the Fc fragment comprises an amino acid sequence at least 97% identical to SEQ ID NO: 8. In some embodiments, the Fc fragment comprises an amino acid sequence at least 98% identical to SEQ ID NO: 8. In some embodiments, the Fc fragment comprises an amino acid sequence at least 99% identical to SEQ ID NO: 8.

In some embodiments, the Fc fragment comprises an amino acid sequence at least 80%, at least 85%, at least 90%, at least 95%, at least 96%, at least 97%, at least 98%, or at least 99% identical to SEQ ID NO: 9. In some embodiments, the Fc fragment comprises an amino acid sequence identical to SEQ ID NO: 9. In some embodiments, the Fc fragment comprises an amino acid sequence at least 80% identical to SEQ ID NO: 9. In some embodiments, the Fc fragment comprises an amino acid sequence at least 85% identical to SEQ ID NO: 9. In some embodiments, the Fc fragment comprises an amino acid sequence at least 90% identical to SEQ ID NO: 9. In some embodiments, the Fc fragment comprises an amino acid sequence at least 95% identical to SEQ ID NO: 9. In some embodiments, the Fc fragment comprises an amino acid sequence at least 96% identical to SEQ ID NO: 9. In some embodiments, the Fc fragment comprises an amino acid sequence at least 97% identical to SEQ ID NO: 9. In some embodiments, the Fc fragment comprises an amino acid sequence at least 98% identical to SEQ ID NO: 9. In some embodiments, the Fc fragment comprises an amino acid sequence at least 99% identical to SEQ ID NO: 9.

In some embodiments, the Fc fragment comprises an amino acid sequence at least 80%, at least 85%, at least 90%, at least 95%, at least 96%, at least 97%, at least 98%, or at least 99% identical to SEQ ID NO: 57. In some embodiments, the Fc fragment comprises an amino acid sequence identical to SEQ ID NO: 57. In some embodiments, the Fc fragment comprises an amino acid sequence at least 80% identical to SEQ ID NO: 57. In some embodiments, the Fc fragment comprises an amino acid sequence at least 85% identical to SEQ ID NO: 57. In some embodiments, the Fc fragment comprises an amino acid sequence at least 90% identical to SEQ ID NO: 57. In some embodiments, the Fc fragment comprises an amino acid sequence at least 95% identical to SEQ ID NO: 57. In some embodiments, the Fc fragment comprises an amino acid sequence at least 96% identical to SEQ ID NO: 57. In some embodiments, the Fc fragment comprises an amino acid sequence at least 97% identical to SEQ ID NO: 57. In some embodiments, the Fc fragment comprises an amino acid sequence at least 98% identical to SEQ ID NO: 57. In some embodiments, the Fc fragment comprises an amino acid sequence at least 99% identical to SEQ ID NO: 57.

In some embodiments, the Fc fragment comprises an amino acid sequence at least 80%, at least 85%, at least 90%, at least 95%, at least 96%, at least 97%, at least 98%, or at least 99% identical to SEQ ID NO: 58. In some embodiments, the Fc fragment comprises an amino acid sequence identical to SEQ ID NO: 58. In some embodiments, the Fc fragment comprises an amino acid sequence at least 80% identical to SEQ ID NO: 58. In some embodiments, the Fc fragment comprises an amino acid sequence at least 85% identical to SEQ ID NO: 58. In some embodiments, the Fc fragment comprises an amino acid sequence at least 90% identical to SEQ ID NO: 58. In some embodiments, the Fc fragment comprises an amino acid sequence at least 95% identical to SEQ ID NO: 58. In some embodiments, the Fc fragment comprises an amino acid sequence at least 96% identical to SEQ ID NO: 58. In some embodiments, the Fc fragment comprises an amino acid sequence at least 97% identical to SEQ ID NO: 58. In some embodiments, the Fc fragment comprises an amino acid sequence at least 98% identical to SEQ ID NO: 58. In some embodiments, the Fc fragment comprises an amino acid sequence at least 99% identical to SEQ ID NO: 58.

In some embodiments, the Fc fragment comprises an amino acid sequence at least 80%, at least 85%, at least 90%, at least 95%, at least 96%, at least 97%, at least 98%, or at least 99% identical to SEQ ID NO: 59. In some embodiments, the Fc fragment comprises an amino acid sequence identical to SEQ ID NO: 59. In some embodiments, the Fc fragment comprises an amino acid sequence at least 80% identical to SEQ ID NO: 59. In some embodiments, the Fc fragment comprises an amino acid sequence at least 85% identical to SEQ ID NO: 59. In some embodiments, the Fc fragment comprises an amino acid sequence at least 90% identical to SEQ ID NO: 59. In some embodiments, the Fc fragment comprises an amino acid sequence at least 95% identical to SEQ ID NO: 59. In some embodiments, the Fc fragment comprises an amino acid sequence at least 96% identical to SEQ ID NO: 59. In some embodiments, the Fc fragment comprises an amino acid sequence at least 97% identical to SEQ ID NO: 59. In some embodiments, the Fc fragment comprises an amino acid sequence at least 98% identical to SEQ ID NO: 59. In some embodiments, the Fc fragment comprises an amino acid sequence at least 99% identical to SEQ ID NO: 59.

In some embodiments, the Fc fragment comprises an amino acid sequence at least 80%, at least 85%, at least 90%, at least 95%, at least 96%, at least 97%, at least 98%, or at least 99% identical to SEQ ID NO: 60. In some embodiments, the Fc fragment comprises an amino acid sequence identical to SEQ ID NO: 60. In some embodiments, the Fc fragment comprises an amino acid sequence at least 80% identical to SEQ ID NO: 60. In some embodiments, the Fc fragment comprises an amino acid sequence at least 85% identical to SEQ ID NO: 60. In some embodiments, the Fc fragment comprises an amino acid sequence at least 90% identical to SEQ ID NO: 60. In some embodiments, the Fc fragment comprises an amino acid sequence at least 95% identical to SEQ ID NO: 60. In some embodiments, the Fc fragment comprises an amino acid sequence at least 96% identical to SEQ ID NO: 60. In some embodiments, the Fc fragment comprises an amino acid sequence at least 97% identical to SEQ ID NO: 60. In some embodiments, the Fc fragment comprises an amino acid sequence at least 98% identical to SEQ ID NO: 60. In some embodiments, the Fc fragment comprises an amino acid sequence at least 99% identical to SEQ ID NO: 60.

In some embodiments, the Fc fragment comprises an amino acid sequence at least 80%, at least 85%, at least 90%, at least 95%, at least 96%, at least 97%, at least 98%, or at least 99% identical to SEQ ID NO: 61. In some embodiments, the Fc fragment comprises an amino acid sequence identical to SEQ ID NO: 61. In some embodiments, the Fc fragment comprises an amino acid sequence at least 80% identical to SEQ ID NO: 61. In some embodiments, the Fc fragment comprises an amino acid sequence at least 85% identical to SEQ ID NO: 61. In some embodiments, the Fc fragment comprises an amino acid sequence at least 90% identical to SEQ ID NO: 61. In some embodiments, the Fc fragment comprises an amino acid sequence at least 95% identical to SEQ ID NO: 61. In some embodiments, the Fc fragment comprises an amino acid sequence at least 96% identical to SEQ ID NO: 61. In some embodiments, the Fc fragment comprises an amino acid sequence at least 97% identical to SEQ ID NO: 61. In some embodiments, the Fc fragment comprises an amino acid sequence at least 98% identical to SEQ ID NO: 61. In some embodiments, the Fc fragment comprises an amino acid sequence at least 99% identical to SEQ ID NO: 61.

In some embodiments, the Fc fragment comprises an amino acid sequence at least 80%, at least 85%, at least 90%, at least 95%, at least 96%, at least 97%, at least 98%, or at least 99% identical to SEQ ID NO: 62. In some embodiments, the Fc fragment comprises an amino acid sequence identical to SEQ ID NO: 62. In some embodiments, the Fc fragment comprises an amino acid sequence at least 80% identical to SEQ ID NO: 62. In some embodiments, the Fc fragment comprises an amino acid sequence at least 85% identical to SEQ ID NO: 62. In some embodiments, the Fc fragment comprises an amino acid sequence at least 90% identical to SEQ ID NO: 62. In some embodiments, the Fc fragment comprises an amino acid sequence at least 95% identical to SEQ ID NO: 62. In some embodiments, the Fc fragment comprises an amino acid sequence at least 96% identical to SEQ ID NO: 62. In some embodiments, the Fc fragment comprises an amino acid sequence at least 97% identical to SEQ ID NO: 62. In some embodiments, the Fc fragment comprises an amino acid sequence at least 98% identical to SEQ ID NO: 62. In some embodiments, the Fc fragment comprises an amino acid sequence at least 99% identical to SEQ ID NO: 62.

In some embodiments, the Fc fragment comprises an amino acid sequence at least 80%, at least 85%, at least 90%, at least 95%, at least 96%, at least 97%, at least 98%, or at least 99% identical to SEQ ID NO: 63. In some embodiments, the Fc fragment comprises an amino acid sequence identical to SEQ ID NO: 63. In some embodiments, the Fc fragment comprises an amino acid sequence at least 80% identical to SEQ ID NO: 63. In some embodiments, the Fc fragment comprises an amino acid sequence at least 85% identical to SEQ ID NO: 63. In some embodiments, the Fc fragment comprises an amino acid sequence at least 90% identical to SEQ ID NO: 63. In some embodiments, the Fc fragment comprises an amino acid sequence at least 95% identical to SEQ ID NO: 63. In some embodiments, the Fc fragment comprises an amino acid sequence at least 96% identical to SEQ ID NO: 63. In some embodiments, the Fc fragment comprises an amino acid sequence at least 97% identical to SEQ ID NO: 63. In some embodiments, the Fc fragment comprises an amino acid sequence at least 98% identical to SEQ ID NO: 63. In some embodiments, the Fc fragment comprises an amino acid sequence at least 99% identical to SEQ ID NO: 63.

In some embodiments, the Fc fragment comprises an amino acid sequence at least 80%, at least 85%, at least 90%, at least 95%, at least 96%, at least 97%, at least 98%, or at least 99% identical to SEQ ID NO: 64. In some embodiments, the Fc fragment comprises an amino acid sequence identical to SEQ ID NO: 64. In some embodiments, the Fc fragment comprises an amino acid sequence at least 80% identical to SEQ ID NO: 64. In some embodiments, the Fc fragment comprises an amino acid sequence at least 85% identical to SEQ ID NO: 64. In some embodiments, the Fc fragment comprises an amino acid sequence at least 90% identical to SEQ ID NO: 64. In some embodiments, the Fc fragment comprises an amino acid sequence at least 95% identical to SEQ ID NO: 64. In some embodiments, the Fc fragment comprises an amino acid sequence at least 96% identical to SEQ ID NO: 64. In some embodiments, the Fc fragment comprises an amino acid sequence at least 97% identical to SEQ ID NO: 64. In some embodiments, the Fc fragment comprises an amino acid sequence at least 98% identical to SEQ ID NO: 64. In some embodiments, the Fc fragment comprises an amino acid sequence at least 99% identical to SEQ ID NO: 64.

In some embodiments, the Fc fragment comprises an amino acid sequence at least 80%, at least 85%, at least 90%, at least 95%, at least 96%, at least 97%, at least 98%, or at least 99% identical to SEQ ID NO: 65. In some embodiments, the Fc fragment comprises an amino acid sequence identical to SEQ ID NO: 65. In some embodiments, the Fc fragment comprises an amino acid sequence at least 80% identical to SEQ ID NO: 65. In some embodiments, the Fc fragment comprises an amino acid sequence at least 85% identical to SEQ ID NO: 65. In some embodiments, the Fc fragment comprises an amino acid sequence at least 90% identical to SEQ ID NO: 65. In some embodiments, the Fc fragment comprises an amino acid sequence at least 95% identical to SEQ ID NO: 65. In some embodiments, the Fc fragment comprises an amino acid sequence at least 96% identical to SEQ ID NO: 65. In some embodiments, the Fc fragment comprises an amino acid sequence at least 97% identical to SEQ ID NO: 65. In some embodiments, the Fc fragment comprises an amino acid sequence at least 98% identical to SEQ ID NO: 65. In some embodiments, the Fc fragment comprises an amino acid sequence at least 99% identical to SEQ ID NO: 65.

In some embodiments, the Fc fragment comprises an amino acid sequence at least 80%, at least 85%, at least 90%, at least 95%, at least 96%, at least 97%, at least 98%, or at least 99% identical to SEQ ID NO: 66. In some embodiments, the Fc fragment comprises an amino acid sequence identical to SEQ ID NO: 66. In some embodiments, the Fc fragment comprises an amino acid sequence at least 80% identical to SEQ ID NO: 66. In some embodiments, the Fc fragment comprises an amino acid sequence at least 85% identical to SEQ ID NO: 66. In some embodiments, the Fc fragment comprises an amino acid sequence at least 90% identical to SEQ ID NO: 66. In some embodiments, the Fc fragment comprises an amino acid sequence at least 95% identical to SEQ ID NO: 66. In some embodiments, the Fc fragment comprises an amino acid sequence at least 96% identical to SEQ ID NO: 66. In some embodiments, the Fc fragment comprises an amino acid sequence at least 97% identical to SEQ ID NO: 66. In some embodiments, the Fc fragment comprises an amino acid sequence at least 98% identical to SEQ ID NO: 66. In some embodiments, the Fc fragment comprises an amino acid sequence at least 99% identical to SEQ ID NO: 66.

In some embodiments, the Fc fragment comprises an amino acid sequence at least 80%, at least 85%, at least 90%, at least 95%, at least 96%, at least 97%, at least 98%, or at least 99% identical to SEQ ID NO: 67. In some embodiments, the Fc fragment comprises an amino acid sequence identical to SEQ ID NO: 67. In some embodiments, the Fc fragment comprises an amino acid sequence at least 80% identical to SEQ ID NO: 67. In some embodiments, the Fc fragment comprises an amino acid sequence at least 85% identical to SEQ ID NO: 67. In some embodiments, the Fc fragment comprises an amino acid sequence at least 90% identical to SEQ ID NO: 67. In some embodiments, the Fc fragment comprises an amino acid sequence at least 95% identical to SEQ ID NO: 67. In some embodiments, the Fc fragment comprises an amino acid sequence at least 96% identical to SEQ ID NO: 67. In some embodiments, the Fc fragment comprises an amino acid sequence at least 97% identical to SEQ ID NO: 67. In some embodiments, the Fc fragment comprises an amino acid sequence at least 98% identical to SEQ ID NO: 67. In some embodiments, the Fc fragment comprises an amino acid sequence at least 99% identical to SEQ ID NO: 67.

In some embodiments, the Fc fragment comprises an amino acid sequence identical to any one of SEQ ID NOs: 1-9 or 57-67.

In some embodiments, the Fc fragment comprises an amino acid sequence identical to SEQ ID NO: 1. In some embodiments, the Fc fragment comprises an amino acid sequence identical to SEQ ID NO: 2. In some embodiments, the Fc fragment comprises an amino acid sequence identical to SEQ ID NO: 3. In some embodiments, the Fc fragment comprises an amino acid sequence identical to SEQ ID NO: 4. In some embodiments, the Fc fragment comprises an amino acid sequence identical to SEQ ID NO: 5.

In some embodiments, the Fc fragment comprises an amino acid sequence identical to SEQ ID NO: 6. In some embodiments, the Fc fragment comprises an amino acid sequence identical to SEQ ID NO: 7. In some embodiments, the Fc fragment comprises an amino acid sequence identical to SEQ ID NO: 8. In some embodiments, the Fc fragment comprises an amino acid sequence identical to SEQ ID NO: 9.

In some embodiments, the Fc fragment comprises an amino acid sequence identical to SEQ ID NO: 57. In some embodiments, the Fc fragment comprises an amino acid sequence identical to SEQ ID NO: 58. In some embodiments, the Fc fragment comprises an amino acid sequence identical to SEQ ID NO: 59. In some embodiments, the Fc fragment comprises an amino acid sequence identical to SEQ ID NO: 60. In some embodiments, the Fc fragment comprises an amino acid sequence identical to SEQ ID NO: 61. In some embodiments, the Fc fragment comprises an amino acid sequence identical to SEQ ID NO: 62.

In some embodiments, the Fc fragment comprises an amino acid sequence identical to SEQ ID NO: 63. In some embodiments, the Fc fragment comprises an amino acid sequence identical to SEQ ID NO: 64. In some embodiments, the Fc fragment comprises an amino acid sequence identical to SEQ ID NO: 65. In some embodiments, the Fc fragment comprises an amino acid sequence identical to SEQ ID NO: 66. In some embodiments, the Fc fragment comprises an amino acid sequence identical to SEQ ID NO: 67.

In some embodiments, the Fc fusion molecule comprises an albumin. In some embodiments, the albumin comprises an amino acid sequence at least 80%, at least 85%, at least 90%, at least 95%, at least 96%, at least 97%, at least 98%, or at least 99% identical to SEQ ID NO: 10. In some embodiments, the albumin comprises an amino acid sequence identical to SEQ ID NO: 10. In some embodiments, the albumin comprises an amino acid sequence at least 80% identical to SEQ ID NO: 10. In some embodiments, the albumin comprises an amino acid sequence at least 85% identical to SEQ ID NO: 10. In some embodiments, the albumin comprises an amino acid sequence at least 90% identical to SEQ ID NO: 10. In some embodiments, the albumin comprises an amino acid sequence at least 95% identical to SEQ ID NO: 10. In some embodiments, the albumin comprises an amino acid sequence at least 96% identical to SEQ ID NO: 10. In some embodiments, the albumin comprises an amino acid sequence at least 97% identical to SEQ ID NO: 10. In some embodiments, the albumin comprises an amino acid sequence at least 98% identical to SEQ ID NO: 10. In some embodiments, the albumin comprises an amino acid sequence at least 99% identical to SEQ ID NO: 10.

In some embodiments, the Fc fusion molecule comprises an albumin binding domain. In some embodiments, the albumin binding domain comprises an amino acid sequence at least 80%, at least 85%, at least 90%, at least 95%, at least 96%, at least 97%, at least 98%, or at least 99% identical to SEQ ID NO: 11. In some embodiments, the albumin binding domain comprises an amino acid sequence identical to SEQ ID NO: 11. In some embodiments, the albumin binding domain comprises an amino acid sequence at least 80% identical to SEQ ID NO: 11. In some embodiments, the albumin binding domain comprises an amino acid sequence at least 85% identical to SEQ ID NO: 11. In some embodiments, the albumin binding domain comprises an amino acid sequence at least 90% identical to SEQ ID NO: 11. In some embodiments, the albumin binding domain comprises an amino acid sequence at least 95% identical to SEQ ID NO: 11. In some embodiments, the albumin binding domain comprises an amino acid sequence at least 96% identical to SEQ ID NO: 11. In some embodiments, the albumin binding domain comprises an amino acid sequence at least 97% identical to SEQ ID NO: 11. In some embodiments, the albumin binding domain comprises an amino acid sequence at least 98% identical to SEQ ID NO: 11. In some embodiments, the albumin binding domain comprises an amino acid sequence at least 99% identical to SEQ ID NO: 11.

In some embodiments, the albumin binding domain comprises an amino acid sequence at least 80%, at least 85%, at least 90%, at least 95%, at least 96%, at least 97%, at least 98%, or at least 99% identical to SEQ ID NO: 12. In some embodiments, the albumin binding domain comprises an amino acid sequence identical to SEQ ID NO: 12. In some embodiments, the albumin binding domain comprises an amino acid sequence at least 80% identical to SEQ ID NO: 12. In some embodiments, the albumin binding domain comprises an amino acid sequence at least 85% identical to SEQ ID NO: 12. In some embodiments, the albumin binding domain comprises an amino acid sequence at least 90% identical to SEQ ID NO: 12. In some embodiments, the albumin binding domain comprises an amino acid sequence at least 95% identical to SEQ ID NO: 12. In some embodiments, the albumin binding domain comprises an amino acid sequence at least 96% identical to SEQ ID NO: 12. In some embodiments, the albumin binding domain comprises an amino acid sequence at least 97% identical to SEQ ID NO: 12. In some embodiments, the albumin binding domain comprises an amino acid sequence at least 98% identical to SEQ ID NO: 12. In some embodiments, the albumin binding domain comprises an amino acid sequence at least 99% identical to SEQ ID NO: 12.

In some embodiments, the linker comprises glycine and serine. In some embodiments, the linker comprises one or more repeating units of GGGGS (SEQ ID NO: 13). In some embodiments, the linker comprises at least 2, at least 3, at least 4, at least 5, at least 6, at least 7, or at least 8 repeating units of GGGGS (SEQ ID NO: 13).

In some embodiments, the linker comprises at least 2 repeating units of GGGGS (SEQ ID NO: 13). In some embodiments, the linker comprises at least 3 repeating units of GGGGS (SEQ ID NO: 13). In some embodiments, the linker comprises at least 4 repeating units of GGGGS (SEQ ID NO: 13). In some embodiments, the linker comprises at least 5 repeating units of GGGGS (SEQ ID NO: 13). In some embodiments, the linker comprises at least 6 repeating units of GGGGS (SEQ ID NO: 13). In some embodiments, the linker comprises at least 7 repeating units of GGGGS (SEQ ID NO: 13). In some embodiments, the linker comprises at least 8 repeating units of GGGGS (SEQ ID NO: 13).

In some embodiments, the linker comprises 2 repeating units of GGGGS (SEQ ID NO: 13). In some embodiments, the linker comprises 3 repeating units of GGGGS (SEQ ID NO: 13). In some embodiments, the linker comprises 4 repeating units of GGGGS (SEQ ID NO: 13). In some embodiments, the linker comprises 5 repeating units of GGGGS (SEQ ID NO: 13). In some embodiments, the linker comprises 6 repeating units of GGGGS (SEQ ID NO: 13). In some embodiments, the linker comprises 7 repeating units of GGGGS (SEQ ID NO: 13). In some embodiments, the linker comprises 8 repeating units of GGGGS (SEQ ID NO: 13).

In some embodiments, the linker comprises a sequence according to any one of SEQ ID NOs: 13-15. In some embodiments, the linker comprises a sequence according to SEQ ID NO: 13. In some embodiments, the linker comprises a sequence according to SEQ ID NO: 14. In some embodiments, the linker comprises a sequence according to SEQ ID NO: 15.

In some embodiments, the Fc fragment is derived from an IgG1, IgG2 or IgG4 immunoglobulin domain. In some embodiments, the Fc fragment is derived from an IgG1 immunoglobulin domain. In some embodiments, the Fc fragment is derived from an IgG2 immunoglobulin domain. In some embodiments, the Fc fragment is derived from an IgG4 immunoglobulin domain.

In some embodiments, the Fc fusion molecule comprises, from N-terminus to C-terminus, the Fc fragment, the linker, and the albumin or albumin binding domain. In some embodiments, the Fc fusion molecule comprises, from N-terminus to C-terminus, the Fc fragment, the linker, and the albumin. In some embodiments, the Fc fusion molecule comprises, from N-terminus to C-terminus, the Fc fragment, the linker, and the albumin binding domain.

In some embodiments, the Fc fusion molecule comprises, from C-terminus to N-terminus, the Fc fragment, the linker, and the albumin or albumin binding domain. In some embodiments, the Fc fusion molecule comprises, from C-terminus to N-terminus, the Fc fragment, the linker, and the albumin. In some embodiments, the Fc fusion molecule comprises, from C-terminus to N-terminus, the Fc fragment, the linker, and the albumin binding domain.

In one aspect, the present invention provides, among other things, an Fc fusion molecule that binds to FcRn comprising an amino acid sequence at least 80%, at least 85%, at least 90%, at least 95%, at least 96%, at least 97%, at least 98%, or at least 99% identical to any one of SEQ ID NOs: 16-55. In one aspect, the present invention provides, among other things, an Fc fusion molecule that binds to FcRn comprising an amino acid sequence at least 80%, at least 85%, at least 90%, at least 95%, at least 96%, at least 97%, at least 98%, or at least 99% identical to any one of SEQ ID NOs: 16-55 or 68-73. In some embodiments, the Fc fusion molecule comprises an amino acid sequence at least 80%, at least 85%, at least 90%, at least 95%, at least 96%, at least 97%, at least 98%, or at least 99% identical to any one of SEQ ID NOs: 28-55 or 68-73. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 16. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 17. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 18. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 19. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 20.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 21. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 22. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 23. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 24. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 25.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 26. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 27. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 28. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 29. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 30.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 31. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 32. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 33. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 34. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 35.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 36. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 37. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 38. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 39. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 40.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 41. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 42. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 43. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 44. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 45.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 46. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 47. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 48. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 49. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 50.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 51. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 52. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 53. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 54. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 55.

In some embodiments, the Fc fusion molecule comprises an amino acid sequence at least 80%, at least 85%, at least 90%, at least 95%, at least 96%, at least 97%, at least 98%, or at least 99% identical to SEQ ID NO: 68. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 69. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 70. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 71. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 72. In some embodiments, the Fc fusion molecule comprises an amino acid sequence at least 80%, at least 85%, at least 90%, at least 95%, at least 96%, at least 97%, at least 98%, or at least 99% identical to SEQ ID NO: 73.

In one aspect, the present invention provides, among other things, an Fc fusion molecule that binds to FcRn comprising an amino acid sequence identical to any one of SEQ ID NOs: 16-55. In one aspect, the present invention provides, among other things, an Fc fusion molecule that binds to FcRn comprising an amino acid sequence identical to any one of SEQ ID NOs: 16-55 or 68-73. In some embodiments, the Fc fusion molecule comprises an amino acid sequence identical to any one of SEQ ID NOs: 28-55 or 68-73. In some embodiments, the Fc fusion molecule comprises an amino acid sequence identical to SEQ ID NO: 16. In some embodiments, the Fc fusion molecule comprises an amino acid sequence identical to SEQ ID NO: 17. In some embodiments, the Fc fusion molecule comprises an amino acid sequence identical to SEQ ID NO: 18. In some embodiments, the Fc fusion molecule comprises an amino acid sequence identical to SEQ ID NO: 19. In some embodiments, the Fc fusion molecule comprises an amino acid sequence identical to SEQ ID NO: 20.

In some embodiments, the Fc fusion molecule comprises an amino acid sequence identical to SEQ ID NO: 21. In some embodiments, the Fc fusion molecule comprises an amino acid sequence identical to SEQ ID NO: 22. In some embodiments, the Fc fusion molecule comprises an amino acid sequence identical to SEQ ID NO: 23. In some embodiments, the Fc fusion molecule comprises an amino acid sequence identical to SEQ ID NO: 24. In some embodiments, the Fc fusion molecule comprises an amino acid sequence identical to SEQ ID NO: 25.

In some embodiments, the Fc fusion molecule comprises an amino acid sequence identical to SEQ ID NO: 26. In some embodiments, the Fc fusion molecule comprises an amino acid sequence identical to SEQ ID NO: 27. In some embodiments, the Fc fusion molecule comprises an amino acid sequence identical to SEQ ID NO: 28. In some embodiments, the Fc fusion molecule comprises an amino acid sequence identical to SEQ ID NO: 29. In some embodiments, the Fc fusion molecule comprises an amino acid sequence identical to SEQ ID NO: 30.

In some embodiments, the Fc fusion molecule comprises an amino acid sequence identical to SEQ ID NO: 31. In some embodiments, the Fc fusion molecule comprises an amino acid sequence identical to SEQ ID NO: 32. In some embodiments, the Fc fusion molecule comprises an amino acid sequence identical to SEQ ID NO: 33. In some embodiments, the Fc fusion molecule comprises an amino acid sequence identical to SEQ ID NO: 34. In some embodiments, the Fc fusion molecule comprises an amino acid sequence identical to SEQ ID NO: 35.

In some embodiments, the Fc fusion molecule comprises an amino acid sequence identical to SEQ ID NO: 36. In some embodiments, the Fc fusion molecule comprises an amino acid sequence identical to SEQ ID NO: 37. In some embodiments, the Fc fusion molecule comprises an amino acid sequence identical to SEQ ID NO: 38. In some embodiments, the Fc fusion molecule comprises an amino acid sequence identical to SEQ ID NO: 39. In some embodiments, the Fc fusion molecule comprises an amino acid sequence identical to SEQ ID NO: 40.

In some embodiments, the Fc fusion molecule comprises an amino acid sequence identical to SEQ ID NO: 41. In some embodiments, the Fc fusion molecule comprises an amino acid sequence identical to SEQ ID NO: 42. In some embodiments, the Fc fusion molecule comprises an amino acid sequence identical to SEQ ID NO: 43. In some embodiments, the Fc fusion molecule comprises an amino acid sequence identical to SEQ ID NO: 44. In some embodiments, the Fc fusion molecule comprises an amino acid sequence identical to SEQ ID NO: 45.

In some embodiments, the Fc fusion molecule comprises an amino acid sequence identical to SEQ ID NO: 46. In some embodiments, the Fc fusion molecule comprises an amino acid sequence identical to SEQ ID NO: 47. In some embodiments, the Fc fusion molecule comprises an amino acid sequence identical to SEQ ID NO: 48. In some embodiments, the Fc fusion molecule comprises an amino acid sequence identical to SEQ ID NO: 49. In some embodiments, the Fc fusion molecule comprises an amino acid sequence identical to SEQ ID NO: 50.

In some embodiments, the Fc fusion molecule comprises an amino acid sequence identical to SEQ ID NO: 51. In some embodiments, the Fc fusion molecule comprises an amino acid sequence identical to SEQ ID NO: 52. In some embodiments, the Fc fusion molecule comprises an amino acid sequence identical to SEQ ID NO: 53. In some embodiments, the Fc fusion molecule comprises an amino acid sequence identical to SEQ ID NO: 54. In some embodiments, the Fc fusion molecule comprises an amino acid sequence identical to SEQ ID NO: 55.

In some embodiments, the Fc fusion molecule comprises an amino acid sequence identical to SEQ ID NO: 68. In some embodiments, the Fc fusion molecule comprises an amino acid sequence identical to SEQ ID NO: 69. In some embodiments, the Fc fusion molecule comprises an amino acid sequence identical to SEQ ID NO: 70. In some embodiments, the Fc fusion molecule comprises an amino acid sequence identical to SEQ ID NO: 71. In some embodiments, the Fc fusion molecule comprises an amino acid sequence identical to SEQ ID NO: 72. In some embodiments, the Fc fusion molecule comprises an amino acid sequence identical to SEQ ID NO: 73.

In some embodiments, the Fc fusion molecule further comprises a second Fc fragment. In some embodiments, the second Fc fragment is an Fc fragment described herein.

In some embodiments, the second Fc fragment comprises the same amino acid substitutions of the first Fc fragment. In some embodiments, the first Fc fragment and the second Fc fragment are further modified to promote heterodimerization. In some embodiments, the first Fc fragment and the second Fc fragment further comprise one or more amino acid substitutions selected from Y349C, S354C, T366S, T366W, T366Y, L368A, Y407T, Y407V.

In some embodiments, the first Fc fragment further comprises amino acid substitutions Y349C, T366S, L368A, and Y407V, and the second Fc fragment further comprises amino acid substitutions S354C and T366W. In some embodiments, the second Fc fragment further comprises amino acid substitutions Y349C, T366S, L368A, and Y407V, and the first Fc fragment further comprises amino acid substitutions S354C and T366W. In some embodiments, the first Fc fragment further comprises amino acid substitutions T366S, L368A, and Y407V, and the second Fc fragment further comprises amino acid substitution T266W. In some embodiments, the second Fc fragment further comprises amino acid substitutions T366S, L368A, and Y407V, and the first Fc fragment further comprises amino acid substitution T266W.

In some embodiments, the second Fc fragment comprises an amino acid sequence at least 80%, at least 85%, at least 90%, at least 95%, at least 96%, at least 97%, at least 98%, or at least 99% identical to any one of SEQ ID NOs: 1-9. In some embodiments, the second Fc fragment comprises an amino acid sequence identical to any one of SEQ ID NOs: 1-9.

Described herein, in certain embodiments, are Fc fusion molecules that binds neonatal Fc receptor (FcRn) comprising from N-terminus to C-terminus or C-terminus to N-terminus: a) an Fc fragment comprising a sequence having at least 80% sequence identity to the amino acid sequence of any one of SEQ ID NOs: 1-9; b) a linker; and c) an albumin or an albumin binding domain comprising a sequence having at least 80% sequence identity to the amino acid sequence of any one of SEQ ID NOs: 10-12. In some embodiments, the Fc fragment comprises a sequence having at least 80% sequence identity to the amino acid sequence to SEQ ID NO: 1, the linker comprises a sequence according to SEQ ID NO: 13, and the albumin or albumin binding domain comprises a sequence having at least 80% sequence identity to the amino acid sequence according to SEQ ID NO: 10. In some embodiments, the Fc fragment comprises a sequence having at least 80% sequence identity to the amino acid sequence to SEQ ID NO: 1, the linker comprises a sequence according to SEQ ID NO: 14, and the albumin or albumin binding domain comprises a sequence having at least 80% sequence identity to the amino acid sequence according to SEQ ID NO: 10. In some embodiments, the Fc fragment comprises a sequence having at least 80% sequence identity to the amino acid sequence to SEQ ID NO: 1, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 1, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 4 or SEQ ID NO: 5, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 4 or SEQ ID NO: 5, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 10. In some embodiments, the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 12. In some embodiments, the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 3, the linker comprises a sequence according to SEQ ID NO: 15, and the albumin or albumin binding domain comprises a sequence having at least 80% sequence identity to the amino acid sequence according to SEQ ID NO: 12. In some embodiments, the Fc fragment comprises a sequence having at least 80% sequence identity to the amino acid sequence to SEQ ID NO: 3, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 6 or SEQ ID NO: 7, the linker comprises a sequence according to SEQ ID NO: 13, and the albumin or albumin binding domain comprises a sequence having at least 80% sequence identity to the amino acid sequence according to SEQ ID NO: 12. In some embodiments, the Fc fragment comprises a sequence having at least 80% sequence identity to the amino acid sequence to SEQ ID NO: 6 or SEQ ID NO: 7, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 6 or SEQ ID NO: 7, the linker comprises a sequence according to SEQ ID NO: 15, and the albumin or albumin binding domain comprises a sequence having at least 80% sequence identity to the amino acid sequence according to SEQ ID NO: 12. In some embodiments, the Fc fragment comprises a sequence having at least 80% sequence identity to the amino acid sequence to SEQ ID NO: 6 or SEQ ID NO: 7, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 2, the linker comprises a sequence according to SEQ ID NO: 13, and the albumin or albumin binding domain comprises a sequence having at least 80% sequence identity to the amino acid sequence according to SEQ ID NO: 10. In some embodiments, the Fc fragment comprises a sequence having at least 80% sequence identity to the amino acid sequence to SEQ ID NO: 8 or SEQ ID NO: 9, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 8 or SEQ ID NO: 9, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 2, the linker comprises a sequence according to SEQ ID NO: 13, and the albumin or albumin binding domain comprises a sequence having at least 80% sequence identity to the amino acid sequence according to SEQ ID NO: 11. In some embodiments, the Fc fragment comprises a sequence having at least 80% sequence identity to the amino acid sequence to SEQ ID NO: 2, the linker comprises a sequence according to SEQ ID NO: 15, and the albumin or albumin binding domain comprises a sequence having at least 80% sequence identity to the amino acid sequence according to SEQ ID NO: 11. In some embodiments, the Fc fusion molecule further comprises a second Fc fragment comprising a sequence having at least 80% sequence identity to the amino acid sequence of any one of SEQ ID NOs: 1-9.

Described herein, in certain embodiments, are Fc fusion molecules that binds neonatal Fc receptor (FcRn) comprising a sequence having at least 80% sequence identity to any one of SEQ ID NOs: 16-55 or 68-73. In some embodiments, the Fc fusion molecule binds to FcRn with a K_{D} of less than or equal to about 1×10⁻⁸ M, at pH 6.0 or pH 7.4 as measured by surface plasmon resonance (SPR). In some embodiments, the Fc fusion molecule comprises a half-life at least 2 fold longer as compared to an Fc fusion molecule not comprising an albumin or an albumin binding domain. In some embodiments, the Fc fusion molecule comprises a half-life at least 2 fold longer as compared to an Fc fragment. In some embodiments, the albumin or the albumin binding domain comprises a sequence having at least 80% sequence identity to the amino acid sequence of any one of SEQ ID NOs: 10-12 any one of SEQ ID NOs: 10-12.

In some embodiments, the Fc fusion molecule reduces IgG levels to less than 60%, to less than 50%, to less than 40%, to less than 30%, or to less than 25%. In some embodiments, the Fc fusion molecule reduces IgG levels to less than 60%. In some embodiments, the Fc fusion molecule reduces IgG levels to less than 50%. In some embodiments, the Fc fusion molecule reduces IgG levels to less than 40%. In some embodiments, the Fc fusion molecule reduces IgG levels to less than 30%. In some embodiments, the Fc fusion molecule reduces IgG levels to less than 25%.

Described herein, in certain embodiments, are pharmaceutical compositions, comprising the Fc fusion molecule described herein and a pharmaceutically acceptable carrier.

Described herein, in certain embodiments, are methods of treating a disease or disorder in a patient in need thereof, the method comprising administering to the patient an effective amount of the Fc fusion molecule described herein or a pharmaceutical composition described herein. In some embodiments, the disease or disorder is an autoimmune disease. In some embodiments, the disease or disorder is generalized myasthenia gravis (gMG), chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenic purpura, primary Sjogren's Syndrome, systemic lupus erythematosus, rheumatoid arthritis, bullous pemphigoid, pemphigus foliaceus, pemphigus vulgaris, or cutaneous lupus erythematosus. In some embodiments, the method reduces disease severity in a patient and wherein disease severity is assessed by an gMG Disease Severity Outcome Measure.

Described herein, in certain embodiments, are methods for treating a pathology associated with elevated levels of an IgG in a patient in need thereof, the method comprising administering to the patient a therapeutically effective amount the isolated Fc fragment described herein or a pharmaceutical composition described herein.

Described herein, in certain embodiments, are methods of reducing biological activity of an IgG in a patient in need thereof, the method comprising administering to the patient a therapeutically effective amount of the Fc fusion molecule described herein or a pharmaceutical composition described herein. In some embodiments, the disease is an autoimmune disease.

Described herein, in certain embodiments, are methods of preventing a disorder in a patient in need thereof, the method comprising administering to the patient a therapeutically effective amount the Fc fusion molecule described herein or a pharmaceutical composition described herein; wherein the disorder is an unwanted side-effect of a therapeutic antibody.

In one aspect, the present invention provides, among other things, a method of reducing IgG levels in a patient in need thereof, wherein the method comprises administering a therapeutically effective amount of the Fc fusion molecule described herein, or the pharmaceutical composition described herein.

In some embodiments, administration of the Fc fusion molecule reduces IgG levels to less than 60%, to less than 50%, to less than 40%, to less than 30%, or to less than 25% for at least 5 days, 7 days, 10 days, 15 days, or 20 days post administration.

In some embodiments, administration of the Fc fusion molecule reduces IgG levels to less than 60%. In some embodiments, administration of the Fc fusion molecule reduces IgG levels to less than 50%. In some embodiments, administration of the Fc fusion molecule reduces IgG levels to less than 40%. In some embodiments, administration of the Fc fusion molecule reduces IgG levels to less than 30%. In some embodiments, administration of the Fc fusion molecule reduces IgG levels to less than 25%.

In some embodiments, the IgG levels are reduced for at least 5 days post-administration. In some embodiments, the IgG levels are reduced for at least 7 days post-administration. In some embodiments, the IgG levels are reduced for at least 10 days post-administration. In some embodiments, the IgG levels are reduced for at least 15 days post-administration. In some embodiments, the IgG levels are reduced for at least 20 days post-administration.

In some embodiments, the IgG levels are reduced as compared to a baseline. In some embodiments, the baseline is the IgG level in the patient prior to administration. In some embodiments, the baseline is the IgG level in a comparable patient without the administration of the Fc fusion molecule described herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1D depict exemplary formats of bivalent and monovalent Fc fusion molecules. FIG. 1A depicts an exemplary bivalent Fc fusion molecule comprising an N-terminal albumin or an albumin binding domain (ABD), a linker, and a C-terminal Fc domain (Format 1). FIG. 1B depicts an exemplary bivalent Fc fusion molecule comprising an N-terminal Fc domain, a linker, and a C-terminal albumin or an albumin binding domain (ABD) (Format 2). FIG. 1C depicts an exemplary monovalent Fc fusion molecule comprising an N-terminal albumin or an albumin binding domain (ABD), a linker, and a C-terminal Fc domain (Format 3). FIG. 1D depicts an exemplary monovalent Fc fusion molecule comprising a C-terminal Fc domain, a linker, and an N-terminal albumin or an albumin binding domain (ABD) (Format 4).

FIGS. 2A-2D depict SPR kinetic analyses of bivalent Fc fusion molecules to human serum albumin (HSA). FIG. 2A depicts a sensorgram of the titration of HSA on Fc Fusion Molecule 5 immobilized on a CM5 chip at pH 7.4. FIG. 2B depicts a sensorgram of the titration of HSA on Fc Fusion Molecule 5 immobilized on a CM5 chip at pH 6.0. FIG. 2C depicts a sensorgram of the titration of HSA on Fc Fusion Molecule 6 immobilized on a CM5 chip at pH 7.4. FIG. 2D depicts a sensorgram of the titration of HSA on Fc Fusion Molecule 6 immobilized on a CM5 chip at pH 6.0. The dark grey and light grey lines represent the data and fit, respectively.

FIGS. 3A-3D depict SPR kinetic analyses of bivalent Fc fusion molecules to Human FCRN (huFCRN). FIG. 3A depicts a sensorgram of the titration of huFCRN on Fc Fusion Molecule 5 immobilized on a CM5 chip at pH 7.4. FIG. 3B depicts a sensorgram of the titration of huFCRN on Fc Fusion Molecule 5 immobilized on a CM5 chip at pH 6.0. FIG. 3C depicts a sensorgram of the titration of huFCRN on Fc Fusion Molecule 6 immobilized on a CM5 chip at pH 7.4. FIG. 3D depicts a sensorgram of the titration of huFCRN on Fc Fusion Molecule 6 immobilized on a CM5 chip at pH 6.0.

FIGS. 4A-4D depict SPR kinetic analyses of monovalent Fc fusion molecules to human serum albumin (HSA). FIG. 4A depicts a sensorgram of the titration of HSA on Fc Fusion Molecule 11 immobilized on a CM5 chip at pH 7.4. FIG. 4B depicts a sensorgram of the titration of HSA on Fc Fusion Molecule 11 immobilized on a CM5 chip at pH 6.0. FIG. 4C depicts a sensorgram of the titration of HSA on Fc Fusion Molecule 12 immobilized on a CM5 chip at pH 7.4. FIG. 4D depicts a sensorgram of the titration of HSA on Fc Fusion Molecule 12 immobilized on a CM5 chip at pH 6.0.

FIGS. 5A-5D depict SPR kinetic analyses of monovalent Fc fusion molecules to Human FCRN (huFCRN). FIG. 5A depicts a sensorgram of the titration of huFCRN on Fc Fusion Molecule 11 immobilized on a CM5 chip at pH 7.4. FIG. 5B depicts a sensorgram of the titration of huFCRN on Fc Fusion Molecule 11 immobilized on a CM5 chip at pH 6.0.

FIG. 5C depicts a sensorgram of the titration of huFCRN on Fc Fusion Molecule 12 immobilized on a CM5 chip at pH 7.4. FIG. 5D depicts a sensorgram of the titration of huFCRN on Fc Fusion Molecule 12 immobilized on a CM5 chip at pH 6.0.

FIG. 6 depicts a schema of the pharmacokinetic study performed in mice.

FIGS. 7A-7E depict data of the pharmacokinetic (PK) and pharmacodynamic (PD) profiles for the various Fc fusion molecules including PD effect of the Fc fusion molecules as determined by human IVIG tracer reduction (FIGS. 7C-7E).

FIG. 8 depicts exemplary graphs showing the degradation of FcRn-GFP in HEK cells in the presence of exemplary Fc fusion molecules.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

Any numerical values used in this application are meant to cover any variations within the standard deviation or normal fluctuations appreciated by one of ordinary skill in the relevant art.

As used herein, unless otherwise indicated, the term “antibody” is understood to mean an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). The three CDRs, referred to as CDR1, CDR2, and CDR3, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM. These classes are also designated α, δ, ε, γ, and μ, respectively. Several of these may be further divided into subclasses (isotypes), e.g., IgG1, IgG2, IgG3, IgG4, IgA1, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an Fc fragment) and its binding partner (e.g., FcRn). Unless indicated otherwise, as used herein, “affinity” refers to intrinsic binding affinity, which reflects a 1:1 interaction between members of a binding pair (e.g., an Fc fragment and FcRn). Affinity is indirectly proportional to K_{D}.

The term “kd” (sec-1), as used herein, refers to the dissociation rate constant of a particular antibody-antigen or protein-protein interaction. This value is also referred to as the koff value.

The term “ka” (M-1×sec-1), as used herein, refers to the association rate constant of a particular antibody-antigen interaction or protein-protein. This value is also referred to as the kon value.

The term “KD” or “K_{D}”) (M), as used herein, refers to the dissociation equilibrium constant of a particular antibody-antigen or protein-protein interaction. KD=kd/ka. In some embodiments, the affinity of a protein is described in terms of the KD for an interaction between such protein and its binding partner. For clarity, as known in the art, a smaller KD value indicates a higher affinity interaction, while a larger KD value indicates a lower affinity interaction.

The term “measurable KD” or “measurable K_{D}” as used herein, means a KD value that is less than 1M, less than 0.1M, less than 0.01M, less than 0.001M, less than 1×10⁻⁴ M, less than 1×10⁻⁵ M, or less than 1×10⁻⁶ M.

The term “KA” (M-1), as used herein, refers to the association equilibrium constant of a particular antibody-antigen or protein-protein interaction. KA=ka/kd.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

### Fc Fusion Molecules

Described herein, in certain embodiments, are Fc fusion molecules that bind neonatal Fc receptor (FcRn).

### Fc Fragments

Described herein, in certain embodiments, are Fc fusion molecules that bind neonatal Fc receptor (FcRn) comprising an Fc fragment. In some embodiments, the Fc fusion molecule comprises an Fc fragment comprising a sequence having at least 80% sequence identity with an amino acid sequence according to any one of SEQ ID NOs: 1-9 or 57-67. In some embodiments, the Fc fusion molecule comprises an Fc fragment comprising a sequence having at least 85% sequence identity with an amino acid sequence according to any one of SEQ ID NOs: 1-9 or 57-67. In some embodiments, the Fc fusion molecule comprises an Fc fragment comprising a sequence having at least 90% sequence identity with an amino acid sequence according to any one of SEQ ID NOs: 1-9 or 57-67. In some embodiments, the Fc fusion molecule comprises an Fc fragment comprising a sequence having at least 95% sequence identity with an amino acid sequence according to any one of SEQ ID NOs: 1-9 or 57-67. In some embodiments, the Fc fusion molecule comprises an Fc fragment comprising a sequence having at least 96% sequence identity with an amino acid sequence according to any one of SEQ ID NOs: 1-9 or 57-67. In some embodiments, the Fc fusion molecule comprises an Fc fragment comprising a sequence having at least 97% sequence identity with an amino acid sequence according to any one of SEQ ID NOs: 1-9 or 57-67. In some embodiments, the Fc fusion molecule comprises an Fc fragment comprising a sequence having at least 98% sequence identity with an amino acid sequence according to any one of SEQ ID NOs: 1-9 or 57-67. In some embodiments, the Fc fusion molecule comprises an Fc fragment comprising a sequence having at least 99% sequence identity with an amino acid sequence according to any one of SEQ ID NOs: 1-9 or 57-67. In some embodiments, the Fc fusion molecule comprises an Fc fragment comprising a sequence according to any one of SEQ ID NOs: 1-9 or 57-67. Amino acid sequences of exemplary Fc fragments are shown in Table 1A.

TABLE 1A
Exemplary Fc Fragments.

| Identifier | Description | Amino Acid sequence |
|---|---|---|
| WT human IgG1 Fc | Human IgG1 | DKTHTCPPCPAPELLGGPSVFLFPPKPKDTLMISRTPEVTCVVVDV |
| (SEQ ID NO: 56) | (221-446) | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPG |
| Efgartigimod (Efgart) | hIgG1_Fc_ | DKTHTCPPCPAPELLGGPSVFLFPPKPKDTLYITREPEVTCVVVDV |
| Fc Fragment 1 | M252Y/S254T/ | SHEDPEVKFNWYVDGVEVHNAKTKPREEQYNSTYRVVSVLTVLHQD |
| (SEQ ID NO: 1) | T256E/H433K/ | WLNGKEYKCKVSNKALPAPIEKTISKAKGQPREPQVYTLPPSRDEL |
| | N434F | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPG |
| Fc195 | IgG1 | DKTHTCPPCPAPELLGGPSVFLFPPKPKDTLYITREPEVTCVVVDV |
| Fc Fragment 2 | Fc_M252Y/ | SHEDPEVKFNWYVDGVEVHNAKTKPREEQYNSTYRVVSVLTVLHQD |
| (SEQ ID NO: 2) | S254T/T256E/ | WLNGKEYKCKVSNKALPAPIEKTISKAKGQPREPQVYTLPPSRDEL |
| | M428L/H433K/ | TKNQVSLTCLVKGFYPSDIAVEWESNGQPENNYKTTPPVLDSDGSF |
| | N434F | FLYSKLTVDKSRWQQGNVFSCSVLHEALKFHYTQKSLSLSPG |
| Fc197 | IgG1 | DKTHTCPPCPAPELLGGPSVFLFPPKPKDTLYITREPEVTCVVVDV |
| Fc Fragment 3 | Fc_M252Y/S254T/ | SHEDPEVKFNWYVDGVEVHNAKTKPREEQYNSTYRVVSVLTVLHQD |
| (SEQ ID NO: 3) | T256E/H433K/ | WLNGKEYKCKVSNKALPAPIEKTISKAKGQPREPQVYTLPPSRDEL |
| | N434Y | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPG |
| Efgart(H) | hIgG1_Fc_M252Y/ | DKTHTCPPCPAPELLGGPSVFLFPPKPKDTLYITREPEVTCVVVDV |
| Fc Fragment 4 | S254T/T256E/ | SHEDPEVKFNWYVDGVEVHNAKTKPREEQYNSTYRVVSVLTVLHQD |
| (SEQ ID NO: 4) | Y349C/T366S/ | WLNGKEYKCKVSNKALPAPIEKTISKAKGQPREPQVCTLPPSRDEL |
| | L368A/Y407V/ | TKNQVSLSCAVKGFYPSDIAVEWESNGQPENNYKTTPPVLDSDGSF |
| | H433K/N434F | FLVSKLTVDKSRWQQGNVFSCSVMHEALKFHYTQKSLSLSPG |
| Efgart(K) | hIgG1_Fc_M252Y/ | DKTHTCPPCPAPELLGGPSVFLFPPKPKDTLYITREPEVTCVVVDV |
| Fc Fragment 5 | S254T/T256E/ | SHEDPEVKFNWYVDGVEVHNAKTKPREEQYNSTYRVVSVLTVLHQD |
| (SEQ ID NO: 5) | S354C/T366W/ | WLNGKEYKCKVSNKALPAPIEKTISKAKGQPREPQVYTLPPCRDEL |
| | H433K/N434F | TKNQVSLWCLVKGFYPSDIAVEWESNGQPENNYKTTPPVLDSDGSF |
| | | FLYSKLTVDKSRWQQGNVFSCSVMHEALKFHYTQKSLSLSPG |
| Fc197(H) | hIgG1_Fc_M252Y/ | DKTHTCPPCPAPELLGGPSVFLFPPKPKDTLYITREPEVTCVVVDV |
| Fc Fragment 6 | S254T/T256E/ | SHEDPEVKFNWYVDGVEVHNAKTKPREEQYNSTYRVVSVLTVLHQD |
| (SEQ ID NO: 6) | Y349C/T366S/ | WLNGKEYKCKVSNKALPAPIEKTISKAKGQPREPQVCTLPPSRDEL |
| | L368A/Y407V/ | TKNQVSLSCAVKGFYPSDIAVEWESNGQPENNYKTTPPVLDSDGSF |
| | H433K/N434Y | FLVSKLTVDKSRWQQGNVFSCSVMHEALKYHYTQKSLSLSPG |
| Fc197(K) | hIgG1_Fc_M252Y/ | DKTHTCPPCPAPELLGGPSVFLFPPKPKDTLYITREPEVTCVVVDV |
| Fc Fragment 7 | S254T/T256E/ | SHEDPEVKFNWYVDGVEVHNAKTKPREEQYNSTYRVVSVLTVLHQD |
| (SEQ ID NO: 7) | S354C/T366W/ | WLNGKEYKCKVSNKALPAPIEKTISKAKGQPREPQVYTLPPCRDEL |
| | H433K/N434Y | TKNQVSLWCLVKGFYPSDIAVEWESNGQPENNYKTTPPVLDSDGSF |
| | | FLYSKLTVDKSRWQQGNVFSCSVMHEALKYHYTQKSLSLSPG |
| Fc195(H) | hIgG1_Fc_M252Y/ | DKTHTCPPCPAPELLGGPSVFLFPPKPKDTLYITREPEVTCVVVDV |
| Fc Fragment 8 | S254T/T256E/ | SHEDPEVKFNWYVDGVEVHNAKTKPREEQYNSTYRVVSVLTVLHQD |
| (SEQ ID NO: 8) | Y349C/T366S/ | WLNGKEYKCKVSNKALPAPIEKTISKAKGQPREPQVCTLPPSRDEL |
| | L368A/Y407V/ | TKNQVSLSCAVKGFYPSDIAVEWESNGQPENNYKTTPPVLDSDGSF |
| | M428L/H433K/ | FLVSKLTVDKSRWQQGNVFSCSVLHEALKFHYTQKSLSLSPG |
| | N434F | |
| Fc195(K) | hIgG1_Fc_M252Y/ | DKTHTCPPCPAPELLGGPSVFLFPPKPKDTLYITREPEVTCVVVDV |
| Fc Fragment 9 | S254T/T256E/ | SHEDPEVKFNWYVDGVEVHNAKTKPREEQYNSTYRVVSVLTVLHQD |
| (SEQ ID NO: 9) | S354C/T366W/ | WLNGKEYKCKVSNKALPAPIEKTISKAKGQPREPQVYTLPPCRDEL |
| | M428L/H433K/ | TKNQVSLWCLVKGFYPSDIAVEWESNGQPENNYKTTPPVLDSDGSF |
| | N434F | FLYSKLTVDKSRWQQGNVFSCSVLHEALKFHYTQKSLSLSPG |
| Fc Fragment 10 | IgG1 | DKTHTCPPCPAPELLGGPSVFLFPPKPKDTLYITREPEVTCVVVDV |
| (SEQ ID NO: 57) | Fc_M252Y/S254T/ | SHEDPEVKFNWYVDGVEVHNAKTKPREEQYNSTYRVVSVLTVLHQD |
| | T256E/M428L/ | WLNGKEYKCKVSNKALPAPIEKTISKAKGQPREPQVYTLPPSRDEL |
| | H433K/N434Y | TKNQVSLTCLVKGFYPSDIAVEWESNGQPENNYKTTPPVLDSDGSF |
| | | FLYSKLTVDKSRWQQGNVFSCSVLHEALKYHYTQKSLSLSPG |
| Fc Fragment 11 | IgG1 | DKTHTCPPCPAPELLGGPSVFLFPPKPKDTLYITREPEVTCVVVDV |
| (SEQ ID NO: 58) | Fc_M252Y/S254T/ | SHEDPEVKFNWYVDGVEVHNAKTKPREEQYNSTYRVVSVLTVLHQD |
| | T256E/M428L/ | WLNGKEYKCKVSNKALPAPIEKTISKAKGQPREPQVYTLPPSRDEL |
| | N434F | TKNQVSLTCLVKGFYPSDIAVEWESNGQPENNYKTTPPVLDSDGSF |
| | | FLYSKLTVDKSRWQQGNVFSCSVLHEALHFHYTQKSLSLSPG |
| Fc Fragment 12 | IgG1 | DKTHTCPPCPAPELLGGPSVFLFPPKPKDTLYITREPEVTCVVVDV |
| (SEQ ID NO: 59) | Fc_M252Y/S254T/ | SHEDPEVKFNWYVDGVEVHNAKTKPREEQYNSTYRVVSVLIVLHQD |
| | T256E/H433R/ | WLNGKEYKCKVSNKALPAPIEKTISKAKGQPREPQVYTLPPSRDEL |
| | N434Y | TKNQVSLTCLVKGFYPSDIAVEWESNGQPENNYKTTPPVLDSDGSF |
| | | FLYSKLTVDKSRWQQGNVFSCSVMHEALRYHYTQKSLSLSPG |
| Fc Fragment 13 | IgG1 | DKTHTCPPCPAPELLGGPSVFLFPPKPKDTLYITREPEVTCVVVDV |
| (SEQ ID NO: 60) | Fc_M252Y/S254T/ | SHEDPEVKFNWYVDGVEVHNAKTKPREEQYNSTYRVVSVLTVLHQD |
| | T256E/H433R/ | WLNGKEYKCKVSNKALPAPIEKTISKAKGQPREPQVYTLPPSRDEL |
| | N434F | TKNQVSLTCLVKGFYPSDIAVEWESNGQPENNYKTTPPVLDSDGSF |
| | | FLYSKLTVDKSRWQQGNVFSCSVMHEALRFHYTQKSLSLSPG |
| Fc Fragment 14 | IgG1 | DKTHTCPPCPAPELLGGPSVFLFPPKPKDTLYITREPEVTCVVVDV |
| (SEQ ID NO: 61) | Fc_M252Y/S254T/ | SHEDPEVKFNWYVDGVEVHNAKTKPREEQYNSTYRVVSVLTVLHQD |
| | T256E/M428L/ | WLNGKEYKCKVSNKALPAPIEKTISKAKGQPREPQVYTLPPSRDEL |
| | H433R/N434F | TKNQVSLTCLVKGFYPSDIAVEWESNGQPENNYKTTPPVLDSDGSF |
| | | FLYSKLTVDKSRWQQGNVFSCSVLHEALRFHYTQKSLSLSPG |
| Fc Fragment 15 | IgG1 | DKTHTCPPCPAPELLGGPSVFLFPPKPKDTLYITREPEVTCVVVDV |
| (SEQ ID NO: 62) | Fc_M252Y/S254T/ | SHEDPEVKFNWYVDGVEVHNAKTKPREEQYNSTYRVVSVLTVDHQD |
| | T256E/L309D/ | WLNGKEYKCKVSNKALPAPIEKTISKAKGQPREPQVYTLPPSRDEL |
| | H433K/N434F | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPG |
| Fc Fragment 16 | IgG1 | DKTHTCPPCPAPELLGGPSVFLFPPKPKDTLYITREPEVTCVVVDV |
| (SEQ ID NO: 63) | Fc_M252Y/S254T/ | SHEDPEVKFNWYVDGVEVHNAKTKPREEQYNSTYRVVSVLTVDHQD |
| | T256E/L309D/ | WLNGKEYKCKVSNKALPAPIEKTISKAKGQPREPQVYTLPPSRDEL |
| | H433K/N434Y | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPG |
| Fc Fragment 17 | IgG1 | DKTHTCPPCPAPELLGGPSVFLFPPKPKDTLYITREPEVTCVVVDV |
| (SEQ ID NO: 64) | Fc_M252Y/S254T/ | SHEDPEVKFNWYVDGVEVHNAKTKPREEQYNSTYRVVSVLTVDHQD |
| | T256E/L309D/ | WLNGKEYKCKVSNKALPAPIEKTISKAKGQPREPQVYTLPPSRDEL |
| | M428L/H433K/ | TKNQVSLTCLVKGFYPSDIAVEWESNGQPENNYKTTPPVLDSDGSF |
| | N434F | FLYSKLTVDKSRWQQGNVFSCSVLHEALKFHYTQKSLSLSPG |
| Fc Fragment 18 | IgG1 | DKTHTCPPCPAPELLGGPSVFLFPPKPKDTLYITREPEVTCVVVDV |
| (SEQ ID NO: 65) | Fc_M252Y/S254T/ | SHEDPEVKFNWYVDGVEVHNAKTKPREEQYNSTYRVVSVLTVDHQD |
| | T256E/L309D/ | WLNGKEYKCKVSNKALPAPIEKTISKAKGQPREPQVYTLPPSRDEL |
| | M428L/H433K/ | TKNQVSLTCLVKGFYPSDIAVEWESNGQPENNYKTTPPVLDSDGSF |
| | N434Y | FLYSKLTVDKSRWQQGNVFSCSVLHEALKYHYTQKSLSLSPG |
| Fc Fragment 19 | IgG1 | DKTHTCPPCPAPELLGGPSVFLFPPKPKDTLYITREPEVTCVVVDV |
| (SEQ ID NO: 66) | Fc_M252Y/S254T/ | SHEDPEVKFNWYVDGVEVHNAKTKPREEQYNSTYRVVSVLTVDHKD |
| | T256E/L309D/ | WLNGKEYKCKVSNKALPAPIEKTISKAKGQPREPQVYTLPPSRDEL |
| | Q311K/H433K/ | TKNQVSLTCLVKGFYPSDIAVEWESNGQPENNYKTTPPVLDSDGSF |
| | N434F | FLYSKLTVDKSRWQQGNVFSCSVMHEALKFHYTQKSLSLSPG |
| Fc Fragment 20 | IgG1 | DKTHTCPPCPAPELLGGPSVFLFPPKPKDTLYITREPEVTCVVVDV |
| (SEQ ID NO: 67) | Fc_M252Y/S254T/ | SHEDPEVKFNWYVDGVEVHNAKTKPREEQYNSTYRVVSVLTVDHKD |
| | T256E/L309D/ | WLNGKEYKCKVSNKALPAPIEKTISKAKGQPREPQVYTLPPSRDEL |
| | Q311K/H433K/ | TKNQVSLTCLVKGFYPSDIAVEWESNGQPENNYKTTPPVLDSDGSF |
| | N434Y | FLYSKLTVDKSRWQQGNVFSCSVMHEALKYHYTQKSLSLSPG |

In some embodiments, the Fc fusion molecules comprise a modified Fc or Fc fragment comprising one or more modifications. In some embodiments, the Fc fragment is an IgG subclass IgG1, IgG2 or IgG4. In some embodiments, the one or more modifications are located in an Fc from IgG1 (e.g., human IgG1 (hIgG1). In some embodiments, the one or more modifications are located in an Fc from IgG4 (e.g., human IgG4 (hIgG4). In some embodiments, the one or more modifications are located in an Fc from IgG2. In some embodiments, the one or more modifications promote selective binding of Fc-gamma receptors.

In some embodiments, the Fc fragment is modified using knobs-into-holes, strand-exchange engineered domain (SEED), or steric modification technology. In some embodiments, the Fc fragment is modified to promote Fc heterodimerization. In some embodiments, the Fc fragment comprises a variant (e.g., a knobs-into-hole variant). In some embodiments, the Fc fragment comprises a variant of the amino acid sequence set forth in SEQ ID NO: 56, wherein the variant comprises at least one amino acid substitution selected from Y349C, S354C, T366S, T366W, L368A, Y407V, M428L, H433K, N434F, and N434Y. In some embodiments, the Fc fragment comprises a variant of the amino acid sequence set forth in SEQ ID NO: 56, wherein the variant comprises at least one amino acid substitution selected from Y349C, S354C, T366S, T366W, T366Y, L368A, Y407T, Y407V, M428L, H433K, N434F, and N434Y.

In some embodiments, the Fc fragment binds neonatal Fc receptor (FcRn) from human, cyno, mouse, or rat. In some embodiments, the Fc fragment comprises a variant of the amino acid sequence set forth in SEQ ID NO: 56, wherein the variant comprises at least one amino acid substitution selected from M428L, H433R and N434Y. In some embodiments, the isolated Fc fragment comprises additional amino acid substitutions at one or more of amino acid positions 252, 254, and 256 of SEQ ID NO: 56. In some embodiments, the additional amino acid substitutions are M252Y, S254T, and/or T256E.

In some embodiments, the Fc fragment comprises a variant of the amino acid sequence set forth in SEQ ID NO: 56, wherein the variant comprises an amino acid substitution at position 428. In some embodiments, the amino acid substitution is M428L. In some embodiments, the Fc fragment comprises additional amino acid substitutions at one or more of amino acid positions 252, 254, 256, 433, and 434 of SEQ ID NO: 56. In some embodiments, the additional amino acid substitutions are M252Y, S254T, T256E, H433K, H433R, N434F and/or N434Y.

In some embodiments, the Fc fragment comprises a variant of the amino acid sequence set forth in SEQ ID NO: 56, wherein the variant comprises an amino acid substitution H433R. In some embodiments, the additional amino acid substitutions are at one or more of amino acid positions 252, 254, 256, 428, and 434 of SEQ ID NO: 56. In some embodiments, the one or more additional amino acid substitutions are M252Y, S254T, T256E, M428L, N434F and/or N434Y.

In some embodiments, the Fc fragment comprises a variant of the amino acid sequence set forth in SEQ ID NO: 56, wherein the variant comprises an amino acid substitution N434Y. In some embodiments, the Fc fragment comprises an additional amino acid substitution at one or more of amino acid positions 252, 254, 256, 428, and 433 of SEQ ID NO: 56. In some embodiments, the amino acid substitutions are M252Y, S254T, T256E, M428L, H433K, and/or H433R.

In some embodiments, the Fc fragment comprises a variant of the amino acid sequence set forth in SEQ ID NO: 56, wherein the variant comprises an amino acid substitution H433K, and additional amino acid substitutions at one or more of amino acid positions 252, 254, 256, 428, and 434. In some embodiments, the additional amino acid substitutions are M252Y, S254T, T256E, M428L, N434F and/or N434Y.

In some embodiments the Fc fragment comprises a variant of the amino acid sequence set forth in SEQ ID NO: 56, wherein the variant comprises amino acid substitutions (i) N434Y and (ii) H433R or H433K. In some embodiments, the variant further comprises amino acid substitutions of M252Y, S254T, and T256E. In some embodiments, the variant further comprises the amino acid substitution of M428L. In some embodiments, the variant comprises amino acid substitutions of M252Y, S254T, T256E, M428L, H433K, and N434Y. In some embodiments, the variant comprises amino acid substitutions of M252Y, S254T, T256E, H433K, and N434Y.

In some embodiments the Fc fragment comprises a variant of the amino acid sequence set forth in SEQ ID NO: 56, wherein the variant comprises amino acid substitutions N434Y and H433R. In some embodiments, the variant further comprises amino acid substitutions of M252Y, S254T, and T256E. In some embodiments, the variant further comprises the amino acid substitution of M428L. In some embodiments, the variant comprises amino acid substitutions of M252Y, S254T, T256E, H433R, and N434Y.

In some embodiments the Fc fragment comprises a variant of the amino acid sequence set forth in SEQ ID NO: 56, wherein the variant comprises amino acid substitutions N434Y and H433K. In some embodiments, the variant further comprises amino acid substitutions of M252Y, S254T, and T256E. In some embodiments, the variant further comprises the amino acid substitution of M428L.

In some embodiments, the Fc fragment comprises a variant of the amino acid sequence set forth in SEQ ID NO: 56, wherein the variant comprises amino acid substitutions of M428L and N434F. In some embodiments, the variant further comprises amino acid substitutions of M252Y, S254T, and T256E. In some embodiments, the variant further comprises the amino acid substitution of H433R or H433K. In some embodiments, the variant comprises amino acid substitutions of M428L, N434F, M252Y, S254T, T256E, and H433R. In some embodiments, the variant comprises amino acid substitutions of M428L, N434F, M252Y, S254T, T256E, and H433K. In some embodiments, the variant comprises amino acid substitutions of M428L, N434F, M252Y, S254T, and T256E.

In some embodiments, the Fc fragment comprises a variant of the amino acid sequence set forth in SEQ ID NO: 56, wherein the variant comprises amino acid substitutions of H433R and N434F. In some embodiments, the variant further comprises amino acid substitutions of M252Y, S254T, and T256E. In some embodiments, the variant further comprises the amino acid substitution of M428L. In some embodiments, the variant comprises amino acid substitutions of M252Y, S254T, T256E, H433R, and N434F.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. In certain aspects, the Fc fragments disclosed herein comprise the C-terminal 226 amino acids of the full human IgG Fc region. An Fc can be of the class IgG, and may be further divided into subclasses (isotypes), e.g., IgG₁, IgG₂, IgG₃, and IgG₄. In certain aspects, the Fc fragments described herein are of the IgG₁, subclass. In certain aspects, the Fc fragments described herein are variants of the human IgG₁ Fc region set forth in SEQ ID NO: 1. In certain aspects, the Fc fragments described herein are of the IgG₂ subclass. In certain aspects, the Fc fragments described herein are of the IgG₄, subclass.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). The term also includes the neonatal receptor, FcRn, which is responsible for the transfer of maternal IgGs to the fetus (Guyer et al., J. Immunol. 117:587 (1976); and Kim et al., J. Immunol. 24:249 (1994)). The Fc fragments described herein selectively bind FcRn. In certain aspects, the Fc fragments selectively bind a mammalian FcRn, including cyno, rat and/or mouse FcRn.

Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in DaBron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

Modifications in the CH2 domain can affect the binding of FcRs to the Fc. A number of amino acid modifications in the Fc region are known in the art for selectively altering the affinity of the Fc for different Fc gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123);
F243L (Stewart R, Thom G, Levens M, et al. Protein Eng Des Sel. 2011 September; 24(9):671-8.), S298A/E333A/K334A (Shields R L, Namenuk A K, Hong K, et al. J Biol Chem. 2001 Mar. 2; 276(9):6591-604);
S239D/I332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S, et al. Mol Immunol. 2008 September; 45(15):3926-33);
S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I33 2E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, the Fc fragment comprises one or more modifications selected from the group consisting of: S298A, E333A, K334A, K326A, F243L, R292P, Y300L, V305I, P396L, F243L, R292P, Y300L, L235V, P396L, F243L, S239D, 1332E, A330L, S267E, L328F, D265S, S239E, K326A, A327H, G237F, K326E, G236A, D270L, H268D, S324T, L234F, N325L, V266L, and S267D. In some embodiments, the one or more modifications is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W.

In some embodiments, the Fc fragment comprises a specific combination of amino acid substitutions selected from the group consisting of: L234A/L235A; V234A/G237A; L235A/G237A/E318A; S228P/L236E; H268Q/V309L/A330S/A331S; C220S/C226S/C229S/P238S; C226S/C229S/E3233P/L235V/L235A; L234F/L235E/P331S; C226S/P230S; L234A/G237A; L234A/L235A/G237A; Q311R/M428L; and L234A/L235A/P329G.

In some embodiments, the Fc fragment comprises additional amino acid residues at the N-terminus or the C-terminus. In some embodiments, the Fc fragments provided for herein comprises a C-terminal lysine residue. In some embodiments, the Fc fragment has an additional 1, 2, 3, 4 or 5 amino acid residues at the N-terminus and/or the C-terminus.

In some embodiments, the Fc fragments described herein comprise at least one galactose residue in the oligosaccharide attached to the Fc. Such antibody variants may have improved CDC function.

In some embodiments, the Fc fragments described herein comprise one or more alterations that improve or diminish C1q binding and/or CDC.

In some embodiments, the Fc fragments comprise one or more amino acid substitutions, wherein the one or more substitutions result in a decrease in one or more of ADCC activity, ADCP activity, or CDC activity compared with the Fc without the one or more substitutions.

In some embodiments, the Fc fragments are produced by recombinant cells engineered to express the desired constant domains.

In some embodiments, the Fc fragments disclosed herein are variants of (e.g., comprise one or more amino acid substitutions as compared to) SEQ ID NO: 56, wherein the one or more substitutions result in a measurable K_{D} at pH 7.4 compared to an Fc fragment of SEQ ID NO: 56, which does not detectably bind FcRn at pH 7.4 without the one or more substitutions.

In certain embodiments, the one or more amino acid substitutions results in increased Fc fragment half-life at pH 6.0 compared to an Fc fragment comprising a wild-type Fc region.

In some embodiments, the Fc fragment comprises an amino acid sequence at least 80%, at least 85%, at least 90%, at least 95%, at least 96%, at least 97%, at least 98%, or at least 99% identical to SEQ ID NO: 1. In some embodiments, the Fc fragment comprises an amino acid sequence identical to SEQ ID NO: 1. In some embodiments, the Fc fragment comprises an amino acid sequence at least 80% identical to SEQ ID NO: 1. In some embodiments, the Fc fragment comprises an amino acid sequence at least 85% identical to SEQ ID NO: 1. In some embodiments, the Fc fragment comprises an amino acid sequence at least 90% identical to SEQ ID NO: 1. In some embodiments, the Fc fragment comprises an amino acid sequence at least 95% identical to SEQ ID NO: 1. In some embodiments, the Fc fragment comprises an amino acid sequence at least 96% identical to SEQ ID NO: 1. In some embodiments, the Fc fragment comprises an amino acid sequence at least 97% identical to SEQ ID NO: 1. In some embodiments, the Fc fragment comprises an amino acid sequence at least 98% identical to SEQ ID NO: 1. In some embodiments, the Fc fragment comprises an amino acid sequence at least 99% identical to SEQ ID NO: 1.

In some embodiments, the Fc fragment comprises an amino acid sequence at least 80%, at least 85%, at least 90%, at least 95%, at least 96%, at least 97%, at least 98%, or at least 99% identical to SEQ ID NO: 2. In some embodiments, the Fc fragment comprises an amino acid sequence identical to SEQ ID NO: 2. In some embodiments, the Fc fragment comprises an amino acid sequence at least 80% identical to SEQ ID NO: 2. In some embodiments, the Fc fragment comprises an amino acid sequence at least 85% identical to SEQ ID NO: 2. In some embodiments, the Fc fragment comprises an amino acid sequence at least 90% identical to SEQ ID NO: 2. In some embodiments, the Fc fragment comprises an amino acid sequence at least 95% identical to SEQ ID NO: 2. In some embodiments, the Fc fragment comprises an amino acid sequence at least 96% identical to SEQ ID NO: 2. In some embodiments, the Fc fragment comprises an amino acid sequence at least 97% identical to SEQ ID NO: 2. In some embodiments, the Fc fragment comprises an amino acid sequence at least 98% identical to SEQ ID NO: 2. In some embodiments, the Fc fragment comprises an amino acid sequence at least 99% identical to SEQ ID NO: 2.

In some embodiments, the Fc fragment comprises an amino acid sequence at least 80%, at least 85%, at least 90%, at least 95%, at least 96%, at least 97%, at least 98%, or at least 99% identical to SEQ ID NO: 3. In some embodiments, the Fc fragment comprises an amino acid sequence identical to SEQ ID NO: 3. In some embodiments, the Fc fragment comprises an amino acid sequence at least 80% identical to SEQ ID NO: 3. In some embodiments, the Fc fragment comprises an amino acid sequence at least 85% identical to SEQ ID NO: 3. In some embodiments, the Fc fragment comprises an amino acid sequence at least 90% identical to SEQ ID NO: 3. In some embodiments, the Fc fragment comprises an amino acid sequence at least 95% identical to SEQ ID NO: 3. In some embodiments, the Fc fragment comprises an amino acid sequence at least 96% identical to SEQ ID NO: 3. In some embodiments, the Fc fragment comprises an amino acid sequence at least 97% identical to SEQ ID NO: 3. In some embodiments, the Fc fragment comprises an amino acid sequence at least 98% identical to SEQ ID NO: 3. In some embodiments, the Fc fragment comprises an amino acid sequence at least 99% identical to SEQ ID NO: 3.

In some embodiments, the Fc fragment comprises an amino acid sequence at least 80%, at least 85%, at least 90%, at least 95%, at least 96%, at least 97%, at least 98%, or at least 99% identical to SEQ ID NO: 4. In some embodiments, the Fc fragment comprises an amino acid sequence identical to SEQ ID NO: 4. In some embodiments, the Fc fragment comprises an amino acid sequence at least 80% identical to SEQ ID NO: 4. In some embodiments, the Fc fragment comprises an amino acid sequence at least 85% identical to SEQ ID NO: 4. In some embodiments, the Fc fragment comprises an amino acid sequence at least 90% identical to SEQ ID NO: 4. In some embodiments, the Fc fragment comprises an amino acid sequence at least 95% identical to SEQ ID NO: 4. In some embodiments, the Fc fragment comprises an amino acid sequence at least 96% identical to SEQ ID NO: 4. In some embodiments, the Fc fragment comprises an amino acid sequence at least 97% identical to SEQ ID NO: 4. In some embodiments, the Fc fragment comprises an amino acid sequence at least 98% identical to SEQ ID NO: 4. In some embodiments, the Fc fragment comprises an amino acid sequence at least 99% identical to SEQ ID NO: 4.

In some embodiments, the Fc fragment comprises an amino acid sequence at least 80%, at least 85%, at least 90%, at least 95%, at least 96%, at least 97%, at least 98%, or at least 99% identical to SEQ ID NO: 5. In some embodiments, the Fc fragment comprises an amino acid sequence identical to SEQ ID NO: 5. In some embodiments, the Fc fragment comprises an amino acid sequence at least 80% identical to SEQ ID NO: 5. In some embodiments, the Fc fragment comprises an amino acid sequence at least 85% identical to SEQ ID NO: 5. In some embodiments, the Fc fragment comprises an amino acid sequence at least 90% identical to SEQ ID NO: 5. In some embodiments, the Fc fragment comprises an amino acid sequence at least 95% identical to SEQ ID NO: 5. In some embodiments, the Fc fragment comprises an amino acid sequence at least 96% identical to SEQ ID NO: 5. In some embodiments, the Fc fragment comprises an amino acid sequence at least 97% identical to SEQ ID NO: 5. In some embodiments, the Fc fragment comprises an amino acid sequence at least 98% identical to SEQ ID NO: 5. In some embodiments, the Fc fragment comprises an amino acid sequence at least 99% identical to SEQ ID NO: 5.

In some embodiments, the Fc fragment comprises an amino acid sequence at least 80%, at least 85%, at least 90%, at least 95%, at least 96%, at least 97%, at least 98%, or at least 99% identical to SEQ ID NO: 6. In some embodiments, the Fc fragment comprises an amino acid sequence identical to SEQ ID NO: 6. In some embodiments, the Fc fragment comprises an amino acid sequence at least 80% identical to SEQ ID NO: 6. In some embodiments, the Fc fragment comprises an amino acid sequence at least 85% identical to SEQ ID NO: 6. In some embodiments, the Fc fragment comprises an amino acid sequence at least 90% identical to SEQ ID NO: 6. In some embodiments, the Fc fragment comprises an amino acid sequence at least 95% identical to SEQ ID NO: 6. In some embodiments, the Fc fragment comprises an amino acid sequence at least 96% identical to SEQ ID NO: 6. In some embodiments, the Fc fragment comprises an amino acid sequence at least 97% identical to SEQ ID NO: 6. In some embodiments, the Fc fragment comprises an amino acid sequence at least 98% identical to SEQ ID NO: 6. In some embodiments, the Fc fragment comprises an amino acid sequence at least 99% identical to SEQ ID NO: 6.

In some embodiments, the Fc fragment comprises an amino acid sequence at least 80%, at least 85%, at least 90%, at least 95%, at least 96%, at least 97%, at least 98%, or at least 99% identical to SEQ ID NO: 7. In some embodiments, the Fc fragment comprises an amino acid sequence identical to SEQ ID NO: 7. In some embodiments, the Fc fragment comprises an amino acid sequence at least 80% identical to SEQ ID NO: 7. In some embodiments, the Fc fragment comprises an amino acid sequence at least 85% identical to SEQ ID NO: 7. In some embodiments, the Fc fragment comprises an amino acid sequence at least 90% identical to SEQ ID NO: 7. In some embodiments, the Fc fragment comprises an amino acid sequence at least 95% identical to SEQ ID NO: 7. In some embodiments, the Fc fragment comprises an amino acid sequence at least 96% identical to SEQ ID NO: 7. In some embodiments, the Fc fragment comprises an amino acid sequence at least 97% identical to SEQ ID NO: 7. In some embodiments, the Fc fragment comprises an amino acid sequence at least 98% identical to SEQ ID NO: 7. In some embodiments, the Fc fragment comprises an amino acid sequence at least 99% identical to SEQ ID NO: 7.

In some embodiments, the Fc fragment comprises an amino acid sequence at least 80%, at least 85%, at least 90%, at least 95%, at least 96%, at least 97%, at least 98%, or at least 99% identical to SEQ ID NO: 8. In some embodiments, the Fc fragment comprises an amino acid sequence identical to SEQ ID NO: 8. In some embodiments, the Fc fragment comprises an amino acid sequence at least 80% identical to SEQ ID NO: 8. In some embodiments, the Fc fragment comprises an amino acid sequence at least 85% identical to SEQ ID NO: 8. In some embodiments, the Fc fragment comprises an amino acid sequence at least 90% identical to SEQ ID NO: 8. In some embodiments, the Fc fragment comprises an amino acid sequence at least 95% identical to SEQ ID NO: 8. In some embodiments, the Fc fragment comprises an amino acid sequence at least 96% identical to SEQ ID NO: 8. In some embodiments, the Fc fragment comprises an amino acid sequence at least 97% identical to SEQ ID NO: 8. In some embodiments, the Fc fragment comprises an amino acid sequence at least 98% identical to SEQ ID NO: 8. In some embodiments, the Fc fragment comprises an amino acid sequence at least 99% identical to SEQ ID NO: 8.

In some embodiments, the Fc fragment comprises an amino acid sequence at least 80%, at least 85%, at least 90%, at least 95%, at least 96%, at least 97%, at least 98%, or at least 99% identical to SEQ ID NO: 9. In some embodiments, the Fc fragment comprises an amino acid sequence identical to SEQ ID NO: 9. In some embodiments, the Fc fragment comprises an amino acid sequence at least 80% identical to SEQ ID NO: 9. In some embodiments, the Fc fragment comprises an amino acid sequence at least 85% identical to SEQ ID NO: 9. In some embodiments, the Fc fragment comprises an amino acid sequence at least 90% identical to SEQ ID NO: 9. In some embodiments, the Fc fragment comprises an amino acid sequence at least 95% identical to SEQ ID NO: 9. In some embodiments, the Fc fragment comprises an amino acid sequence at least 96% identical to SEQ ID NO: 9. In some embodiments, the Fc fragment comprises an amino acid sequence at least 97% identical to SEQ ID NO: 9. In some embodiments, the Fc fragment comprises an amino acid sequence at least 98% identical to SEQ ID NO: 9. In some embodiments, the Fc fragment comprises an amino acid sequence at least 99% identical to SEQ ID NO: 9.

In some embodiments, the Fc fragment comprises an amino acid sequence at least 80%, at least 85%, at least 90%, at least 95%, at least 96%, at least 97%, at least 98%, or at least 99% identical to SEQ ID NO: 57. In some embodiments, the Fc fragment comprises an amino acid sequence identical to SEQ ID NO: 57. In some embodiments, the Fc fragment comprises an amino acid sequence at least 80% identical to SEQ ID NO: 57. In some embodiments, the Fc fragment comprises an amino acid sequence at least 85% identical to SEQ ID NO: 57. In some embodiments, the Fc fragment comprises an amino acid sequence at least 90% identical to SEQ ID NO: 57. In some embodiments, the Fc fragment comprises an amino acid sequence at least 95% identical to SEQ ID NO: 57. In some embodiments, the Fc fragment comprises an amino acid sequence at least 96% identical to SEQ ID NO: 57. In some embodiments, the Fc fragment comprises an amino acid sequence at least 97% identical to SEQ ID NO: 57. In some embodiments, the Fc fragment comprises an amino acid sequence at least 98% identical to SEQ ID NO: 57. In some embodiments, the Fc fragment comprises an amino acid sequence at least 99% identical to SEQ ID NO: 57.

In some embodiments, the Fc fragment comprises an amino acid sequence at least 80%, at least 85%, at least 90%, at least 95%, at least 96%, at least 97%, at least 98%, or at least 99% identical to SEQ ID NO: 58. In some embodiments, the Fc fragment comprises an amino acid sequence identical to SEQ ID NO: 58. In some embodiments, the Fc fragment comprises an amino acid sequence at least 80% identical to SEQ ID NO: 58. In some embodiments, the Fc fragment comprises an amino acid sequence at least 85% identical to SEQ ID NO: 58. In some embodiments, the Fc fragment comprises an amino acid sequence at least 90% identical to SEQ ID NO: 58. In some embodiments, the Fc fragment comprises an amino acid sequence at least 95% identical to SEQ ID NO: 58. In some embodiments, the Fc fragment comprises an amino acid sequence at least 96% identical to SEQ ID NO: 58. In some embodiments, the Fc fragment comprises an amino acid sequence at least 97% identical to SEQ ID NO: 58. In some embodiments, the Fc fragment comprises an amino acid sequence at least 98% identical to SEQ ID NO: 58. In some embodiments, the Fc fragment comprises an amino acid sequence at least 99% identical to SEQ ID NO: 58.

In some embodiments, the Fc fragment comprises an amino acid sequence at least 80%, at least 85%, at least 90%, at least 95%, at least 96%, at least 97%, at least 98%, or at least 99% identical to SEQ ID NO: 59. In some embodiments, the Fc fragment comprises an amino acid sequence identical to SEQ ID NO: 59. In some embodiments, the Fc fragment comprises an amino acid sequence at least 80% identical to SEQ ID NO: 59. In some embodiments, the Fc fragment comprises an amino acid sequence at least 85% identical to SEQ ID NO: 59. In some embodiments, the Fc fragment comprises an amino acid sequence at least 90% identical to SEQ ID NO: 59. In some embodiments, the Fc fragment comprises an amino acid sequence at least 95% identical to SEQ ID NO: 59. In some embodiments, the Fc fragment comprises an amino acid sequence at least 96% identical to SEQ ID NO: 59. In some embodiments, the Fc fragment comprises an amino acid sequence at least 97% identical to SEQ ID NO: 59. In some embodiments, the Fc fragment comprises an amino acid sequence at least 98% identical to SEQ ID NO: 59. In some embodiments, the Fc fragment comprises an amino acid sequence at least 99% identical to SEQ ID NO: 59.

In some embodiments, the Fc fragment comprises an amino acid sequence at least 80%, at least 85%, at least 90%, at least 95%, at least 96%, at least 97%, at least 98%, or at least 99% identical to SEQ ID NO: 60. In some embodiments, the Fc fragment comprises an amino acid sequence identical to SEQ ID NO: 60. In some embodiments, the Fc fragment comprises an amino acid sequence at least 80% identical to SEQ ID NO: 60. In some embodiments, the Fc fragment comprises an amino acid sequence at least 85% identical to SEQ ID NO: 60. In some embodiments, the Fc fragment comprises an amino acid sequence at least 90% identical to SEQ ID NO: 60. In some embodiments, the Fc fragment comprises an amino acid sequence at least 95% identical to SEQ ID NO: 60. In some embodiments, the Fc fragment comprises an amino acid sequence at least 96% identical to SEQ ID NO: 60. In some embodiments, the Fc fragment comprises an amino acid sequence at least 97% identical to SEQ ID NO: 60. In some embodiments, the Fc fragment comprises an amino acid sequence at least 98% identical to SEQ ID NO: 60. In some embodiments, the Fc fragment comprises an amino acid sequence at least 99% identical to SEQ ID NO: 60.

In some embodiments, the Fc fragment comprises an amino acid sequence at least 80%, at least 85%, at least 90%, at least 95%, at least 96%, at least 97%, at least 98%, or at least 99% identical to SEQ ID NO: 61. In some embodiments, the Fc fragment comprises an amino acid sequence identical to SEQ ID NO: 61. In some embodiments, the Fc fragment comprises an amino acid sequence at least 80% identical to SEQ ID NO: 61. In some embodiments, the Fc fragment comprises an amino acid sequence at least 85% identical to SEQ ID NO: 61. In some embodiments, the Fc fragment comprises an amino acid sequence at least 90% identical to SEQ ID NO: 61. In some embodiments, the Fc fragment comprises an amino acid sequence at least 95% identical to SEQ ID NO: 61. In some embodiments, the Fc fragment comprises an amino acid sequence at least 96% identical to SEQ ID NO: 61. In some embodiments, the Fc fragment comprises an amino acid sequence at least 97% identical to SEQ ID NO: 61. In some embodiments, the Fc fragment comprises an amino acid sequence at least 98% identical to SEQ ID NO: 61. In some embodiments, the Fc fragment comprises an amino acid sequence at least 99% identical to SEQ ID NO: 61.

In some embodiments, the Fc fragment comprises an amino acid sequence at least 80%, at least 85%, at least 90%, at least 95%, at least 96%, at least 97%, at least 98%, or at least 99% identical to SEQ ID NO: 62. In some embodiments, the Fc fragment comprises an amino acid sequence identical to SEQ ID NO: 62. In some embodiments, the Fc fragment comprises an amino acid sequence at least 80% identical to SEQ ID NO: 62. In some embodiments, the Fc fragment comprises an amino acid sequence at least 85% identical to SEQ ID NO: 62. In some embodiments, the Fc fragment comprises an amino acid sequence at least 90% identical to SEQ ID NO: 62. In some embodiments, the Fc fragment comprises an amino acid sequence at least 95% identical to SEQ ID NO: 62. In some embodiments, the Fc fragment comprises an amino acid sequence at least 96% identical to SEQ ID NO: 62. In some embodiments, the Fc fragment comprises an amino acid sequence at least 97% identical to SEQ ID NO: 62. In some embodiments, the Fc fragment comprises an amino acid sequence at least 98% identical to SEQ ID NO: 62. In some embodiments, the Fc fragment comprises an amino acid sequence at least 99% identical to SEQ ID NO: 62.

In some embodiments, the Fc fragment comprises an amino acid sequence at least 80%, at least 85%, at least 90%, at least 95%, at least 96%, at least 97%, at least 98%, or at least 99% identical to SEQ ID NO: 63. In some embodiments, the Fc fragment comprises an amino acid sequence identical to SEQ ID NO: 63. In some embodiments, the Fc fragment comprises an amino acid sequence at least 80% identical to SEQ ID NO: 63. In some embodiments, the Fc fragment comprises an amino acid sequence at least 85% identical to SEQ ID NO: 63. In some embodiments, the Fc fragment comprises an amino acid sequence at least 90% identical to SEQ ID NO: 63. In some embodiments, the Fc fragment comprises an amino acid sequence at least 95% identical to SEQ ID NO: 63. In some embodiments, the Fc fragment comprises an amino acid sequence at least 96% identical to SEQ ID NO: 63. In some embodiments, the Fc fragment comprises an amino acid sequence at least 97% identical to SEQ ID NO: 63. In some embodiments, the Fc fragment comprises an amino acid sequence at least 98% identical to SEQ ID NO: 63. In some embodiments, the Fc fragment comprises an amino acid sequence at least 99% identical to SEQ ID NO: 63.

In some embodiments, the Fc fragment comprises an amino acid sequence at least 80%, at least 85%, at least 90%, at least 95%, at least 96%, at least 97%, at least 98%, or at least 99% identical to SEQ ID NO: 64. In some embodiments, the Fc fragment comprises an amino acid sequence identical to SEQ ID NO: 64. In some embodiments, the Fc fragment comprises an amino acid sequence at least 80% identical to SEQ ID NO: 64. In some embodiments, the Fc fragment comprises an amino acid sequence at least 85% identical to SEQ ID NO: 64. In some embodiments, the Fc fragment comprises an amino acid sequence at least 90% identical to SEQ ID NO: 64. In some embodiments, the Fc fragment comprises an amino acid sequence at least 95% identical to SEQ ID NO: 64. In some embodiments, the Fc fragment comprises an amino acid sequence at least 96% identical to SEQ ID NO: 64. In some embodiments, the Fc fragment comprises an amino acid sequence at least 97% identical to SEQ ID NO: 64. In some embodiments, the Fc fragment comprises an amino acid sequence at least 98% identical to SEQ ID NO: 64. In some embodiments, the Fc fragment comprises an amino acid sequence at least 99% identical to SEQ ID NO: 64.

In some embodiments, the Fc fragment comprises an amino acid sequence at least 80%, at least 85%, at least 90%, at least 95%, at least 96%, at least 97%, at least 98%, or at least 99% identical to SEQ ID NO: 65. In some embodiments, the Fc fragment comprises an amino acid sequence identical to SEQ ID NO: 65. In some embodiments, the Fc fragment comprises an amino acid sequence at least 80% identical to SEQ ID NO: 65. In some embodiments, the Fc fragment comprises an amino acid sequence at least 85% identical to SEQ ID NO: 65. In some embodiments, the Fc fragment comprises an amino acid sequence at least 90% identical to SEQ ID NO: 65. In some embodiments, the Fc fragment comprises an amino acid sequence at least 95% identical to SEQ ID NO: 65. In some embodiments, the Fc fragment comprises an amino acid sequence at least 96% identical to SEQ ID NO: 65. In some embodiments, the Fc fragment comprises an amino acid sequence at least 97% identical to SEQ ID NO: 65. In some embodiments, the Fc fragment comprises an amino acid sequence at least 98% identical to SEQ ID NO: 65. In some embodiments, the Fc fragment comprises an amino acid sequence at least 99% identical to SEQ ID NO: 65.

In some embodiments, the Fc fragment comprises an amino acid sequence at least 80%, at least 85%, at least 90%, at least 95%, at least 96%, at least 97%, at least 98%, or at least 99% identical to SEQ ID NO: 66. In some embodiments, the Fc fragment comprises an amino acid sequence identical to SEQ ID NO: 66. In some embodiments, the Fc fragment comprises an amino acid sequence at least 80% identical to SEQ ID NO: 66. In some embodiments, the Fc fragment comprises an amino acid sequence at least 85% identical to SEQ ID NO: 66. In some embodiments, the Fc fragment comprises an amino acid sequence at least 90% identical to SEQ ID NO: 66. In some embodiments, the Fc fragment comprises an amino acid sequence at least 95% identical to SEQ ID NO: 66. In some embodiments, the Fc fragment comprises an amino acid sequence at least 96% identical to SEQ ID NO: 66. In some embodiments, the Fc fragment comprises an amino acid sequence at least 97% identical to SEQ ID NO: 66. In some embodiments, the Fc fragment comprises an amino acid sequence at least 98% identical to SEQ ID NO: 66. In some embodiments, the Fc fragment comprises an amino acid sequence at least 99% identical to SEQ ID NO: 66.

In some embodiments, the Fc fragment comprises an amino acid sequence at least 80%, at least 85%, at least 90%, at least 95%, at least 96%, at least 97%, at least 98%, or at least 99% identical to SEQ ID NO: 67. In some embodiments, the Fc fragment comprises an amino acid sequence identical to SEQ ID NO: 67. In some embodiments, the Fc fragment comprises an amino acid sequence at least 80% identical to SEQ ID NO: 67. In some embodiments, the Fc fragment comprises an amino acid sequence at least 85% identical to SEQ ID NO: 67. In some embodiments, the Fc fragment comprises an amino acid sequence at least 90% identical to SEQ ID NO: 67. In some embodiments, the Fc fragment comprises an amino acid sequence at least 95% identical to SEQ ID NO: 67. In some embodiments, the Fc fragment comprises an amino acid sequence at least 96% identical to SEQ ID NO: 67. In some embodiments, the Fc fragment comprises an amino acid sequence at least 97% identical to SEQ ID NO: 67. In some embodiments, the Fc fragment comprises an amino acid sequence at least 98% identical to SEQ ID NO: 67. In some embodiments, the Fc fragment comprises an amino acid sequence at least 99% identical to SEQ ID NO: 67.

In some embodiments, the Fc fragment comprises an amino acid sequence identical to any one of SEQ ID NOs: 1-9 or 57-67.

In some embodiments, the Fc fragment comprises an amino acid sequence identical to SEQ ID NO: 1. In some embodiments, the Fc fragment comprises an amino acid sequence identical to SEQ ID NO: 2. In some embodiments, the Fc fragment comprises an amino acid sequence identical to SEQ ID NO: 3. In some embodiments, the Fc fragment comprises an amino acid sequence identical to SEQ ID NO: 4. In some embodiments, the Fc fragment comprises an amino acid sequence identical to SEQ ID NO: 5.

In some embodiments, the Fc fragment comprises an amino acid sequence identical to SEQ ID NO: 6. In some embodiments, the Fc fragment comprises an amino acid sequence identical to SEQ ID NO: 7. In some embodiments, the Fc fragment comprises an amino acid sequence identical to SEQ ID NO: 8. In some embodiments, the Fc fragment comprises an amino acid sequence identical to SEQ ID NO: 9.

In some embodiments, the Fc fragment comprises an amino acid sequence identical to SEQ ID NO: 57. In some embodiments, the Fc fragment comprises an amino acid sequence identical to SEQ ID NO: 58. In some embodiments, the Fc fragment comprises an amino acid sequence identical to SEQ ID NO: 59. In some embodiments, the Fc fragment comprises an amino acid sequence identical to SEQ ID NO: 60. In some embodiments, the Fc fragment comprises an amino acid sequence identical to SEQ ID NO: 61. In some embodiments, the Fc fragment comprises an amino acid sequence identical to SEQ ID NO: 62.

In some embodiments, the Fc fragment comprises an amino acid sequence identical to SEQ ID NO: 63. In some embodiments, the Fc fragment comprises an amino acid sequence identical to SEQ ID NO: 64. In some embodiments, the Fc fragment comprises an amino acid sequence identical to SEQ ID NO: 65. In some embodiments, the Fc fragment comprises an amino acid sequence identical to SEQ ID NO: 66. In some embodiments, the Fc fragment comprises an amino acid sequence identical to SEQ ID NO: 67.

### Linkers

Linker sequences can be provided in the Fc fusion molecules described herein to separate the different components (e.g., albumin or albumin binding domain, Fc fragment).

In some embodiments, the linker is a peptide linker. In some embodiments, the peptide linker comprises a hinge region or a portion thereof. In some embodiments, the hinge region is derived from an IgG hinge region. In some embodiments, the hinge region is derived from a human IgG hinge region.

In some embodiments, the peptide linker comprises a flexible peptide linker. In some embodiments, the peptide linker comprises a rigid peptide linker. In some embodiments, the peptide linker comprises a cleavable peptide linker. In some embodiments the linker is repeated once. In some embodiments the linker is repeated more than once.

In some embodiments, the linker comprises at least 5 to about 50 amino acids. In some embodiments, the linker comprises about 5 to about 50 amino acids, about 5 to about 45 amino acids, about 5 to about 40 amino acids, about 5 to about 35 amino acids, about 5 to about 30 amino acids, about 5 to about 25 amino acids, about 5 to about 20 amino acids, about 5 to about 15 amino acids, about 5 to about 10 amino acids, about 10 to about 50 amino acids, about 15 to about 50 amino acids, about 20 to about 50 amino acids, about 25 to about 50 amino acids, about 30 to about 50 amino acids, about 35 to about 50 amino acids, about 40 to about 50 amino acids, or about 45 to about 50 amino acids.

In some embodiments, the linker comprises a sequence according to any one of SEQ ID NOs: 13-15. In some embodiments, the linker comprises a sequence according to any one of SEQ ID NOs: 13-15 or 74-75. In some embodiments, the linker comprises a sequence according to SEQ ID NO: 13. In some embodiments, the linker comprises a sequence according to SEQ ID NO: 14. In some embodiments, the linker comprises a sequence according to SEQ ID NO: 15.

In some embodiments, the linker comprises a sequence according to any one of SEQ ID NOs: 74-75. In some embodiments, the linker comprises a sequence according to SEQ ID NO: 74. In some embodiments, the linker comprises a sequence according to SEQ ID NO: 75.

In some embodiments, the linker comprises a sequence selected from the group consisting of (GS)ₙ, (G2S)ₙ, (G3S)ₙ, (G4S)ₙ, and (G)ₙ, and wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 6 to 8, from 8 to 20, from 10 to 20, from 12 to 20, from 14 to 20, from 16 to 20, or from 18 to 20. In some embodiments, n is an integer greater than or equal to 6. In some embodiments, n is an integer greater than or equal to 8. In some embodiments, n is 6. In some embodiments, n is 8.

In some embodiments, the linker comprises (G4S)ₙ, wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 6 to 8, from 8 to 20, from 10 to 20, from 12 to 20, from 14 to 20, from 16 to 20, or from 18 to 20. In some embodiments, n is an integer greater than or equal to 6. In some embodiments, n is an integer greater than or equal to 8. In some embodiments, n is 6. In some embodiments, n is 8.

In some embodiments, the linker comprises (G4S)₁. In some embodiments, the linker comprises (G4S)₂. In some embodiments, the linker comprises (G4S)₃. In some embodiments, the linker comprises (G4S)₄. In some embodiments, the linker comprises (G4S)₅. In some embodiments, the linker comprises (G4S)₆. In some embodiments, the linker comprises (G4S)₇. In some embodiments, the linker comprises (G4S)₈.

In some embodiments, the linker comprises a sequence of GGGGG (SEQ ID NO: 77). In some embodiments, the linker comprises a sequence consisting of (GGGGG)n (SEQ ID NO: 77), and wherein n is an integer from 2 to 6.

In some embodiments, the linker comprises a sequence selected from GGSGGD (SEQ ID NO: 78) or GGSGGE (SEQ ID NO: 79). In some embodiments, the linker comprises a sequence selected from the group consisting of (GGSGGD)ₙ (SEQ ID NO: 78) or (GGSGGE)ₙ (SEQ ID NO: 79), and wherein n is an integer from 2 to 6.

In some embodiments, the linker comprises a sequence selected from the group consisting of GGGSGSGGGGS (SEQ ID NO: 80) and GGGGGPGGGGP (SEQ ID NO: 81). In some embodiments, the linker comprises a sequence selected from the group consisting of (GGGSGSGGGGS)ₙ (SEQ ID NO: 80) and (GGGGGPGGGGP)ₙ (SEQ ID NO: 81), and wherein n is an integer from 1 to 3.

In some embodiments, the linker comprises a sequence selected from the group consisting of GGGGGG (SEQ ID NO: 82) and GGGGGGGG (SEQ ID NO: 83).

In some embodiments, the linker comprises a sequence selected from the group consisting of (GX)n, (GGX)n, (GGGX)n, (GGGGX)n (SEQ ID NO: 84), and (GzX)n, wherein z is between 1 and 20, and wherein n is at least 8. In some embodiments, z is between 2 and 18, 2 and 16, 2 and 14, 2 and 12, 2 and 10, 2 and 8, 2 and 6, 2 and 4, 4 and 20, 6 and 20, 8 and 20, 10 and 20, 12 and 20, 14 and 20, 16 and 20, or 18 and 20. In some embodiments, X is serine, aspartic acid, glutamic acid, threonine, or proline.

In some embodiments, the linker comprises an amino acid sequence selected from Table 1B.

TABLE 1B
Amino acid sequences of exemplary linkers.

| Identifier | Amino Acid sequence |
|---|---|
| G₄S | GGGGS |
| (SEQ ID NO: 13) | |
| (G₄S)₇ | GGGGSGGGGSGGGGSGGGGSGGGGSGGGGS |
| (SEQ ID NO: 14) | GGGGS |
| (G₄S)₃ | GGGGSGGGGSGGGGS |
| (SEQ ID NO: 15) | |
| (G₄S)₆ | GGGGSGGGGSGGGGSGGGGSGGGGSGGGGS |
| (SEQ ID NO: 74) | |
| (G₄S)₈ | GGGGSGGGGSGGGGSGGGGSGGGGSGGGGS |
| (SEQ ID NO: 75) | GGGGSGGGGS |

### Half-Life Extension Domain

In some embodiments, the Fc fusion molecule comprises an Fc fragment and a half-life extension domain. In some embodiments, the Fc fusion molecule comprises an Fc fragment, a linker, and a half-life extension domain.

In some embodiments, the half-life extension domain comprises an albumin. In some embodiments, the half-life extension moiety comprises human serum albumin. In some embodiments, the half-life extension domain comprises a protein or fragment thereof that binds to an albumin. In some embodiments, the half-life extension domain comprises a protein or fragment thereof that binds to human serum albumin (HSA).

In some embodiments, the half-life extension domain comprises an anti-HSA antibody or fragment thereof. In some embodiments, the half-life extension domain comprises an anti-HSA Fab. In some embodiments, the half-life extension domain comprises an anti-HSA Fab′. In some embodiments, the half-life extension domain comprises an anti-HSA F(ab)′2. In some embodiments, the half-life extension domain comprises an anti-HSA variable fragment (Fv). In some embodiments, the half-life extension domain comprises an anti-HSA single-domain antibody (sdAb). In some embodiments, the half-life extension domain comprises an anti-HSA VHH. In some embodiments, the half-life extension domain comprises an anti-HSA VNAR. In some embodiments, the half-life extension domain comprises an anti-HSA single chain variable fragment (scFv). In some embodiments, the half-life extension domain comprises an anti-HSA single chain Fab (scFab).

In some embodiments, the half-life extension domain comprises one or more lipids. Lipids can comprise various lengths, chemistries, linkers, and conjugates that are apparent to a person of ordinary skill in the art. In some embodiments, the half-life extension domain comprises a saturated fatty acid. In some embodiments, the saturated fatty acid is linked to the Fc fragment through its carboxylate group. In some embodiments, the saturated fatty acid comprises a C₆₋₁₈ saturated fatty acid (e.g., a C₆, C₁₂, C₁₆, or Cis saturated fatty acid). In some embodiments, the half-life extension domain comprises a saturated fatty diacid. In some embodiments, the saturated fatty diacid is linked to the Fc fragment through its carboxylate group. In some embodiments, the saturated fatty diacid comprises a C₆₋₁₈ saturated fatty diacid (e.g., a C₆, C₁₂, C₁₆, or Cis saturated fatty diacid).

In some embodiments, the half-life extension domain comprises one or more polyethylene glycols (PEGs). PEGs can comprise various lengths, various molecular weights, and various linkers that are apparent to a person of ordinary skill in the art. In some embodiments, the PEG is monodisperse. In some embodiments, the monodisperse PEG has 12-48 ethylene glycol repeats (e.g., 12-24 repeats, 12-36 repeats, 24-48 repeats, 24-36 repeats, or 36-48 repeats). In some embodiments, the PEG is polydisperse. In some embodiments, the polydisperse PEG has a molecular weight of 500 to 50,000 daltons (e.g., 500-1,000 Da, 500-5,000 Da, 500-20,000 Da, 500-40,000 Da, 1,000-5,000 Da, 1,000-20,000 Da, 1,000-40,000 Da, 1,000-50,000 Da, 5,000-10,000 Da, 5,000-20,000 Da, 5,000-40,000 Da, 5,000-50,000 Da, 10,000-20,000 Da, 10,000-40,000 Da, 10,000-50,000 Da, 20,000-40,000 Da, or 20,000-50,000 Da). In some embodiments, the polydisperse PEG has a molecular weight of 0.5 kDa, 1 kDa, 5 kDa, 10 kDa, 15 kDa, 20 kDa, 25 kDa, 30 kDa, 35 kDa, 40 kDa, 45 kDa, or 50 kDa.

In some embodiments, the half-life extension domain comprises a polydisperse PEG with a molecular weight of 1 kDa. In some embodiments, the half-life extension domain comprises a polydisperse PEG with a molecular weight of 5 kDa. In some embodiments, the half-life extension domain comprises a polydisperse PEG with a molecular weight of 10 kDa. In some embodiments, the half-life extension domain comprises a polydisperse PEG with a molecular weight of 15 kDa. In some embodiments, the half-life extension domain comprises a polydisperse PEG with a molecular weight of 20 kDa. In some embodiments, the half-life extension domain comprises a polydisperse PEG with a molecular weight of 25 kDa. In some embodiments, the half-life extension domain comprises a polydisperse PEG with a molecular weight of 30 kDa. In some embodiments, the half-life extension domain comprises a polydisperse PEG with a molecular weight of 35 kDa. In some embodiments, the half-life extension domain comprises a polydisperse PEG with a molecular weight of 40 kDa. In some embodiments, the half-life extension domain comprises a polydisperse PEG with a molecular weight of 50 kDa.

In some embodiments, the half-life extension domain comprises a half-life extending polypeptide. Half-life extending polypeptides can comprise various lengths, chemistries, linkers, and conjugates that are apparent to a person of ordinary skill in the art. In some embodiment, the half-life extending polypeptide comprises repeated units of Pro-Ala-Ser (PAS). In some embodiments, the half-life extending polypeptide comprises 50-500 repeated PAS units (e.g., 50-100 PAS units, 50-200 PAS units, 100-200 PAS units, 100-500 PAS units, or 200-500 PAS units).

In some embodiments, the half-life extending polypeptide comprises an XTEN™ polypeptide. XTEN is described in US20150037359A1, the contents of which are incorporated by reference herein in its entirety.

### Antigen Binding Domains

In some embodiments, the Fc fusion molecule comprises an Fc fragment and an antigen binding domain. In some embodiments, the Fc fusion molecule comprises an Fc fragment, a linker, and an antigen binding domain.

In some embodiments, the antigen binding domain comprises a Fab, a Fab′, a F(ab)′2, a variable fragment (Fv), a single-domain antibody (sdAb), a VHH, a VNAR, a single chain variable fragment (scFv), or a single chain Fab (scFab). In some embodiments, the antigen binding domain comprises a Fab. In some embodiments, the antigen binding domain comprises a Fab′. In some embodiments, the antigen binding domain comprises a F(ab)′2. In some embodiments, the antigen binding domain comprises a Fv. In some embodiments, the antigen binding domain comprises a light chain variable domain. In some embodiments, the antigen binding domain comprises a heavy chain variable domain. In some embodiments, the antigen binding domain comprises a sdAb. In some embodiments, the antigen binding domain comprises a VHH. In some embodiments, the antigen binding domain comprises a VNAR. In some embodiments, the antigen binding domain comprises a scFv. In some embodiments, the antigen binding domain comprises a scFab.

In some embodiments, the antigen binding domain is monospecific. In some embodiments, the antigen binding domain is bispecific. In some embodiments, the antigen binding domain is multi-specific.

In some embodiments, the antigen binding domain targets a non-human antigen. In some embodiments, a non-human antigen is a protein or fragment that is not normally expressed by humans but may be found in humans. In some embodiments, a non-human antigen comprises a protein or fragment thereof expressed by pathogens. In some embodiments, the non-human antigen is a bacterial antigen. In some embodiments, a non-human antigen is a viral antigen.

In some embodiments, the antigen binding domain targets a human antigen. In some embodiments, the human antigen is associated with an autoimmune disease. In some embodiments, the human antigen is IgG. In some embodiments, the human antigen is IgE. In some embodiments, the human antigen is IgM. In some embodiments, the human antigen is IgA. In some embodiments, the human antigen is IgD.

In some embodiments, the human antigen is human serum albumin.

### Albumin and Albumin Binding Domains

Provided herein are Fc fusion molecules comprising an albumin or albumin binding domain.

In some embodiments, the Fc fusion molecule comprises an Fc fragment and an albumin. In some embodiments, the albumin is human serum albumin. In some embodiments, the albumin comprises an amino acid sequence at least 80% identical to SEQ ID NO: 10. In some embodiments, the albumin comprises an amino acid sequence at least 85% identical to SEQ ID NO: 10. In some embodiments, the albumin comprises an amino acid sequence at least 90% identical to SEQ ID NO: 10. In some embodiments, the albumin comprises an amino acid sequence at least 95% identical to SEQ ID NO: 10. In some embodiments, the albumin comprises an amino acid sequence at least 96% identical to SEQ ID NO: 10. In some embodiments, the albumin comprises an amino acid sequence at least 97% identical to SEQ ID NO: 10. In some embodiments, the albumin comprises an amino acid sequence at least 98% identical to SEQ ID NO: 10. In some embodiments, the albumin comprises an amino acid sequence at least 99% identical to SEQ ID NO: 10. In some embodiments, the albumin comprises an amino acid sequence identical to SEQ ID NO: 10.

In some embodiments, the Fc fusion molecule comprises an Fc fragment and an albumin binding domain. In some embodiments, the albumin binding domain binds to human serum albumin. In some embodiments, the albumin binding domain comprises an albumin binding domain described in WO2012175400A1, the contents of which are incorporated herein by reference in its entirety.

In some embodiments, the albumin binding domain comprises an amino acid sequence at least 80% identical to SEQ ID NO: 11. In some embodiments, the albumin binding domain comprises an amino acid sequence at least 85% identical to SEQ ID NO: 11. In some embodiments, the albumin binding domain comprises an amino acid sequence at least 90% identical to SEQ ID NO: 11. In some embodiments, the albumin binding domain comprises an amino acid sequence at least 95% identical to SEQ ID NO: 11. In some embodiments, the albumin binding domain comprises an amino acid sequence at least 96% identical to SEQ ID NO: 11. In some embodiments, the albumin binding domain comprises an amino acid sequence at least 97% identical to SEQ ID NO: 11. In some embodiments, the albumin binding domain comprises an amino acid sequence at least 98% identical to SEQ ID NO: 11. In some embodiments, the albumin binding domain comprises an amino acid sequence at least 99% identical to SEQ ID NO: 11. In some embodiments, the albumin binding domain comprises an amino acid sequence identical to SEQ ID NO: 11.

In some embodiments, the albumin binding domain comprises an amino acid substitution of F32A relative to SEQ ID NO: 11. In some embodiments, the albumin binding domain comprises an amino acid sequence of SEQ ID NO: 76.

In some embodiments, the albumin binding domain comprises an amino acid sequence at least 80% identical to SEQ ID NO: 12. In some embodiments, the albumin binding domain comprises an amino acid sequence at least 85% identical to SEQ ID NO: 12. In some embodiments, the albumin binding domain comprises an amino acid sequence at least 90% identical to SEQ ID NO: 12. In some embodiments, the albumin binding domain comprises an amino acid sequence at least 95% identical to SEQ ID NO: 12. In some embodiments, the albumin binding domain comprises an amino acid sequence at least 96% identical to SEQ ID NO: 12. In some embodiments, the albumin binding domain comprises an amino acid sequence at least 97% identical to SEQ ID NO: 12. In some embodiments, the albumin binding domain comprises an amino acid sequence at least 98% identical to SEQ ID NO: 12. In some embodiments, the albumin binding domain comprises an amino acid sequence at least 99% identical to SEQ ID NO: 12. In some embodiments, the albumin binding domain comprises an amino acid sequence identical to SEQ ID NO: 12.

In some embodiments, the albumin or albumin binding domain comprises a sequence having at least 80% sequence identity with an amino acid sequence according to any one of SEQ ID NOs: 10-12. In some embodiments, the albumin or albumin binding domain comprises a sequence having at least 85% sequence identity with an amino acid sequence according to any one of SEQ ID NOs: 10-12. In some embodiments, the albumin or albumin binding domain comprises a sequence having at least 90% sequence identity with an amino acid sequence according to any one of SEQ ID NOs: 10-12. In some embodiments, the albumin or albumin binding domain comprises a sequence having at least 95% sequence identity with an amino acid sequence according to any one of SEQ ID NOs: 10-12. In some embodiments, the albumin or albumin binding domain comprises a sequence having at least 96% sequence identity with an amino acid sequence according to any one of SEQ ID NOs: 10-12. In some embodiments, the albumin or albumin binding domain comprises a sequence having at least 97% sequence identity with an amino acid sequence according to any one of SEQ ID NOs: 10-12. In some embodiments, the albumin or albumin binding domain comprises a sequence having at least 98% sequence identity with an amino acid sequence according to any one of SEQ ID NOs: 10-12. In some embodiments, the albumin or albumin binding domain comprises a sequence having at least 99% sequence identity with an amino acid sequence according to any one of SEQ ID NOs: 10-12. In some embodiments, the albumin or albumin binding domain comprises a sequence according to any one of SEQ ID NOs: 10-12.

Exemplary amino acid sequences of albumin or albumin binding domains are shown in Table 1C.

TABLE 1C
Exemplary albumin or albumin binding domains.

| Identifier | Description | Amino Acid sequence |
|---|---|---|
| HSA | Human Serum | DAHKSEVAHRFKDLGEENFKALVLIAFAQYLQQCPFEDHVKLVNEVTE |
| (SEQ ID NO: 10) | Albumin | FAKTCVADESAENCDKSLHTLFGDKLCTVATLRETYGEMADCCAKQEP |
| | | ERNECFLQHKDDNPNLPRLVRPEVDVMCTAFHDNEETFLKKYLYEIAR |
| | | RHPYFYAPELLFFAKRYKAAFTECCQAADKAACLLPKLDELRDEGKAS |
| | | SAKQRLKCASLQKFGERAFKAWAVARLSQRFPKAEFAEVSKLVTDLTK |
| | | VHTECCHGDLLECADDRADLAKYICENQDSISSKLKECCEKPLLEKSH |
| | | CIAEVENDEMPADLPSLAADFVESKDVCKNYAEAKDVFLGMFLYEYAR |
| | | RHPDYSVVLLLRLAKTYETTLEKCCAAADPHECYAKVFDEFKPLVEEP |
| | | QNLIKQNCELFEQLGEYKFQNALLVRYTKKVPQVSTPTLVEVSRNLGK |
| | | VGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTA |
| | | LVELVKHKPKATKEQLKAVMDDFAAFVEKCCKADDKETCFAEEGKKLV |
| | | AASQAALGL |
| ABD1 | Albumin | EVQLLESGGGLVQPGGSLRLSCAASGFTFRSFGMSWVRQAPGKGPEWV |
| (SEQ ID NO: 11) | binding VHH | SSISGSGSDTLYADSVKGRFTISRDNSKNTLYLQMNSLRPEDTAVYYC |
| | domain | TIGGSLSRSSQGTLVTVSS |
| ABD2 | Albumin | EVQLVESGGGLVQPGNSLRLSCAASGFTFSSFGMSWVRQAPGKGLEWV |
| (SEQ ID NO: 12) | binding VHH | SSISGSGSDTLYADSVKGRFTISRDNAKTTLYLQMNSLRPEDTAVYYC |
| | domain | TIGGSLSRSSQGTLVTVSS |
| ABD1 (F32A) | ABD1 with | EVQLLESGGGLVQPGGSLRLSCAASGFTFRSAGMSWVRQAPGKGPEWV |
| (SEQ ID NO: 76) | F32A mutation | SSISGSGSDTLYADSVKGRFTISRDNSKNTLYLQMNSLRPEDTAVYYC |
| | | TIGGSLSRSSQGTLVTVSS |

### Fc Fusion Molecule Format

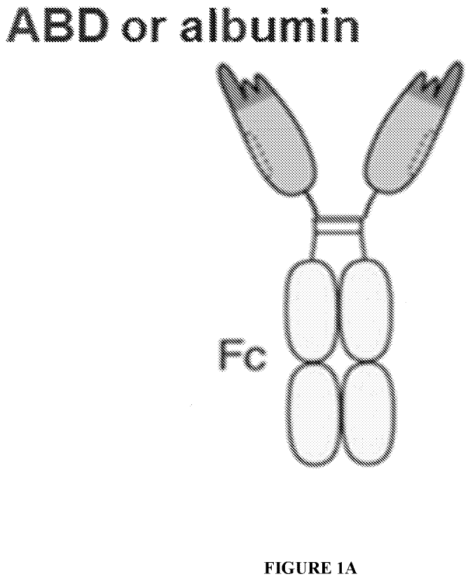

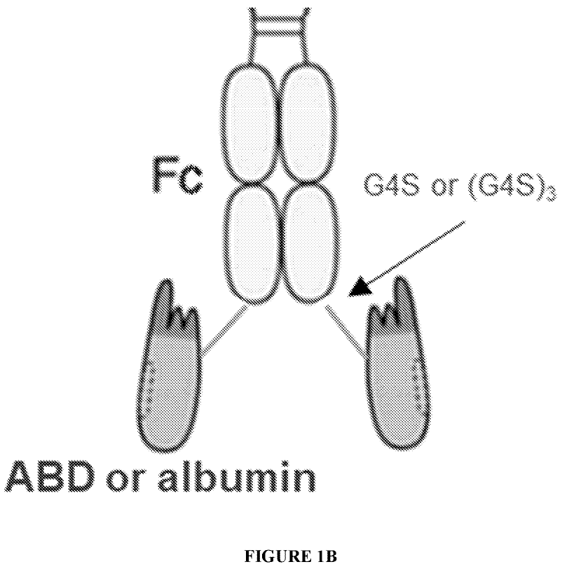

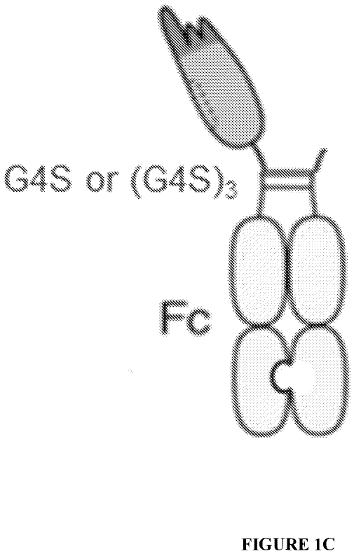

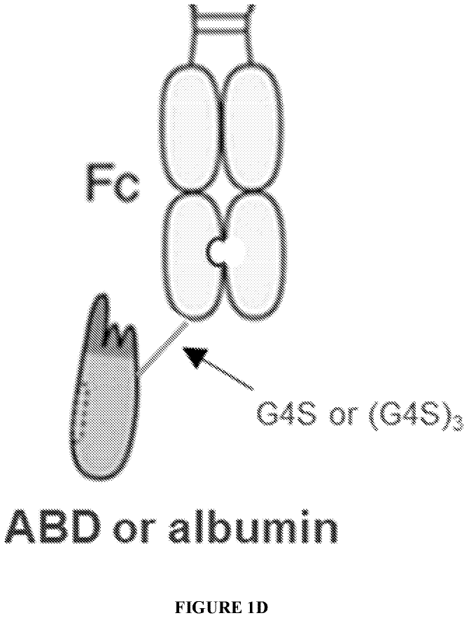

In some embodiments, the Fc fusion molecules described herein comprise an Fc fragment and a second domain. In some embodiments, the Fc fusion molecules comprise an Fc fragment, a linker, and a second domain. In some embodiments, the Fc fusion molecules comprise a format described in FIG. 1.

In some embodiments, the Fc fusion molecule comprises, from N-terminus to C-terminus, an Fc fragment, a linker, and an antigen binding domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from N-terminus to C-terminus, a second Fc fragment, a second linker, and a second antigen binding domain. In some embodiments, the first antigen binding domain and the second antigen binding domain are identical.

In some embodiments, the Fc fusion molecule comprises, from N-terminus to C-terminus, an Fc fragment, a linker, and a half-life extension domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from N-terminus to C-terminus, a second Fc fragment, a second linker, and a second half-life extension domain. In some embodiments, the first half-life extension domain and the second half-life extension domain are identical.

In some embodiments, the Fc fusion molecule comprises, from N-terminus to C-terminus, an Fc fragment, a linker, and an albumin. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from N-terminus to C-terminus, a second Fc fragment, a second linker, and a second albumin. In some embodiments, the first albumin and the second albumin are identical.

In some embodiments, the Fc fusion molecule comprises, from N-terminus to C-terminus, an Fc fragment, a linker, and an albumin binding domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from N-terminus to C-terminus, a second Fc fragment, a second linker, and a second albumin binding domain. In some embodiments, the first albumin binding domain and the second albumin binding domain are identical.

In some embodiments, the Fc fusion molecule comprises, from C-terminus to N-terminus, an Fc fragment, a linker, and an antigen binding domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from C-terminus to N-terminus, a second Fc fragment, a second linker, and a second antigen binding domain. In some embodiments, the first antigen binding domain and the second antigen binding domain are identical.

In some embodiments, the Fc fusion molecule comprises, from C-terminus to N-terminus, an Fc fragment, a linker, and a half-life extension domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from C-terminus to N-terminus, a second Fc fragment, a second linker, and a second half-life extension domain. In some embodiments, the first half-life extension domain and the second half-life extension domain are identical.

In some embodiments, the Fc fusion molecule comprises, from C-terminus to N-terminus, an Fc fragment, a linker, and an albumin. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from C-terminus to N-terminus, a second Fc fragment, a second linker, and a second albumin. In some embodiments, the first albumin and the second albumin are identical.

In some embodiments, the Fc fusion molecule comprises, from C-terminus to N-terminus, an Fc fragment, a linker, and an albumin binding domain. In some embodiments, the Fc fusion molecule forms a homodimer. In some embodiments, the Fc fusion molecule forms a heterodimer with a second Fc fragment. In some embodiments, the Fc fusion molecule forms a heterodimer with a second Fc fusion molecule comprising, from C-terminus to N-terminus, a second Fc fragment, a second linker, and a second albumin binding domain. In some embodiments, the first albumin binding domain and the second albumin binding domain are identical. In one aspect, the present invention provides, among other things, a bivalent Fc fusion molecule comprising two polypeptides, wherein each polypeptide comprises a half-life extension domain to the N-terminus of an Fc fragment.

In one aspect, the present invention provides, among other things, a bivalent Fc fusion molecule comprising two polypeptides, wherein each polypeptide comprises an anti-HSA VHH fused to the N-terminus of an Fc fragment.

In one aspect, the present invention provides, among other things, a bivalent Fc fusion molecule comprising two polypeptides, wherein each polypeptide comprises an albumin binding domain fused to the N-terminus of an Fc fragment.

In one aspect, the present invention provides, among other things, a bivalent Fc fusion molecule comprising two polypeptides, wherein each polypeptide comprises an albumin fused to the N-terminus of an Fc fragment.

In one aspect, the present invention provides, among other things, a bivalent Fc fusion molecule comprising two polypeptides, wherein each polypeptide comprises a half-life extension domain fused to the C-terminus of an Fc fragment.

In one aspect, the present invention provides, among other things, a bivalent Fc fusion molecule comprising two polypeptides, wherein each polypeptide comprises an anti-HSA VHH fused to the C-terminus of an Fc fragment.

In one aspect, the present invention provides, among other things, a bivalent Fc fusion molecule comprising two polypeptides, wherein each polypeptide comprises an albumin binding domain fused to the C-terminus of an Fc fragment.

In one aspect, the present invention provides, among other things, a bivalent Fc fusion molecule comprising two polypeptides, wherein each polypeptide comprises an albumin fused to the C-terminus of an Fc fragment.

In one aspect, the present invention provides, among other things, a monovalent Fc fusion molecule comprising two polypeptides, wherein the first polypeptide comprises a half-life extension domain fused to the N-terminus of a first Fc region, and the second polypeptide comprises a second Fc fragment.

In one aspect, the present invention provides, among other things, a monovalent Fc fusion molecule comprising two polypeptides, wherein the first polypeptide comprises an anti-HSA VHH fused to the N-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an albumin binding domain fused to the N-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an albumin fused to the N-terminus of a first Fc fragment, and the second polypeptide comprises a second Fc fragment.

In one aspect, the present invention provides, among other things, a monovalent Fc fusion molecule comprising two polypeptides, wherein the first polypeptide comprises a half-life extension domain fused to the C-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an anti-HSA VHH fused to the C-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an albumin binding domain fused to the C-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an albumin fused to the C-terminus of a first Fc fragment, and the second polypeptide comprises a second Fc fragment.

In one aspect, the present invention provides, among other things, a monovalent Fc fusion molecule comprising two polypeptides, wherein the first polypeptide comprises an anti-HSA VHH fused to the C-terminus of a first Fc fragment, and the second polypeptide comprises a second Fc fragment, wherein the first Fc fragment and the second Fc fragment comprise M252Y, S254T, T256E, H433K, and N434Y compared to an amino acid sequence set forth in SEQ ID NO: 56.

In one aspect, the present invention provides, among other things, a monovalent Fc fusion molecule comprising two polypeptides, wherein the first polypeptide comprises an anti-HSA VHH fused to the N-terminus of a first Fc fragment, and the second polypeptide comprises a second Fc fragment, wherein the first Fc fragment and the second Fc fragment comprise M252Y, S254T, T256E, H433K, and N434Y compared to an amino acid sequence set forth in SEQ ID NO: 56.

In one aspect, the present invention provides, among other things, a monovalent Fc fusion molecule comprising two polypeptides, wherein the first polypeptide comprises an anti-HSA VHH fused to the C-terminus of a first Fc fragment, and the second polypeptide comprises a second Fc fragment, wherein the first Fc fragment and the second Fc fragment comprise M252Y, S254T, T256E, M428L, H433K, and N434F compared to an amino acid sequence set forth in SEQ ID NO: 56.

In one aspect, the present invention provides, among other things, a monovalent Fc fusion molecule comprising two polypeptides, wherein the first polypeptide comprises an anti-HSA VHH fused to the N-terminus of a first Fc fragment, and the second polypeptide comprises a second Fc fragment, wherein the first Fc fragment and the second Fc fragment comprise M252Y, S254T, T256E, M428L, H433K, and N434F compared to an amino acid sequence set forth in SEQ ID NO: 56.

In some embodiments, the first Fc fragment and the second Fc fragment are identical. In some embodiments the first Fc fragment and the second Fc fragment are further modified to promote heterodimerization.

In some embodiments, the first Fc fragment and the second Fc fragment further comprise one or more amino acid substitutions selected from Y349C, S354C, T366S, T366W, T366Y, L368A, Y407T, Y407V. In some embodiments, the first Fc fragment further comprises amino acid substitutions Y349C, T366S, L368A, and Y407V, and the second Fc fragment further comprises amino acid substitutions S354C and T366W In some embodiments, the second Fc fragment further comprises amino acid substitutions Y349C, T366S, L368A, and Y407V, and the first Fc fragment further comprises amino acid substitutions S354C and T366W In some embodiments, the first Fc fragment further comprises amino acid substitutions T366S, L368A, and Y407V, and the second Fc fragment further comprises amino acid substitution T266W. In some embodiments, the second Fc fragment further comprises amino acid substitutions T366S, L368A, and Y407V, and the first Fc fragment further comprises amino acid substitution T266W.

Provided herein are Fc fusion molecules. Amino acid sequences of exemplary Fc fusion molecules and their components (e.g., albumin or albumin binding domain, Fc fragments, linkers) are provided in Table 1D.

TABLE 1D
Fc Fusion Molecules

| | | | Secondary Chain | N-terminal | | C-terminal |
|---|---|---|---|---|---|---|
| Name | Description | Full Sequence | (Knob into Hole) | Sequence | Linker | Sequence |
| Fc Fusion | Efgart-G4S- | DKTHTCPPCPAPELLG | N/A | DKTHTCPPCP | GGGGS | DAHKSEVAHRF |
| Molecule 1 | HSA | GPSVFLFPPKPKDTLY | | APELLGGPSV | (SEQ | KDLGEENFKAL |
| (SEQ ID | | ITREPEVTCVVVDVSH | | FLFPPKPKDT | ID | VLIAFAQYLQQ |
| NO: 16) | | EDPEVKFNWYVDGVEV | | LYITREPEVT | NO: | CPFEDHVKLVN |
| | | HNAKTKPREEQYNSTY | | CVVVDVSHED | 13) | EVTEFAKTCVA |
| | | RVVSVLIVLHQDWLNG | | PEVKFNWYVD | | DESAENCDKSL |
| | | KEYKCKVSNKALPAPI | | GVEVHNAKTK | | HTLFGDKLCTV |
| | | EKTISKAKGQPREPQV | | PREEQYNSTY | | ATLRETYGEMA |
| | | YTLPPSRDELTKNQVS | | RVVSVLTVLH | | DCCAKQEPERN |
| | | LTCLVKGFYPSDIAVE | | QDWLNGKEYK | | ECFLQHKDDNP |
| | | WESNGQPENNYKITPP | | CKVSNKALPA | | NLPRLVRPEVD |
| | | VLDSDGSFFLYSKLTV | | PIEKTISKAK | | VMCTAFHDNEE |
| | | DKSRWQQGNVFSCSVM | | GQPREPQVYT | | TFLKKYLYEIA |
| | | HEALKFHYTQKSLSLS | | LPPSRDELTK | | RRHPYFYAPEL |
| | | PGGGGGSDAHKSEVAH | | NQVSLTCLVK | | LFFAKRYKAAF |
| | | RFKDLGEENFKALVLI | | GFYPSDIAVE | | TECCQAADKAA |
| | | AFAQYLQQCPFEDHVK | | WESNGQPENN | | CLLPKLDELRD |
| | | LVNEVTEFAKTCVADE | | YKTTPPVLDS | | EGKASSAKQRL |
| | | SAENCDKSLHTLFGDK | | DGSFFLYSKL | | KCASLQKFGER |
| | | LCTVATLRETYGEMAD | | TVDKSRWQQG | | AFKAWAVARLS |
| | | CCAKQEPERNECFLQH | | NVFSCSVMHE | | QRFPKAEFAEV |
| | | KDDNPNLPRLVRPEVD | | ALKFHYTQKS | | SKLVTDLTKVH |
| | | VMCTAFHDNEETFLKK | | LSLSPG | | TECCHGDLLEC |
| | | YLYEIARRHPYFYAPE | | (SEQ ID | | ADDRADLAKYI |
| | | LLFFAKRYKAAFTECC | | NO: 1) | | CENQDSISSKI |
| | | QAADKAACLLPKLDEL | | | | KECCEKPLLEK |
| | | RDEGKASSAKQRLKCA | | | | SHCIAEVENDE |
| | | SLQKFGERAFKAWAVA | | | | MPADLPSLAAD |
| | | RLSQRFPKAEFAEVSK | | | | FVESKDVCKNY |
| | | LVTDLTKVHTECCHGD | | | | AEAKDVFLGMF |
| | | LLECADDRADLAKYIC | | | | LYEYARRHPDY |
| | | ENQDSISSKLKECCEK | | | | SVVLLLRLAKT |
| | | PLLEKSHCIAEVENDE | | | | YETTLEKCCAA |
| | | MPADLPSLAADFVESK | | | | ADPHECYAKVF |
| | | DVCKNYAEAKDVFLGM | | | | DEFKPLVEEPQ |
| | | FLYEYARRHPDYSVVL | | | | NLIKQNCELFE |
| | | LLRLAKTYETTLEKCC | | | | QLGEYKFQNAL |
| | | AAADPHECYAKVFDEF | | | | LVRYTKKVPQV |
| | | KPLVEEPQNLIKQNCE | | | | STPTLVEVSRN |
| | | LFEQLGEYKFQNALLV | | | | LGKVGSKCCKH |
| | | RYTKKVPQVSTPTLVE | | | | PEAKRMPCAED |
| | | VSRNLGKVGSKCCKHP | | | | YLSVVLNQLCV |
| | | EAKRMPCAEDYLSVVL | | | | LHEKTPVSDRV |
| | | NQLCVLHEKTPVSDRV | | | | TKCCTESLVNR |
| | | TKCCTESLVNRRPCFS | | | | RPCFSALEVDE |
| | | ALEVDETYVPKEFNAE | | | | TYVPKEFNAET |
| | | TFTFHADICTLSEKER | | | | FTFHADICTLS |
| | | QIKKQTALVELVKHKP | | | | EKERQIKKQTA |
| | | KATKEQLKAVMDDFAA | | | | LVELVKHKPKA |
| | | FVEKCCKADDKETCFA | | | | TKEQLKAVMDD |
| | | EEGKKLVAASQAALGL | | | | FAAFVEKCCKA |
| | | | | | | DDKETCFAEEG |
| | | | | | | KKLVAASQAAL |
| | | | | | | GL (SEQ ID |
| | | | | | | NO: 10) |
| Fc Fusion | HSA-G4S- | DAHKSEVAHRFKDLGE | N/A | DAHKSEVAHR | GGGGS | DKTHTCPPCPA |
| Molecule 2 | Efgart | ENFKALVLIAFAQYLQ | | FKDLGEENFK | (SEQ | PELLGGPSVFL |
| (SEQ ID | | QCPFEDHVKLVNEVTE | | ALVLIAFAQY | ID | FPPKPKDTLYI |
| NO: 17) | | FAKTCVADESAENCDK | | LQQCPFEDHV | NO: | TREPEVTCVVV |
| | | SLHTLFGDKLCTVATL | | KLVNEVTEFA | 13) | DVSHEDPEVKF |
| | | RETYGEMADCCAKQEP | | KTCVADESAE | | NWYVDGVEVHN |
| | | ERNECFLQHKDDNPNL | | NCDKSLHTLF | | AKTKPREEQYN |
| | | PRLVRPEVDVMCTAFH | | GDKLCTVATL | | STYRVVSVLTV |
| | | DNEETFLKKYLYEIAR | | RETYGEMADC | | LHQDWLNGKEY |
| | | RHPYFYAPELLFFAKR | | CAKQEPERNE | | KCKVSNKALPA |
| | | YKAAFTECCQAADKAA | | CFLQHKDDNP | | PIEKTISKAKG |
| | | CLLPKLDELRDEGKAS | | NLPRLVRPEV | | QPREPQVYTLP |
| | | SAKQRLKCASLQKFGE | | DVMCTAFHDN | | PSRDELTKNQV |
| | | RAFKAWAVARLSQRFP | | EETFLKKYLY | | SLTCLVKGFYP |
| | | KAEFAEVSKLVTDLTK | | EIARRHPYFY | | SDIAVEWESNG |
| | | VHTECCHGDLLECADD | | APELLFFAKR | | QPENNYKTTPP |
| | | RADLAKYICENQDSIS | | YKAAFTECCQ | | VLDSDGSFFLY |
| | | SKLKECCEKPLLEKSH | | AADKAACLLP | | SKLTVDKSRWQ |
| | | CIAEVENDEMPADLPS | | KLDELRDEGK | | QGNVFSCSVMH |
| | | LAADFVESKDVCKNYA | | ASSAKQRLKC | | EALKFHYTQKS |
| | | EAKDVFLGMELYEYAR | | ASLQKFGERA | | LSLSPG (SEQ |
| | | RHPDYSVVLLLRLAKT | | FKAWAVARLS | | ID NO: 1) |
| | | YETTLEKCCAAADPHE | | QRFPKAEFAE | | |
| | | CYAKVFDEFKPLVEEP | | VSKLVTDLTK | | |
| | | QNLIKQNCELFEQLGE | | VHTECCHGDL | | |
| | | YKFQNALLVRYTKKVP | | LECADDRADL | | |
| | | QVSTPTLVEVSRNLGK | | AKYICENQDS | | |
| | | VGSKCCKHPEAKRMPC | | ISSKLKECCE | | |
| | | AEDYLSVVLNQLCVLH | | KPLLEKSHCI | | |
| | | EKTPVSDRVTKCCTES | | AEVENDEMPA | | |
| | | LVNRRPCFSALEVDET | | DLPSLAADFV | | |
| | | YVPKEFNAETFTFHAD | | ESKDVCKNYA | | |
| | | ICTLSEKERQIKKQTA | | EAKDVFLGMF | | |
| | | LVELVKHKPKATKEQL | | LYEYARRHPD | | |
| | | KAVMDDFAAFVEKCCK | | YSVVLLLRLA | | |
| | | ADDKETCFAEEGKKLV | | KTYETTLEKC | | |
| | | AASQAALGLGGGGSDK | | CAAADPHECY | | |
| | | THTCPPCPAPELLGGP | | AKVFDEFKPL | | |
| | | SVFLFPPKPKDTLYIT | | VEEPQNLIKQ | | |
| | | REPEVTCVVVDVSHED | | NCELFEQLGE | | |
| | | PEVKFNWYVDGVEVHN | | YKFQNALLVR | | |
| | | AKTKPREEQYNSTYRV | | YTKKVPQVST | | |
| | | VSVLTVLHQDWLNGKE | | PTLVEVSRNL | | |
| | | YKCKVSNKALPAPIEK | | GKVGSKCCKH | | |
| | | TISKAKGQPREPQVYT | | PEAKRMPCAE | | |
| | | LPPSRDELTKNQVSLT | | DYLSVVLNQL | | |
| | | CLVKGFYPSDIAVEWE | | CVLHEKTPVS | | |
| | | SNGQPENNYKTTPPVL | | DRVTKCCTES | | |
| | | DSDGSFFLYSKLTVDK | | LVNRRPCFSA | | |
| | | SRWQQGNVFSCSVMHE | | LEVDETYVPK | | |
| | | ALKFHYTQKSLSLSPG | | EFNAETFTFH | | |
| | | | | ADICTLSEKE | | |
| | | | | RQIKKQTALV | | |
| | | | | ELVKHKPKAT | | |
| | | | | KEQLKAVMDD | | |
| | | | | FAAFVEKCCK | | |
| | | | | ADDKETCFAE | | |
| | | | | EGKKLVAASQ | | |
| | | | | AALGL | | |
| | | | | (SEQ ID | | |
| | | | | NO: 10) | | |
| Fc Fusion | Efgart- | DKTHTCPPCPAPELLG | N/A | DKTHTCPPCP | GGGGS | DAHKSEVAHRF |
| Molecule 3 | (G4S)7-HSA | GPSVFLFPPKPKDTLY | | APELLGGPSV | GGGGS | KDLGEENFKAL |
| (SEQ ID | | ITREPEVTCVVVDVSH | | FLFPPKPKDT | GGGGS | VLIAFAQYLQQ |
| NO: 18) | | EDPEVKFNWYVDGVEV | | LYITREPEVT | GGGGS | CPFEDHVKLVN |
| | | HNAKTKPREEQYNSTY | | CVVVDVSHED | GGGGS | EVTEFAKTCVA |
| | | RVVSVLTVLHQDWLNG | | PEVKFNWYVD | GGGGS | DESAENCDKSL |
| | | KEYKCKVSNKALPAPI | | GVEVHNAKTK | GGGGS | HTLFGDKLCTV |
| | | EKTISKAKGQPREPQV | | PREEQYNSTY | (SEQ | ATLRETYGEMA |
| | | YTLPPSRDELTKNQVS | | RVVSVLTVLH | ID | DCCAKQEPERN |
| | | LTCLVKGFYPSDIAVE | | QDWLNGKEYK | NO: | ECFLQHKDDNP |
| | | WESNGQPENNYKTTPP | | CKVSNKALPA | 14) | NLPRLVRPEVD |
| | | VLDSDGSFFLYSKLTV | | PIEKTISKAK | | VMCTAFHDNEE |
| | | DKSRWQQGNVFSCSVM | | GQPREPQVYT | | TFLKKYLYEIA |
| | | HEALKFHYTQKSLSLS | | LPPSRDELTK | | RRHPYFYAPEL |
| | | PGGGGGSGGGGSGGGG | | NQVSLTCLVK | | LFFAKRYKAAF |
| | | SGGGGSGGGGSGGGGS | | GFYPSDIAVE | | TECCQAADKAA |
| | | GGGGSDAHKSEVAHRF | | WESNGQPENN | | CLLPKLDELRD |
| | | KDLGEENFKALVLIAF | | YKTTPPVLDS | | EGKASSAKQRL |
| | | AQYLQQCPFEDHVKLV | | DGSFFLYSKL | | KCASLQKFGER |
| | | NEVTEFAKTCVADESA | | TVDKSRWQQG | | AFKAWAVARLS |
| | | ENCDKSLHTLFGDKLC | | NVFSCSVMHE | | QRFPKAEFAEV |
| | | TVATLRETYGEMADCC | | ALKFHYTQKS | | SKLVTDLTKVH |
| | | AKQEPERNECFLQHKD | | LSLSPG | | TECCHGDLLEC |
| | | DNPNLPRLVRPEVDVM | | (SEQ ID | | ADDRADLAKYI |
| | | CTAFHDNEETFLKKYL | | NO: 1) | | CENQDSISSKL |
| | | YEIARRHPYFYAPELL | | | | KECCEKPLLEK |
| | | FFAKRYKAAFTECCQA | | | | SHCIAEVENDE |
| | | ADKAACLLPKLDELRD | | | | MPADLPSLAAD |
| | | EGKASSAKQRLKCASL | | | | FVESKDVCKNY |
| | | QKFGERAFKAWAVARL | | | | AEAKDVFLGMF |
| | | SQRFPKAEFAEVSKLV | | | | LYEYARRHPDY |
| | | TDLTKVHTECCHGDLL | | | | SVVLLLRLAKT |
| | | ECADDRADLAKYICEN | | | | YETTLEKCCAA |
| | | QDSISSKLKECCEKPL | | | | ADPHECYAKVF |
| | | LEKSHCIAEVENDEMP | | | | DEFKPLVEEPQ |
| | | ADLPSLAADFVESKDV | | | | NLIKQNCELFE |
| | | CKNYAEAKDVFLGMFL | | | | QLGEYKFQNAL |
| | | YEYARRHPDYSVVLLL | | | | LVRYTKKVPQV |
| | | RLAKTYETTLEKCCAA | | | | STPTLVEVSRN |
| | | ADPHECYAKVFDEFKP | | | | LGKVGSKCCKH |
| | | LVEEPQNLIKQNCELF | | | | PEAKRMPCAED |
| | | EQLGEYKFQNALLVRY | | | | YLSVVLNQLCV |
| | | TKKVPQVSTPTLVEVS | | | | LHEKTPVSDRV |
| | | RNLGKVGSKCCKHPEA | | | | TKCCTESLVNR |
| | | KRMPCAEDYLSVVLNQ | | | | RPCFSALEVDE |
| | | LCVLHEKTPVSDRVTK | | | | TYVPKEFNAET |
| | | CCTESLVNRRPCFSAL | | | | FTFHADICTLS |
| | | EVDETYVPKEFNAETF | | | | EKERQIKKQTA |
| | | TFHADICTLSEKERQI | | | | LVELVKHKPKA |
| | | KKQTALVELVKHKPKA | | | | TKEQLKAVMDD |
| | | TKEQLKAVMDDFAAFV | | | | FAAFVEKCCKA |
| | | EKCCKADDKETCFAEE | | | | DDKETCFAEEG |
| | | GKKLVAASQAALGL | | | | KKLVAASQAAL |
| | | | | | | GL (SEQ ID |
| | | | | | | NO: 10) |
| Fc Fusion | HSA- | DAHKSEVAHRFKDLGE | N/A | DAHKSEVAHR | GGGGS | DKTHTCPPCPA |
| Molecule 4 | (G4S)7- | ENFKALVLIAFAQYLQ | | FKDLGEENFK | GGGGS | PELLGGPSVFL |
| (SEQ ID | Efgart | QCPFEDHVKLVNEVTE | | ALVLIAFAQY | GGGGS | FPPKPKDTLYI |
| NO: 19) | | FAKTCVADESAENCDK | | LQQCPFEDHV | GGGGS | TREPEVTCVVV |
| | | SLHTLFGDKLCTVATL | | KLVNEVTEFA | GGGGS | DVSHEDPEVKF |
| | | RETYGEMADCCAKQEP | | KTCVADESAE | GGGGS | NWYVDGVEVHN |
| | | ERNECFLQHKDDNPNL | | NCDKSLHTLF | GGGGS | AKTKPREEQYN |
| | | PRLVRPEVDVMCTAFH | | GDKLCTVATL | (SEQ | STYRVVSVLIV |
| | | DNEETFLKKYLYEIAR | | RETYGEMADC | ID | LHQDWLNGKEY |
| | | RHPYFYAPELLFFAKR | | CAKQEPERNE | NO: | KCKVSNKALPA |
| | | YKAAFTECCQAADKAA | | CFLQHKDDNP | 14) | PIEKTISKAKG |
| | | CLLPKLDELRDEGKAS | | NLPRLVRPEV | | QPREPQVYTLP |
| | | SAKQRLKCASLQKFGE | | DVMCTAFHDN | | PSRDELTKNQV |
| | | RAFKAWAVARLSQRFP | | EETFLKKYLY | | SLTCLVKGFYP |
| | | KAEFAEVSKLVTDLTK | | EIARRHPYFY | | SDIAVEWESNG |
| | | VHTECCHGDLLECADD | | APELLFFAKR | | QPENNYKTTPP |
| | | RADLAKYICENQDSIS | | YKAAFTECCQ | | VLDSDGSFFLY |
| | | SKLKECCEKPLLEKSH | | AADKAACLLP | | SKLTVDKSRWQ |
| | | CIAEVENDEMPADLPS | | KLDELRDEGK | | QGNVFSCSVMH |
| | | LAADFVESKDVCKNYA | | ASSAKQRLKC | | EALKFHYTQKS |
| | | EAKDVFLGMFLYEYAR | | ASLQKFGERA | | LSLSPG (SEQ |
| | | RHPDYSVVLLLRLAKT | | FKAWAVARLS | | ID NO: 1) |
| | | YETTLEKCCAAADPHE | | QRFPKAEFAE | | |
| | | CYAKVFDEFKPLVEEP | | VSKLVTDLTK | | |
| | | QNLIKQNCELFEQLGE | | VHTECCHGDL | | |
| | | YKFQNALLVRYTKKVP | | LECADDRADL | | |
| | | QVSTPTLVEVSRNLGK | | AKYICENQDS | | |
| | | VGSKCCKHPEAKRMPC | | ISSKLKECCE | | |
| | | AEDYLSVVLNQLCVLH | | KPLLEKSHCI | | |
| | | EKTPVSDRVTKCCTES | | AEVENDEMPA | | |
| | | LVNRRPCFSALEVDET | | DLPSLAADFV | | |
| | | YVPKEFNAETFTFHAD | | ESKDVCKNYA | | |
| | | ICTLSEKERQIKKQTA | | EAKDVFLGMF | | |
| | | LVELVKHKPKATKEQL | | LYEYARRHPD | | |
| | | KAVMDDFAAFVEKCCK | | YSVVLLLRLA | | |
| | | ADDKETCFAEEGKKLV | | KTYETTLEKC | | |
| | | AASQAALGLGGGGSGG | | CAAADPHECY | | |
| | | GGSGGGGSGGGGSGGG | | AKVFDEFKPL | | |
| | | GSGGGGSGGGGSDKTH | | VEEPQNLIKQ | | |
| | | TCPPCPAPELLGGPSV | | NCELFEQLGE | | |
| | | FLFPPKPKDTLYITRE | | YKFQNALLVR | | |
| | | PEVTCVVVDVSHEDPE | | YTKKVPQVST | | |
| | | VKFNWYVDGVEVHNAK | | PTLVEVSRNL | | |
| | | TKPREEQYNSTYRVVS | | GKVGSKCCKH | | |
| | | VLTVLHQDWLNGKEYK | | PEAKRMPCAE | | |
| | | CKVSNKALPAPIEKTI | | DYLSVVLNQL | | |
| | | SKAKGQPREPQVYTLP | | CVLHEKTPVS | | |
| | | PSRDELTKNQVSLTCL | | DRVTKCCTES | | |
| | | VKGFYPSDIAVEWESN | | LVNRRPCFSA | | |
| | | GQPENNYKTTPPVLDS | | LEVDETYVPK | | |
| | | DGSFFLYSKLTVDKSR | | EFNAETFTFH | | |
| | | WQQGNVFSCSVMHEAL | | ADICTLSEKE | | |
| | | KFHYTQKSLSLSPG | | RQIKKQTALV | | |
| | | | | ELVKHKPKAT | | |
| | | | | KEQLKAVMDD | | |
| | | | | FAAFVEKCCK | | |
| | | | | ADDKETCFAE | | |
| | | | | EGKKLVAASQ | | |
| | | | | AALGL (SEQ | | |
| | | | | ID NO: 10) | | |
| Fc Fusion | ABD1-G4S- | EVQLLESGGGLVQPGG | N/A | EVQLLESGGG | GGGGS | DKTHTCPPCPA |
| Molecule 5 | Efgart | SLRLSCAASGFTFRSF | | LVQPGGSLRL | (SEQ | PELLGGPSVFL |
| (SEQ ID | | GMSWVRQAPGKGPEWV | | SCAASGFTFR | ID | FPPKPKDTLYI |
| NO: 20) | | SSISGSGSDTLYADSV | | SFGMSWVRQA | NO: | TREPEVTCVVV |
| | | KGRFTISRDNSKNTLY | | PGKGPEWVSS | 13) | DVSHEDPEVKF |
| | | LQMNSLRPEDTAVYYC | | ISGSGSDTLY | | NWYVDGVEVHN |
| | | TIGGSLSRSSQGTLVT | | ADSVKGRFTI | | AKTKPREEQYN |
| | | VSSGGGGSDKTHTCPP | | SRDNSKNTLY | | STYRVVSVLTV |
| | | CPAPELLGGPSVFLFP | | LQMNSLRPED | | LHQDWLNGKEY |
| | | PKPKDTLYITREPEVT | | TAVYYCTIGG | | KCKVSNKALPA |
| | | CVVVDVSHEDPEVKFN | | SLSRSSQGTL | | PIEKTISKAKG |
| | | WYVDGVEVHNAKTKPR | | VTVSS (SEQ | | QPREPQVYTLP |
| | | EEQYNSTYRVVSVLTV | | ID NO: 11) | | PSRDELTKNQV |
| | | LHQDWLNGKEYKCKVS | | | | SLTCLVKGFYP |
| | | NKALPAPIEKTISKAK | | | | SDIAVEWESNG |
| | | GQPREPQVYTLPPSRD | | | | QPENNYKTTPP |
| | | ELTKNQVSLTCLVKGF | | | | VLDSDGSFFLY |
| | | YPSDIAVEWESNGQPE | | | | SKLTVDKSRWQ |
| | | NNYKTTPPVLDSDGSF | | | | QGNVFSCSVMH |
| | | FLYSKLTVDKSRWQQG | | | | EALKFHYTQKS |
| | | NVFSCSVMHEALKFHY | | | | LSLSPG (SEQ |
| | | TQKSLSLSPG | | | | ID NO: 1) |
| Fc Fusion | Efgart-G4S- | DKTHTCPPCPAPELLG | N/A | DKTHTCPPCP | GGGGS | EVQLLESGGGL |
| Molecule 6 | ABD1 | GPSVFLFPPKPKDTLY | | APELLGGPSV | (SEQ | VQPGGSLRLSC |
| (SEQ ID | | ITREPEVTCVVVDVSH | | FLFPPKPKDT | ID | AASGFTFRSFG |
| NO: 21) | | EDPEVKFNWYVDGVEV | | LYITREPEVT | NO: | MSWVRQAPGKG |
| | | HNAKTKPREEQYNSTY | | CVVVDVSHED | 13) | PEWVSSISGSG |
| | | RVVSVLTVLHQDWLNG | | PEVKFNWYVD | | SDTLYADSVKG |
| | | KEYKCKVSNKALPAPI | | GVEVHNAKTK | | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVM | | GQPREPQVYT | | |
| | | HEALKFHYTQKSLSLS | | LPPSRDELTK | | |
| | | PGGGGGSEVQLLESGG | | NQVSLTCLVK | | |
| | | GLVQPGGSLRLSCAAS | | GFYPSDIAVE | | |
| | | GFTFRSFGMSWVRQAP | | WESNGQPENN | | |
| | | GKGPEWVSSISGSGSD | | YKTTPPVLDS | | |
| | | TLYADSVKGRFTISRD | | DGSFFLYSKL | | |
| | | NSKNTLYLQMNSLRPE | | TVDKSRWQQG | | |
| | | DTAVYYCTIGGSLSRS | | NVFSCSVMHE | | |
| | | SQGTLVTVSS | | ALKFHYTQKS | | |
| | | | | LSLSPG | | |
| | | | | (SEQ ID | | |
| | | | | NO: 1) | | |
| Fc Fusion | ABD1- | EVQLLESGGGLVQPGG | N/A | EVQLLESGGG | GGGGS | DKTHTCPPCPA |
| Molecule 7 | (G4S)3- | SLRLSCAASGFTFRSF | | LVQPGGSLRL | GGGGS | PELLGGPSVFL |
| (SEQ ID | Efgart | GMSWVRQAPGKGPEWV | | SCAASGFTFR | GGGGS | FPPKPKDTLYI |
| NO: 22) | | SSISGSGSDTLYADSV | | SFGMSWVRQA | (SEQ | TREPEVTCVVV |
| | | KGRFTISRDNSKNTLY | | PGKGPEWVSS | ID | DVSHEDPEVKF |
| | | LQMNSLRPEDTAVYYC | | ISGSGSDTLY | NO: | NWYVDGVEVHN |
| | | TIGGSLSRSSQGTLVT | | ADSVKGRFTI | 15) | AKTKPREEQYN |
| | | VSSGGGGSGGGGSGGG | | SRDNSKNTLY | | STYRVVSVLIV |
| | | GSDKTHTCPPCPAPEL | | LQMNSLRPED | | LHQDWLNGKEY |
| | | LGGPSVFLFPPKPKDT | | TAVYYCTIGG | | KCKVSNKALPA |
| | | LYITREPEVTCVVVDV | | SLSRSSQGTL | | PIEKTISKAKG |
| | | SHEDPEVKFNWYVDGV | | VTVSS (SEQ | | QPREPQVYTLP |
| | | EVHNAKTKPREEQYNS | | ID NO: 11) | | PSRDELTKNQV |
| | | TYRVVSVLTVLHQDWL | | | | SLTCLVKGFYP |
| | | NGKEYKCKVSNKALPA | | | | SDIAVEWESNG |
| | | PIEKTISKAKGQPREP | | | | QPENNYKTTPP |
| | | QVYTLPPSRDELTKNQ | | | | VLDSDGSFFLY |
| | | VSLTCLVKGFYPSDIA | | | | SKLTVDKSRWQ |
| | | VEWESNGQPENNYKTT | | | | QGNVFSCSVMH |
| | | PPVLDSDGSFFLYSKL | | | | EALKFHYTQKS |
| | | TVDKSRWQQGNVFSCS | | | | LSLSPG (SEQ |
| | | VMHEALKFHYTQKSLS | | | | ID NO: 1) |
| | | LSPG | | | | |
| Fc Fusion | Efgart- | DKTHTCPPCPAPELLG | N/A | DKTHTCPPCP | GGGGS | EVQLLESGGGL |
| Molecule 8 | (G4S)3- | GPSVFLFPPKPKDTLY | | APELLGGPSV | GGGGS | VQPGGSLRLSC |
| (SEQ ID | ABD1 | ITREPEVTCVVVDVSH | | FLFPPKPKDT | GGGGS | AASGFTFRSFG |
| NO: 23) | | EDPEVKFNWYVDGVEV | | LYITREPEVT | (SEQ | MSWVRQAPGKG |
| | | HNAKTKPREEQYNSTY | | CVVVDVSHED | ID | PEWVSSISGSG |
| | | RVVSVLTVLHQDWLNG | | PEVKFNWYVD | NO: | SDTLYADSVKG |
| | | KEYKCKVSNKALPAPI | | GVEVHNAKTK | 15) | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVM | | GQPREPQVYT | | |
| | | HEALKFHYTQKSLSLS | | LPPSRDELTK | | |
| | | PGGGGGSGGGGSGGGG | | NQVSLTCLVK | | |
| | | SEVQLLESGGGLVQPG | | GFYPSDIAVE | | |
| | | GSLRLSCAASGFTFRS | | WESNGQPENN | | |
| | | FGMSWVRQAPGKGPEW | | YKTTPPVLDS | | |
| | | VSSISGSGSDTLYADS | | DGSFFLYSKL | | |
| | | VKGRFTISRDNSKNTL | | TVDKSRWQQG | | |
| | | YLQMNSLRPEDTAVYY | | NVFSCSVMHE | | |
| | | CTIGGSLSRSSQGTLV | | ALKFHYTQKS | | |
| | | TVSS | | LSLSPG | | |
| | | | | (SEQ ID | | |
| | | | | NO: 1) | | |
| Fc Fusion | ABD1-G4S- | EVQLLESGGGLVQPGG | DKTHTCPPCPAPEL | EVQLLESGGG | GGGGS | DKTHTCPPCPA |
| Molecule 9 | Efgart(KIH) | SLRLSCAASGFTFRSF | LGGPSVFLFPPKPK | LVQPGGSLRL | (SEQ | PELLGGPSVFL |
| (SEQ ID | | GMSWVRQAPGKGPEWV | DTLYITREPEVTCV | SCAASGFTFR | ID | FPPKPKDTLYI |
| NO: 24) | | SSISGSGSDTLYADSV | VVDVSHEDPEVKFN | SFGMSWVRQA | NO: | TREPEVTCVVV |
| | | KGRFTISRDNSKNTLY | WYVDGVEVHNAKTK | PGKGPEWVSS | 13) | DVSHEDPEVKF |
| | | LQMNSLRPEDTAVYYC | PREEQYNSTYRVVS | ISGSGSDTLY | | NWYVDGVEVHN |
| | | TIGGSLSRSSQGTLVT | VLTVLHQDWLNGKE | ADSVKGRFTI | | AKTKPREEQYN |
| | | VSSGGGGSDKTHTCPP | YKCKVSNKALPAPI | SRDNSKNTLY | | STYRVVSVLTV |
| | | CPAPELLGGPSVFLFP | EKTISKAKGQPREP | LQMNSLRPED | | LHQDWLNGKEY |
| | | PKPKDTLYITREPEVT | QVYTLPPCRDELTK | TAVYYCTIGG | | KCKVSNKALPA |
| | | CVVVDVSHEDPEVKFN | NQVSLWCLVKGFYP | SLSRSSQGTL | | PIEKTISKAKG |
| | | WYVDGVEVHNAKTKPR | SDIAVEWESNGQPE | VTVSS (SEQ | | QPREPQVCTLP |
| | | EEQYNSTYRVVSVLTV | NNYKTTPPVLDSDG | ID NO: 11) | | PSRDELTKNQV |
| | | LHQDWLNGKEYKCKVS | SFFLYSKLTVDKSR | | | SLSCAVKGFYP |
| | | NKALPAPIEKTISKAK | WQQGNVFSCSVMHE | | | SDIAVEWESNG |
| | | GQPREPQVCTLPPSRD | ALKFHYTQKSLSLS | | | QPENNYKTTPP |
| | | ELTKNQVSLSCAVKGF | PG (SEQ ID NO: | | | VLDSDGSFFLV |
| | | YPSDIAVEWESNGQPE | 5) | | | SKLTVDKSRWQ |
| | | NNYKTTPPVLDSDGSF | | | | QGNVFSCSVMH |
| | | FLVSKLTVDKSRWQQG | | | | EALKFHYTQKS |
| | | NVFSCSVMHEALKFHY | | | | LSLSPG (SEQ |
| | | TQKSLSLSPG | | | | ID NO: 4) |
| Fc Fusion | Efgart(KIH)- | DKTHTCPPCPAPELLG | DKTHTCPPCPAPEL | DKTHTCPPCP | GGGGS | EVQLLESGGGL |
| Molecule | G4S-ABD1 | GPSVFLFPPKPKDTLY | LGGPSVFLFPPKPK | APELLGGPSV | (SEQ | VQPGGSLRLSC |
| 10 | | ITREPEVTCVVVDVSH | DTLYITREPEVTCV | FLFPPKPKDT | ID | AASGFTFRSFG |
| (SEQ ID | | EDPEVKFNWYVDGVEV | VVDVSHEDPEVKFN | LYITREPEVT | NO: | MSWVRQAPGKG |
| NO: 25) | | HNAKTKPREEQYNSTY | WYVDGVEVHNAKTK | CVVVDVSHED | 13) | PEWVSSISGSG |
| | | RVVSVLTVLHQDWLNG | PREEQYNSTYRVVS | PEVKFNWYVD | | SDTLYADSVKG |
| | | KEYKCKVSNKALPAPI | VLTVLHQDWLNGKE | GVEVHNAKTK | | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVM | NNYKTTPPVLDSDG | GQPREPQVCT | | |
| | | HEALKFHYTQKSLSLS | SFFLYSKLTVDKSR | LPPSRDELTK | | |
| | | PGGGGGSEVQLLESGG | WQQGNVFSCSVMHE | NQVSLSCAVK | | |
| | | GLVQPGGSLRLSCAAS | ALKFHYTQKSLSLS | GFYPSDIAVE | | |
| | | GFTFRSFGMSWVRQAP | PG (SEQ ID NO: | WESNGQPENN | | |
| | | GKGPEWVSSISGSGSD | 5) | YKTTPPVLDS | | |
| | | TLYADSVKGRFTISRD | | DGSFFLVSKL | | |
| | | NSKNTLYLQMNSLRPE | | TVDKSRWQQG | | |
| | | DTAVYYCTIGGSLSRS | | NVFSCSVMHE | | |
| | | SQGTLVTVSS | | ALKFHYTQKS | | |
| | | | | LSLSPG | | |
| | | | | (SEQ ID | | |
| | | | | NO: 4) | | |
| Fc Fusion | ABD1- | EVQLLESGGGLVQPGG | DKTHTCPPCPAPEL | EVQLLESGGG | GGGGS | DKTHTCPPCPA |
| Molecule | (G4S)3- | SLRLSCAASGFTFRSF | LGGPSVFLFPPKPK | LVQPGGSLRL | GGGGS | PELLGGPSVFL |
| 11 | Efgart(KIH) | GMSWVRQAPGKGPEWV | DTLYITREPEVTCV | SCAASGFTFR | GGGGS | FPPKPKDTLYI |
| (SEQ ID | | SSISGSGSDTLYADSV | VVDVSHEDPEVKFN | SFGMSWVRQA | (SEQ | TREPEVTCVVV |
| NO: 26) | | KGRFTISRDNSKNTLY | WYVDGVEVHNAKTK | PGKGPEWVSS | ID | DVSHEDPEVKF |
| | | LQMNSLRPEDTAVYYC | PREEQYNSTYRVVS | ISGSGSDTLY | NO: | NWYVDGVEVHN |
| | | TIGGSLSRSSQGTLVT | VLTVLHQDWLNGKE | ADSVKGRFTI | 15) | AKTKPREEQYN |
| | | VSSGGGGSGGGGSGGG | YKCKVSNKALPAPI | SRDNSKNTLY | | STYRVVSVLTV |
| | | GSDKTHTCPPCPAPEL | EKTISKAKGQPREP | LQMNSLRPED | | LHQDWLNGKEY |
| | | LGGPSVFLFPPKPKDT | QVYTLPPCRDELTK | TAVYYCTIGG | | KCKVSNKALPA |
| | | LYITREPEVTCVVVDV | NQVSLWCLVKGFYP | SLSRSSQGTL | | PIEKTISKAKG |
| | | SHEDPEVKFNWYVDGV | SDIAVEWESNGQPE | VTVSS (SEQ | | QPREPQVCTLP |
| | | EVHNAKTKPREEQYNS | NNYKTTPPVLDSDG | ID NO: 11) | | PSRDELTKNQV |
| | | TYRVVSVLIVLHQDWL | SFFLYSKLTVDKSR | | | SLSCAVKGFYP |
| | | NGKEYKCKVSNKALPA | WQQGNVFSCSVMHE | | | SDIAVEWESNG |
| | | PIEKTISKAKGQPREP | ALKFHYTQKSLSLS | | | QPENNYKTTPP |
| | | QVCTLPPSRDELTKNQ | PG (SEQ ID NO: | | | VLDSDGSFFLV |
| | | VSLSCAVKGFYPSDIA | 5) | | | SKLTVDKSRWQ |
| | | VEWESNGQPENNYKTT | | | | QGNVFSCSVMH |
| | | PPVLDSDGSFFLVSKL | | | | EALKFHYTQKS |
| | | TVDKSRWQQGNVFSCS | | | | LSLSPG (SEQ |
| | | VMHEALKFHYTQKSLS | | | | ID NO: 4) |
| | | LSPG | | | | |
| Fc Fusion | Efgart(KIH)- | DKTHTCPPCPAPELLG | DKTHTCPPCPAPEL | DKTHTCPPCP | GGGGS | EVQLLESGGGL |
| Molecule | (G4S)3- | GPSVFLFPPKPKDTLY | LGGPSVFLFPPKPK | APELLGGPSV | GGGGS | VQPGGSLRLSC |
| 12 | ABD1 | ITREPEVTCVVVDVSH | DTLYITREPEVTCV | FLFPPKPKDT | GGGGS | AASGFTFRSFG |
| (SEQ ID | | EDPEVKFNWYVDGVEV | VVDVSHEDPEVKFN | LYITREPEVT | (SEQ | MSWVRQAPGKG |
| NO: 27) | | HNAKTKPREEQYNSTY | WYVDGVEVHNAKTK | CVVVDVSHED | ID | PEWVSSISGSG |
| | | RVVSVLTVLHQDWLNG | PREEQYNSTYRVVS | PEVKFNWYVD | NO: | SDTLYADSVKG |
| | | KEYKCKVSNKALPAPI | VLTVLHQDWLNGKE | GVEVHNAKTK | 15) | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVM | NNYKTTPPVLDSDG | GQPREPQVCT | | |
| | | HEALKFHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SEVQLLESGGGLVQPG | ALKFHYTQKSLSLS | GFYPSDIAVE | | |
| | | GSLRLSCAASGFTFRS | PG (SEQ ID NO: | WESNGQPENN | | |
| | | FGMSWVRQAPGKGPEW | 5) | YKTTPPVLDS | | |
| | | VSSISGSGSDTLYADS | | DGSFFLVSKL | | |
| | | VKGRFTISRDNSKNTL | | TVDKSRWQQG | | |
| | | YLQMNSLRPEDTAVYY | | NVFSCSVMHE | | |
| | | CTIGGSLSRSSQGTLV | | ALKFHYTQKS | | |
| | | TVSS | | LSLSPG | | |
| | | | | (SEQ ID | | |
| | | | | NO: 4) | | |
| Fc Fusion | HSA-G4S- | DAHKSEVAHRFKDLGE | N/A | DAHKSEVAHR | GGGGS | DKTHTCPPCPA |
| Molecule | Fc197 | ENFKALVLIAFAQYLQ | | FKDLGEENFK | (SEQ | PELLGGPSVFL |
| 13 | | QCPFEDHVKLVNEVTE | | ALVLIAFAQY | ID | FPPKPKDTLYI |
| (SEQ ID | | FAKTCVADESAENCDK | | LQQCPFEDHV | NO: | TREPEVTCVVV |
| NO: 28) | | SLHTLFGDKLCTVATL | | KLVNEVTEFA | 13) | DVSHEDPEVKF |
| | | RETYGEMADCCAKQEP | | KTCVADESAE | | NWYVDGVEVHN |
| | | ERNECFLQHKDDNPNL | | NCDKSLHTLF | | AKTKPREEQYN |
| | | PRLVRPEVDVMCTAFH | | GDKLCTVATL | | STYRVVSVLTV |
| | | DNEETFLKKYLYEIAR | | RETYGEMADC | | LHQDWLNGKEY |
| | | RHPYFYAPELLFFAKR | | CAKQEPERNE | | KCKVSNKALPA |
| | | YKAAFTECCQAADKAA | | CFLQHKDDNP | | PIEKTISKAKG |
| | | CLLPKLDELRDEGKAS | | NLPRLVRPEV | | QPREPQVYTLP |
| | | SAKQRLKCASLQKFGE | | DVMCTAFHDN | | PSRDELTKNQV |
| | | RAFKAWAVARLSQRFP | | EETFLKKYLY | | SLTCLVKGFYP |
| | | KAEFAEVSKLVTDLTK | | EIARRHPYFY | | SDIAVEWESNG |
| | | VHTECCHGDLLECADD | | APELLFFAKR | | QPENNYKTTPP |
| | | RADLAKYICENQDSIS | | YKAAFTECCQ | | VLDSDGSFFLY |
| | | SKLKECCEKPLLEKSH | | AADKAACLLP | | SKLTVDKSRWQ |
| | | CIAEVENDEMPADLPS | | KLDELRDEGK | | QGNVFSCSVMH |
| | | LAADFVESKDVCKNYA | | ASSAKQRLKC | | EALKYHYTQKS |
| | | EAKDVFLGMFLYEYAR | | ASLQKFGERA | | LSLSPG (SEQ |
| | | RHPDYSVVLLLRLAKT | | FKAWAVARLS | | ID NO: 3) |
| | | YETTLEKCCAAADPHE | | QRFPKAEFAE | | |
| | | CYAKVFDEFKPLVEEP | | VSKLVTDLTK | | |
| | | QNLIKQNCELFEQLGE | | VHTECCHGDL | | |
| | | YKFQNALLVRYTKKVP | | LECADDRADL | | |
| | | QVSTPTLVEVSRNLGK | | AKYICENQDS | | |
| | | VGSKCCKHPEAKRMPC | | ISSKLKECCE | | |
| | | AEDYLSVVLNQLCVLH | | KPLLEKSHCI | | |
| | | EKTPVSDRVTKCCTES | | AEVENDEMPA | | |
| | | LVNRRPCFSALEVDET | | DLPSLAADFV | | |
| | | YVPKEFNAETFTFHAD | | ESKDVCKNYA | | |
| | | ICTLSEKERQIKKQTA | | EAKDVFLGMF | | |
| | | LVELVKHKPKATKEQL | | LYEYARRHPD | | |
| | | KAVMDDFAAFVEKCCK | | YSVVLLLRLA | | |
| | | ADDKETCFAEEGKKLV | | KTYETTLEKC | | |
| | | AASQAALGLGGGGSDK | | CAAADPHECY | | |
| | | THTCPPCPAPELLGGP | | AKVFDEFKPL | | |
| | | SVFLFPPKPKDTLYIT | | VEEPQNLIKQ | | |
| | | REPEVTCVVVDVSHED | | NCELFEQLGE | | |
| | | PEVKFNWYVDGVEVHN | | YKFQNALLVR | | |
| | | AKTKPREEQYNSTYRV | | YTKKVPQVST | | |
| | | VSVLTVLHQDWLNGKE | | PTLVEVSRNL | | |
| | | YKCKVSNKALPAPIEK | | GKVGSKCCKH | | |
| | | TISKAKGQPREPQVYT | | PEAKRMPCAE | | |
| | | LPPSRDELTKNQVSLT | | DYLSVVLNQL | | |
| | | CLVKGFYPSDIAVEWE | | CVLHEKTPVS | | |
| | | SNGQPENNYKTTPPVL | | DRVTKCCTES | | |
| | | DSDGSFFLYSKLTVDK | | LVNRRPCFSA | | |
| | | SRWQQGNVFSCSVMHE | | LEVDETYVPK | | |
| | | ALKYHYTQKSLSLSPG | | EFNAETFTFH | | |
| | | | | ADICTLSEKE | | |
| | | | | RQIKKQTALV | | |
| | | | | ELVKHKPKAT | | |
| | | | | KEQLKAVMDD | | |
| | | | | FAAFVEKCCK | | |
| | | | | ADDKETCFAE | | |
| | | | | EGKKLVAASQ | | |
| | | | | AALGL (SEQ | | |
| | | | | ID NO: 10) | | |
| Fc Fusion | Fc197-G4S- | DKTHTCPPCPAPELLG | N/A | DKTHTCPPCP | GGGGS | DAHKSEVAHRF |
| Molecule | HSA | GPSVFLFPPKPKDTLY | | APELLGGPSV | (SEQ | KDLGEENFKAL |
| 14 | | ITREPEVTCVVVDVSH | | FLFPPKPKDT | ID | VLIAFAQYLQQ |
| (SEQ ID | | EDPEVKFNWYVDGVEV | | LYITREPEVT | NO: | CPFEDHVKLVN |
| NO: 29) | | HNAKTKPREEQYNSTY | | CVVVDVSHED | 13) | EVTEFAKTCVA |
| | | RVVSVLTVLHQDWLNG | | PEVKFNWYVD | | DESAENCDKSL |
| | | KEYKCKVSNKALPAPI | | GVEVHNAKTK | | HTLFGDKLCTV |
| | | EKTISKAKGQPREPQV | | PREEQYNSTY | | ATLRETYGEMA |
| | | YTLPPSRDELTKNQVS | | RVVSVLTVLH | | DCCAKQEPERN |
| | | LTCLVKGFYPSDIAVE | | QDWLNGKEYK | | ECFLQHKDDNP |
| | | WESNGQPENNYKTTPP | | CKVSNKALPA | | NLPRLVRPEVD |
| | | VLDSDGSFFLYSKLTV | | PIEKTISKAK | | VMCTAFHDNEE |
| | | DKSRWQQGNVFSCSVM | | GQPREPQVYT | | TFLKKYLYEIA |
| | | HEALKYHYTQKSLSLS | | LPPSRDELTK | | RRHPYFYAPEL |
| | | PGGGGGSDAHKSEVAH | | NQVSLTCLVK | | LFFAKRYKAAF |
| | | RFKDLGEENFKALVLI | | GFYPSDIAVE | | TECCQAADKAA |
| | | AFAQYLQQCPFEDHVK | | WESNGQPENN | | CLLPKLDELRD |
| | | LVNEVTEFAKTCVADE | | YKTTPPVLDS | | EGKASSAKQRL |
| | | SAENCDKSLHTLFGDK | | DGSFFLYSKL | | KCASLQKFGER |
| | | LCTVATLRETYGEMAD | | TVDKSRWQQG | | AFKAWAVARLS |
| | | CCAKQEPERNECFLQH | | NVFSCSVMHE | | QRFPKAEFAEV |
| | | KDDNPNLPRLVRPEVD | | ALKYHYTQKS | | SKLVTDLTKVH |
| | | VMCTAFHDNEETFLKK | | LSLSPG | | TECCHGDLLEC |
| | | YLYEIARRHPYFYAPE | | (SEQ ID | | ADDRADLAKYI |
| | | LLFFAKRYKAAFTECC | | NO: 3) | | CENQDSISSKL |
| | | QAADKAACLLPKLDEL | | | | KECCEKPLLEK |
| | | RDEGKASSAKQRLKCA | | | | SHCIAEVENDE |
| | | SLQKFGERAFKAWAVA | | | | MPADLPSLAAD |
| | | RLSQRFPKAEFAEVSK | | | | FVESKDVCKNY |
| | | LVTDLTKVHTECCHGD | | | | AEAKDVFLGMF |
| | | LLECADDRADLAKYIC | | | | LYEYARRHPDY |
| | | ENQDSISSKLKECCEK | | | | SVVLLLRLAKT |
| | | PLLEKSHCIAEVENDE | | | | YETTLEKCCAA |
| | | MPADLPSLAADFVESK | | | | ADPHECYAKVF |
| | | DVCKNYAEAKDVFLGM | | | | DEFKPLVEEPQ |
| | | FLYEYARRHPDYSVVL | | | | NLIKQNCELFE |
| | | LLRLAKTYETTLEKCC | | | | QLGEYKFQNAL |
| | | AAADPHECYAKVFDEF | | | | LVRYTKKVPQV |
| | | KPLVEEPQNLIKQNCE | | | | STPTLVEVSRN |
| | | LFEQLGEYKFQNALLV | | | | LGKVGSKCCKH |
| | | RYTKKVPQVSTPTLVE | | | | PEAKRMPCAED |
| | | VSRNLGKVGSKCCKHP | | | | YLSVVLNQLCV |
| | | EAKRMPCAEDYLSVVL | | | | LHEKTPVSDRV |
| | | NQLCVLHEKTPVSDRV | | | | TKCCTESLVNR |
| | | TKCCTESLVNRRPCFS | | | | RPCFSALEVDE |
| | | ALEVDETYVPKEFNAE | | | | TYVPKEFNAET |
| | | TFTFHADICTLSEKER | | | | FTFHADICTLS |
| | | QIKKQTALVELVKHKP | | | | EKERQIKKQTA |
| | | KATKEQLKAVMDDFAA | | | | LVELVKHKPKA |
| | | FVEKCCKADDKETCFA | | | | TKEQLKAVMDD |
| | | EEGKKLVAASQAALGL | | | | FAAFVEKCCKA |
| | | | | | | DDKETCFAEEG |
| | | | | | | KKLVAASQAAL |
| | | | | | | GL (SEQ ID |
| | | | | | | NO: 10) |
| Fc Fusion | Fc197-G4S- | DKTHTCPPCPAPELLG | N/A | DKTHTCPPCP | GGGGS | EVQLVESGGGL |
| Molecule | ABD2 | GPSVFLFPPKPKDTLY | | APELLGGPSV | (SEQ | VQPGNSLRLSC |
| 15 | | ITREPEVTCVVVDVSH | | FLFPPKPKDT | ID | AASGFTFSSFG |
| (SEQ ID | | EDPEVKFNWYVDGVEV | | LYITREPEVT | NO: | MSWVRQAPGKG |
| NO: 30) | | HNAKTKPREEQYNSTY | | CVVVDVSHED | 13) | LEWVSSISGSG |
| | | RVVSVLTVLHQDWLNG | | PEVKFNWYVD | | SDTLYADSVKG |
| | | KEYKCKVSNKALPAPI | | GVEVHNAKTK | | RFTISRDNAKT |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 12) |
| | | DKSRWQQGNVFSCSVM | | GQPREPQVYT | | |
| | | HEALKYHYTQKSLSLS | | LPPSRDELTK | | |
| | | PGGGGGSEVQLVESGG | | NQVSLTCLVK | | |
| | | GLVQPGNSLRLSCAAS | | GFYPSDIAVE | | |
| | | GFTFSSFGMSWVRQAP | | WESNGQPENN | | |
| | | GKGLEWVSSISGSGSD | | YKTTPPVLDS | | |
| | | TLYADSVKGRFTISRD | | DGSFFLYSKL | | |
| | | NAKTTLYLQMNSLRPE | | TVDKSRWQQG | | |
| | | DTAVYYCTIGGSLSRS | | NVFSCSVMHE | | |
| | | SQGTLVTVSS | | ALKYHYTQKS | | |
| | | | | LSLSPG | | |
| | | | | (SEQ ID | | |
| | | | | NO: 3) | | |
| Fc Fusion | Fc197-G4S- | DKTHTCPPCPAPELLG | N/A | DKTHTCPPCP | GGGGS | EVQLLESGGGL |
| Molecule | ABD1 | GPSVFLFPPKPKDTLY | | APELLGGPSV | (SEQ | VQPGGSLRLSC |
| 16 | | ITREPEVTCVVVDVSH | | FLFPPKPKDT | ID | AASGFTFRSFG |
| (SEQ ID | | EDPEVKFNWYVDGVEV | | LYITREPEVT | NO: | MSWVRQAPGKG |
| NO: 31) | | HNAKTKPREEQYNSTY | | CVVVDVSHED | 13) | PEWVSSISGSG |
| | | RVVSVLTVLHQDWLNG | | PEVKFNWYVD | | SDTLYADSVKG |
| | | KEYKCKVSNKALPAPI | | GVEVHNAKTK | | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVM | | GQPREPQVYT | | |
| | | HEALKYHYTQKSLSLS | | LPPSRDELTK | | |
| | | PGGGGGSEVQLLESGG | | NQVSLTCLVK | | |
| | | GLVQPGGSLRLSCAAS | | GFYPSDIAVE | | |
| | | GFTFRSFGMSWVRQAP | | WESNGQPENN | | |
| | | GKGPEWVSSISGSGSD | | YKTTPPVLDS | | |
| | | TLYADSVKGRFTISRD | | DGSFFLYSKL | | |
| | | NSKNTLYLQMNSLRPE | | TVDKSRWQQG | | |
| | | DTAVYYCTIGGSLSRS | | NVFSCSVMHE | | |
| | | SQGTLVTVSS | | ALKYHYTQKS | | |
| | | | | LSLSPG | | |
| | | | | (SEQ ID | | |
| | | | | NO: 3) | | |
| Fc Fusion | Fc197- | DKTHTCPPCPAPELLG | N/A | DKTHTCPPCP | GGGGS | EVQLVESGGGL |
| Molecule | 3G4S-ABD2 | GPSVFLFPPKPKDTLY | | APELLGGPSV | GGGGS | VQPGNSLRLSC |
| 17 | | ITREPEVTCVVVDVSH | | FLFPPKPKDT | GGGGS | AASGFTFSSFG |
| (SEQ ID | | EDPEVKFNWYVDGVEV | | LYITREPEVT | (SEQ | MSWVRQAPGKG |
| NO: 32) | | HNAKTKPREEQYNSTY | | CVVVDVSHED | ID | LEWVSSISGSG |
| | | RVVSVLTVLHQDWLNG | | PEVKFNWYVD | NO: | SDTLYADSVKG |
| | | KEYKCKVSNKALPAPI | | GVEVHNAKTK | 15) | RFTISRDNAKT |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 12) |
| | | DKSRWQQGNVFSCSVM | | GQPREPQVYT | | |
| | | HEALKYHYTQKSLSLS | | LPPSRDELTK | | |
| | | PGGGGGSGGGGSGGGG | | NQVSLTCLVK | | |
| | | SEVQLVESGGGLVQPG | | GFYPSDIAVE | | |
| | | NSLRLSCAASGFTFSS | | WESNGQPENN | | |
| | | FGMSWVRQAPGKGLEW | | YKTTPPVLDS | | |
| | | VSSISGSGSDTLYADS | | DGSFFLYSKL | | |
| | | VKGRFTISRDNAKTTL | | TVDKSRWQQG | | |
| | | YLQMNSLRPEDTAVYY | | NVFSCSVMHE | | |
| | | CTIGGSLSRSSQGTLV | | ALKYHYTQKS | | |
| | | TVSS | | LSLSPG | | |
| | | | | (SEQ ID | | |
| | | | | NO: 3) | | |
| Fc Fusion | Fc197- | DKTHTCPPCPAPELLG | N/A | DKTHTCPPCP | GGGGS | EVQLLESGGGL |
| Molecule | 3G4S-ABD1 | GPSVFLFPPKPKDTLY | | APELLGGPSV | GGGGS | VQPGGSLRLSC |
| 18 | | ITREPEVTCVVVDVSH | | FLFPPKPKDT | GGGGS | AASGFTFRSFG |
| (SEQ ID | | EDPEVKFNWYVDGVEV | | LYITREPEVT | (SEQ | MSWVRQAPGKG |
| NO: 33) | | HNAKTKPREEQYNSTY | | CVVVDVSHED | ID | PEWVSSISGSG |
| | | RVVSVLTVLHQDWLNG | | PEVKFNWYVD | NO: | SDTLYADSVKG |
| | | KEYKCKVSNKALPAPI | | GVEVHNAKTK | 15) | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVM | | GQPREPQVYT | | |
| | | HEALKYHYTQKSLSLS | | LPPSRDELTK | | |
| | | PGGGGGSGGGGSGGGG | | NQVSLTCLVK | | |
| | | SEVQLLESGGGLVQPG | | GFYPSDIAVE | | |
| | | GSLRLSCAASGFTFRS | | WESNGQPENN | | |
| | | FGMSWVRQAPGKGPEW | | YKTTPPVLDS | | |
| | | VSSISGSGSDTLYADS | | DGSFFLYSKL | | |
| | | VKGRFTISRDNSKNTL | | TVDKSRWQQG | | |
| | | YLQMNSLRPEDTAVYY | | NVFSCSVMHE | | |
| | | CTIGGSLSRSSQGTLV | | ALKYHYTQKS | | |
| | | TVSS | | LSLSPG | | |
| | | | | (SEQ ID | | |
| | | | | NO: 3) | | |
| Fc Fusion | Fc197(KIH)- | DKTHTCPPCPAPELLG | DKTHTCPPCPAPEL | DKTHTCPPCP | GGGGS | EVQLVESGGGL |
| Molecule | G4S-ABD2 | GPSVFLFPPKPKDTLY | LGGPSVFLFPPKPK | APELLGGPSV | (SEQ | VQPGNSLRLSC |
| 19 | | ITREPEVTCVVVDVSH | DTLYITREPEVTCV | FLFPPKPKDT | ID | AASGFTFSSFG |
| (SEQ ID | | EDPEVKFNWYVDGVEV | VVDVSHEDPEVKFN | LYITREPEVT | NO: | MSWVRQAPGKG |
| NO: 34) | | HNAKTKPREEQYNSTY | WYVDGVEVHNAKTK | CVVVDVSHED | 13) | LEWVSSISGSG |
| | | RVVSVLTVLHQDWLNG | PREEQYNSTYRVVS | PEVKFNWYVD | | SDTLYADSVKG |
| | | KEYKCKVSNKALPAPI | VLTVLHQDWLNGKE | GVEVHNAKTK | | RFTISRDNAKT |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 12) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSEVQLVESGG | WQQGNVFSCSVMHE | NQVSLSCAVK | | |
| | | GLVQPGNSLRLSCAAS | ALKYHYTQKSLSLS | GFYPSDIAVE | | |
| | | GFTFSSFGMSWVRQAP | PG (SEQ ID NO: | WESNGQPENN | | |
| | | GKGLEWVSSISGSGSD | 7) | YKTTPPVLDS | | |
| | | TLYADSVKGRFTISRD | | DGSFFLVSKL | | |
| | | NAKTTLYLQMNSLRPE | | TVDKSRWQQG | | |
| | | DTAVYYCTIGGSLSRS | | NVFSCSVMHE | | |
| | | SQGTLVTVSS | | ALKYHYTQKS | | |
| | | | | LSLSPG | | |
| | | | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | G4S-ABD1 | GPSVFLFPPKPKDTLY | LGGPSVFLFPPKPK | APELLGGPSV | (SEQ | VQPGGSLRLSC |
| 20 | | ITREPEVTCVVVDVSH | DTLYITREPEVTCV | FLFPPKPKDT | ID | AASGFTFRSFG |
| (SEQ ID | | EDPEVKFNWYVDGVEV | VVDVSHEDPEVKFN | LYITREPEVT | NO: | MSWVRQAPGKG |
| NO: 35) | | HNAKTKPREEQYNSTY | WYVDGVEVHNAKTK | CVVVDVSHED | 13) | PEWVSSISGSG |
| | | RVVSVLTVLHQDWLNG | PREEQYNSTYRVVS | PEVKFNWYVD | | SDTLYADSVKG |
| | | KEYKCKVSNKALPAPI | VLTVLHQDWLNGKE | GVEVHNAKTK | | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSEVQLLESGG | WQQGNVFSCSVMHE | NQVSLSCAVK | | |
| | | GLVQPGGSLRLSCAAS | ALKYHYTQKSLSLS | GFYPSDIAVE | | |
| | | GFTFRSFGMSWVRQAP | PG (SEQ ID NO: | WESNGQPENN | | |
| | | GKGPEWVSSISGSGSD | 7) | YKTTPPVLDS | | |
| | | TLYADSVKGRFTISRD | | DGSFFLVSKL | | |
| | | NSKNTLYLQMNSLRPE | | TVDKSRWQQG | | |
| | | DTAVYYCTIGGSLSRS | | NVFSCSVMHE | | |
| | | SQGTLVTVSS | | ALKYHYTQKS | | |
| | | | | LSLSPG | | |
| | | | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLVESGGGL |
| Molecule | 3G4S-ABD2 | GPSVFLFPPKPKDTLY | LGGPSVFLFPPKPK | APELLGGPSV | GGGGS | VQPGNSLRLSC |
| 21 | | ITREPEVTCVVVDVSH | DTLYITREPEVTCV | FLFPPKPKDT | GGGGS | AASGFTFSSFG |
| (SEQ ID | | EDPEVKFNWYVDGVEV | VVDVSHEDPEVKEN | LYITREPEVT | (SEQ | MSWVRQAPGKG |
| NO: 36) | | HNAKTKPREEQYNSTY | WYVDGVEVHNAKTK | CVVVDVSHED | ID | LEWVSSISGSG |
| | | RVVSVLTVLHQDWLNG | PREEQYNSTYRVVS | PEVKFNWYVD | NO: | SDTLYADSVKG |
| | | KEYKCKVSNKALPAPI | VLTVLHQDWLNGKE | GVEVHNAKTK | 15) | RFTISRDNAKT |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 12) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SEVQLVESGGGLVQPG | ALKYHYTQKSLSLS | GFYPSDIAVE | | |
| | | NSLRLSCAASGFTFSS | PG (SEQ ID NO: | WESNGQPENN | | |
| | | FGMSWVRQAPGKGLEW | 7) | YKTTPPVLDS | | |
| | | VSSISGSGSDTLYADS | | DGSFFLVSKL | | |
| | | VKGRFTISRDNAKTTL | | TVDKSRWQQG | | |
| | | YLQMNSLRPEDTAVYY | | NVFSCSVMHE | | |
| | | CTIGGSLSRSSQGTLV | | ALKYHYTQKS | | |
| | | TVSS | | LSLSPG | | |
| | | | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | 3G4S-ABD1 | GPSVFLFPPKPKDTLY | LGGPSVFLFPPKPK | APELLGGPSV | GGGGS | VQPGGSLRLSC |
| 22 | | ITREPEVTCVVVDVSH | DTLYITREPEVTCV | FLFPPKPKDT | GGGGS | AASGFTFRSFG |
| (SEQ ID | | EDPEVKFNWYVDGVEV | VVDVSHEDPEVKFN | LYITREPEVT | (SEQ | MSWVRQAPGKG |
| NO: 37) | | HNAKTKPREEQYNSTY | WYVDGVEVHNAKTK | CVVVDVSHED | ID | PEWVSSISGSG |
| | | RVVSVLTVLHQDWLNG | PREEQYNSTYRVVS | PEVKFNWYVD | NO: | SDTLYADSVKG |
| | | KEYKCKVSNKALPAPI | VLTVLHQDWLNGKE | GVEVHNAKTK | 15) | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SEVQLLESGGGLVQPG | ALKYHYTQKSLSLS | GFYPSDIAVE | | |
| | | GSLRLSCAASGFTFRS | PG (SEQ ID NO: | WESNGQPENN | | |
| | | FGMSWVRQAPGKGPEW | 7) | YKTTPPVLDS | | |
| | | VSSISGSGSDTLYADS | | DGSFFLVSKL | | |
| | | VKGRFTISRDNSKNTL | | TVDKSRWQQG | | |
| | | YLQMNSLRPEDTAVYY | | NVFSCSVMHE | | |
| | | CTIGGSLSRSSQGTLV | | ALKYHYTQKS | | |
| | | TVSS | | LSLSPG | | |
| | | | | (SEQ ID | | |
| | | | | NO: 6) | | |
| Fc Fusion | ABD2-G4S- | EVQLVESGGGLVQPGN | N/A | EVQLVESGGG | GGGGS | DKTHTCPPCPA |
| Molecule | Fc197 | SLRLSCAASGFTFSSF | | LVQPGNSLRL | (SEQ | PELLGGPSVFL |
| 23 | | GMSWVRQAPGKGLEWV | | SCAASGFTFS | ID | FPPKPKDTLYI |
| (SEQ ID | | SSISGSGSDTLYADSV | | SFGMSWVRQA | NO: | TREPEVTCVVV |
| NO: 38) | | KGRFTISRDNAKTTLY | | PGKGLEWVSS | 13) | DVSHEDPEVKF |
| | | LQMNSLRPEDTAVYYC | | ISGSGSDTLY | | NWYVDGVEVHN |
| | | TIGGSLSRSSQGTLVT | | ADSVKGRFTI | | AKTKPREEQYN |
| | | VSSGGGGSDKTHTCPP | | SRDNAKTTLY | | STYRVVSVLIV |
| | | CPAPELLGGPSVFLFP | | LQMNSLRPED | | LHQDWLNGKEY |
| | | PKPKDTLYITREPEVT | | TAVYYCTIGG | | KCKVSNKALPA |
| | | CVVVDVSHEDPEVKFN | | SLSRSSQGTL | | PIEKTISKAKG |
| | | WYVDGVEVHNAKTKPR | | VTVSS (SEQ | | QPREPQVYTLP |
| | | EEQYNSTYRVVSVLTV | | ID NO: 12) | | PSRDELTKNQV |
| | | LHQDWLNGKEYKCKVS | | | | SLTCLVKGFYP |
| | | NKALPAPIEKTISKAK | | | | SDIAVEWESNG |
| | | GQPREPQVYTLPPSRD | | | | QPENNYKTTPP |
| | | ELTKNQVSLTCLVKGF | | | | VLDSDGSFFLY |
| | | YPSDIAVEWESNGQPE | | | | SKLTVDKSRWQ |
| | | NNYKTTPPVLDSDGSF | | | | QGNVFSCSVMH |
| | | FLYSKLTVDKSRWQQG | | | | EALKYHYTQKS |
| | | NVFSCSVMHEALKYHY | | | | LSLSPG (SEQ |
| | | TQKSLSLSPG | | | | ID NO: 3) |
| Fc Fusion | ABD1-G4S- | EVQLLESGGGLVQPGG | N/A | EVQLLESGGG | GGGGS | DKTHTCPPCPA |
| Molecule | Fc197 | SLRLSCAASGFTFRSF | | LVQPGGSLRL | (SEQ | PELLGGPSVFL |
| 24 | | GMSWVRQAPGKGPEWV | | SCAASGFTFR | ID | FPPKPKDTLYI |
| (SEQ ID | | SSISGSGSDTLYADSV | | SFGMSWVRQA | NO: | TREPEVTCVVV |
| NO: 39) | | KGRFTISRDNSKNTLY | | PGKGPEWVSS | 13) | DVSHEDPEVKF |
| | | LQMNSLRPEDTAVYYC | | ISGSGSDTLY | | NWYVDGVEVHN |
| | | TIGGSLSRSSQGTLVT | | ADSVKGRFTI | | AKTKPREEQYN |
| | | VSSGGGGSDKTHTCPP | | SRDNSKNTLY | | STYRVVSVLIV |
| | | CPAPELLGGPSVFLFP | | LQMNSLRPED | | LHQDWLNGKEY |
| | | PKPKDTLYITREPEVT | | TAVYYCTIGG | | KCKVSNKALPA |
| | | CVVVDVSHEDPEVKFN | | SLSRSSQGTL | | PIEKTISKAKG |
| | | WYVDGVEVHNAKTKPR | | VTVSS (SEQ | | QPREPQVYTLP |
| | | EEQYNSTYRVVSVLIV | | ID NO: 11) | | PSRDELTKNQV |
| | | LHQDWLNGKEYKCKVS | | | | SLTCLVKGFYP |
| | | NKALPAPIEKTISKAK | | | | SDIAVEWESNG |
| | | GQPREPQVYTLPPSRD | | | | QPENNYKTTPP |
| | | ELTKNQVSLTCLVKGF | | | | VLDSDGSFFLY |
| | | YPSDIAVEWESNGQPE | | | | SKLTVDKSRWQ |
| | | NNYKTTPPVLDSDGSF | | | | QGNVFSCSVMH |
| | | FLYSKLTVDKSRWQQG | | | | EALKYHYTQKS |
| | | NVFSCSVMHEALKYHY | | | | LSLSPG (SEQ |
| | | TQKSLSLSPG | | | | ID NO: 3) |
| Fc Fusion | ABD2- | EVQLVESGGGLVQPGN | N/A | EVQLVESGGG | GGGGS | DKTHTCPPCPA |
| Molecule | 3G4S-Fc197 | SLRLSCAASGFTFSSF | | LVQPGNSLRL | GGGGS | PELLGGPSVFL |
| 25 | | GMSWVRQAPGKGLEWV | | SCAASGFTFS | GGGGS | FPPKPKDTLYI |
| (SEQ ID | | SSISGSGSDTLYADSV | | SFGMSWVRQA | (SEQ | TREPEVTCVVV |
| NO: 40) | | KGRFTISRDNAKTTLY | | PGKGLEWVSS | ID | DVSHEDPEVKF |
| | | LQMNSLRPEDTAVYYC | | ISGSGSDTLY | NO: | NWYVDGVEVHN |
| | | TIGGSLSRSSQGTLVT | | ADSVKGRFTI | 15) | AKTKPREEQYN |
| | | VSSGGGGSGGGGSGGG | | SRDNAKTTLY | | STYRVVSVLIV |
| | | GSDKTHTCPPCPAPEL | | LQMNSLRPED | | LHQDWLNGKEY |
| | | LGGPSVFLFPPKPKDT | | TAVYYCTIGG | | KCKVSNKALPA |
| | | LYITREPEVTCVVVDV | | SLSRSSQGTL | | PIEKTISKAKG |
| | | SHEDPEVKFNWYVDGV | | VTVSS (SEQ | | QPREPQVYTLP |
| | | EVHNAKTKPREEQYNS | | ID NO: 12) | | PSRDELTKNQV |
| | | TYRVVSVLIVLHQDWL | | | | SLTCLVKGFYP |
| | | NGKEYKCKVSNKALPA | | | | SDIAVEWESNG |
| | | PIEKTISKAKGQPREP | | | | QPENNYKTTPP |
| | | QVYTLPPSRDELTKNQ | | | | VLDSDGSFFLY |
| | | VSLTCLVKGFYPSDIA | | | | SKLTVDKSRWQ |
| | | VEWESNGQPENNYKTT | | | | QGNVFSCSVMH |
| | | PPVLDSDGSFFLYSKL | | | | EALKYHYTQKS |
| | | TVDKSRWQQGNVFSCS | | | | LSLSPG (SEQ |
| | | VMHEALKYHYTQKSLS | | | | ID NO: 3) |
| | | LSPG | | | | |
| Fc Fusion | ABD1- | EVQLLESGGGLVQPGG | N/A | EVQLLESGGG | GGGGS | DKTHTCPPCPA |
| Molecule | 3G4S-Fc197 | SLRLSCAASGFTFRSF | | LVQPGGSLRL | GGGGS | PELLGGPSVFL |
| 26 | | GMSWVRQAPGKGPEWV | | SCAASGFTFR | GGGGS | FPPKPKDILYI |
| (SEQ ID | | SSISGSGSDTLYADSV | | SFGMSWVRQA | (SEQ | TREPEVTCVVV |
| NO: 41) | | KGRFTISRDNSKNTLY | | PGKGPEWVSS | ID | DVSHEDPEVKF |
| | | LQMNSLRPEDTAVYYC | | ISGSGSDTLY | NO: | NWYVDGVEVHN |
| | | TIGGSLSRSSQGTLVT | | ADSVKGRFTI | 15) | AKTKPREEQYN |
| | | VSSGGGGSGGGGSGGG | | SRDNSKNTLY | | STYRVVSVLTV |
| | | GSDKTHTCPPCPAPEL | | LQMNSLRPED | | LHQDWLNGKEY |
| | | LGGPSVFLFPPKPKDT | | TAVYYCTIGG | | KCKVSNKALPA |
| | | LYITREPEVTCVVVDV | | SLSRSSQGTL | | PIEKTISKAKG |
| | | SHEDPEVKFNWYVDGV | | VTVSS (SEQ | | QPREPQVYTLP |
| | | EVHNAKTKPREEQYNS | | ID NO: 11) | | PSRDELTKNQV |
| | | TYRVVSVLTVLHQDWL | | | | SLTCLVKGFYP |
| | | NGKEYKCKVSNKALPA | | | | SDIAVEWESNG |
| | | PIEKTISKAKGQPREP | | | | QPENNYKTTPP |
| | | QVYTLPPSRDELTKNQ | | | | VLDSDGSFFLY |
| | | VSLTCLVKGFYPSDIA | | | | SKLTVDKSRWQ |
| | | VEWESNGQPENNYKTT | | | | QGNVFSCSVMH |
| | | PPVLDSDGSFFLYSKL | | | | EALKYHYTQKS |
| | | TVDKSRWQQGNVFSCS | | | | LSLSPG (SEQ |
| | | VMHEALKYHYTQKSLS | | | | ID NO: 3) |
| | | LSPG | | | | |
| Fc Fusion | ABD2-G4S- | EVQLVESGGGLVQPGN | DKTHTCPPCPAPEL | EVQLVESGGG | GGGGS | DKTHTCPPCPA |
| Molecule | Fc197(KIH) | SLRLSCAASGFTFSSF | LGGPSVFLFPPKPK | LVQPGNSLRL | (SEQ | PELLGGPSVFL |
| 27 | | GMSWVRQAPGKGLEWV | DTLYITREPEVTCV | SCAASGFTFS | ID | FPPKPKDTLYI |
| (SEQ ID | | SSISGSGSDTLYADSV | VVDVSHEDPEVKFN | SFGMSWVRQA | NO: | TREPEVTCVVV |
| NO: 42) | | KGRFTISRDNAKTTLY | WYVDGVEVHNAKTK | PGKGLEWVSS | 13) | DVSHEDPEVKF |
| | | LQMNSLRPEDTAVYYC | PREEQYNSTYRVVS | ISGSGSDTLY | | NWYVDGVEVHN |
| | | TIGGSLSRSSQGTLVT | VLTVLHQDWLNGKE | ADSVKGRFTI | | AKTKPREEQYN |
| | | VSSGGGGSDKTHTCPP | YKCKVSNKALPAPI | SRDNAKTTLY | | STYRVVSVLIV |
| | | CPAPELLGGPSVFLFP | EKTISKAKGQPREP | LQMNSLRPED | | LHQDWLNGKEY |
| | | PKPKDTLYITREPEVT | QVYTLPPCRDELTK | TAVYYCTIGG | | KCKVSNKALPA |
| | | CVVVDVSHEDPEVKFN | NQVSLWCLVKGFYP | SLSRSSQGTL | | PIEKTISKAKG |
| | | WYVDGVEVHNAKTKPR | SDIAVEWESNGQPE | VTVSS (SEQ | | QPREPQVCTLP |
| | | EEQYNSTYRVVSVLIV | NNYKTTPPVLDSDG | ID NO: 12) | | PSRDELTKNQV |
| | | LHQDWLNGKEYKCKVS | SFFLYSKLTVDKSR | | | SLSCAVKGFYP |
| | | NKALPAPIEKTISKAK | WQQGNVFSCSVMHE | | | SDIAVEWESNG |
| | | GQPREPQVCTLPPSRD | ALKYHYTQKSLSLS | | | QPENNYKTTPP |
| | | ELTKNQVSLSCAVKGF | PG (SEQ ID NO: | | | VLDSDGSFFLV |
| | | YPSDIAVEWESNGQPE | 7) | | | SKLTVDKSRWQ |
| | | NNYKTTPPVLDSDGSF | | | | QGNVFSCSVMH |
| | | FLVSKLTVDKSRWQQG | | | | EALKYHYTQKS |
| | | NVFSCSVMHEALKYHY | | | | LSLSPG (SEQ |
| | | TQKSLSLSPG | | | | ID NO: 6) |
| Fc Fusion | ABD1-G4S- | EVQLLESGGGLVQPGG | DKTHTCPPCPAPEL | EVQLLESGGG | GGGGS | DKTHTCPPCPA |
| Molecule | Fc197(KIH) | SLRLSCAASGFTFRSF | LGGPSVFLFPPKPK | LVQPGGSLRL | (SEQ | PELLGGPSVFL |
| 28 | | GMSWVRQAPGKGPEWV | DTLYITREPEVTCV | SCAASGFTFR | ID | FPPKPKDTLYI |
| (SEQ ID | | SSISGSGSDTLYADSV | VVDVSHEDPEVKFN | SFGMSWVRQA | NO: | TREPEVTCVVV |
| NO: 43) | | KGRFTISRDNSKNTLY | WYVDGVEVHNAKTK | PGKGPEWVSS | 13) | DVSHEDPEVKF |
| | | LQMNSLRPEDTAVYYC | PREEQYNSTYRVVS | ISGSGSDTLY | | NWYVDGVEVHN |
| | | TIGGSLSRSSQGTLVT | VLTVLHQDWLNGKE | ADSVKGRFTI | | AKTKPREEQYN |
| | | VSSGGGGSDKTHTCPP | YKCKVSNKALPAPI | SRDNSKNTLY | | STYRVVSVLIV |
| | | CPAPELLGGPSVFLFP | EKTISKAKGQPREP | LQMNSLRPED | | LHQDWLNGKEY |
| | | PKPKDTLYITREPEVT | QVYTLPPCRDELTK | TAVYYCTIGG | | KCKVSNKALPA |
| | | CVVVDVSHEDPEVKFN | NQVSLWCLVKGFYP | SLSRSSQGTL | | PIEKTISKAKG |
| | | WYVDGVEVHNAKTKPR | SDIAVEWESNGQPE | VTVSS (SEQ | | QPREPQVCTLP |
| | | EEQYNSTYRVVSVLIV | NNYKTTPPVLDSDG | ID NO: 11) | | PSRDELTKNQV |
| | | LHQDWLNGKEYKCKVS | SFFLYSKLTVDKSR | | | SLSCAVKGFYP |
| | | NKALPAPIEKTISKAK | WQQGNVFSCSVMHE | | | SDIAVEWESNG |
| | | GQPREPQVCTLPPSRD | ALKYHYTQKSLSLS | | | QPENNYKTTPP |
| | | ELTKNQVSLSCAVKGF | PG (SEQ ID NO: | | | VLDSDGSFFLV |
| | | YPSDIAVEWESNGQPE | 7) | | | SKLTVDKSRWQ |
| | | NNYKTTPPVLDSDGSF | | | | QGNVFSCSVMH |
| | | FLVSKLTVDKSRWQQG | | | | EALKYHYTQKS |
| | | NVFSCSVMHEALKYHY | | | | LSLSPG (SEQ |
| | | TQKSLSLSPG | | | | ID NO: 6) |
| Fc Fusion | ABD2- | EVQLVESGGGLVQPGN | DKTHTCPPCPAPEL | EVQLVESGGG | GGGGS | DKTHTCPPCPA |
| Molecule | 3G4S- | SLRLSCAASGFTFSSF | LGGPSVFLFPPKPK | LVQPGNSLRL | GGGGS | PELLGGPSVFL |
| 29 | Fc197(KIH) | GMSWVRQAPGKGLEWV | DTLYITREPEVTCV | SCAASGFTFS | GGGGS | FPPKPKDTLYI |
| (SEQ ID | | SSISGSGSDTLYADSV | VVDVSHEDPEVKFN | SFGMSWVRQA | (SEQ | TREPEVTCVVV |
| NO: 44) | | KGRFTISRDNAKTTLY | WYVDGVEVHNAKTK | PGKGLEWVSS | ID | DVSHEDPEVKF |
| | | LQMNSLRPEDTAVYYC | PREEQYNSTYRVVS | ISGSGSDILY | NO: | NWYVDGVEVHN |
| | | TIGGSLSRSSQGTLVT | VLTVLHQDWLNGKE | ADSVKGRFTI | 15) | AKTKPREEQYN |
| | | VSSGGGGSGGGGSGGG | YKCKVSNKALPAPI | SRDNAKTTLY | | STYRVVSVLTV |
| | | GSDKTHTCPPCPAPEL | EKTISKAKGQPREP | LQMNSLRPED | | LHQDWLNGKEY |
| | | LGGPSVFLFPPKPKDT | QVYTLPPCRDELTK | TAVYYCTIGG | | KCKVSNKALPA |
| | | LYITREPEVTCVVVDV | NQVSLWCLVKGFYP | SLSRSSQGTL | | PIEKTISKAKG |
| | | SHEDPEVKFNWYVDGV | SDIAVEWESNGQPE | VTVSS (SEQ | | QPREPQVCTLP |
| | | EVHNAKTKPREEQYNS | NNYKTTPPVLDSDG | ID NO: 12) | | PSRDELTKNQV |
| | | TYRVVSVLTVLHQDWL | SFFLYSKLTVDKSR | | | SLSCAVKGFYP |
| | | NGKEYKCKVSNKALPA | WQQGNVFSCSVMHE | | | SDIAVEWESNG |
| | | PIEKTISKAKGQPREP | ALKYHYTQKSLSLS | | | QPENNYKTTPP |
| | | QVCTLPPSRDELTKNQ | PG (SEQ ID NO: | | | VLDSDGSFFLV |
| | | VSLSCAVKGFYPSDIA | 7) | | | SKLTVDKSRWQ |
| | | VEWESNGQPENNYKIT | | | | QGNVFSCSVMH |
| | | PPVLDSDGSFFLVSKL | | | | EALKYHYTQKS |
| | | TVDKSRWQQGNVFSCS | | | | LSLSPG (SEQ |
| | | VMHEALKYHYTQKSLS | | | | ID NO: 6) |
| | | LSPG | | | | |
| Fc Fusion | ABD1- | EVQLLESGGGLVQPGG | DKTHTCPPCPAPEL | EVQLLESGGG | GGGGS | DKTHTCPPCPA |
| Molecule | 3G4S- | SLRLSCAASGFTFRSF | LGGPSVFLFPPKPK | LVQPGGSLRL | GGGGS | PELLGGPSVFL |
| 30 | Fc197(KIH) | GMSWVRQAPGKGPEWV | DTLYITREPEVTCV | SCAASGFTFR | GGGGS | FPPKPKDTLYI |
| (SEQ ID | | SSISGSGSDTLYADSV | VVDVSHEDPEVKFN | SFGMSWVRQA | (SEQ | TREPEVTCVVV |
| NO: 45) | | KGRFTISRDNSKNTLY | WYVDGVEVHNAKTK | PGKGPEWVSS | ID | DVSHEDPEVKF |
| | | LQMNSLRPEDTAVYYC | PREEQYNSTYRVVS | ISGSGSDTLY | NO: | NWYVDGVEVHN |
| | | TIGGSLSRSSQGTLVT | VLTVLHQDWLNGKE | ADSVKGRFTI | 15) | AKTKPREEQYN |
| | | VSSGGGGSGGGGSGGG | YKCKVSNKALPAPI | SRDNSKNTLY | | STYRVVSVLTV |
| | | GSDKTHTCPPCPAPEL | EKTISKAKGQPREP | LQMNSLRPED | | LHQDWLNGKEY |
| | | LGGPSVFLFPPKPKDT | QVYTLPPCRDELTK | TAVYYCTIGG | | KCKVSNKALPA |
| | | LYITREPEVTCVVVDV | NQVSLWCLVKGFYP | SLSRSSQGTL | | PIEKTISKAKG |
| | | SHEDPEVKFNWYVDGV | SDIAVEWESNGQPE | VTVSS (SEQ | | QPREPQVCTLP |
| | | EVHNAKTKPREEQYNS | NNYKTTPPVLDSDG | ID NO: 11) | | PSRDELTKNQV |
| | | TYRVVSVLTVLHQDWL | SFFLYSKLTVDKSR | | | SLSCAVKGFYP |
| | | NGKEYKCKVSNKALPA | WQQGNVFSCSVMHE | | | SDIAVEWESNG |
| | | PIEKTISKAKGQPREP | ALKYHYTQKSLSLS | | | QPENNYKTTPP |
| | | QVCTLPPSRDELTKNQ | PG (SEQ ID NO: | | | VLDSDGSFFLV |
| | | VSLSCAVKGFYPSDIA | 7) | | | SKLTVDKSRWQ |
| | | VEWESNGQPENNYKTT | | | | QGNVFSCSVMH |
| | | PPVLDSDGSFFLVSKL | | | | EALKYHYTQKS |
| | | TVDKSRWQQGNVFSCS | | | | LSLSPG (SEQ |
| | | VMHEALKYHYTQKSLS | | | | ID NO: 6) |
| | | LSPG | | | | |
| Fc Fusion | HSA-G4S- | DAHKSEVAHRFKDLGE | N/A | DAHKSEVAHR | GGGGS | DKTHTCPPCPA |
| Molecule | Fc195 | ENFKALVLIAFAQYLQ | | FKDLGEENFK | (SEQ | PELLGGPSVFL |
| 31 | | QCPFEDHVKLVNEVTE | | ALVLIAFAQY | ID | FPPKPKDTLYI |
| (SEQ ID | | FAKTCVADESAENCDK | | LQQCPFEDHV | NO: | TREPEVTCVVV |
| NO: 46) | | SLHTLFGDKLCTVATL | | KLVNEVTEFA | 13) | DVSHEDPEVKF |
| | | RETYGEMADCCAKQEP | | KTCVADESAE | | NWYVDGVEVHN |
| | | ERNECFLQHKDDNPNL | | NCDKSLHTLF | | AKTKPREEQYN |
| | | PRLVRPEVDVMCTAFH | | GDKLCTVATL | | STYRVVSVLTV |
| | | DNEETFLKKYLYEIAR | | RETYGEMADC | | LHQDWLNGKEY |
| | | RHPYFYAPELLFFAKR | | CAKQEPERNE | | KCKVSNKALPA |
| | | YKAAFTECCQAADKAA | | CFLQHKDDNP | | PIEKTISKAKG |
| | | CLLPKLDELRDEGKAS | | NLPRLVRPEV | | QPREPQVYTLP |
| | | SAKQRLKCASLQKFGE | | DVMCTAFHDN | | PSRDELTKNQV |
| | | RAFKAWAVARLSQRFP | | EETFLKKYLY | | SLTCLVKGFYP |
| | | KAEFAEVSKLVTDLTK | | EIARRHPYFY | | SDIAVEWESNG |
| | | VHTECCHGDLLECADD | | APELLFFAKR | | QPENNYKTTPP |
| | | RADLAKYICENQDSIS | | YKAAFTECCQ | | VLDSDGSFFLY |
| | | SKLKECCEKPLLEKSH | | AADKAACLLP | | SKLTVDKSRWQ |
| | | CIAEVENDEMPADLPS | | KLDELRDEGK | | QGNVFSCSVLH |
| | | LAADFVESKDVCKNYA | | ASSAKQRLKC | | EALKFHYTQKS |
| | | EAKDVFLGMFLYEYAR | | ASLQKFGERA | | LSLSPG (SEQ |
| | | RHPDYSVVLLLRLAKT | | FKAWAVARLS | | ID NO: 2) |
| | | YETTLEKCCAAADPHE | | QRFPKAEFAE | | |
| | | CYAKVFDEFKPLVEEP | | VSKLVTDLTK | | |
| | | QNLIKQNCELFEQLGE | | VHTECCHGDL | | |
| | | YKFQNALLVRYTKKVP | | LECADDRADL | | |
| | | QVSTPTLVEVSRNLGK | | AKYICENQDS | | |
| | | VGSKCCKHPEAKRMPC | | ISSKLKECCE | | |
| | | AEDYLSVVLNQLCVLH | | KPLLEKSHCI | | |
| | | EKTPVSDRVTKCCTES | | AEVENDEMPA | | |
| | | LVNRRPCFSALEVDET | | DLPSLAADFV | | |
| | | YVPKEFNAETFTFHAD | | ESKDVCKNYA | | |
| | | ICTLSEKERQIKKQTA | | EAKDVFLGMF | | |
| | | LVELVKHKPKATKEQL | | LYEYARRHPD | | |
| | | KAVMDDFAAFVEKCCK | | YSVVLLLRLA | | |
| | | ADDKETCFAEEGKKLV | | KTYETTLEKC | | |
| | | AASQAALGLGGGGSDK | | CAAADPHECY | | |
| | | THTCPPCPAPELLGGP | | AKVFDEFKPL | | |
| | | SVFLFPPKPKDTLYIT | | VEEPQNLIKQ | | |
| | | REPEVTCVVVDVSHED | | NCELFEQLGE | | |
| | | PEVKFNWYVDGVEVHN | | YKFQNALLVR | | |
| | | AKTKPREEQYNSTYRV | | YTKKVPQVST | | |
| | | VSVLIVLHQDWLNGKE | | PTLVEVSRNL | | |
| | | YKCKVSNKALPAPIEK | | GKVGSKCCKH | | |
| | | TISKAKGQPREPQVYT | | PEAKRMPCAE | | |
| | | LPPSRDELTKNQVSLT | | DYLSVVLNQL | | |
| | | CLVKGFYPSDIAVEWE | | CVLHEKTPVS | | |
| | | SNGQPENNYKTTPPVL | | DRVTKCCTES | | |
| | | DSDGSFFLYSKLTVDK | | LVNRRPCFSA | | |
| | | SRWQQGNVFSCSVLHE | | LEVDETYVPK | | |
| | | ALKFHYTQKSLSLSPG | | EFNAETFTFH | | |
| | | | | ADICTLSEKE | | |
| | | | | RQIKKQTALV | | |
| | | | | ELVKHKPKAT | | |
| | | | | KEQLKAVMDD | | |
| | | | | FAAFVEKCCK | | |
| | | | | ADDKETCFAE | | |
| | | | | EGKKLVAASQ | | |
| | | | | AALGL (SEQ | | |
| | | | | ID NO: 10) | | |
| Fc Fusion | Fc195-G4S- | DKTHTCPPCPAPELLG | N/A | DKTHTCPPCP | GGGGS | DAHKSEVAHRF |
| Molecule | HSA | GPSVFLFPPKPKDTLY | | APELLGGPSV | (SEQ | KDLGEENFKAL |
| 32 | | ITREPEVTCVVVDVSH | | FLFPPKPKDT | ID | VLIAFAQYLQQ |
| (SEQ ID | | EDPEVKFNWYVDGVEV | | LYITREPEVT | NO: | CPFEDHVKLVN |
| NO: 47) | | HNAKTKPREEQYNSTY | | CVVVDVSHED | 13) | EVTEFAKTCVA |
| | | RVVSVLTVLHQDWLNG | | PEVKFNWYVD | | DESAENCDKSL |
| | | KEYKCKVSNKALPAPI | | GVEVHNAKTK | | HTLFGDKLCTV |
| | | EKTISKAKGQPREPQV | | PREEQYNSTY | | ATLRETYGEMA |
| | | YTLPPSRDELTKNQVS | | RVVSVLTVLH | | DCCAKQEPERN |
| | | LTCLVKGFYPSDIAVE | | QDWLNGKEYK | | ECFLQHKDDNP |
| | | WESNGQPENNYKITPP | | CKVSNKALPA | | NLPRLVRPEVD |
| | | VLDSDGSFFLYSKLTV | | PIEKTISKAK | | VMCTAFHDNEE |
| | | DKSRWQQGNVFSCSVL | | GQPREPQVYT | | TFLKKYLYEIA |
| | | HEALKFHYTQKSLSLS | | LPPSRDELTK | | RRHPYFYAPEL |
| | | PGGGGGSDAHKSEVAH | | NQVSLTCLVK | | LFFAKRYKAAF |
| | | RFKDLGEENFKALVLI | | GFYPSDIAVE | | TECCQAADKAA |
| | | AFAQYLQQCPFEDHVK | | WESNGQPENN | | CLLPKLDELRD |
| | | LVNEVTEFAKTCVADE | | YKTTPPVLDS | | EGKASSAKQRL |
| | | SAENCDKSLHTLFGDK | | DGSFFLYSKL | | KCASLQKFGER |
| | | LCTVATLRETYGEMAD | | TVDKSRWQQG | | AFKAWAVARLS |
| | | CCAKQEPERNECFLQH | | NVFSCSVLHE | | QRFPKAEFAEV |
| | | KDDNPNLPRLVRPEVD | | ALKFHYTQKS | | SKLVTDLTKVH |
| | | VMCTAFHDNEETFLKK | | LSLSPG | | TECCHGDLLEC |
| | | YLYEIARRHPYFYAPE | | (SEQ ID | | ADDRADLAKYI |
| | | LLFFAKRYKAAFTECC | | NO: 2) | | CENQDSISSKL |
| | | QAADKAACLLPKLDEL | | | | KECCEKPLLEK |
| | | RDEGKASSAKQRLKCA | | | | SHCIAEVENDE |
| | | SLQKFGERAFKAWAVA | | | | MPADLPSLAAD |
| | | RLSQRFPKAEFAEVSK | | | | FVESKDVCKNY |
| | | LVTDLTKVHTECCHGD | | | | AEAKDVFLGMF |
| | | LLECADDRADLAKYIC | | | | LYEYARRHPDY |
| | | ENQDSISSKLKECCEK | | | | SVVLLLRLAKT |
| | | PLLEKSHCIAEVENDE | | | | YETTLEKCCAA |
| | | MPADLPSLAADFVESK | | | | ADPHECYAKVF |
| | | DVCKNYAEAKDVFLGM | | | | DEFKPLVEEPQ |
| | | FLYEYARRHPDYSVVL | | | | NLIKQNCELFE |
| | | LLRLAKTYETTLEKCC | | | | QLGEYKFQNAL |
| | | AAADPHECYAKVFDEF | | | | LVRYTKKVPQV |
| | | KPLVEEPQNLIKQNCE | | | | STPTLVEVSRN |
| | | LFEQLGEYKFQNALLV | | | | LGKVGSKCCKH |
| | | RYTKKVPQVSTPTLVE | | | | PEAKRMPCAED |
| | | VSRNLGKVGSKCCKHP | | | | YLSVVLNQLCV |
| | | EAKRMPCAEDYLSVVL | | | | LHEKTPVSDRV |
| | | NQLCVLHEKTPVSDRV | | | | TKCCTESLVNR |
| | | TKCCTESLVNRRPCFS | | | | RPCFSALEVDE |
| | | ALEVDETYVPKEFNAE | | | | TYVPKEFNAET |
| | | TFTFHADICTLSEKER | | | | FTFHADICTLS |
| | | QIKKQTALVELVKHKP | | | | EKERQIKKQTA |
| | | KATKEQLKAVMDDFAA | | | | LVELVKHKPKA |
| | | FVEKCCKADDKETCFA | | | | TKEQLKAVMDD |
| | | EEGKKLVAASQAALGL | | | | FAAFVEKCCKA |
| | | | | | | DDKETCFAEEG |
| | | | | | | KKLVAASQAAL |
| | | | | | | GL (SEQ ID |
| | | | | | | NO: 10) |
| Fc Fusion | Fc195(KIH)- | DKTHTCPPCPAPELLG | DKTHTCPPCPAPEL | DKTHTCPPCP | GGGGS | EVQLLESGGGL |
| Molecule | G4S-ABD1 | GPSVFLFPPKPKDTLY | LGGPSVFLFPPKPK | APELLGGPSV | (SEQ | VQPGGSLRLSC |
| 33 | | ITREPEVTCVVVDVSH | DTLYITREPEVTCV | FLFPPKPKDT | ID | AASGFTFRSFG |
| (SEQ ID | | EDPEVKFNWYVDGVEV | VVDVSHEDPEVKFN | LYITREPEVT | NO: | MSWVRQAPGKG |
| NO: 48) | | HNAKTKPREEQYNSTY | WYVDGVEVHNAKTK | CVVVDVSHED | 13) | PEWVSSISGSG |
| | | RVVSVLIVLHQDWLNG | PREEQYNSTYRVVS | PEVKFNWYVD | | SDTLYADSVKG |
| | | KEYKCKVSNKALPAPI | VLTVLHQDWLNGKE | GVEVHNAKTK | | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVL | NNYKTTPPVLDSDG | GQPREPQVCT | | |
| | | HEALKFHYTQKSLSLS | SFFLYSKLTVDKSR | LPPSRDELTK | | |
| | | PGGGGGSEVQLLESGG | WQQGNVFSCSVLHE | NQVSLSCAVK | | |
| | | GLVQPGGSLRLSCAAS | ALKFHYTQKSLSLS | GFYPSDIAVE | | |
| | | GFTFRSFGMSWVRQAP | PG (SEQ ID NO: | WESNGQPENN | | |
| | | GKGPEWVSSISGSGSD | 9) | YKTTPPVLDS | | |
| | | TLYADSVKGRFTISRD | | DGSFFLVSKL | | |
| | | NSKNTLYLQMNSLRPE | | TVDKSRWQQG | | |
| | | DTAVYYCTIGGSLSRS | | NVFSCSVLHE | | |
| | | SQGTLVTVSS | | ALKFHYTQKS | | |
| | | | | LSLSPG | | |
| | | | | (SEQ ID | | |
| | | | | NO: 8) | | |
| Fc Fusion | Fc195(KIH)- | DKTHTCPPCPAPELLG | DKTHTCPPCPAPEL | DKTHTCPPCP | GGGGS | EVQLLESGGGL |
| Molecule | 3G4S-ABD1 | GPSVFLFPPKPKDTLY | LGGPSVFLFPPKPK | APELLGGPSV | GGGGS | VQPGGSLRLSC |
| 34 | | ITREPEVTCVVVDVSH | DTLYITREPEVTCV | FLFPPKPKDT | GGGGS | AASGFTFRSFG |
| (SEQ ID | | EDPEVKFNWYVDGVEV | VVDVSHEDPEVKFN | LYITREPEVT | (SEQ | MSWVRQAPGKG |
| NO: 49) | | HNAKTKPREEQYNSTY | WYVDGVEVHNAKTK | CVVVDVSHED | ID | PEWVSSISGSG |
| | | RVVSVLTVLHQDWLNG | PREEQYNSTYRVVS | PEVKFNWYVD | NO: | SDTLYADSVKG |
| | | KEYKCKVSNKALPAPI | VLTVLHQDWLNGKE | GVEVHNAKTK | 15) | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVL | NNYKTTPPVLDSDG | GQPREPQVCT | | |
| | | HEALKFHYTQKSLSLS | SFFLYSKLTVDKSR | LPPSRDELTK | | |
| | | PGGGGGSGGGGSGGGG | WQQGNVFSCSVLHE | NQVSLSCAVK | | |
| | | SEVQLLESGGGLVQPG | ALKFHYTQKSLSLS | GFYPSDIAVE | | |
| | | GSLRLSCAASGFTFRS | PG (SEQ ID NO: | WESNGQPENN | | |
| | | FGMSWVRQAPGKGPEW | 9) | YKTTPPVLDS | | |
| | | VSSISGSGSDILYADS | | DGSFFLVSKL | | |
| | | VKGRFTISRDNSKNTL | | TVDKSRWQQG | | |
| | | YLQMNSLRPEDTAVYY | | NVFSCSVLHE | | |
| | | CTIGGSLSRSSQGTLV | | ALKFHYTQKS | | |
| | | TVSS | | LSLSPG | | |
| | | | | (SEQ ID | | |
| | | | | NO: 8) | | |
| Fc Fusion | ABD1-G4S- | EVQLLESGGGLVQPGG | N/A | EVQLLESGGG | GGGGS | DKTHTCPPCPA |
| Molecule | Fc195 | SLRLSCAASGFTFRSF | | LVQPGGSLRL | (SEQ | PELLGGPSVFL |
| 35 | | GMSWVRQAPGKGPEWV | | SCAASGFTFR | ID | FPPKPKDTLYI |
| (SEQ ID | | SSISGSGSDTLYADSV | | SFGMSWVRQA | NO: | TREPEVTCVVV |
| NO: 50) | | KGRFTISRDNSKNTLY | | PGKGPEWVSS | 13) | DVSHEDPEVKF |
| | | LQMNSLRPEDTAVYYC | | ISGSGSDTLY | | NWYVDGVEVHN |
| | | TIGGSLSRSSQGTLVT | | ADSVKGRFTI | | AKTKPREEQYN |
| | | VSSGGGGSDKTHTCPP | | SRDNSKNTLY | | STYRVVSVLIV |
| | | CPAPELLGGPSVFLFP | | LQMNSLRPED | | LHQDWLNGKEY |
| | | PKPKDTLYITREPEVT | | TAVYYCTIGG | | KCKVSNKALPA |
| | | CVVVDVSHEDPEVKFN | | SLSRSSQGTL | | PIEKTISKAKG |
| | | WYVDGVEVHNAKTKPR | | VTVSS (SEQ | | QPREPQVYTLP |
| | | EEQYNSTYRVVSVLTV | | ID NO: 11) | | PSRDELTKNQV |
| | | LHQDWLNGKEYKCKVS | | | | SLTCLVKGFYP |
| | | NKALPAPIEKTISKAK | | | | SDIAVEWESNG |
| | | GQPREPQVYTLPPSRD | | | | QPENNYKTTPP |
| | | ELTKNQVSLTCLVKGF | | | | VLDSDGSFFLY |
| | | YPSDIAVEWESNGQPE | | | | SKLTVDKSRWQ |
| | | NNYKTTPPVLDSDGSF | | | | QGNVFSCSVLH |
| | | FLYSKLTVDKSRWQQG | | | | EALKFHYTQKS |
| | | NVFSCSVLHEALKFHY | | | | LSLSPG (SEQ |
| | | TQKSLSLSPG | | | | ID NO: 2) |
| Fc Fusion | ABD1-G4S- | EVQLLESGGGLVQPGG | DKTHTCPPCPAPEL | EVQLLESGGG | GGGGS | DKTHTCPPCPA |
| Molecule | Fc195(KIH) | SLRLSCAASGFTFRSF | LGGPSVFLFPPKPK | LVQPGGSLRL | (SEQ | PELLGGPSVFL |
| 36 | | GMSWVRQAPGKGPEWV | DTLYITREPEVTCV | SCAASGFTFR | ID | FPPKPKDTLYI |
| (SEQ ID | | SSISGSGSDTLYADSV | VVDVSHEDPEVKFN | SFGMSWVRQA | NO: | TREPEVTCVVV |
| NO: 51) | | KGRFTISRDNSKNTLY | WYVDGVEVHNAKTK | PGKGPEWVSS | 13) | DVSHEDPEVKF |
| | | LQMNSLRPEDTAVYYC | PREEQYNSTYRVVS | ISGSGSDTLY | | NWYVDGVEVHN |
| | | TIGGSLSRSSQGTLVT | VLTVLHQDWLNGKE | ADSVKGRFTI | | AKTKPREEQYN |
| | | VSSGGGGSDKTHTCPP | YKCKVSNKALPAPI | SRDNSKNTLY | | STYRVVSVLIV |
| | | CPAPELLGGPSVFLFP | EKTISKAKGQPREP | LQMNSLRPED | | LHQDWLNGKEY |
| | | PKPKDTLYITREPEVT | QVYTLPPCRDELTK | TAVYYCTIGG | | KCKVSNKALPA |
| | | CVVVDVSHEDPEVKFN | NQVSLWCLVKGFYP | SLSRSSQGTL | | PIEKTISKAKG |
| | | WYVDGVEVHNAKTKPR | SDIAVEWESNGQPE | VTVSS (SEQ | | QPREPQVCTLP |
| | | EEQYNSTYRVVSVLTV | NNYKTTPPVLDSDG | ID NO: 11) | | PSRDELTKNQV |
| | | LHQDWLNGKEYKCKVS | SFFLYSKLTVDKSR | | | SLSCAVKGFYP |
| | | NKALPAPIEKTISKAK | WQQGNVFSCSVLHE | | | SDIAVEWESNG |
| | | GQPREPQVCTLPPSRD | ALKFHYTQKSLSLS | | | QPENNYKTTPP |
| | | ELTKNQVSLSCAVKGF | PG (SEQ ID NO: | | | VLDSDGSFFLV |
| | | YPSDIAVEWESNGQPE | 9) | | | SKLTVDKSRWQ |
| | | NNYKTTPPVLDSDGSF | | | | QGNVFSCSVLH |
| | | FLVSKLTVDKSRWQQG | | | | EALKFHYTQKS |
| | | NVFSCSVLHEALKFHY | | | | LSLSPG (SEQ |
| | | TQKSLSLSPG | | | | ID NO: 8) |
| Fc Fusion | Fc195-G4S- | DKTHTCPPCPAPELLG | N/A | DKTHTCPPCP | GGGGS | EVQLLESGGGL |
| Molecule | ABD1 | GPSVFLFPPKPKDTLY | | APELLGGPSV | (SEQ | VQPGGSLRLSC |
| 37 | | ITREPEVTCVVVDVSH | | FLFPPKPKDT | ID | AASGFTFRSFG |
| (SEQ ID | | EDPEVKFNWYVDGVEV | | LYITREPEVT | NO: | MSWVRQAPGKG |
| NO: 52) | | HNAKTKPREEQYNSTY | | CVVVDVSHED | 13) | PEWVSSISGSG |
| | | RVVSVLIVLHQDWLNG | | PEVKFNWYVD | | SDTLYADSVKG |
| | | KEYKCKVSNKALPAPI | | GVEVHNAKTK | | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVL | | GQPREPQVYT | | |
| | | HEALKFHYTQKSLSLS | | LPPSRDELTK | | |
| | | PGGGGGSEVQLLESGG | | NQVSLTCLVK | | |
| | | GLVQPGGSLRLSCAAS | | GFYPSDIAVE | | |
| | | GFTFRSFGMSWVRQAP | | WESNGQPENN | | |
| | | GKGPEWVSSISGSGSD | | YKTTPPVLDS | | |
| | | TLYADSVKGRFTISRD | | DGSFFLYSKL | | |
| | | NSKNTLYLQMNSLRPE | | TVDKSRWQQG | | |
| | | DTAVYYCTIGGSLSRS | | NVFSCSVLHE | | |
| | | SQGTLVTVSS | | ALKFHYTQKS | | |
| | | | | LSLSPG | | |
| | | | | (SEQ ID | | |
| | | | | NO: 2) | | |
| Fc Fusion | Fc195- | DKTHTCPPCPAPELLG | N/A | DKTHTCPPCP | GGGGS | EVQLLESGGGL |
| Molecule | 3G4S-ABD1 | GPSVFLFPPKPKDTLY | | APELLGGPSV | GGGGS | VQPGGSLRLSC |
| 38 | | ITREPEVTCVVVDVSH | | FLFPPKPKDT | GGGGS | AASGFTFRSFG |
| (SEQ ID | | EDPEVKFNWYVDGVEV | | LYITREPEVT | (SEQ | MSWVRQAPGKG |
| NO: 53) | | HNAKTKPREEQYNSTY | | CVVVDVSHED | ID | PEWVSSISGSG |
| | | RVVSVLTVLHQDWLNG | | PEVKFNWYVD | NO: | SDTLYADSVKG |
| | | KEYKCKVSNKALPAPI | | GVEVHNAKTK | 15) | RFTISRDNSKN |
| | | EKTISKAKGQPREPQV | | PREEQYNSTY | | TLYLQMNSLRP |
| | | YTLPPSRDELTKNQVS | | RVVSVLTVLH | | EDTAVYYCTIG |
| | | LTCLVKGFYPSDIAVE | | QDWLNGKEYK | | GSLSRSSQGTL |
| | | WESNGQPENNYKTTPP | | CKVSNKALPA | | VTVSS (SEQ |
| | | VLDSDGSFFLYSKLTV | | PIEKTISKAK | | ID NO: 11) |
| | | DKSRWQQGNVFSCSVL | | GQPREPQVYT | | |
| | | HEALKFHYTQKSLSLS | | LPPSRDELTK | | |
| | | PGGGGGSGGGGSGGGG | | NQVSLTCLVK | | |
| | | SEVQLLESGGGLVQPG | | GFYPSDIAVE | | |
| | | GSLRLSCAASGFTFRS | | WESNGQPENN | | |
| | | FGMSWVRQAPGKGPEW | | YKTTPPVLDS | | |
| | | VSSISGSGSDTLYADS | | DGSFFLYSKL | | |
| | | VKGRFTISRDNSKNTL | | TVDKSRWQQG | | |
| | | YLQMNSLRPEDTAVYY | | NVFSCSVLHE | | |
| | | CTIGGSLSRSSQGTLV | | ALKFHYTQKS | | |
| | | TVSS | | LSLSPG | | |
| | | | | (SEQ ID | | |
| | | | | NO: 2) | | |
| Fc Fusion | ABD1- | EVQLLESGGGLVQPGG | N/A | EVQLLESGGG | GGGGS | DKTHTCPPCPA |
| Molecule | 3G4S-Fc195 | SLRLSCAASGFTFRSF | | LVQPGGSLRL | GGGGS | PELLGGPSVFL |
| 39 | | GMSWVRQAPGKGPEWV | | SCAASGFTFR | GGGGS | FPPKPKDTLYI |
| (SEQ ID | | SSISGSGSDTLYADSV | | SFGMSWVRQA | (SEQ | TREPEVTCVVV |
| NO: 54) | | KGRFTISRDNSKNTLY | | PGKGPEWVSS | ID | DVSHEDPEVKF |
| | | LQMNSLRPEDTAVYYC | | ISGSGSDTLY | NO: | NWYVDGVEVHN |
| | | TIGGSLSRSSQGTLVT | | ADSVKGRFTI | 15) | AKTKPREEQYN |
| | | VSSGGGGSGGGGSGGG | | SRDNSKNTLY | | STYRVVSVLTV |
| | | GSDKTHTCPPCPAPEL | | LQMNSLRPED | | LHQDWLNGKEY |
| | | LGGPSVFLFPPKPKDT | | TAVYYCTIGG | | KCKVSNKALPA |
| | | LYITREPEVTCVVVDV | | SLSRSSQGTL | | PIEKTISKAKG |
| | | SHEDPEVKFNWYVDGV | | VTVSS (SEQ | | QPREPQVYTLP |
| | | EVHNAKTKPREEQYNS | | ID NO: 11) | | PSRDELTKNQV |
| | | TYRVVSVLIVLHQDWL | | | | SLTCLVKGFYP |
| | | NGKEYKCKVSNKALPA | | | | SDIAVEWESNG |
| | | PIEKTISKAKGQPREP | | | | QPENNYKTTPP |
| | | QVYTLPPSRDELTKNQ | | | | VLDSDGSFFLY |
| | | VSLTCLVKGFYPSDIA | | | | SKLTVDKSRWQ |
| | | VEWESNGQPENNYKTT | | | | QGNVFSCSVLH |
| | | PPVLDSDGSFFLYSKL | | | | EALKFHYTQKS |
| | | TVDKSRWQQGNVFSCS | | | | LSLSPG (SEQ |
| | | VLHEALKFHYTQKSLS | | | | ID NO: 2) |
| | | LSPG | | | | |
| Fc Fusion | ABD1- | EVQLLESGGGLVQPGG | DKTHTCPPCPAPEL | EVQLLESGGG | GGGGS | DKTHTCPPCPA |
| Molecule | 3G4S- | SLRLSCAASGFTFRSF | LGGPSVFLFPPKPK | LVQPGGSLRL | GGGGS | PELLGGPSVFL |
| 40 | Fc195(KIH) | GMSWVRQAPGKGPEWV | DTLYITREPEVTCV | SCAASGFTFR | GGGGS | FPPKPKDTLYI |
| (SEQ ID | | SSISGSGSDTLYADSV | VVDVSHEDPEVKFN | SFGMSWVRQA | (SEQ | TREPEVTCVVV |
| NO: 55) | | KGRFTISRDNSKNTLY | WYVDGVEVHNAKTK | PGKGPEWVSS | ID | DVSHEDPEVKF |
| | | LQMNSLRPEDTAVYYC | PREEQYNSTYRVVS | ISGSGSDTLY | NO: | NWYVDGVEVHN |
| | | TIGGSLSRSSQGTLVT | VLTVLHQDWLNGKE | ADSVKGRFTI | 15) | AKTKPREEQYN |
| | | VSSGGGGSGGGGSGGG | YKCKVSNKALPAPI | SRDNSKNTLY | | STYRVVSVLTV |
| | | GSDKTHTCPPCPAPEL | EKTISKAKGQPREP | LQMNSLRPED | | LHQDWLNGKEY |
| | | LGGPSVFLFPPKPKDT | QVYTLPPCRDELTK | TAVYYCTIGG | | KCKVSNKALPA |
| | | LYITREPEVTCVVVDV | NQVSLWCLVKGFYP | SLSRSSQGTL | | PIEKTISKAKG |
| | | SHEDPEVKFNWYVDGV | SDIAVEWESNGQPE | VTVSS (SEQ | | QPREPQVCTLP |
| | | EVHNAKTKPREEQYNS | NNYKTTPPVLDSDG | ID NO: 11) | | PSRDELTKNQV |
| | | TYRVVSVLIVLHQDWL | SFFLYSKLTVDKSR | | | SLSCAVKGFYP |
| | | NGKEYKCKVSNKALPA | WQQGNVFSCSVLHE | | | SDIAVEWESNG |
| | | PIEKTISKAKGQPREP | ALKFHYTQKSLSLS | | | QPENNYKTTPP |
| | | QVCTLPPSRDELTKNQ | PG (SEQ ID NO: | | | VLDSDGSFFLV |
| | | VSLSCAVKGFYPSDIA | 9) | | | SKLTVDKSRWQ |
| | | VEWESNGQPENNYKIT | | | | QGNVFSCSVLH |
| | | PPVLDSDGSFFLVSKL | | | | EALKFHYTQKS |
| | | TVDKSRWQQGNVFSCS | | | | LSLSPG (SEQ |
| | | VLHEALKFHYTQKSLS | | | | ID NO: 8) |
| | | LSPG | | | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)6- | GPSVFLFPPKPKDTLY | LGGPSVFLFPPKPK | APELLGGPSV | GGGGS | VQPGGSLRLSC |
| 41 | ABD1 | ITREPEVTCVVVDVSH | DTLYITREPEVTCV | FLFPPKPKDT | GGGGS | AASGFTFRSFG |
| (SEQ ID | | EDPEVKFNWYVDGVEV | VVDVSHEDPEVKFN | LYITREPEVT | GGGGS | MSWVRQAPGKG |
| NO: 68) | | HNAKTKPREEQYNSTY | WYVDGVEVHNAKTK | CVVVDVSHED | GGGGS | PEWVSSISGSG |
| | | RVVSVLTVLHQDWLNG | PREEQYNSTYRVVS | PEVKFNWYVD | GGGGS | SDTLYADSVKG |
| | | KEYKCKVSNKALPAPI | VLTVLHQDWLNGKE | GVEVHNAKTK | (SEQ | RFTISRDNSKN |
| | | EKTISKAKGQPREPQV | YKCKVSNKALPAPI | PREEQYNSTY | ID | TLYLQMNSLRP |
| | | CTLPPSRDELTKNQVS | EKTISKAKGQPREP | RVVSVLTVLH | NO: | EDTAVYYCTIG |
| | | LSCAVKGFYPSDIAVE | QVYTLPPCRDELTK | QDWLNGKEYK | 74) | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 11) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SGGGGSGGGGSGGGGS | ALKYHYTQKSLSLS | GFYPSDIAVE | | |
| | | EVQLLESGGGLVQPGG | PG (SEQ ID NO: | WESNGQPENN | | |
| | | SLRLSCAASGFTFRSF | 7) | YKTTPPVLDS | | |
| | | GMSWVRQAPGKGPEWV | | DGSFFLVSKL | | |
| | | SSISGSGSDTLYADSV | | TVDKSRWQQG | | |
| | | KGRFTISRDNSKNTLY | | NVFSCSVMHE | | |
| | | LQMNSLRPEDTAVYYC | | ALKYHYTQKS | | |
| | | TIGGSLSRSSQGTLVT | | LSLSPG | | |
| | | VSS | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)8- | GPSVFLFPPKPKDTLY | LGGPSVFLFPPKPK | APELLGGPSV | GGGGS | VQPGGSLRLSC |
| 42 | ABD1 | ITREPEVTCVVVDVSH | DTLYITREPEVTCV | FLFPPKPKDT | GGGGS | AASGFTFRSFG |
| (SEQ ID | | EDPEVKFNWYVDGVEV | VVDVSHEDPEVKFN | LYITREPEVT | GGGGS | MSWVRQAPGKG |
| NO: 69) | | HNAKTKPREEQYNSTY | WYVDGVEVHNAKTK | CVVVDVSHED | GGGGS | PEWVSSISGSG |
| | | RVVSVLTVLHQDWLNG | PREEQYNSTYRVVS | PEVKFNWYVD | GGGGS | SDTLYADSVKG |
| | | KEYKCKVSNKALPAPI | VLTVLHQDWLNGKE | GVEVHNAKTK | GGGGS | RFTISRDNSKN |
| | | EKTISKAKGQPREPQV | YKCKVSNKALPAPI | PREEQYNSTY | GGGGS | TLYLQMNSLRP |
| | | CTLPPSRDELTKNQVS | EKTISKAKGQPREP | RVVSVLTVLH | (SEQ | EDTAVYYCTIG |
| | | LSCAVKGFYPSDIAVE | QVYTLPPCRDELTK | QDWLNGKEYK | ID | GSLSRSSQGTL |
| | | WESNGQPENNYKTTPP | NQVSLWCLVKGFYP | CKVSNKALPA | NO: | VTVSS (SEQ |
| | | VLDSDGSFFLVSKLTV | SDIAVEWESNGQPE | PIEKTISKAK | 75) | ID NO: 11) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVESCSVMHE | NQVSLSCAVK | | |
| | | SGGGGSGGGGSGGGGS | ALKYHYTQKSLSLS | GFYPSDIAVE | | |
| | | GGGGSGGGGSEVQLLE | PG (SEQ ID NO: | WESNGQPENN | | |
| | | SGGGLVQPGGSLRLSC | 7) | YKTTPPVLDS | | |
| | | AASGFTFRSFGMSWVR | | DGSFFLVSKL | | |
| | | QAPGKGPEWVSSISGS | | TVDKSRWQQG | | |
| | | GSDTLYADSVKGRFTI | | NVFSCSVMHE | | |
| | | SRDNSKNTLYLQMNSL | | ALKYHYTQKS | | |
| | | RPEDTAVYYCTIGGSL | | LSLSPG | | |
| | | SRSSQGTLVTVSS | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)3- | GPSVFLFPPKPKDTLY | LGGPSVFLFPPKPK | APELLGGPSV | GGGGS | VQPGGSLRLSC |
| 43 | ABD1 | ITREPEVTCVVVDVSH | DTLYITREPEVTCV | FLFPPKPKDT | GGGGS | AASGFTFRSAG |
| (SEQ ID | (F32A) | EDPEVKFNWYVDGVEV | VVDVSHEDPEVKFN | LYITREPEVT | (SEQ | MSWVRQAPGKG |
| NO: 70) | | HNAKTKPREEQYNSTY | WYVDGVEVHNAKTK | CVVVDVSHED | ID | PEWVSSISGSG |
| | | RVVSVLTVLHQDWLNG | PREEQYNSTYRVVS | PEVKFNWYVD | NO: | SDTLYADSVKG |
| | | KEYKCKVSNKALPAPI | VLTVLHQDWLNGKE | GVEVHNAKTK | 15) | RFTISRDNSKN |
| | | EKTISKAKGQPREPQV | YKCKVSNKALPAPI | PREEQYNSTY | | TLYLQMNSLRP |
| | | CTLPPSRDELTKNQVS | EKTISKAKGQPREP | RVVSVLTVLH | | EDTAVYYCTIG |
| | | LSCAVKGFYPSDIAVE | QVYTLPPCRDELTK | QDWLNGKEYK | | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 76) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SEVQLLESGGGLVQPG | ALKYHYTQKSLSLS | GFYPSDIAVE | | |
| | | GSLRLSCAASGFTFRS | PG (SEQ ID NO: | WESNGQPENN | | |
| | | AGMSWVRQAPGKGPEW | 7) | YKTTPPVLDS | | |
| | | VSSISGSGSDTLYADS | | DGSFFLVSKL | | |
| | | VKGRFTISRDNSKNTL | | TVDKSRWQQG | | |
| | | YLQMNSLRPEDTAVYY | | NVFSCSVMHE | | |
| | | CTIGGSLSRSSQGTLV | | ALKYHYTQKS | | |
| | | TVSS | | LSLSPG | | |
| | | | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)6- | GPSVFLFPPKPKDTLY | LGGPSVFLFPPKPK | APELLGGPSV | GGGGS | VQPGGSLRLSC |
| 44 | ABD1 | ITREPEVTCVVVDVSH | DTLYITREPEVTCV | FLFPPKPKDT | GGGGS | AASGFTFRSAG |
| (SEQ ID | (F32A) | EDPEVKFNWYVDGVEV | VVDVSHEDPEVKFN | LYITREPEVT | GGGGS | MSWVRQAPGKG |
| NO: 71) | | HNAKTKPREEQYNSTY | WYVDGVEVHNAKTK | CVVVDVSHED | GGGGS | PEWVSSISGSG |
| | | RVVSVLTVLHQDWLNG | PREEQYNSTYRVVS | PEVKFNWYVD | GGGGS | SDTLYADSVKG |
| | | KEYKCKVSNKALPAPI | VLTVLHQDWLNGKE | GVEVHNAKTK | (SEQ | RFTISRDNSKN |
| | | EKTISKAKGQPREPQV | YKCKVSNKALPAPI | PREEQYNSTY | ID | TLYLQMNSLRP |
| | | CTLPPSRDELTKNQVS | EKTISKAKGQPREP | RVVSVLTVLH | NO: | EDTAVYYCTIG |
| | | LSCAVKGFYPSDIAVE | QVYTLPPCRDELTK | QDWLNGKEYK | 74) | GSLSRSSQGTL |
| | | WESNGQPENNYKTTPP | NQVSLWCLVKGFYP | CKVSNKALPA | | VTVSS (SEQ |
| | | VLDSDGSFFLVSKLTV | SDIAVEWESNGQPE | PIEKTISKAK | | ID NO: 76) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SGGGGSGGGGSGGGGS | ALKYHYTQKSLSLS | GFYPSDIAVE | | |
| | | EVQLLESGGGLVQPGG | PG (SEQ ID NO: | WESNGQPENN | | |
| | | SLRLSCAASGFTFRSA | 7) | YKTTPPVLDS | | |
| | | GMSWVRQAPGKGPEWV | | DGSFFLVSKL | | |
| | | SSISGSGSDTLYADSV | | TVDKSRWQQG | | |
| | | KGRFTISRDNSKNTLY | | NVFSCSVMHE | | |
| | | LQMNSLRPEDTAVYYC | | ALKYHYTQKS | | |
| | | TIGGSLSRSSQGTLVT | | LSLSPG | | |
| | | VSS | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)7- | GPSVFLFPPKPKDTLY | LGGPSVFLFPPKPK | APELLGGPSV | GGGGS | VQPGGSLRLSC |
| 45 | ABD1 | ITREPEVTCVVVDVSH | DTLYITREPEVTCV | FLFPPKPKDT | GGGGS | AASGFTFRSAG |
| (SEQ ID | (F32A) | EDPEVKFNWYVDGVEV | VVDVSHEDPEVKFN | LYITREPEVT | GGGGS | MSWVRQAPGKG |
| NO: 72) | | HNAKTKPREEQYNSTY | WYVDGVEVHNAKTK | CVVVDVSHED | GGGGS | PEWVSSISGSG |
| | | RVVSVLTVLHQDWLNG | PREEQYNSTYRVVS | PEVKFNWYVD | GGGGS | SDTLYADSVKG |
| | | KEYKCKVSNKALPAPI | VLTVLHQDWLNGKE | GVEVHNAKTK | GGGGS | RFTISRDNSKN |
| | | EKTISKAKGQPREPQV | YKCKVSNKALPAPI | PREEQYNSTY | (SEQ | TLYLQMNSLRP |
| | | CTLPPSRDELTKNQVS | EKTISKAKGQPREP | RVVSVLTVLH | ID | EDTAVYYCTIG |
| | | LSCAVKGFYPSDIAVE | QVYTLPPCRDELTK | QDWLNGKEYK | NO: | GSLSRSSQGTL |
| | | WESNGQPENNYKTTPP | NQVSLWCLVKGFYP | CKVSNKALPA | 14) | VTVSS (SEQ |
| | | VLDSDGSFFLVSKLTV | SDIAVEWESNGQPE | PIEKTISKAK | | ID NO: 76) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SGGGGSGGGGSGGGGS | ALKYHYTQKSLSLS | GFYPSDIAVE | | |
| | | GGGGSEVQLLESGGGL | PG (SEQ ID NO: | WESNGQPENN | | |
| | | VQPGGSLRLSCAASGF | 7) | YKTTPPVLDS | | |
| | | TFRSAGMSWVRQAPGK | | DGSFFLVSKL | | |
| | | GPEWVSSISGSGSDTL | | TVDKSRWQQG | | |
| | | YADSVKGRFTISRDNS | | NVFSCSVMHE | | |
| | | KNTLYLQMNSLRPEDT | | ALKYHYTQKS | | |
| | | AVYYCTIGGSLSRSSQ | | LSLSPG | | |
| | | GTLVTVSS | | (SEQ ID | | |
| | | | | NO: 6) | | |
| Fc Fusion | Fc197(KIH)- | DKTHTCPPCPAPELLG | DKTHTCPPCPAPEL | DKTHTCPPCP | GGGGS | EVQLLESGGGL |
| Molecule | (G4S)8- | GPSVFLFPPKPKDTLY | LGGPSVFLFPPKPK | APELLGGPSV | GGGGS | VQPGGSLRLSC |
| 46 | ABD1 | ITREPEVTCVVVDVSH | DTLYITREPEVTCV | FLFPPKPKDT | GGGGS | AASGFTFRSAG |
| (SEQ ID | (F32A) | EDPEVKFNWYVDGVEV | VVDVSHEDPEVKFN | LYITREPEVT | GGGGS | MSWVRQAPGKG |
| NO: 73) | | HNAKTKPREEQYNSTY | WYVDGVEVHNAKTK | CVVVDVSHED | GGGGS | PEWVSSISGSG |
| | | RVVSVLTVLHQDWLNG | PREEQYNSTYRVVS | PEVKFNWYVD | GGGGS | SDTLYADSVKG |
| | | KEYKCKVSNKALPAPI | VLTVLHQDWLNGKE | GVEVHNAKTK | GGGGS | RFTISRDNSKN |
| | | EKTISKAKGQPREPQV | YKCKVSNKALPAPI | PREEQYNSTY | GGGGS | TLYLQMNSLRP |
| | | CTLPPSRDELTKNQVS | EKTISKAKGQPREP | RVVSVLTVLH | (SEQ | EDTAVYYCTIG |
| | | LSCAVKGFYPSDIAVE | QVYTLPPCRDELTK | QDWLNGKEYK | ID | GSLSRSSQGTL |
| | | WESNGQPENNYKTTPP | NQVSLWCLVKGFYP | CKVSNKALPA | NO: | VTVSS (SEQ |
| | | VLDSDGSFFLVSKLTV | SDIAVEWESNGQPE | PIEKTISKAK | 75) | ID NO: 76) |
| | | DKSRWQQGNVFSCSVM | NNYKTTPPVLDSDG | GQPREPQVCT | | |
| | | HEALKYHYTQKSLSLS | SFFLYSKLTVDKSR | LPPSRDELTK | | |
| | | PGGGGGSGGGGSGGGG | WQQGNVFSCSVMHE | NQVSLSCAVK | | |
| | | SGGGGSGGGGSGGGGS | ALKYHYTQKSLSLS | GFYPSDIAVE | | |
| | | GGGGSGGGGSEVQLLE | PG (SEQ ID NO: | WESNGQPENN | | |
| | | SGGGLVQPGGSLRLSC | 7) | YKTTPPVLDS | | |
| | | AASGFTFRSAGMSWVR | | DGSFFLVSKL | | |
| | | QAPGKGPEWVSSISGS | | TVDKSRWQQG | | |
| | | GSDTLYADSVKGRFTI | | NVFSCSVMHE | | |
| | | SRDNSKNTLYLQMNSL | | ALKYHYTQKS | | |
| | | RPEDTAVYYCTIGGSL | | LSLSPG | | |
| | | SRSSQGTLVTVSS | | (SEQ ID | | |
| | | | | NO: 6) | | |

Further described herein, in certain embodiments, are Fc fusion molecules that bind neonatal Fc receptor (FcRn) comprising a sequence having at least 80% sequence identity to any one of SEQ ID NOs: 16-55. In some embodiments, the Fc fusion molecule comprises a sequence having at least 85% sequence identity with an amino acid sequence according to any one of SEQ ID NOs: 16-55. In some embodiments, the Fc fusion molecule comprises a sequence having at least 90% sequence identity with an amino acid sequence according to any one of SEQ ID NOs: 16-55. In some embodiments, the Fc fusion molecule comprises a sequence having at least 95% sequence identity with an amino acid sequence according to any one of SEQ ID NOs: 16-55. In some embodiments, the Fc fusion molecule comprises a sequence having at least 96% sequence identity with an amino acid sequence according to any one of SEQ ID NOs: 16-55. In some embodiments, the Fc fusion molecule comprises a sequence having at least 97% sequence identity with an amino acid sequence according to any one of SEQ ID NOs: 16-55. In some embodiments, the Fc fusion molecule comprises a sequence having at least 98% sequence identity with an amino acid sequence according to any one of SEQ ID NOs: 16-55. In some embodiments, the Fc fusion molecule comprises a sequence having at least 99% sequence identity with an amino acid sequence according to any one of SEQ ID NOs: 16-55. In some embodiments, the Fc fusion molecule comprises a sequence according to any one of SEQ ID NOs: 16-55.

In some embodiments, the Fc fusion molecule comprises a sequence having at least 80% sequence identity to any one of SEQ ID NOs: 16-55 or 68-73. In some embodiments, the Fc fusion molecule comprises a sequence having at least 85% sequence identity with an amino acid sequence according to any one of SEQ ID NOs: 16-55 or 68-73. In some embodiments, the Fc fusion molecule comprises a sequence having at least 90% sequence identity with an amino acid sequence according to any one of SEQ ID NOs: 16-55 or 68-73. In some embodiments, the Fc fusion molecule comprises a sequence having at least 95% sequence identity with an amino acid sequence according to any one of SEQ ID NOs: 16-55 or 68-73. In some embodiments, the Fc fusion molecule comprises a sequence having at least 96% sequence identity with an amino acid sequence according to any one of SEQ ID NOs: 16-55 or 68-73. In some embodiments, the Fc fusion molecule comprises a sequence having at least 97% sequence identity with an amino acid sequence according to any one of SEQ ID NOs: 16-55 or 68-73. In some embodiments, the Fc fusion molecule comprises a sequence having at least 98% sequence identity with an amino acid sequence according to any one of SEQ ID NOs: 16-55 or 68-73. In some embodiments, the Fc fusion molecule comprises a sequence having at least 99% sequence identity with an amino acid sequence according to any one of SEQ ID NOs: 16-55 or 68-73. In some embodiments, the Fc fusion molecule comprises a sequence according to any one of SEQ ID NOs: 16-55 or 68-73.

In some embodiments, the Fc fusion molecule comprises a sequence having at least 80% sequence identity to any one of SEQ ID NOs: 28-55 or 68-73. In some embodiments, the Fc fusion molecule comprises a sequence having at least 85% sequence identity with an amino acid sequence according to any one of SEQ ID NOs: 28-55 or 68-73. In some embodiments, the Fc fusion molecule comprises a sequence having at least 90% sequence identity with an amino acid sequence according to any one of SEQ ID NOs: 28-55 or 68-73. In some embodiments, the Fc fusion molecule comprises a sequence having at least 95% sequence identity with an amino acid sequence according to any one of SEQ ID NOs: 28-55 or 68-73. In some embodiments, the Fc fusion molecule comprises a sequence having at least 96% sequence identity with an amino acid sequence according to any one of SEQ ID NOs: 28-55 or 68-73. In some embodiments, the Fc fusion molecule comprises a sequence having at least 97% sequence identity with an amino acid sequence according to any one of SEQ ID NOs: 28-55 or 68-73. In some embodiments, the Fc fusion molecule comprises a sequence having at least 98% sequence identity with an amino acid sequence according to any one of SEQ ID NOs: 28-55 or 68-73. In some embodiments, the Fc fusion molecule comprises a sequence having at least 99% sequence identity with an amino acid sequence according to any one of SEQ ID NOs: 28-55 or 68-73. In some embodiments, the Fc fusion molecule comprises a sequence according to any one of SEQ ID NOs: 28-55 or 68-73.

In some embodiments, the Fc fusion molecule comprises a sequence having at least 80% sequence identity to any one of SEQ ID NOs: 28-45. In some embodiments, the Fc fusion molecule comprises a sequence having at least 85% sequence identity with an amino acid sequence according to any one of SEQ ID NOs: 28-45. In some embodiments, the Fc fusion molecule comprises a sequence having at least 90% sequence identity with an amino acid sequence according to any one of SEQ ID NOs: 28-45. In some embodiments, the Fc fusion molecule comprises a sequence having at least 95% sequence identity with an amino acid sequence according to any one of SEQ ID NOs: 28-45. In some embodiments, the Fc fusion molecule comprises a sequence having at least 96% sequence identity with an amino acid sequence according to any one of SEQ ID NOs: 28-45. In some embodiments, the Fc fusion molecule comprises a sequence having at least 97% sequence identity with an amino acid sequence according to any one of SEQ ID NOs: 28-45. In some embodiments, the Fc fusion molecule comprises a sequence having at least 98% sequence identity with an amino acid sequence according to any one of SEQ ID NOs: 28-45. In some embodiments, the Fc fusion molecule comprises a sequence having at least 99% sequence identity with an amino acid sequence according to any one of SEQ ID NOs: 28-45. In some embodiments, the Fc fusion molecule comprises a sequence according to any one of SEQ ID NOs: 28-45.

In some embodiments, the Fc fusion molecule comprises a sequence having at least 80% sequence identity to any one of SEQ ID NOs: 46-55. In some embodiments, the Fc fusion molecule comprises a sequence having at least 85% sequence identity with an amino acid sequence according to any one of SEQ ID NOs: 46-55. In some embodiments, the Fc fusion molecule comprises a sequence having at least 90% sequence identity with an amino acid sequence according to any one of SEQ ID NOs: 46-55. In some embodiments, the Fc fusion molecule comprises a sequence having at least 95% sequence identity with an amino acid sequence according to any one of SEQ ID NOs: 46-55. In some embodiments, the Fc fusion molecule comprises a sequence having at least 96% sequence identity with an amino acid sequence according to any one of SEQ ID NOs: 46-55. In some embodiments, the Fc fusion molecule comprises a sequence having at least 97% sequence identity with an amino acid sequence according to any one of SEQ ID NOs: 46-55. In some embodiments, the Fc fusion molecule comprises a sequence having at least 98% sequence identity with an amino acid sequence according to any one of SEQ ID NOs: 46-55. In some embodiments, the Fc fusion molecule comprises a sequence having at least 99% sequence identity with an amino acid sequence according to any one of SEQ ID NOs: 46-55. In some embodiments, the Fc fusion molecule comprises a sequence according to any one of SEQ ID NOs: 46-55.

In some embodiments, the Fc fusion molecule comprises a sequence having at least 80% sequence identity to any one of SEQ ID NOs: 68-73. In some embodiments, the Fc fusion molecule comprises a sequence having at least 85% sequence identity with an amino acid sequence according to any one of SEQ ID NOs: 68-73. In some embodiments, the Fc fusion molecule comprises a sequence having at least 90% sequence identity with an amino acid sequence according to any one of SEQ ID NOs: 68-73. In some embodiments, the Fc fusion molecule comprises a sequence having at least 95% sequence identity with an amino acid sequence according to any one of SEQ ID NOs: 68-73. In some embodiments, the Fc fusion molecule comprises a sequence having at least 96% sequence identity with an amino acid sequence according to any one of SEQ ID NOs: 68-73. In some embodiments, the Fc fusion molecule comprises a sequence having at least 97% sequence identity with an amino acid sequence according to any one of SEQ ID NOs: 68-73. In some embodiments, the Fc fusion molecule comprises a sequence having at least 98% sequence identity with an amino acid sequence according to any one of SEQ ID NOs: 68-73. In some embodiments, the Fc fusion molecule comprises a sequence having at least 99% sequence identity with an amino acid sequence according to any one of SEQ ID NOs: 68-73. In some embodiments, the Fc fusion molecule comprises a sequence according to any one of SEQ ID NOs: 68-73.

In some embodiments, the Fc fusion molecules described herein comprise an extended half-life (i.e., serum half-life). In some embodiments, the Fc fusion molecules described herein comprise a half-life of at least about 14, 28, 42, 56, 70, 84, 96, or more than 96 weeks. In some embodiments, the Fc fusion molecules described herein comprise a half-life in a range of about 14 days to about 96 days, about 14 days to about 84 days, about 14 days to about 70 days, about 14 days to about 56 days, about 14 days to about 42 days, about 14 days to about 28 days, of about 28 days to about 96 days, about 28 days to about 84 days, about 28 days to about 70 days, about 28 days to about 56 days, about 28 days to about 42 days, of about 42 days to about 96 days, about 42 days to about 84 days, about 42 days to about 70 days, or about 42 days to about 56 days. In some embodiments, the Fc fusion molecules described herein comprise a half-life in a range of about 42 days to about 56 days. In some embodiments, the Fc fusion molecules described herein comprise a half-life of at least about 50 days. In some embodiments, the Fc fusion molecules described herein comprise a half-life of about 50 days. Methods of measuring half-life are known in the art. In some embodiments, the half-life is measured in a non-human primate. In some embodiments, the half-life is measured in a human.

In some embodiments, the Fc fusion molecules described herein have a half-life that is at least 20% longer than a comparator molecule. In some embodiments, the comparator molecule is an Fc fusion molecule not comprising an albumin or an albumin binding domain. In some embodiments, the albumin or the albumin domain comprises a sequence having at least 80% sequence identity (e.g., 80%, 85%, 90%, 95%, 97%, 99%, 100%) to the amino acid sequence of any one of SEQ ID NOs: 10-12 any one of SEQ ID NOs: 10-12. In some embodiments, the half-life of the Fc fusion molecules described herein is at least 30%, at least 40%, at least 50%, at least 60%, at least 70%, at least 80%, or at least 90% longer than the half-life of the comparator molecule. In some embodiments, the half-life of the Fc fusion molecules described herein is longer than the half-life of the comparator molecule by at least 2 fold, at least 3 fold, at least 4 fold, at least 5 fold, at least 6 fold, at least 7 fold, at least 8 fold, at least 9 fold, or at least 10 fold.

In some embodiments, the Fc fusion molecule reduces IgG levels to less than 60%, to less than 50%, to less than 40%, to less than 30%, or to less than 25%. In some embodiments, the Fc fusion molecule reduces IgG levels to less than 60%. In some embodiments, the Fc fusion molecule reduces IgG levels to less than 50%. In some embodiments, the Fc fusion molecule reduces IgG levels to less than 40%. In some embodiments, the Fc fusion molecule reduces IgG levels to less than 30%. In some embodiments, the Fc fusion molecule reduces IgG levels to less than 25%.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of the Fc fusion molecule to a target molecule, the terms “bind,” “specific binding,” “specifically binds to,” “specific for,” “selectively binds,” and “selective for” a particular target (e.g., a polypeptide target such as FcRn) means binding that is measurably different from a non-specific or non-selective interaction (e.g., with a non-target molecule). Specific binding can be measured, for example, by measuring binding to a target molecule (e.g., FcRn) and comparing it to binding to a non-target molecule. Specific binding can also be determined by competition with a control molecule that mimics the target molecule. In that case, specific binding is indicated if the binding of the Fc fusion molecule to the target molecule is competitively inhibited by the control molecule. In some embodiments, the affinity of the Fc fusion molecule for a non-target molecule is less than about 50% of the affinity for FcRn (i.e., the K_{D} for FcRn is two times lower than the K_{D} for a non-target). In some embodiments, the affinity of the Fc fusion molecule for a non-target molecule is less than about 40% of the affinity for FcRn. In some embodiments, the affinity of the Fc fusion molecule for a non-target molecule is less than about 30% of the affinity for FcRn. In some embodiments, the affinity of the Fc fusion molecule for a non-target molecule is less than about 20% of the affinity for FcRn. In some embodiments, the affinity the Fc fusion molecule for a non-target molecule is less than about 10% of the affinity for FcRn. In some embodiments, the affinity of the Fc fusion molecule for a non-target molecule is less than about 1% of the affinity for FcRn. In some embodiments, the affinity of the Fc fusion molecule for a non-target molecule is less than about 0.1% of the affinity for FcRn.

In some embodiments, the Fc fusion molecule binds to FcRn at pH 6.0 or pH 7.4 with a KD of less than or equal to about 1×10⁻⁸, 1.1×10⁻⁸, 1.2×10⁻⁸, 1.3×10⁻⁸, 1.4×10⁻⁸, 1.5×10⁻⁸, 1.6×10⁻⁸, 1.7×10⁻⁸, 1.8×10⁻⁸, 1.9×10⁻⁸, 1.95×10⁻⁸, 2×10⁻⁸, 2.5×10⁻⁸, 3×10⁻⁸, 3.5×10⁻⁸, 4×10⁻⁸, 4.5×10⁻⁸, 5×10⁻⁸, 6×10⁻⁸, 7×10⁻⁸, 8×10⁻⁸, 9×10⁻⁸, 1×10⁻⁹, 1.1×10⁻⁹, 1.2×10⁻⁹, 1.3×10⁻⁹, 1.4×10⁻⁹, 1.5×10⁻⁹, 1.6×10⁻⁹, 1.7×10⁻⁹, 1.8×10⁻⁹, 1.9×10⁻⁹, 1.95×10⁻⁹, 2×10⁻⁹, 2.5×10⁻⁹, 3×10⁻⁹, 3.5×10⁻⁹, 4×10⁻⁹, 4.5×10⁻⁹, 5×10⁻⁹, 6×10⁻⁹, 7×10⁻⁹, 8×10⁻⁹, 9×10⁻⁹, 1×10⁻¹⁰, 1.1×10⁻¹⁰, 1.2×10⁻¹⁰, 1.3×10⁻¹⁰, 1.4×10⁻¹⁰, 1.5×10⁻¹⁰, 1.6×10⁻¹⁰, 1.7×10⁻¹⁰, 1.8×10⁻¹⁰, 1.9×10⁻¹⁰, 1.95×10⁻¹⁰, 2×10⁻¹⁰, 2.5×10⁻¹⁰, 3×10⁻¹⁰, 3.5×10⁻¹⁰, 4×10⁻¹⁰, 4.5×10⁻¹⁰, 5×10⁻¹⁰, 6×10⁻¹⁰, 7×10⁻¹⁰, 8×10⁻¹⁰, or 9×10⁻¹⁰ M, as measured by ELISA or SPR or any other suitable method known in the art.

In some embodiments, the Fc fusion molecule binds to FcRn at pH 6.0 or pH 7.4 is between about 1.0-1.1×10⁻⁸ M, 1.1-1.2×10⁻⁸ M, 1.2-1.3×10⁻⁸ M, 1.3-1.4×10⁻⁸ M, 1.4-1.5×10⁻⁸ M, 1.5-1.6×10⁻⁸ M, 1.6-1.7×10⁻⁸ M, 1.7-1.8×10⁻⁸ M, 1.8-1.9×10⁻⁸ M, 1.9-2×10⁻⁸ M, 1-2×10⁻⁸ M, 1-5×10⁻⁸ M, 2-7×10⁻⁸ M, 3-8×10⁻⁸ M, 3-5×10⁻⁸ M, 4-6×10⁻⁸ M, 5-7×10⁻⁸ M, 6-8×10⁻⁸ M, 7-9×10⁻⁸ M, 7-9.9×10⁻⁸ M, 5-9.9×10⁻⁸ M, 1.0-1.1×10⁻⁹ M, 1.1-1.2×10⁻⁹ M, 1.2-1.3×10⁻⁹ M, 1.3-1.4×10⁻⁹ M, 1.4-1.5×10⁻⁹ M, 1.5-1.6×10⁻⁹ M, 1.6-1.7×10⁻⁹ M, 1.7-1.8×10⁻⁹ M, 1.8-1.9×10⁻⁹ M, 1.9-2×10⁻⁹ M, 1-2×10⁻⁹ M, 1-5×10⁻⁹ M, 2-7×10⁻⁹ M, 3-8×10⁻⁹ M, 3-5×10⁻⁹ M, 4-6×10⁻⁹ M, 5-7×10⁻⁹ M, 6-8×10⁻⁹ M, 7-9×10⁻⁹ M, 7-9.9×10⁻⁹ M, or 5-9.9×10⁻⁹ M as measured by ELISA or SPR or any other suitable method known in the art. In some embodiments, the Fc fusion molecule binds to FcRn at pH 6.0 or pH 7.4 with a K_{D} of less than or equal to about 1×10⁻⁸ M, or less than or equal to about 1×10⁻⁹ M as measured by ELISA or SPR or any other suitable method known in the art.

In some embodiments, the Fc fusion molecule binds to albumin (e.g., human serum albumin) at pH 6.0 or pH 7.4 with a KD of less than or equal to about 1×10⁻⁸, 1.1×10⁻⁸, 1.2×10⁻⁸, 1.3×10⁻⁸, 1.4×10⁻⁸, 1.5×10⁻⁸, 1.6×10⁻⁸, 1.7×10⁻⁸, 1.8×10⁻⁸, 1.9×10⁻⁸, 1.95×10⁻⁸, 2×10⁻⁸, 2.5×10⁻⁸, 3×10⁻⁸, 3.5×10⁻⁸, 4×10⁻⁸, 4.5×10⁻⁸, 5×10⁻⁸, 6×10⁻⁸, 7×10⁻⁸, 8×10⁻⁸, 9×10⁻⁸, 1×10⁻⁹, 1.1×10⁻⁹, 1.2×10⁻⁹, 1.3×10⁻⁹, 1.4×10⁻⁹, 1.5×10⁻⁹, 1.6×10⁻⁹, 1.7×10⁻⁹, 1.8×10⁻⁹, 1.9×10⁻⁹, 1.95×10⁻⁹, 2×10⁻⁹, 2.5×10⁻⁹, 3×10⁻⁹, 3.5×10⁻⁹, 4×10⁻⁹, 4.5×10⁻⁹, 5×10⁻⁹, 6×10⁻⁹, 7×10⁻⁹, 8×10⁻⁹, 9×10⁻⁹, 1×10⁻¹⁰, 1.1×10⁻¹⁰, 1.2×10⁻¹⁰, 1.3×10⁻¹⁰, 1.4×10⁻¹⁰, 1.5×10-10, 1.6×10⁻¹⁰, 1.7×10⁻¹⁰, 1.8×10⁻¹⁰, 1.9×10⁻¹⁰, 1.95×10⁻¹⁰, 2×10⁻¹⁰, 2.5×10⁻¹⁰, 3×10-10, 3.5×10⁻¹⁰, 4×10⁻¹⁰, 4.5×10⁻¹⁰, 5×10⁻¹⁰, 6×10⁻¹⁰, 7×10⁻¹⁰, 8×10⁻¹⁰, or 9×10⁻¹⁰ M, as measured by ELISA or SPR or any other suitable method known in the art.

In some embodiments, the Fc fusion molecule binds to albumin (e.g., human serum albumin) at pH 6.0 or pH 7.4 is between about 1.0-1.1×10⁻⁸ M, 1.1-1.2×10⁻⁸ M, 1.2-1.3×10⁻⁸ M, 1.3-1.4×10⁻⁸ M, 1.4-1.5×10⁻⁸ M, 1.5-1.6×10⁻⁸ M, 1.6-1.7×10⁻⁸ M, 1.7-1.8×10⁻⁸ M, 1.8-1.9×10⁻⁸ M, 1.9-2×10⁻⁸ M, 1-2×10⁻⁸ M, 1-5×10⁻⁸ M, 2-7×10⁻⁸ M, 3-8×10⁻⁸ M, 3-5×10⁻⁸ M, 4-6×10⁻⁸ M, 5-7×10⁻⁸ M, 6-8×10⁻⁸ M, 7-9×10⁻⁸ M, 7-9.9×10⁻⁸ M, 5-9.9×10⁻⁸ M, 1.0-1.1×10⁻⁹ M, 1.1-1.2×10⁻⁹ M, 1.2-1.3×10⁻⁹ M, 1.3-1.4×10⁻⁹ M, 1.4-1.5×10⁻⁹ M, 1.5-1.6×10⁻⁹ M, 1.6-1.7×10⁻⁹ M, 1.7-1.8×10⁻⁹ M, 1.8-1.9×10⁻⁹ M, 1.9-2×10⁻⁹ M, 1-2×10⁻⁹ M, 1-5×10⁻⁹ M, 2-7×10⁻⁹ M, 3-8×10⁻⁹ M, 3-5×10⁻⁹ M, 4-6×10⁻⁹ M, 5-7×10⁻⁹ M, 6-8×10⁻⁹ M, 7-9×10⁻⁹ M, 7-9.9×10⁻⁹ M, or 5-9.9×10⁻⁹ M as measured by ELISA or SPR or any other suitable method known in the art. In some embodiments, the Fc fusion molecule binds to albumin at pH 6.0 or pH 7.4 with a K_{D} of less than or equal to about 1×10⁻⁸ M, or less than or equal to about 1×10⁻⁹ M as measured by ELISA or SPR or any other suitable method known in the art.

### Pharmaceutical Compositions

The present disclosure also features pharmaceutical compositions that contain a therapeutically effective amount of the Fc fusion molecules described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In some embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990)).

In some embodiments, a pharmaceutical composition is citrate-free.

In some embodiments, a pharmaceutical composition may contain nanoparticles, e.g., polymeric nanoparticles, liposomes, or micelles.

In some embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a Fc fusion molecules disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In some embodiments, the Fc fusion molecules disclosed herein is administered intravenously or subcutaneously. In some embodiments, the Fc fusion molecules disclosed herein is administered intravenously. In some embodiments, the Fc fusion molecules disclosed herein is administered subcutaneously.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. In some embodiments, the formulation for parenteral administration is citrate-free.

For intravenous or subcutaneous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous or subcutaneous drug delivery formulation may be contained in a syringe, pen, or bag. In some embodiments, the bag is connected to a channel comprising a tube and/or a needle. In some embodiments, the formulation is a lyophilized formulation or a liquid formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration.

A polyol, which acts as a tonicifier and may stabilize the Fc fusion molecules, may also be included in the formulation. The polyol is added to the formulation in an amount which may vary with respect to the desired isotonicity of the formulation. In some embodiments, the aqueous formulation is isotonic. The amount of polyol added may also be altered with respect to the molecular weight of the polyol. For example, a lower amount of a monosaccharide (e.g., mannitol) is added, compared to a disaccharide (such as trehalose). In some embodiments, the polyol which is used in the formulation as a tonicity agent is mannitol.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated Fc fusion molecule and/or minimizes the formation of particulates in the formulation and/or reduces adsorption. In some embodiments, the formulation may include a surfactant which is a polysorbate. In some embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996).

In embodiments, the protein product of the present disclosure is formulated as a liquid formulation. In some embodiments, the liquid formulation is prepared in combination with a sugar at stabilizing levels. In some embodiments, the liquid formulation is prepared in an aqueous carrier. In some embodiments, a stabilizer is added in an amount no greater than that which may result in a viscosity undesirable or unsuitable for intravenous administration. In some embodiments, the sugar is disaccharides, e.g., sucrose. In some embodiments, the liquid formulation may also include one or more of a buffering agent, a surfactant, and a preservative.

In some embodiments, the pH of the liquid formulation is set by addition of a pharmaceutically acceptable acid and/or base. In some embodiments, the pharmaceutically acceptable acid is hydrochloric acid. In some embodiments, the base is sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The Fc fusion molecules may be lyophilized to produce a lyophilized formulation including the proteins and a lyoprotectant. The lyoprotectant may be sugar, e.g., disaccharides. In some embodiments, the lyoprotectant is sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In some embodiments, the protein to sucrose or maltose weight ratio is of from 1:2 to 1:5. In some embodiments, the pH of the formulation, prior to lyophilization, is set by addition of a pharmaceutically acceptable acid and/or base. In some embodiments, the pharmaceutically acceptable acid is hydrochloric acid. In some embodiments, the pharmaceutically acceptable base is sodium hydroxide.

A patient's dose can be tailored to the approximate body weight or surface area of the patient. Other factors in determining the appropriate dosage can include the disease or condition to be treated or prevented, the severity of the disease, the route of administration, and the age, sex, and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

### Methods of Use

In some embodiments, the present application provides methods of contacting FcRn with the Fc fusion molecules described herein. In some embodiments, the FcRn is expressed on a cell surface.

In some embodiments, the present application provides methods of using the Fc fusion molecules described herein for treatment of a disorder or disease in a patient in need thereof. In some embodiments, the present application provides methods of using the Fc fusion molecules described herein to reduce IgG levels in a patient in need thereof. In some embodiments, described herein is a method for treating a patient in need thereof, the method comprising administering to a patient in need thereof a therapeutically effective amount of the Fc fusion molecule or pharmaceutical composition comprising the Fc fusion molecule described herein.

In some embodiments, the present application provides methods of using the Fc fusion molecules described herein for treatment of a disorder or disease in a patient in need thereof. In some embodiments, described herein is a method for treating a patient in need thereof with the Fc fusion molecules as described herein, the method comprising administering to the patient a therapeutically effective amount of the Fc fusion molecules or pharmaceutical composition comprising the Fc fusion molecules described herein. In some embodiments, the present application provides methods of treating a disorder or disease associated with elevated levels of IgG in a subject, by administering the Fc fusion molecules described herein.

In some embodiments, described herein are methods for treating a pathology associated with IgG activity, the method comprising administering to a patient in need thereof a therapeutically effective amount of the Fc fusion molecules or a pharmaceutical composition comprising the Fc fusion molecules described herein.

In some embodiments, described herein are methods for treating a pathology associated with elevated levels of FcRn in a patient in need thereof, the method comprising administering to the patient a therapeutically effective amount of the Fc fusion molecules or a pharmaceutical composition described herein.

In some embodiments, described herein is a method for treating or preventing a disorder or disease related to an antibody (e.g., an autoimmune disease or a disorder related to an unwanted side-effect of a therapeutic antibody) in a patient in need thereof, the method comprising administering to the patient a therapeutically effective amount the Fc fusion molecules disclosed herein or a pharmaceutical composition disclosed herein. In some embodiments, the inflammatory disorder or disease is an autoimmune disease. In some embodiments, the inflammatory disorder or disease is myasthenia gravis (gMG). In some embodiments, the inflammatory disorder or disease is immune thrombocytopenia (ITP).

In some embodiments, described herein is a method for treating a pathology associated with elevated levels of an IgG in a patient in need thereof, the method comprising administering to the patient a therapeutically effective amount the Fc fusion molecules disclosed herein or a pharmaceutical composition disclosed herein.

In some embodiments, described herein is a method of reducing biological activity of an IgG in a patient in need thereof, the method comprising administering to the patient a therapeutically effective amount the Fc fusion molecules disclosed herein or a pharmaceutical composition disclosed herein.

In some embodiments, described herein is a method of treating or preventing autoimmune disease. In some embodiments, the autoimmune disease is caused by auto-reactive antibodies.

In some embodiments, the disease or disorder is generalized myasthenia gravis (gMG), chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenic purpura, primary Sjogren's Syndrome, systemic lupus erythematosus, rheumatoid arthritis, bullous pemphigoid, pemphigus foliaceus, pemphigus vulgaris, or cutaneous lupus erythematosus.

In some embodiments, the disease or disorder is an autoimmune disease.

In some embodiments, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatrical pemphigoid, CREST syndrome, cold agglutinin disease, Crohn's disease, dermatomyositis, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic inflammatory myopathies, idiopathic membranous neuropathy, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen planus, lichen sclerosus, lupus erythematosus, Meniere's disease, mixed connective tissue disease, mucous membrane pemphigoid, multiple sclerosis, type 1 diabetes mellitus, Multifocal motor neuropathy (MMN), myasthenia gravis, paraneoplastic bullous pemphigoid, pemphigoid gestationis, pemphigus vulgaris, pemphigus foliaceus, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjogren's syndrome, solid organ transplant rejection, stiff-man syndrome, systemic lupus erythematosus, takayasu arteritis, toxic epidermal necrolysis (TEN), Stevens Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegner's granulomatosis.

In some embodiments, administration of the Fc fusion molecule reduces IgG levels by at least 50% within 7 days of the first administration. In some embodiments, administration of the Fc fusion molecule reduces IgG levels by at least 50% within 5 days of the first administration. In some embodiments, administration of the Fc fusion molecule reduces IgG levels by at least 50% within 3 days of the first administration. In some embodiments, administration of the Fc fusion molecule reduces IgG levels by at least 50% within 2 days of the first administration. In some embodiments, administration of the Fc fusion molecule reduces IgG levels by at least 50% within 1 day of the first administration.

In some embodiments, administration of the Fc fusion molecule reduces IgG levels by at least 75% within 7 days of the first administration. In some embodiments, administration of the Fc fusion molecule reduces IgG levels by at least 75% within 6 days of the first administration. In some embodiments, administration of the Fc fusion molecule reduces IgG levels by at least 75% within 5 days of the first administration. In some embodiments, administration of the Fc fusion molecule reduces IgG levels by at least 75% within 4 days of the first administration. In some embodiments, administration of the Fc fusion molecule reduces IgG levels by at least 75% within 3 days of the first administration. In some embodiments, administration of the Fc fusion molecule reduces IgG levels by at least 75% within 2 days of the first administration. In some embodiments, administration of the Fc fusion molecule reduces IgG levels by at least 75% within 1 day of the first administration.

In some embodiments, administration of the Fc fusion molecule reduces IgG levels by at least 80% within 7 days of the first administration. In some embodiments, administration of the Fc fusion molecule reduces IgG levels by at least 80% within 6 days of the first administration. In some embodiments, administration of the Fc fusion molecule reduces IgG levels by at least 80% within 5 days of the first administration. In some embodiments, administration of the Fc fusion molecule reduces IgG levels by at least 80% within 4 days of the first administration. In some embodiments, administration of the Fc fusion molecule reduces IgG levels by at least 80% within 3 days of the first administration. In some embodiments, administration of the Fc fusion molecule reduces IgG levels by at least 80% within 2 days of the first administration. In some embodiments, administration of the Fc fusion molecule reduces IgG levels by at least 80% within 1 day of the first administration.

In some embodiments, administration of the Fc fusion molecule reduces IgG levels by at least 90% within 7 days of the first administration. In some embodiments, administration of the Fc fusion molecule reduces IgG levels by at least 90% within 6 days of the first administration. In some embodiments, administration of the Fc fusion molecule reduces IgG levels by at least 90% within 5 days of the first administration. In some embodiments, administration of the Fc fusion molecule reduces IgG levels by at least 90% within 4 days of the first administration. In some embodiments, administration of the Fc fusion molecule reduces IgG levels by at least 90% within 3 days of the first administration. In some embodiments, administration of the Fc fusion molecule reduces IgG levels by at least 90% within 2 days of the first administration. In some embodiments, administration of the Fc fusion molecule reduces IgG levels by at least 90% within 1 day of the first administration.

In some embodiments, administration of the Fc fusion molecule reduces IgG levels to less than 60%, to less than 50%, to less than 40%, to less than 30%, or to less than 25% for at least 5 days, 7 days, 10 days, 15 days, or 20 days post administration.

In some embodiments, administration of the Fc fusion molecule reduces IgG levels to less than 60% for at least 5 days post-administration. In some embodiments, administration of the Fc fusion molecule reduces IgG levels to less than 60% for at least 7 days post-administration. In some embodiments, administration of the Fc fusion molecule reduces IgG levels to less than 60% for at least 10 days post-administration. In some embodiments, administration of the Fc fusion molecule reduces IgG levels to less than 60% for at least 15 days post-administration. In some embodiments, administration of the Fc fusion molecule reduces IgG levels to less than 60% for at least 20 days post-administration.

In some embodiments, administration of the Fc fusion molecule reduces IgG levels to less than 50% for at least 5 days post-administration. In some embodiments, administration of the Fc fusion molecule reduces IgG levels to less than 50% for at least 7 days post-administration. In some embodiments, administration of the Fc fusion molecule reduces IgG levels to less than 50% for at least 10 days post-administration. In some embodiments, administration of the Fc fusion molecule reduces IgG levels to less than 50% for at least 15 days post-administration. In some embodiments, administration of the Fc fusion molecule reduces IgG levels to less than 50% for at least 20 days post-administration.

In some embodiments, administration of the Fc fusion molecule reduces IgG levels to less than 40% for at least 5 days post-administration. In some embodiments, administration of the Fc fusion molecule reduces IgG levels to less than 40% for at least 7 days post-administration. In some embodiments, administration of the Fc fusion molecule reduces IgG levels to less than 40% for at least 10 days post-administration. In some embodiments, administration of the Fc fusion molecule reduces IgG levels to less than 40% for at least 15 days post-administration. In some embodiments, administration of the Fc fusion molecule reduces IgG levels to less than 40% for at least 20 days post-administration.

In some embodiments, administration of the Fc fusion molecule reduces IgG levels to less than 30% for at least 5 days post-administration. In some embodiments, administration of the Fc fusion molecule reduces IgG levels to less than 30% for at least 7 days post-administration. In some embodiments, administration of the Fc fusion molecule reduces IgG levels to less than 30% for at least 10 days post-administration. In some embodiments, administration of the Fc fusion molecule reduces IgG levels to less than 30% for at least 15 days post-administration. In some embodiments, administration of the Fc fusion molecule reduces IgG levels to less than 30% for at least 20 days post-administration.

In some embodiments, administration of the Fc fusion molecule reduces IgG levels to less than 25% for at least 5 days post-administration. In some embodiments, administration of the Fc fusion molecule reduces IgG levels to less than 25% for at least 7 days post-administration. In some embodiments, administration of the Fc fusion molecule reduces IgG levels to less than 25% for at least 10 days post-administration. In some embodiments, administration of the Fc fusion molecule reduces IgG levels to less than 25% for at least 15 days post-administration. In some embodiments, administration of the Fc fusion molecule reduces IgG levels to less than 25% for at least 20 days post-administration.

In some embodiments, the IgG levels are reduced as compared to a baseline. In some embodiments, the baseline is the IgG level in the patient prior to administration. In some embodiments, the baseline is the IgG level in a comparable patient without the administration of the Fc fusion molecule described herein.

### Methods of Preparation

The Fc fusion molecules described above can be made using recombinant DNA technology well known to a skilled person in the art. For example, one or more isolated polynucleotides encoding the Fc fusion molecules can be ligated to other appropriate nucleotide sequences, including, for example, constant region coding sequences, and expression control sequences, to produce conventional gene expression constructs (i.e., expression vectors) encoding the desired Fc fusion molecules. Production of defined gene constructs is within routine skill in the art.

Nucleic acids encoding desired Fc fusion molecules can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the Fc fusion molecules.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In some embodiments, the expression vector optionally contains sequences encoding all or part of a constant region, enabling an entire, or a part of, a heavy or light chain to be expressed. The gene construct can be introduced into eukaryotic host cells using conventional techniques.

In some embodiments, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the Fc fusion molecules.

The Fc fusion molecules can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1: Measuring Binding Kinetics of Fc Fusion Molecules to Albumin and huFcRn Using Surface Plasmon Resonance

A Biacore 8K SPR system equipped with a CM5 Sensor Chip immobilized with an anti-human Fc specific antibody by amine coupling, was used to determine the binding kinetic rate and affinity constants at 25° C. and in a running buffer of Fc fusion molecules pH 6.0 or pH 7.4 (10 mM HEPES, 150 mM NaCl, 3 mM EDTA, 0.05% Surfactant P20). Following a stabilization period in running buffer, the Fc fusion molecules at 20 nM were captured onto flow cell 2 (active) for 60 sec at a flow rate of 30 uL/min. Recombinant in-house Human FcRn (huFCRN) protein or human serum albumin (HSA) was prepared at concentrations of 0, 26.25, 52.5, 125, 250, and 500 nM and injected over flow cell 1 (reference) and flow cell 2 (active) for 120 sec at a flow rate of 30 μL/min. Recombinant Fc molecules were prepared at concentrations of 0, 25, 50, 100, 200, and 400 nM and injected over flow cell 1 (reference) and flow cell 2 (active) for 120 sec at a flow rate of 30 μL/min. Samples were injected in a multi-cycle manner over freshly captured Fc fusion molecule, by regenerating the capture surfaces with injection of 10 mM glycine pH 1.5 for 30 sec at a flow rate of 30 μL/min. The data was processed and analyzed with Biacore Insight Evaluation Software Version 2.0.15.12933 (GE Healthcare) as follows. Responses from flow cell 1 (reference) were subtracted from the responses from flow cell 2 (active). The responses from the two buffer blank injections were then subtracted from the reference subtracted data (2-1) to yield double-referenced data, which were fit to a 1:1 binding model to determine the apparent association (ka) and dissociation rate constants (kd). Their ratio provided the apparent equilibrium dissociation constant or affinity constant (KD=kd/ka).

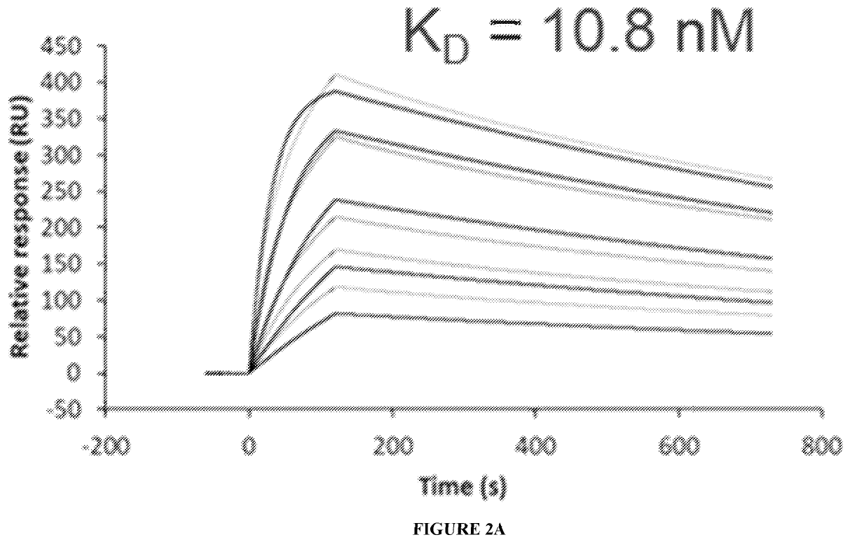

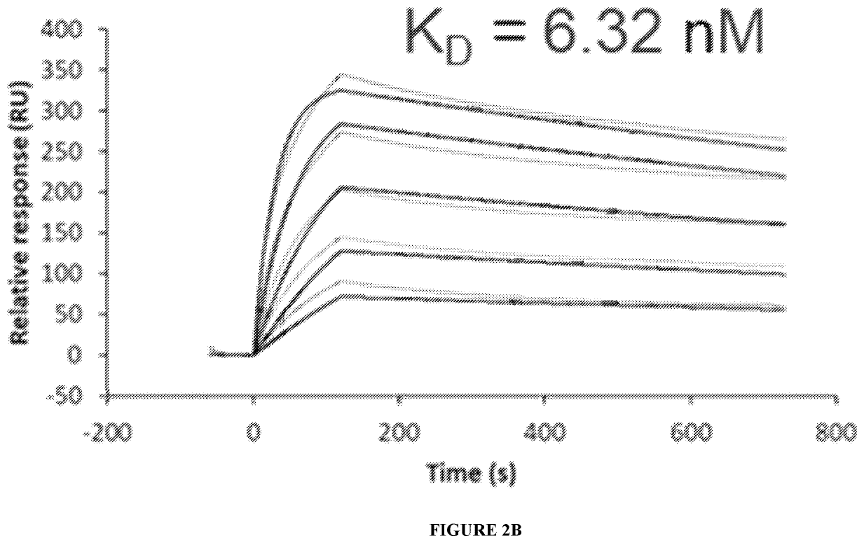

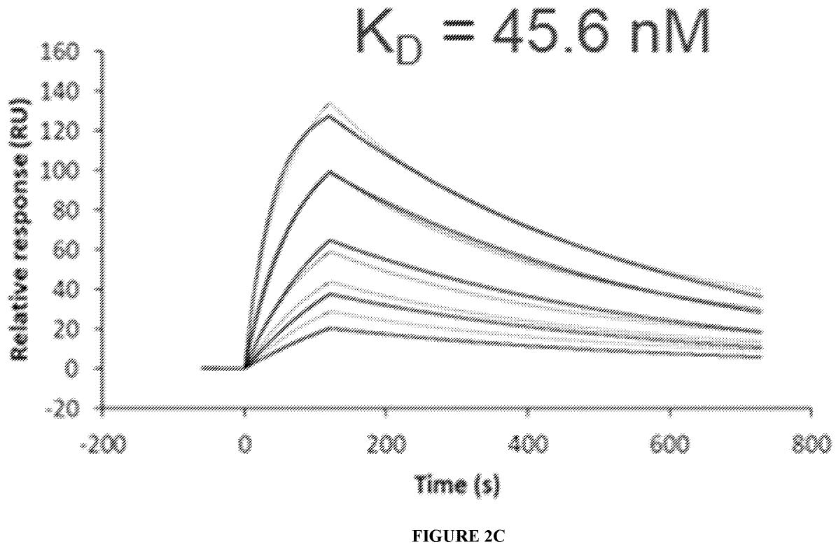

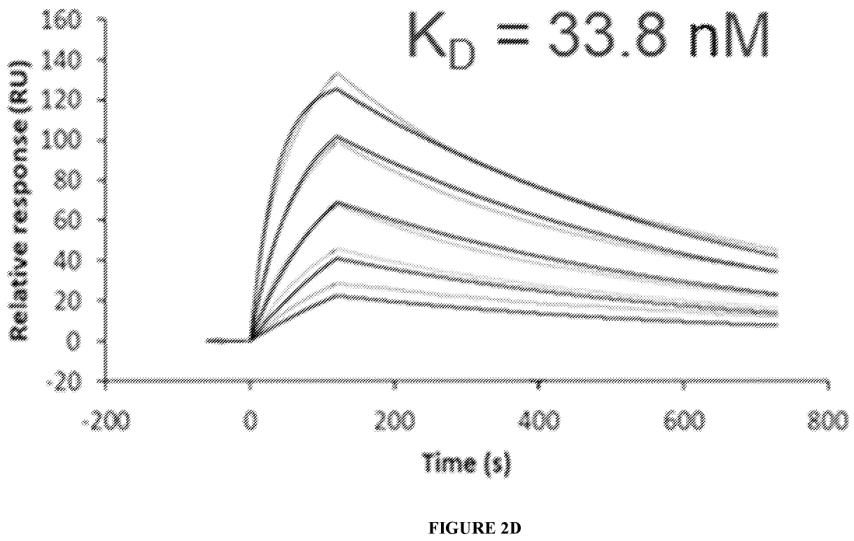

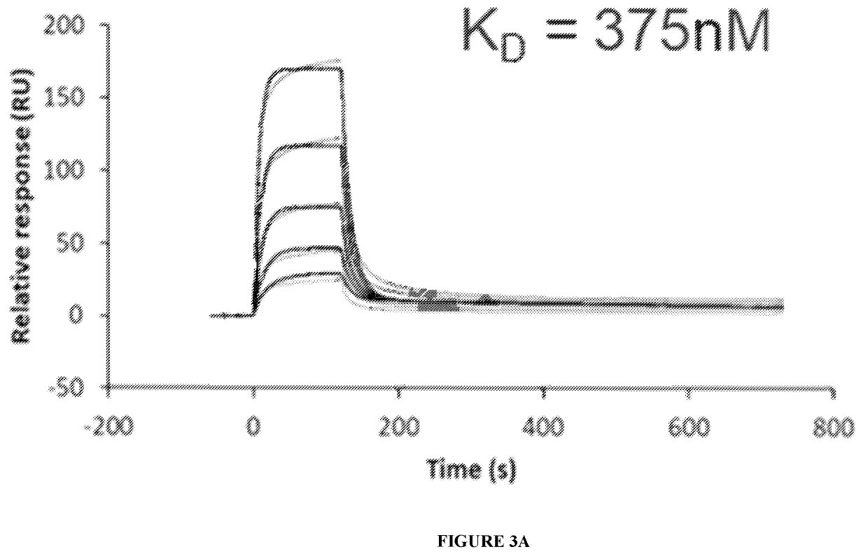

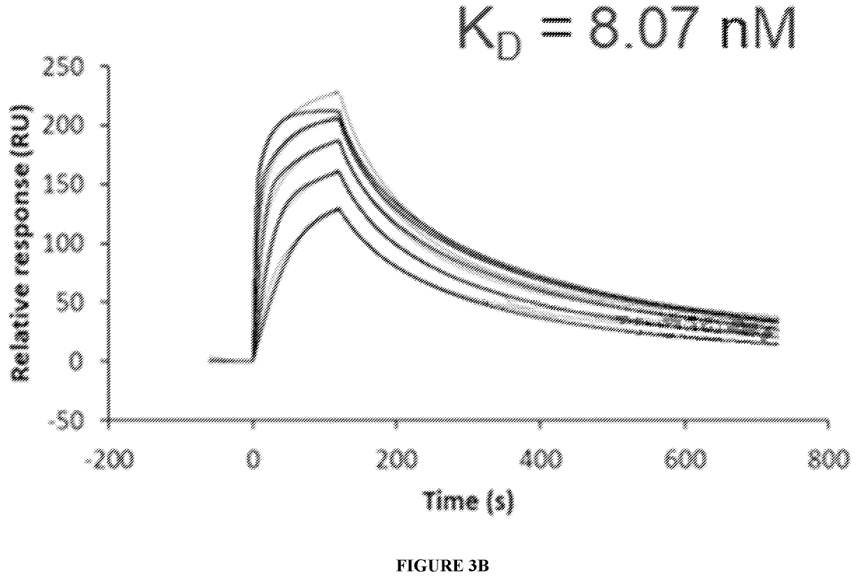

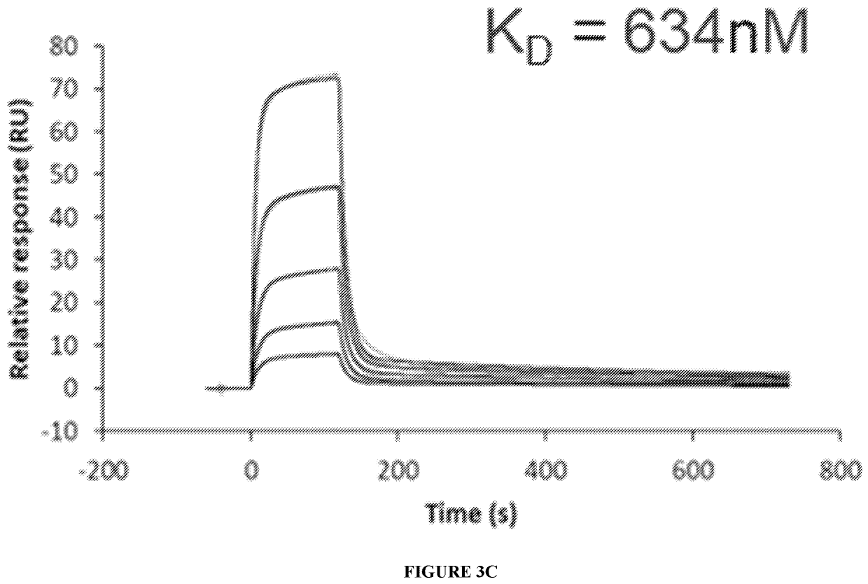

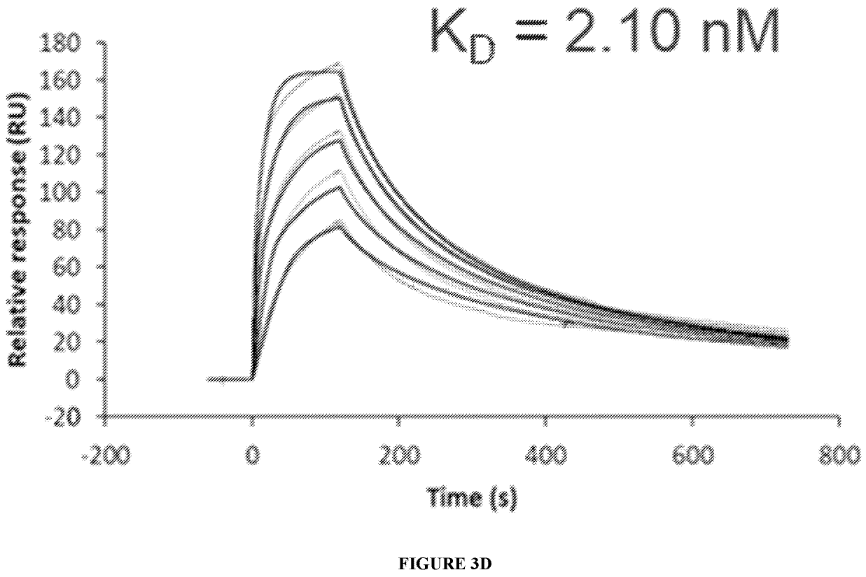

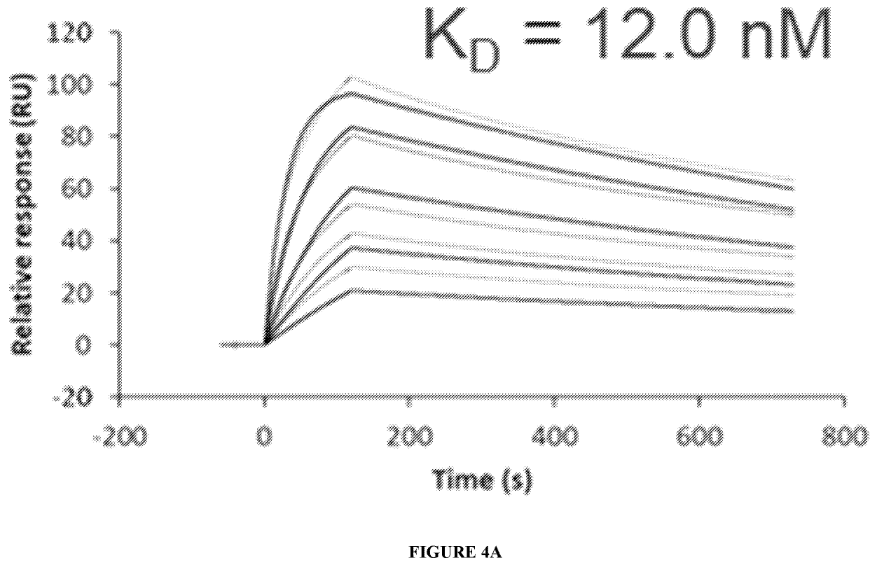

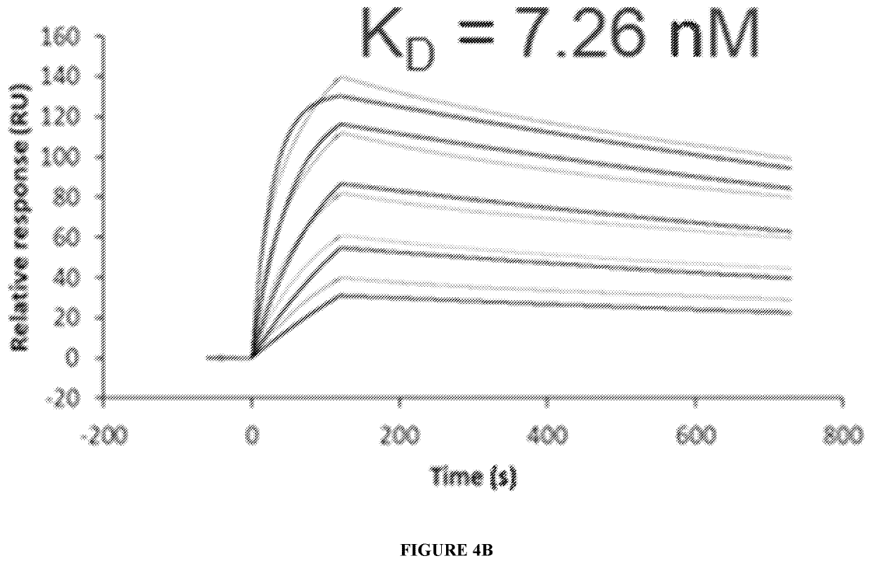

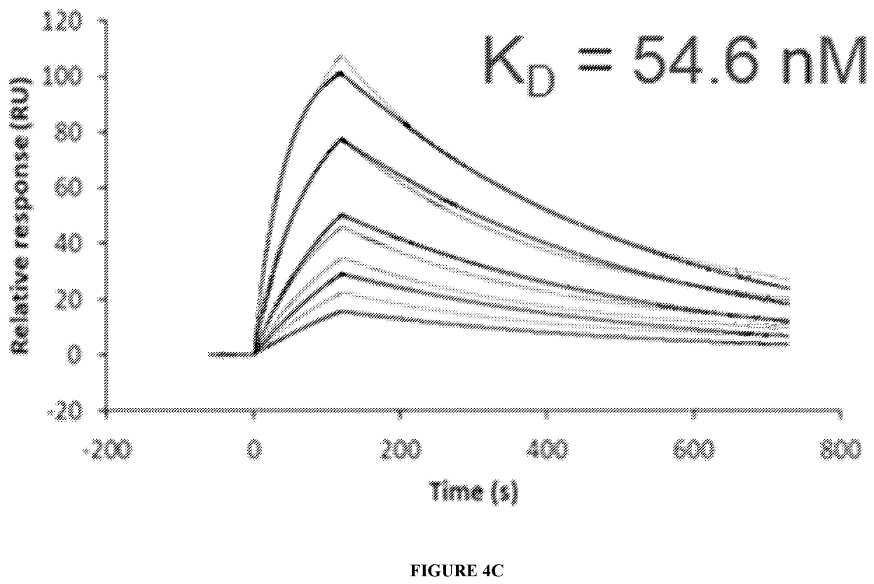

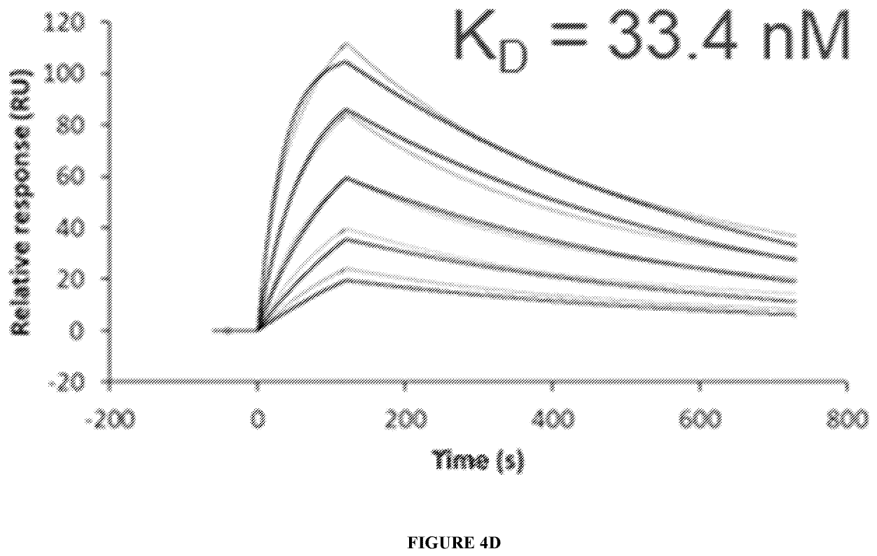

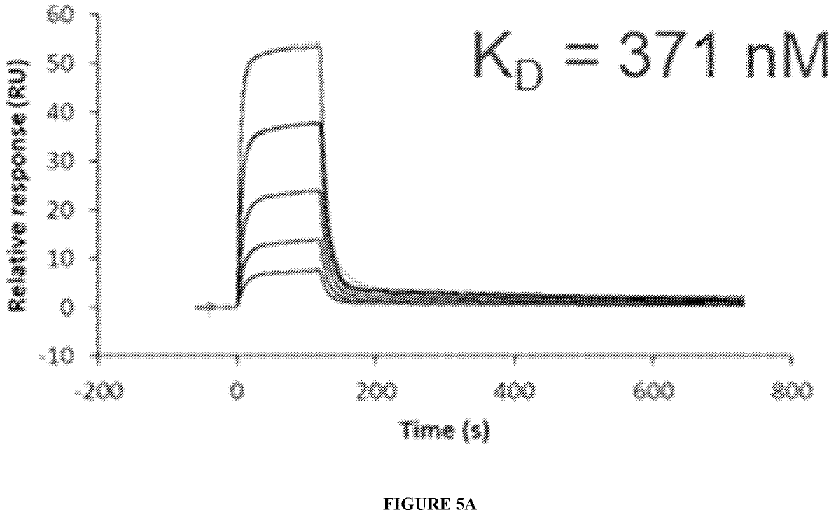

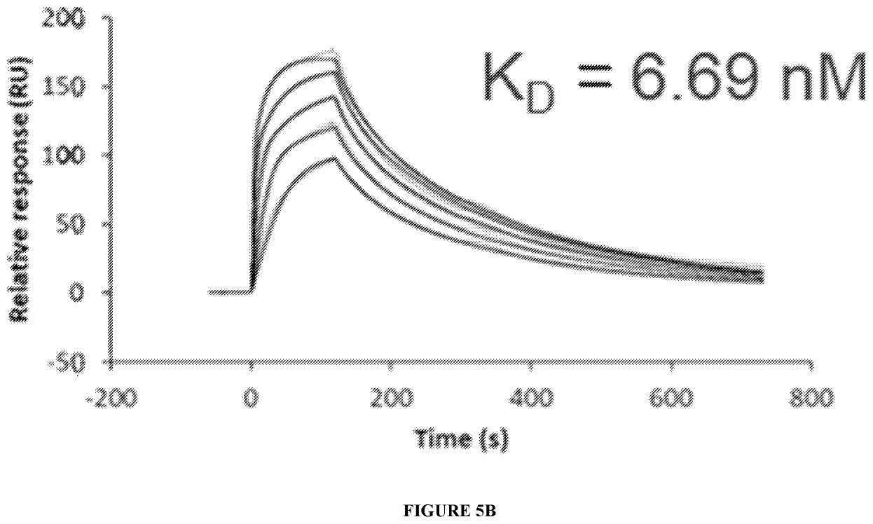

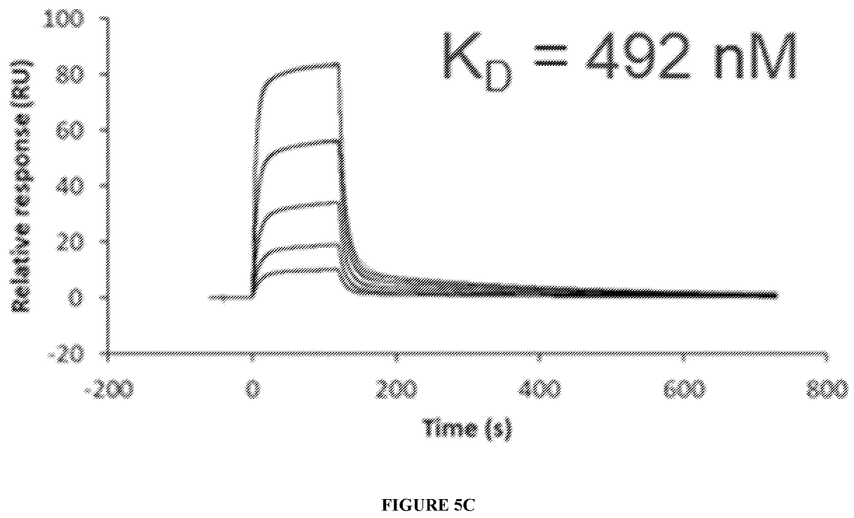

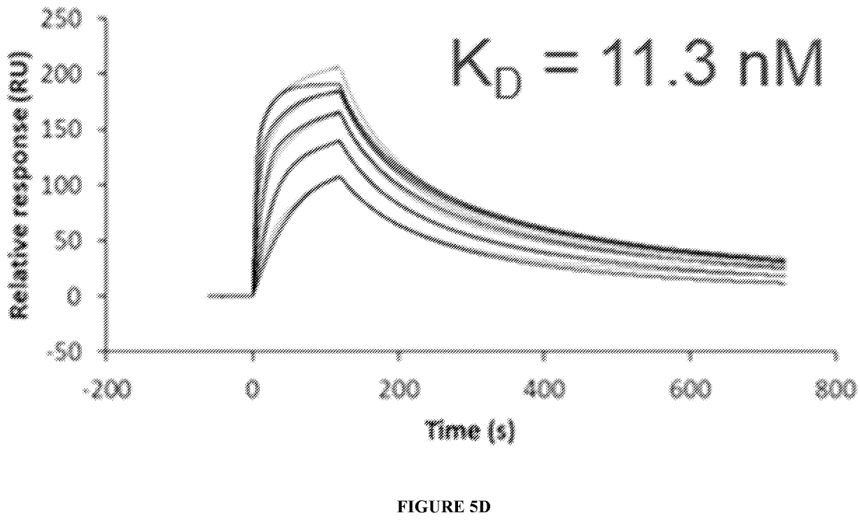

Binding kinetics are shown in Table 2 and FIGS. 2A-2D, 3A-3D, 4A-4D, and 5A-5D.

TABLE 2
Binding Kinetics of HSA and huFcRn from different bivalent
and monovalent Fc fusion molecules at pH 6 and pH 7.4

| | |
|---|---|
| HSA K_{D} (nM) | HuFcRn K_{D} (nM) |

| Sample ID | pH 6.0 | pH 7.4 | pH 6.0 | pH 7.4 |
|---|---|---|---|---|
| Fc Fusion | 6.32 | 10.8 | 8.07 | 375 |
| Molecule 5 | | | | |
| Fc Fusion | 33.8 | 45.6 | 2.10 | 634 |
| Molecule 6 | | | | |
| Fc Fusion | 7.26 | 12.0 | 6.69 | 371 |
| Molecule 11 | | | | |
| Fc Fusion | 33.4 | 54.6 | 11.3 | 492 |
| Molecule 12 | | | | |

### Example 2: Production of Fc Fusion Molecules in CHO Cells

The production of Fc fusion molecules in CHO cell culture was performed as described in the Method section disclosed herein.

Culture yields, the percentage of monomer as determined by analytical size-exclusion chromatography (aSEC), and the amount of LPS as measured in endotoxin units per milligram (EU/mg) are shown in Table 3.

TABLE 3
Production of Fc fusion molecules in CHO cells

| | | | | | | | Fc |
|---|---|---|---|---|---|---|---|
| | Protein | Protein | | aSEC | | | fusion |
| | con. | amount | Culture | Monomer | LPS | | molecule |
| Name | (mg/mL) | (mg) | (ml) | (%) | (EU/mg) | Description | format |
| Fc | 10.09 | 529.52 | 1000 | 100 | <0.20 | ABD1-G4S-Efgart | Format 1 |
| Fusion | | | | | | | |
| Molecule | | | | | | | |
| 5 | | | | | | | |
| Fc | 0.56 | 0.42 | 50 | 100 | <0.20 | ABD1-(G4S)3- | Format 1 |
| Fusion | | | | | | Efgart | |
| Molecule | | | | | | | |
| 7 | | | | | | | |
| Fc | 9.88 | 20.75 | 50 | 100 | <0.20 | ABD1-G4S- | Format 3 |
| Fusion | | | | | | Efgart(H)/Efgart(K) | |
| Molecule | | | | | | | |
| 9 | | | | | | | |
| Fc | 1.99 | 2.19 | 50 | 100 | <0.20 | ABD1-(G4S)3- | Format 3 |
| Fusion | | | | | | Efgart(H)/Efgart(K) | |
| Molecule | | | | | | | |
| 11 | | | | | | | |
| Fc | 10.42 | 187.53 | 1000 | 100 | 0.08 | Efgart-KIH control | |
| Fragment | | | | | | | |
| 4 | | | | | | | |
| Fc | 10.65 | 287.55 | 1000 | 100 | 0.15 | Efgart-G4S-ABD1 | Format 2 |
| Fusion | | | | | | | |
| Molecule | | | | | | | |
| 6 | | | | | | | |
| Fc | 10.73 | 568.65 | 1000 | 100 | 0.02 | Efgart-(G4S)3- | Format 2 |
| Fusion | | | | | | ABD1 | |
| molecule | | | | | | | |
| 8 | | | | | | | |
| Fc | 10.16 | 154.44 | 1000 | 100 | 0.03 | Efgart(H)-G4S- | Format 4 |
| Fusion | | | | | | ABD1/Efgart(K) | |
| Molecule | | | | | | | |
| 10 | | | | | | | |
| Fc | 10.26 | 225.81 | 1000 | 100 | 0.04 | Efgart(H)-(G4S)3- | Format 4 |
| Fusion | | | | | | ABD1/Efgart(K) | |
| Molecule | | | | | | | |
| 12 | | | | | | | |

### Example 3: Properties of Fc Fusion Molecules

Fc fusion molecules were assessed for DNA and insulin reactivity as well as for their propensity to self-associate (methods provided below). Additionally, differential scanning fluorimetry (DSF) and SLS-signal at 266 nm against temperature were used to determine Tm and Tagg of Fc fusion molecules. DSF and SLS were performed as described in the Method section disclosed herein. All corresponding results are shown in Table 4.

A polyreactive ELISA assay was used to screen for DNA and insulin reactivity of Fc fusion molecules. In this assay, plates were coated overnight at 4° C. with DNA (10 μg/ml) (DNA) and 5 μg/ml (insulin) in PBS pH 7.5. Wells were washed with water, blocked with 50 μl of Polyreactivity ELISA buffer (PEB; PBS containing 0.05% Tween-20, 1 mM EDTA) for 1 hour at room temperature, and rinsed three times with water. Serially-diluted Fc fusion molecules in 25 μl were added in quadruplicate to the wells and incubated for 1 hours at room temperature. Plates were washed three times with water, and 25 μl of 10 ng/ml goat anti-human IgG (Fc specific) conjugated to horseradish peroxidase were added to each well. Plates were incubated for 1 hour at room temperature, washed three times with 80 μl of water, and 25 μL of TMB substrate added to each well. Reactions were stopped after approximately 7 minutes by adding 25 μl of 0.18 M orthophosphoric acid to each well, and absorbance was read at 450 nm. DNA- and insulin-binding scores were calculated as the ratio of the ELISA signal of the Fc fusion molecule at 10 μg/ml to the signal of a well containing buffer instead of the primary antibody.

An affinity-capture self-interaction nanoparticle spectroscopy (AC-SINS) assay was performed to determine the propensity of Fc fusion molecules to self-associate. Briefly, 20 nm gold nanoparticles were coated with a mixture of 80% goat anti-human Fc and 20% non-specific goat polyclonal antibodies that were buffer exchanged into 20 mM sodium acetate pH 4.3 and diluted to 0.4 mg/ml. After one hour incubation at room temperature, sites unoccupied on the gold nanoparticles were blocked with thiolated polyethylene glycol (2 kD). The coated nanoparticles were then concentrated 10-fold using a syringe filter and 10 μl were added to 100 μl of mAb at 0.05 mg/ml in PBS pH 7.2. The coated nanoparticles were incubated with the Fc fusion molecule of interest for 2 hrs in a 96-well polypropylene plate and then transferred to a 384-well polystyrene plate and read on a spectrophotometer. The absorbance was read from 450-650 in 2 nm increments. The smoothed max absorbance of the average blank (PBS alone) was subtracted from the smoothed max absorbance of the antibody sample to determine the antibody AC-SINS score.

Most Fc fusion molecules exhibited favorable DNA-binding, insulin-binding, and AC-SINS scores. The DSF analysis showed that Fc Fusion Molecule 7 had a TM value of nearly 80° C., while most remaining samples showed a TM between 60-76° C. Fc Fusion Molecule 7 had the highest Tagg value (72.9° C.) and the other Fc fusion molecules showed a TM between 65.3-72.2° C.

TABLE 4
Characterization of Fc fusion molecules

| | Polyreactive | | AC-SINS | | | | | |
|---|---|---|---|---|---|---|---|---|
| | ELISA | | | Standard | DSF | | SLS | |
| | DNA | Insulin | Δλmax | deviation | T_{M1} | T_{M2} | Tagg | |
| Sample ID | score | score | (nm) | (nm) | (° C.) | (° C.) | (° C.) | Description |
| Fc Fusion | 3 | 1 | 3 | 0.1 | 59.5 | — | 69.8 | ABD1-G4S-Efgart |
| Molecule 5 | | | | | | | | |
| Fc Fusion | 5 | 1 | 1 | 0.2 | 59.4 | 79.7 | 72.9 | ABD1-(G4S)3-Efgart |
| Molecule 7 | | | | | | | | |
| Fc Fusion | 4 | 1 | −1 | 0.7 | 59.3 | 73.7 | 65.3 | ABD1-G4S- |
| Molecule 9 | | | | | | | | Efgart(H)/Efgart(K) |
| Fc Fusion | 11 | 1 | 0 | 0.2 | 60.3 | 76 | 68.4 | ABD1-(G4S)3- |
| Molecule 11 | | | | | | | | Efgart(H)/Efgart(K) |
| Fc Fragment 4 | 6 | 1 | −1 | 0.6 | 59.4 | 75.7 | 72.2 | Efgart-KIH control |
| Fc Fusion | 4 | 1 | −2 | 0.5 | 60.1 | — | 64.5 | Efgart-G4S-ABD1 |
| Molecule 6 | | | | | | | | |
| Fc Fusion | 4 | 1 | 0 | 0.2 | 59.0 | — | 64.1 | Efgart-(G4S)3-ABD1 |
| Molecule 8 | | | | | | | | |
| Fc Fusion | 5 | 1 | −1 | 0.8 | 60.7 | — | 66.6 | Efgart(H)-G4S- |
| Molecule 10 | | | | | | | | ABD1/Efgart(K) |
| Fc Fusion | 6 | 1 | 0 | 0.2 | 59.7 | 73.1 | 66.2 | Efgart(H)-(G4S)3- |
| Molecule 12 | | | | | | | | ABD1/Efgart(K) |

### Example 4: Pharmacokinetics (PK) and Pharmacodynamics (PD) of Fc Fusion Molecules in Mice

This example describes a pharmacokinetic (PK)/pharmacodynamic (PD) study of the Fc fusion molecules in mice.

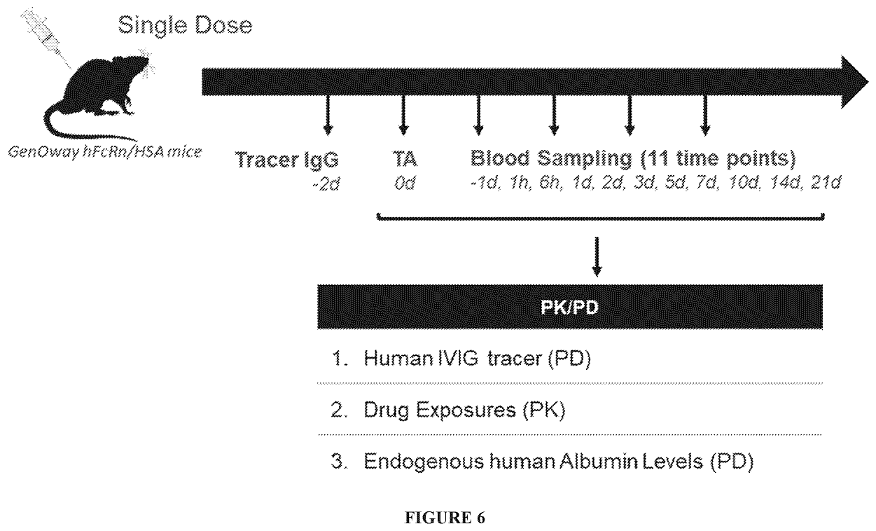

An exemplary work flow is seen in FIG. 6 and a summary of the study parameters is seen in Table 5. Briefly, mice were split into different cohorts. The various fusion molecules were administered to the mice intravenously. An exemplary control Fc fusion molecule (PALO2-173) comprising an Fc fragment and an RSV binding domain, in place of an albumin binding domain, was similarly administered to the mice intravenously. The study period occurred over 21 days with blood sampling taken at 11 time points.

TABLE 5
Study Parameters
Study Parameters

| | |
|---|---|
| Animals | 96 animals (n = 8 × 12 cohorts) |
| Route of Admin | IV infusion |
| Tracer IgG | IVIG, 200 mg/kg |
| Study Period | 21-day study |

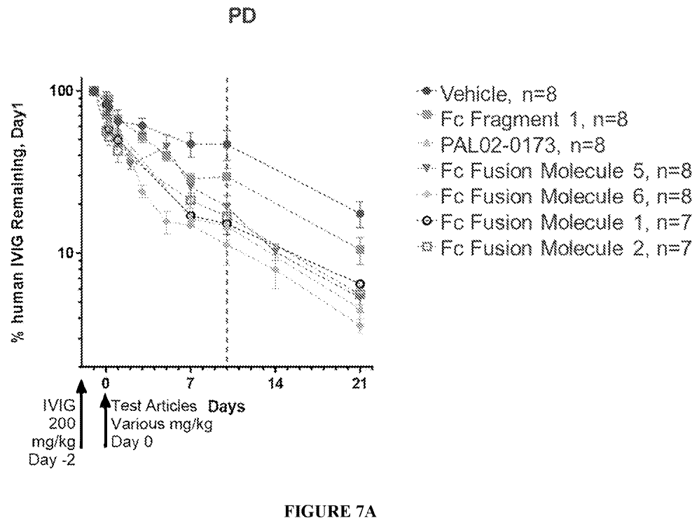

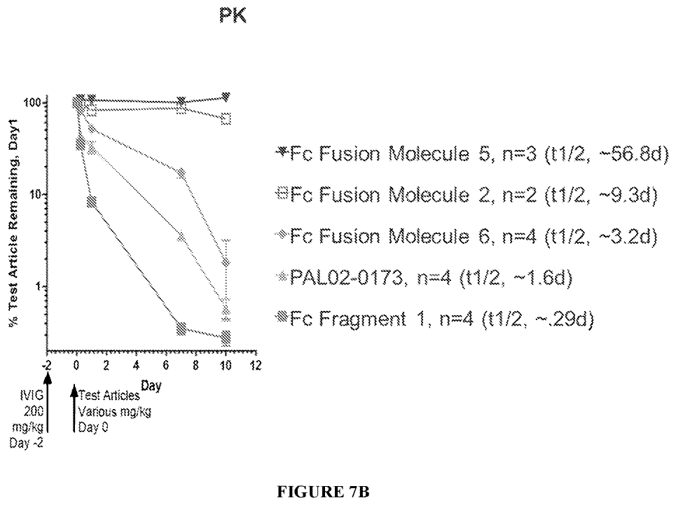

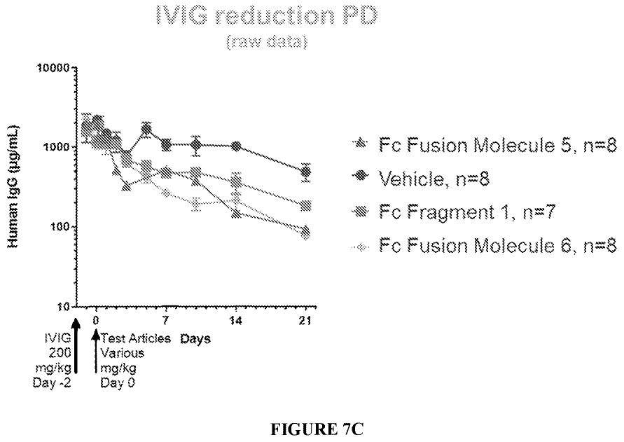

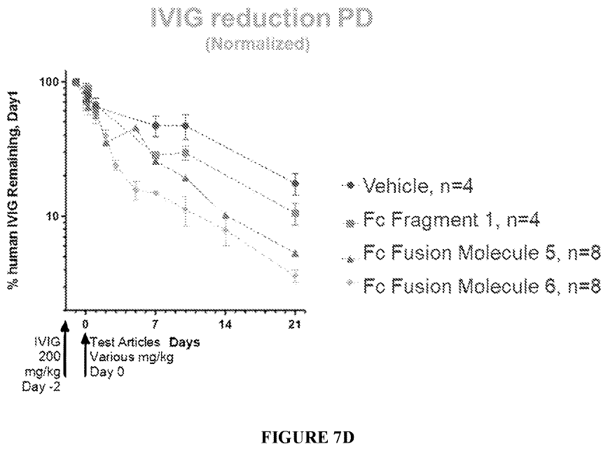

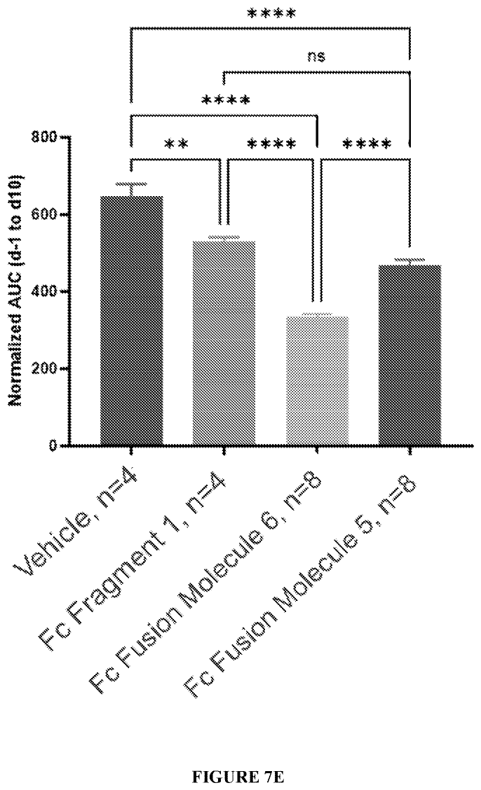

The data is seen in FIGS. 7A-7E. The PK/PD profiles demonstrated Fc Fusion Molecule 6 showed an improved PD effect and prolonged serum exposure superior to Fc Fragment 1 (FIGS. 7A-7B). Fc Fusion Molecule 6 also exhibited a difference compared to Fc Fragment 1 and Fc Fusion Molecule 5 in human IVIG tracer reduction using one-way ANOVA analysis.

### Example 5. Binding Kinetics of Exemplary Fc Fragments to FcRn from Different Species

This example demonstrated the binding kinetics of exemplary Fc Fragments to FcRn from different species at acidic and physiological pH.

The binding kinetics of exemplary Fc fragments to human, cynomolgus monkey, mouse, and rat FcRn were measured using surface plasmon resonance (SPR). The binding affinity (K_{D}) was calculated at both pH 6.0 and pH 7.4 as shown in Table 6.

TABLE 6
Binding Kinetics of Exemplary Fc Fragments

| | HuFcRn KD (M) | | cynoFcRn KD (M) | | mouse FcRn KD (M) | | rat_FcRn KD (M) | |
|---|---|---|---|---|---|---|---|---|
| mAb ID | pH 6 | pH 7.4 | pH 6 | pH 7.4 | pH 6 | pH 7.4 | pH 6 | pH 7.4 |
| Wild-type IgG1 | 7.4E−7* | | 3.3E−7* | | 7.2E−8* | | 6.8E−8* | |
| Fc Fragment 1 | 8.80E−09 | 2.39E−07 | 3.97E−08 | 1.37E−06 | 2.69E−10 | 2.80E−09 | 3.71E−09 | 1.23E−08 |
| Fc Fragment 2 | 8.05E−09 | 2.59E−07 | 3.62E−08 | 1.54E−06 | 1.82E−10 | 2.55E−09 | 3.50E−09 | 1.18E−08 |
| Fc Fragment 3 | 6.74E−09 | 2.10E−07 | 2.89E−08 | 9.48E−07 | 1.72E−10 | 2.38E−09 | 4.25E−09 | 1.49E−08 |
| Fc Fragment 10 | 6.62E−09 | 1.69E−07 | 2.86E−08 | 7.25E−07 | 1.43E−10 | 1.53E−09 | 3.49E−09 | 1.15E−08 |
| Fc Fragment 11 | 8.41E−09 | 2.69E−07 | 3.86E−08 | 1.70E−06 | 2.48E−10 | 4.05E−09 | 2.90E−09 | 1.47E−08 |
| Fc Fragment 12 | 7.15E−09 | 1.66E−07 | 3.01E−08 | 9.00E−07 | 1.94E−10 | 2.83E−09 | 5.41E−09 | 1.73E−08 |
| Fc Fragment 13 | 9.53E−09 | 2.48E−07 | 4.17E−08 | 1.37E−06 | 2.00E−10 | 2.73E−09 | 4.33E−09 | 1.62E−08 |
| Fc Fragment 14 | 8.26E−09 | 2.49E−07 | 3.61E−08 | 1.60E−06 | 2.88E−10 | 2.34E−09 | 3.94E−09 | 1.24E−08 |

| | | | | | |
|---|---|---|---|---|---|
| Fc Fragment 15 | 7.64E−08 | 2.91E−07 | 6.08E−08 | 1.43E−06 | No data |
| Fc Fragment 16 | 5.31E−08 | 3.71E−07 | 4.72E−08 | 1.68E−06 | |
| Fc Fragment 17 | 6.83E−08 | 2.93E−07 | 6.21E−08 | 2.19E−06 | |
| Fc Fragment 18 | 5.87E−08 | 2.62E−07 | 5.12E−08 | 2.11E−06 | |
| Fc Fragment 19 | 8.04E−08 | 2.52E−07 | 1.25E−06 | 1.43E−06 | |
| Fc Fragment 20 | 5.17E−08 | 2.90E−07 | 3.72E−07 | 1.55E−06 | |

*kinetic values were adapted from a reference doi.org/10.1080/19420862.2015.1008353

### Example 6. Exemplary Fc Fragments Block G Binding to Human FcRn

This example demonstrates the ability of exemplary Fc fragments to block IgG binding to human FcRn.

Briefly, the ability of exemplary Fc fragments to block IgG and a comparator Fc Fragment 1, which is Efgartigimod without the C-terminal lysine, binding to human FcRn was measured by competitive binding ELISA assays at pH 6.0. The IC₅₀ values were calculated for the exemplary Fc fragments and are shown in Table 7.

TABLE 7
IC50 Values for Exemplary Fc Fragments

| | | IC50 for IgG | IC50 for |
|---|---|---|---|
| | | blocking (in | Efgartigimod |
| | | triplicate) | inhibition |
| Construct | Mutations | (nM) | (nM) |
| Wild-type IgG1 | N/A | ND | ND |
| Fc Fragment 1 | M252Y/S254T/T256E/H433K/N434F | 2.85 | 4.82 |
| Fc Fragment 2 | M252Y/S254T/T256E/M428L/H433K/N434F | 1.31 | 4.09 |
| Fc Fragment 3 | M252Y/S254T/T256E/H433K/N434Y | 0.77 | 4.59 |
| Fc Fragment 10 | M252Y/S254T/T256E/M428L/H433K/N434Y | 1.48 | 5.03 |
| Fc Fragment 11 | M252Y/S254T/T256E/M428L/N434F | 1.51 | 6.47 |
| Fc Fragment 12 | M252Y/S254T/T256E/H433R/N434Y | 1.41 | 3.73 |
| Fc Fragment 13 | M252Y/S254T/T256E/H433R/N434F | 1.13 | 4.71 |
| Fc Fragment 14 | M252Y/S254T/T256E/M428L/H433R/N434F | 2.09 | 3.85 |
| Fc Fragment 15 | M252Y/S254T/T256E/L309D/H433K/N434F | 30.31 | No Data |
| Fc Fragment 16 | M252Y/S254T/T256E/L309D/H433K/N434Y | 8.45 | |
| Fc Fragment 17 | M252Y/S254T/T256E/L309D/M428L/H433K/ | 10.21 | |
| | N434F | | |
| Fc Fragment 18 | M252Y/S254T/T256E/L309D/M428L/H433K/ | 6.91 | |
| | N434Y | | |
| Fc Fragment 19 | M252Y/S254T/T256E/L309D/Q311K/H433K/ | 7.98 | |
| | N434F | | |
| Fc Fragment 20 | M252Y/S254T/T256E/L309D/Q311K/H433K/ | 5.18 | |
| | N434Y | | |

### Example 7. Linker Length Optimization of Exemplary Fc Fusion Molecules

This example demonstrates the linker length of exemplary Fc fusion molecules can impact the degradation of FcRn in vitro.

Briefly, HEK cells stably expressing FcRn-GFP were seeded and incubated overnight in IgG-depleted media at 37° C. with 5% CO₂. The following day, cells were treated with experimental Fc fusion molecules in the presence or absence of human serum albumin (HSA) at a final concentration of 100 μM. After treatment, cells were harvested, washed with PBS, fixed, and collected for flow cytometry analysis. Viability and GFP signal were assessed using a CytoFLEX LX flow cytometer.

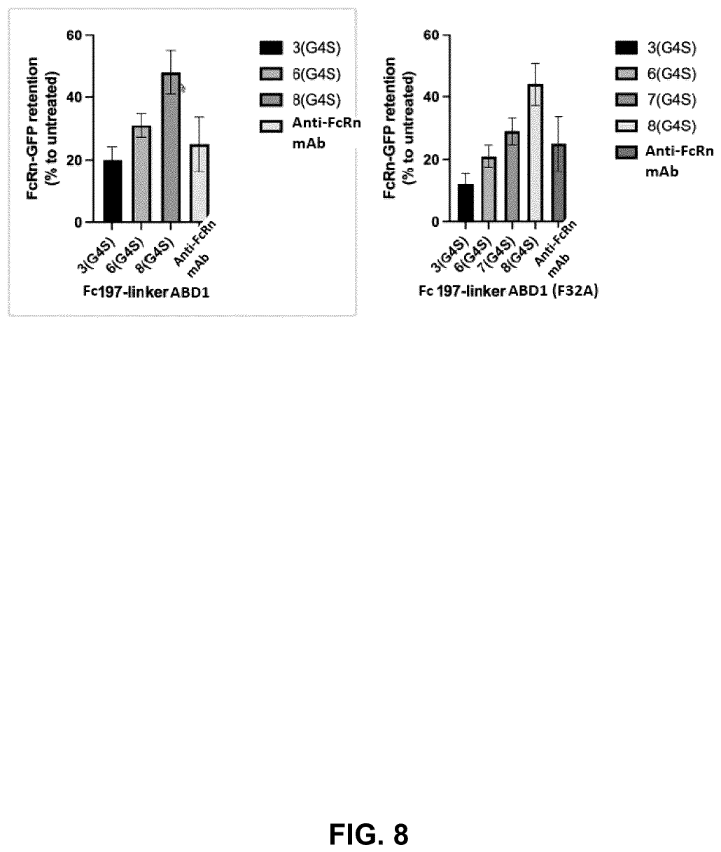

The residual FcRn after treatment with exemplary Fc fusion molecules is shown in FIG. 8 and quantified in Table 8. The results show decreased FcRn degradation with increased linker length regardless of the exemplary albumin binding domain.

TABLE 8
Residual FcRn After Treatment with
Exemplary Fc Fusion Molecules

| | | Average | |
|---|---|---|---|
| | | (n = 3) | ±(95% |
| Test Article | Description | Residual FcRn | CI) |
| Fusion Molecule 22 | Fc197(KIH) - (G₄S)₃ - | 20 | 4.2 |
| | ABD1 | | |
| Fusion Molecule 41 | Fc197(KIH) - (G₄S)₆ - | 31 | 3.9 |
| | ABD1 | | |
| Fusion Molecule 42 | Fc197(KIH) - (G₄S)₈ - | 48 | 7.1 |
| | ABD1 | | |
| Fusion Molecule 43 | Fc197(KIH) - (G₄S)₃ - | 12 | 3.4 |
| | ABD1(F32A) | | |
| Fusion Molecule 44 | Fc197(KIH) - (G₄S)₆ - | 21 | 3.5 |
| | ABD1(F32A) | | |
| Fusion Molecule 45 | Fc197(KIH) - (G₄S)₇ - | 29 | 4.4 |
| | ABD1(F32A) | | |
| Fusion Molecule 46 | Fc197(KIH) - (G₄S)₈ - | 44 | 6.8 |
| | ABD1(F32A) | | |
| anti-FcRn mAb | — | 25 | 8.8 |

INFORMAL SEQUENCE LISTING

| Identifier | Description | Amino Acid sequence |
|---|---|---|
| Efgartigimod (Efgart) | hIgG1_Fc_ | DKTHTCPPCPAPELLGGPSVFLFPPKPKDTLYITREPEVTCVVVDV |
| Fc Fragment 1 | M252Y/S254T/ | SHEDPEVKFNWYVDGVEVHNAKTKPREEQYNSTYRVVSVLTVLHQD |
| (SEQ ID NO: 1) | T256E/H433K/ | WLNGKEYKCKVSNKALPAPIEKTISKAKGQPREPQVYTLPPSRDEL |
| | N434F | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPG |
| Fc195 | IgG1 | DKTHTCPPCPAPELLGGPSVFLFPPKPKDTLYITREPEVTCVVVDV |
| Fc Fragment 2 | Fc_M252Y/ | SHEDPEVKFNWYVDGVEVHNAKTKPREEQYNSTYRVVSVLTVLHQD |
| (SEQ ID NO: 2) | S254T/T256E/ | WLNGKEYKCKVSNKALPAPIEKTISKAKGQPREPQVYTLPPSRDEL |
| | M428L/H433K/ | TKNQVSLTCLVKGFYPSDIAVEWESNGQPENNYKTTPPVLDSDGSF |
| | N434F | FLYSKLTVDKSRWQQGNVFSCSVLHEALKFHYTQKSLSLSPG |
| Fc197 | IgG1 | DKTHTCPPCPAPELLGGPSVFLFPPKPKDTLYITREPEVTCVVVDV |
| Fc Fragment 3 | Fc_M252Y/ | SHEDPEVKFNWYVDGVEVHNAKTKPREEQYNSTYRVVSVLTVLHQD |
| (SEQ ID NO: 3) | S254T/T256E/ | WLNGKEYKCKVSNKALPAPIEKTISKAKGQPREPQVYTLPPSRDEL |
| | H433K/N434Y | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPG |
| Efgart(H) | hIgG1_Fc_ | DKTHTCPPCPAPELLGGPSVFLFPPKPKDTLYITREPEVTCVVVDV |
| Fc Fragment 4 | M252Y/S254T/ | SHEDPEVKFNWYVDGVEVHNAKTKPREEQYNSTYRVVSVLTVLHQD |
| (SEQ ID NO: 4) | T256E/Y349C/ | WLNGKEYKCKVSNKALPAPIEKTISKAKGQPREPQVCTLPPSRDEL |
| | T366S/L368A/ | TKNQVSLSCAVKGFYPSDIAVEWESNGQPENNYKTTPPVLDSDGSF |
| | Y407V/H433K/ | FLVSKLTVDKSRWQQGNVFSCSVMHEALKFHYTQKSLSLSPG |
| | N434F | |
| Efgart(K) | hIgG1_Fc_ | DKTHTCPPCPAPELLGGPSVFLFPPKPKDTLYITREPEVTCVVVDV |
| Fc Fragment 5 | M252Y/S254T/ | SHEDPEVKFNWYVDGVEVHNAKTKPREEQYNSTYRVVSVLTVLHQD |
| (SEQ ID NO: 5) | T256E/S354C/ | WLNGKEYKCKVSNKALPAPIEKTISKAKGQPREPQVYTLPPCRDEL |
| | T366W/H433K/ | TKNQVSLWCLVKGFYPSDIAVEWESNGQPENNYKTTPPVLDSDGSF |
| | N434F | FLYSKLTVDKSRWQQGNVFSCSVMHEALKFHYTQKSLSLSPG |
| Fc197(H) | hIgG1_Fc_ | DKTHTCPPCPAPELLGGPSVFLFPPKPKDTLYITREPEVTCVVVDV |
| Fc Fragment 6 | M252Y/S254T/ | SHEDPEVKFNWYVDGVEVHNAKTKPREEQYNSTYRVVSVLTVLHQD |
| (SEQ ID NO: 6) | T256E/Y349C/ | WLNGKEYKCKVSNKALPAPIEKTISKAKGQPREPQVCTLPPSRDEL |
| | T366S/L368A/ | TKNQVSLSCAVKGFYPSDIAVEWESNGQPENNYKTTPPVLDSDGSF |
| | Y407V/H433K/ | FLVSKLTVDKSRWQQGNVFSCSVMHEALKYHYTQKSLSLSPG |
| | N434Y | |
| Fc197(K) | hIgG1_Fc_ | DKTHTCPPCPAPELLGGPSVFLFPPKPKDTLYITREPEVTCVVVDV |
| Fc Fragment 7 | M252Y/S254T/ | SHEDPEVKFNWYVDGVEVHNAKTKPREEQYNSTYRVVSVLTVLHQD |
| (SEQ ID NO: 7) | T256E/S354C/ | WLNGKEYKCKVSNKALPAPIEKTISKAKGQPREPQVYTLPPCRDEL |
| | T366W/H433K/ | TKNQVSLWCLVKGFYPSDIAVEWESNGQPENNYKTTPPVLDSDGSF |
| | N434Y | FLYSKLTVDKSRWQQGNVFSCSVMHEALKYHYTQKSLSLSPG |
| Fc195(H) | hIgG1_Fc_ | DKTHTCPPCPAPELLGGPSVFLFPPKPKDTLYITREPEVTCVVVDV |
| Fc Fragment 8 | M252Y/S254T/ | SHEDPEVKENWYVDGVEVHNAKTKPREEQYNSTYRVVSVLTVLHQD |
| (SEQ ID NO: 8) | T256E/Y349C/ | WLNGKEYKCKVSNKALPAPIEKTISKAKGQPREPQVCTLPPSRDEL |
| | T366S/L368A/ | TKNQVSLSCAVKGFYPSDIAVEWESNGQPENNYKTTPPVLDSDGSF |
| | Y407V/M428L/ | FLVSKLTVDKSRWQQGNVFSCSVLHEALKFHYTQKSLSLSPG |
| | H433K/N434F | |
| Fc195(K) | hIgG1_Fc_ | DKTHTCPPCPAPELLGGPSVFLFPPKPKDTLYITREPEVTCVVVDV |
| Fc Fragment 9 | M252Y/S254T/ | SHEDPEVKFNWYVDGVEVHNAKTKPREEQYNSTYRVVSVLTVLHQD |
| (SEQ ID NO: 9) | T256E/S354C/ | WLNGKEYKCKVSNKALPAPIEKTISKAKGQPREPQVYTLPPCRDEL |
| | T366W/M428L/ | TKNQVSLWCLVKGFYPSDIAVEWESNGQPENNYKTTPPVLDSDGSF |
| | H433K/N434F | FLYSKLTVDKSRWQQGNVFSCSVLHEALKFHYTQKSLSLSPG |
| HSA | Human Serum | DAHKSEVAHRFKDLGEENFKALVLIAFAQYLQQCPFEDHVKLVNEV |
| (SEQ ID NO: 10) | Albumin | TEFAKTCVADESAENCDKSLHTLFGDKLCTVATLRETYGEMADCCA |
| | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQREPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGL |
| ABD1 | Albumin | EVQLLESGGGLVQPGGSLRLSCAASGFTFRSFGMSWVRQAPGKGPE |
| (SEQ ID NO: 11) | binding VHH | WVSSISGSGSDTLYADSVKGRFTISRDNSKNTLYLQMNSLRPEDTA |
| | domain | VYYCTIGGSLSRSSQGTLVTVSS |
| ABD2 | Albumin | EVQLVESGGGLVQPGNSLRLSCAASGFTFSSFGMSWVRQAPGKGLE |
| (SEQ ID NO: 12) | binding VHH | WVSSISGSGSDTLYADSVKGRFTISRDNAKTTLYLQMNSLRPEDTA |
| | domain | VYYCTIGGSLSRSSQGTLVTVSS |
| G4S | Linker | GGGGS |
| (SEQ ID NO: 13) | | |
| (G4S)7 | Linker | GGGGSGGGGSGGGGSGGGGSGGGGSGGGGSGGGGS |
| (SEQ ID NO: 14) | | |
| (G4S)3 | Linker | GGGGSGGGGSGGGGS |
| (SEQ ID NO: 15) | | |
| Fc Fusion Molecule 1 | Efgart-G4S- | DKTHTCPPCPAPELLGGPSVFLFPPKPKDTLYITREPEVTCVVVDV |
| (SEQ ID NO: 16) | HSA | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPGGGGG |
| | | SDAHKSEVAHRFKDLGEENFKALVLIAFAQYLQQCPFEDHVKLVNE |
| | | VTEFAKTCVADESAENCDKSLHTLFGDKLCTVATLRETYGEMADCC |
| | | AKQEPERNECFLQHKDDNPNLPRLVRPEVDVMCTAFHDNEETFLKK |
| | | YLYEIARRHPYFYAPELLFFAKRYKAAFTECCQAADKAACLLPKLD |
| | | ELRDEGKASSAKQRLKCASLQKFGERAFKAWAVARLSQRFPKAEFA |
| | | EVSKLVTDLTKVHTECCHGDLLECADDRADLAKYICENQDSISSKL |
| | | KECCEKPLLEKSHCIAEVENDEMPADLPSLAADFVESKDVCKNYAE |
| | | AKDVFLGMFLYEYARRHPDYSVVLLLRLAKTYETTLEKCCAAADPH |
| | | ECYAKVFDEFKPLVEEPQNLIKQNCELFEQLGEYKFQNALLVRYTK |
| | | KVPQVSTPTLVEVSRNLGKVGSKCCKHPEAKRMPCAEDYLSVVLNQ |
| | | LCVLHEKTPVSDRVTKCCTESLVNRRPCFSALEVDETYVPKEFNAE |
| | | TFTFHADICTLSEKERQIKKQTALVELVKHKPKATKEQLKAVMDDF |
| | | AAFVEKCCKADDKETCFAEEGKKLVAASQAALGL |
| Fc Fusion Molecule 2 | HSA-G4S- | DAHKSEVAHRFKDLGEENFKALVLIAFAQYLQQCPFEDHVKLVNEV |
| (SEQ ID NO: 17) | Efgart | TEFAKTCVADESAENCDKSLHTLFGDKLCTVATLRETYGEMADCCA |
| | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQRFPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGLGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLIVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALKFHYTQKSLSLSPG |
| Fc Fusion Molecule 3 | Efgart-(G4S)7- | DKTHTCPPCPAPELLGGPSVFLFPPKPKDTLYITREPEVTCVVVDV |
| (SEQ ID NO: 18) | HSA | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPGGGGG |
| | | SGGGGSGGGGSGGGGSGGGGSGGGGSGGGGSDAHKSEVAHRFKDLG |
| | | EENFKALVLIAFAQYLQQCPFEDHVKLVNEVTEFAKTCVADESAEN |
| | | CDKSLHTLFGDKLCTVATLRETYGEMADCCAKQEPERNECFLQHKD |
| | | DNPNLPRLVRPEVDVMCTAFHDNEETFLKKYLYEIARRHPYFYAPE |
| | | LLFFAKRYKAAFTECCQAADKAACLLPKLDELRDEGKASSAKQRLK |
| | | CASLQKFGERAFKAWAVARLSQRFPKAEFAEVSKLVTDLTKVHTEC |
| | | CHGDLLECADDRADLAKYICENQDSISSKLKECCEKPLLEKSHCIA |
| | | EVENDEMPADLPSLAADFVESKDVCKNYAEAKDVFLGMFLYEYARR |
| | | HPDYSVVLLLRLAKTYETTLEKCCAAADPHECYAKVEDEFKPLVEE |
| | | PQNLIKQNCELFEQLGEYKFQNALLVRYTKKVPQVSTPTLVEVSRN |
| | | LGKVGSKCCKHPEAKRMPCAEDYLSVVLNQLCVLHEKTPVSDRVTK |
| | | CCTESLVNRRPCFSALEVDETYVPKEFNAETFTFHADICTLSEKER |
| | | QIKKQTALVELVKHKPKATKEQLKAVMDDFAAFVEKCCKADDKETC |
| | | FAEEGKKLVAASQAALGL |
| Fc Fusion Molecule 4 | HSA-(G4S)7- | DAHKSEVAHRFKDLGEENFKALVLIAFAQYLQQCPFEDHVKLVNEV |
| (SEQ ID NO: 19) | Efgart | TEFAKTCVADESAENCDKSLHTLFGDKLCTVATLRETYGEMADCCA |
| | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQRFPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGLGGGGSGGGGSGGG |
| | | GSGGGGSGGGGSGGGGSGGGGSDKTHTCPPCPAPELLGGPSVFLFP |
| | | PKPKDTLYITREPEVTCVVVDVSHEDPEVKFNWYVDGVEVHNAKTK |
| | | PREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVM |
| | | HEALKFHYTQKSLSLSPG |
| Fc Fusion Molecule 5 | ABD1-G4S- | EVQLLESGGGLVQPGGSLRLSCAASGFTFRSFGMSWVRQAPGKGPE |
| (SEQ ID NO: 20) | Efgart | WVSSISGSGSDTLYADSVKGRFTISRDNSKNTLYLQMNSLRPEDTA |
| | | VYYCTIGGSLSRSSQGTLVTVSSGGGGSDKTHTCPPCPAPELLGGP |
| | | SVFLFPPKPKDTLYITREPEVTCVVVDVSHEDPEVKENWYVDGVEV |
| | | HNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNV |
| | | FSCSVMHEALKFHYTQKSLSLSPG |
| Fc Fusion Molecule 6 | Efgart-G4S- | DKTHTCPPCPAPELLGGPSVFLFPPKPKDTLYITREPEVTCVVVDV |
| (SEQ ID NO: 21) | ABD1 | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule 7 | ABD1- | EVQLLESGGGLVQPGGSLRLSCAASGFTFRSFGMSWVRQAPGKGPE |
| (SEQ ID NO: 22) | (G4S)3-Efgart | WVSSISGSGSDTLYADSVKGRFTISRDNSKNTLYLQMNSLRPEDTA |
| | | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLIVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALKFHYTQKSLSLSPG |
| Fc Fusion Molecule 8 | Efgart-(G4S)3- | DKTHTCPPCPAPELLGGPSVFLFPPKPKDTLYITREPEVTCVVVDV |
| (SEQ ID NO: 23) | ABD1 | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKFHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule 9 | ABD1-G4S- | EVQLLESGGGLVQPGGSLRLSCAASGFTFRSFGMSWVRQAPGKGPE |
| (SEQ ID NO: 24) | Efgart(KIH) | WVSSISGSGSDTLYADSVKGRFTISRDNSKNTLYLQMNSLRPEDTA |
| | | VYYCTIGGSLSRSSQGTLVTVSSGGGGSDKTHTCPPCPAPELLGGP |
| | | SVFLFPPKPKDTLYITREPEVTCVVVDVSHEDPEVKFNWYVDGVEV |
| | | HNAKTKPREEQYNSTYRVVSVLTVLHQDWLNGKEYKCKVSNKALPA |
| | | PIEKTISKAKGQPREPQVCTLPPSRDELTKNQVSLSCAVKGFYPSD |
| | | IAVEWESNGQPENNYKTTPPVLDSDGSFFLVSKLTVDKSRWQQGNV |
| | | FSCSVMHEALKFHYTQKSLSLSPG |
| Fc Fusion Molecule | Efgart(KIH)- | DKTHTCPPCPAPELLGGPSVFLFPPKPKDTLYITREPEVTCVVVDV |
| 10 | G4S-ABD1 | SHEDPEVKFNWYVDGVEVHNAKTKPREEQYNSTYRVVSVLTVLHQD |
| (SEQ ID NO: 25) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKFHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule | ABD1- | EVQLLESGGGLVQPGGSLRLSCAASGFTFRSFGMSWVRQAPGKGPE |
| 11 | (G4S)3- | WVSSISGSGSDTLYADSVKGRFTISRDNSKNTLYLQMNSLRPEDTA |
| (SEQ ID NO: 26) | Efgart(KIH) | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLS |
| | | CAVKGFYPSDIAVEWESNGQPENNYKTTPPVLDSDGSFFLVSKLTV |
| | | DKSRWQQGNVFSCSVMHEALKFHYTQKSLSLSPG |
| Fc Fusion Molecule | Efgart(KIH)- | DKTHTCPPCPAPELLGGPSVFLFPPKPKDTLYITREPEVTCVVVDV |
| 12 | (G4S)3-ABD1 | SHEDPEVKFNWYVDGVEVHNAKTKPREEQYNSTYRVVSVLIVLHQD |
| (SEQ ID NO: 27) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKFHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule | HSA-G4S- | DAHKSEVAHRFKDLGEENFKALVLIAFAQYLQQCPFEDHVKLVNEV |
| 13 | Fc197 | TEFAKTCVADESAENCDKSLHTLFGDKLCTVATLRETYGEMADCCA |
| (SEQ ID NO: 28) | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQRFPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGLGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALKYHYTQKSLSLSPG |
| Fc Fusion Molecule | Fc197-G4S- | DKTHTCPPCPAPELLGGPSVFLFPPKPKDTLYITREPEVTCVVVDV |
| 14 | HSA | SHEDPEVKFNWYVDGVEVHNAKTKPREEQYNSTYRVVSVLTVLHQD |
| (SEQ ID NO: 29) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPGGGGG |
| | | SDAHKSEVAHRFKDLGEENFKALVLIAFAQYLQQCPFEDHVKLVNE |
| | | VTEFAKTCVADESAENCDKSLHTLFGDKLCTVATLRETYGEMADCC |
| | | AKQEPERNECFLQHKDDNPNLPRLVRPEVDVMCTAFHDNEETFLKK |
| | | YLYEIARRHPYFYAPELLFFAKRYKAAFTECCQAADKAACLLPKLD |
| | | ELRDEGKASSAKQRLKCASLQKFGERAFKAWAVARLSQRFPKAEFA |
| | | EVSKLVTDLTKVHTECCHGDLLECADDRADLAKYICENQDSISSKL |
| | | KECCEKPLLEKSHCIAEVENDEMPADLPSLAADFVESKDVCKNYAE |
| | | AKDVFLGMFLYEYARRHPDYSVVLLLRLAKTYETTLEKCCAAADPH |
| | | ECYAKVFDEFKPLVEEPQNLIKQNCELFEQLGEYKFQNALLVRYTK |
| | | KVPQVSTPTLVEVSRNLGKVGSKCCKHPEAKRMPCAEDYLSVVLNQ |
| | | LCVLHEKTPVSDRVTKCCTESLVNRRPCFSALEVDETYVPKEFNAE |
| | | TFTFHADICTLSEKERQIKKQTALVELVKHKPKATKEQLKAVMDDF |
| | | AAFVEKCCKADDKETCFAEEGKKLVAASQAALGL |
| Fc Fusion Molecule | Fc197-G4S- | DKTHTCPPCPAPELLGGPSVFLFPPKPKDTLYITREPEVTCVVVDV |
| 15 | ABD2 | SHEDPEVKFNWYVDGVEVHNAKTKPREEQYNSTYRVVSVLTVLHQD |
| (SEQ ID NO: 30) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPGGGGG |
| | | SEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDT |
| | | AVYYCTIGGSLSRSSQGTLVTVSS |
| Fc Fusion Molecule | Fc197-G4S- | DKTHTCPPCPAPELLGGPSVFLFPPKPKDILYITREPEVTCVVVDV |
| 16 | ABD1 | SHEDPEVKFNWYVDGVEVHNAKTKPREEQYNSTYRVVSVLTVLHQD |
| (SEQ ID NO: 31) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule | Fc197-3G4S- | DKTHTCPPCPAPELLGGPSVFLFPPKPKDTLYITREPEVTCVVVDV |
| 17 | ABD2 | SHEDPEVKFNWYVDGVEVHNAKTKPREEQYNSTYRVVSVLTVLHQD |
| (SEQ ID NO: 32) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPGGGGG |
| | | SGGGGSGGGGSEVQLVESGGGLVQPGNSLRLSCAASGFTFSSEGMS |
| | | WVRQAPGKGLEWVSSISGSGSDTLYADSVKGRFTISRDNAKTTLYL |
| | | QMNSLRPEDTAVYYCTIGGSLSRSSQGTLVTVSS |
| Fc Fusion Molecule | Fc197-3G4S- | DKTHTCPPCPAPELLGGPSVFLFPPKPKDTLYITREPEVTCVVVDV |
| 18 | ABD1 | SHEDPEVKFNWYVDGVEVHNAKTKPREEQYNSTYRVVSVLTVLHQD |
| (SEQ ID NO: 33) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALKYHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule | Fc197(KIH)- | DKTHTCPPCPAPELLGGPSVFLFPPKPKDTLYITREPEVTCVVVDV |
| 19 | G4S-ABD2 | SHEDPEVKFNWYVDGVEVHNAKTKPREEQYNSTYRVVSVLTVLHQD |
| (SEQ ID NO: 34) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKYHYTQKSLSLSPGGGGG |
| | | SEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDT |
| | | AVYYCTIGGSLSRSSQGTLVTVSS |
| Fc Fusion Molecule | Fc197(KIH)- | DKTHTCPPCPAPELLGGPSVFLFPPKPKDTLYITREPEVTCVVVDV |
| 20 | G4S-ABD1 | SHEDPEVKFNWYVDGVEVHNAKTKPREEQYNSTYRVVSVLTVLHQD |
| (SEQ ID NO: 35) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKYHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule | Fc197(KIH)- | DKTHTCPPCPAPELLGGPSVFLFPPKPKDTLYITREPEVTCVVVDV |
| 21 | (G4S)3-ABD2 | SHEDPEVKFNWYVDGVEVHNAKTKPREEQYNSTYRVVSVLTVLHQD |
| (SEQ ID NO: 36) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKYHYTQKSLSLSPGGGGG |
| | | SGGGGSGGGGSEVQLVESGGGLVQPGNSLRLSCAASGFTFSSFGMS |
| | | WVRQAPGKGLEWVSSISGSGSDTLYADSVKGRFTISRDNAKTTLYL |
| | | QMNSLRPEDTAVYYCTIGGSLSRSSQGTLVTVSS |
| Fc Fusion Molecule | Fc197(KIH)- | DKTHTCPPCPAPELLGGPSVFLFPPKPKDTLYITREPEVTCVVVDV |
| 22 | (G4S)3-ABD1 | SHEDPEVKFNWYVDGVEVHNAKTKPREEQYNSTYRVVSVLTVLHQD |
| (SEQ ID NO: 37) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALKYHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule | ABD2-G4S- | EVQLVESGGGLVQPGNSLRLSCAASGFTFSSFGMSWVRQAPGKGLE |
| 23 | Fc197 | WVSSISGSGSDTLYADSVKGRFTISRDNAKTTLYLQMNSLRPEDTA |
| (SEQ ID NO: 38) | | VYYCTIGGSLSRSSQGTLVTVSSGGGGSDKTHTCPPCPAPELLGGP |
| | | SVFLFPPKPKDTLYITREPEVTCVVVDVSHEDPEVKENWYVDGVEV |
| | | HNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNV |
| | | FSCSVMHEALKYHYTQKSLSLSPG |
| Fc Fusion Molecule | ABD1-G4S- | EVQLLESGGGLVQPGGSLRLSCAASGFTFRSFGMSWVRQAPGKGPE |
| 24 | Fc197 | WVSSISGSGSDTLYADSVKGRFTISRDNSKNTLYLQMNSLRPEDTA |
| (SEQ ID NO: 39) | | VYYCTIGGSLSRSSQGTLVTVSSGGGGSDKTHTCPPCPAPELLGGP |
| | | SVFLFPPKPKDTLYITREPEVTCVVVDVSHEDPEVKENWYVDGVEV |
| | | HNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNV |
| | | FSCSVMHEALKYHYTQKSLSLSPG |
| Fc Fusion Molecule | ABD2- | EVQLVESGGGLVQPGNSLRLSCAASGFTFSSFGMSWVRQAPGKGLE |
| 25 | (G4S)3-Fc197 | WVSSISGSGSDTLYADSVKGRFTISRDNAKTTLYLQMNSLRPEDTA |
| (SEQ ID NO: 40) | | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALKYHYTQKSLSLSPG |
| Fc Fusion Molecule | ABD1- | EVQLLESGGGLVQPGGSLRLSCAASGFTFRSFGMSWVRQAPGKGPE |
| 26 | (G4S)3-Fc197 | WVSSISGSGSDTLYADSVKGRFTISRDNSKNTLYLQMNSLRPEDTA |
| (SEQ ID NO: 41) | | VYYCTIGGSLSRSSQGTLVTVSSGGGGGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALKYHYTQKSLSLSPG |
| Fc Fusion Molecule | ABD2-G4S- | EVQLVESGGGLVQPGNSLRLSCAASGFTFSSFGMSWVRQAPGKGLE |
| 27 | Fc197(KIH) | WVSSISGSGSDTLYADSVKGRFTISRDNAKTTLYLQMNSLRPEDTA |
| (SEQ ID NO: 42) | | VYYCTIGGSLSRSSQGTLVTVSSGGGGSDKTHTCPPCPAPELLGGP |
| | | SVFLFPPKPKDTLYITREPEVTCVVVDVSHEDPEVKFNWYVDGVEV |
| | | HNAKTKPREEQYNSTYRVVSVLTVLHQDWLNGKEYKCKVSNKALPA |
| | | PIEKTISKAKGQPREPQVCTLPPSRDELTKNQVSLSCAVKGFYPSD |
| | | IAVEWESNGQPENNYKTTPPVLDSDGSFFLVSKLTVDKSRWQQGNV |
| | | FSCSVMHEALKYHYTQKSLSLSPG |
| Fc Fusion Molecule | ABD1-G4S- | EVQLLESGGGLVQPGGSLRLSCAASGFTFRSFGMSWVRQAPGKGPE |
| 28 | Fc197(KIH) | WVSSISGSGSDTLYADSVKGRFTISRDNSKNTLYLQMNSLRPEDTA |
| (SEQ ID NO: 43) | | VYYCTIGGSLSRSSQGTLVTVSSGGGGSDKTHTCPPCPAPELLGGP |
| | | SVFLFPPKPKDTLYITREPEVTCVVVDVSHEDPEVKENWYVDGVEV |
| | | HNAKTKPREEQYNSTYRVVSVLIVLHQDWLNGKEYKCKVSNKALPA |
| | | PIEKTISKAKGQPREPQVCTLPPSRDELTKNQVSLSCAVKGFYPSD |
| | | IAVEWESNGQPENNYKTTPPVLDSDGSFFLVSKLTVDKSRWQQGNV |
| | | FSCSVMHEALKYHYTQKSLSLSPG |
| Fc Fusion Molecule | ABD2 | EVQLVESGGGLVQPGNSLRLSCAASGFTFSSFGMSWVRQAPGKGLE |
| 29 | (G4S)3- | WVSSISGSGSDTLYADSVKGRFTISRDNAKTTLYLQMNSLRPEDTA |
| (SEQ ID NO: 44) | Fc197(KIH) | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLS |
| | | CAVKGFYPSDIAVEWESNGQPENNYKTTPPVLDSDGSFFLVSKLTV |
| | | DKSRWQQGNVFSCSVMHEALKYHYTQKSLSLSPG |
| Fc Fusion Molecule | ABD1- | EVQLLESGGGLVQPGGSLRLSCAASGFTFRSFGMSWVRQAPGKGPE |
| 30 | (G4S)3- | WVSSISGSGSDTLYADSVKGRFTISRDNSKNTLYLQMNSLRPEDTA |
| (SEQ ID NO: 45) | Fc197(KIH) | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLS |
| | | CAVKGFYPSDIAVEWESNGQPENNYKTTPPVLDSDGSFFLVSKLTV |
| | | DKSRWQQGNVFSCSVMHEALKYHYTQKSLSLSPG |
| Fc Fusion Molecule | HSA-G4S- | DAHKSEVAHRFKDLGEENFKALVLIAFAQYLQQCPFEDHVKLVNEV |
| 31 | Fc195 | TEFAKTCVADESAENCDKSLHTLFGDKLCTVATLRETYGEMADCCA |
| (SEQ ID NO: 46) | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQRFPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGLGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLIVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVLHEALKFHYTQKSLSLSPG |
| Fc Fusion Molecule | Fc195-G4S- | DKTHTCPPCPAPELLGGPSVFLFPPKPKDTLYITREPEVTCVVVDV |
| 32 | HSA | SHEDPEVKFNWYVDGVEVHNAKTKPREEQYNSTYRVVSVLTVLHQD |
| (SEQ ID NO: 47) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVLHEALKFHYTQKSLSLSPGGGGG |
| | | SDAHKSEVAHRFKDLGEENFKALVLIAFAQYLQQCPFEDHVKLVNE |
| | | VTEFAKTCVADESAENCDKSLHTLFGDKLCTVATLRETYGEMADCC |
| | | AKQEPERNECFLQHKDDNPNLPRLVRPEVDVMCTAFHDNEETFLKK |
| | | YLYEIARRHPYFYAPELLFFAKRYKAAFTECCQAADKAACLLPKLD |
| | | ELRDEGKASSAKQRLKCASLQKFGERAFKAWAVARLSQRFPKAEFA |
| | | EVSKLVTDLTKVHTECCHGDLLECADDRADLAKYICENQDSISSKL |
| | | KECCEKPLLEKSHCIAEVENDEMPADLPSLAADFVESKDVCKNYAE |
| | | AKDVFLGMFLYEYARRHPDYSVVLLLRLAKTYETTLEKCCAAADPH |
| | | ECYAKVFDEFKPLVEEPQNLIKQNCELFEQLGEYKFQNALLVRYTK |
| | | KVPQVSTPTLVEVSRNLGKVGSKCCKHPEAKRMPCAEDYLSVVLNQ |
| | | LCVLHEKTPVSDRVTKCCTESLVNRRPCFSALEVDETYVPKEFNAE |
| | | TFTFHADICTLSEKERQIKKQTALVELVKHKPKATKEQLKAVMDDF |
| | | AAFVEKCCKADDKETCFAEEGKKLVAASQAALGL |
| Fc Fusion Molecule | Fc195(KIH)- | DKTHTCPPCPAPELLGGPSVFLFPPKPKDTLYITREPEVTCVVVDV |
| 33 | G4S-ABD1 | SHEDPEVKFNWYVDGVEVHNAKTKPREEQYNSTYRVVSVLTVLHQD |
| (SEQ ID NO: 48) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVLHEALKFHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule | Fc195(KIH)- | DKTHTCPPCPAPELLGGPSVFLFPPKPKDTLYITREPEVTCVVVDV |
| 34 | (G4S)3-ABD1 | SHEDPEVKFNWYVDGVEVHNAKTKPREEQYNSTYRVVSVLTVLHQD |
| (SEQ ID NO: 49) | | WLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVLHEALKFHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule | ABD1-G4S- | EVQLLESGGGLVQPGGSLRLSCAASGFTFRSFGMSWVRQAPGKGPE |
| 35 | Fc195 | WVSSISGSGSDTLYADSVKGRFTISRDNSKNTLYLQMNSLRPEDTA |
| (SEQ ID NO: 50) | | VYYCTIGGSLSRSSQGTLVTVSSGGGGSDKTHTCPPCPAPELLGGP |
| | | SVFLFPPKPKDTLYITREPEVTCVVVDVSHEDPEVKENWYVDGVEV |
| | | HNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNV |
| | | FSCSVLHEALKFHYTQKSLSLSPG |
| Fc Fusion Molecule | ABD1-G4S- | EVQLLESGGGLVQPGGSLRLSCAASGFTFRSFGMSWVRQAPGKGPE |
| 36 | Fc195(KIH) | WVSSISGSGSDTLYADSVKGRFTISRDNSKNTLYLQMNSLRPEDTA |
| (SEQ ID NO: 51) | | VYYCTIGGSLSRSSQGTLVTVSSGGGGSDKTHTCPPCPAPELLGGP |
| | | SVFLFPPKPKDTLYITREPEVTCVVVDVSHEDPEVKFNWYVDGVEV |
| | | HNAKTKPREEQYNSTYRVVSVLTVLHQDWLNGKEYKCKVSNKALPA |
| | | PIEKTISKAKGQPREPQVCTLPPSRDELTKNQVSLSCAVKGFYPSD |
| | | IAVEWESNGQPENNYKTTPPVLDSDGSFFLVSKLTVDKSRWQQGNV |
| | | FSCSVLHEALKFHYTQKSLSLSPG |
| Fc Fusion Molecule | Fc195-G4S- | DKTHTCPPCPAPELLGGPSVFLFPPKPKDTLYITREPEVTCVVVDV |
| 37 | ABD1 | SHEDPEVKFNWYVDGVEVHNAKTKPREEQYNSTYRVVSVLTVLHQD |
| (SEQ ID NO: 52) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVLHEALKFHYTQKSLSLSPGGGGG |
| | | SEVQLLESGGGLVQPGGSLRLSCAASGFTFRSFGMSWVRQAPGKGP |
| | | EWVSSISGSGSDTLYADSVKGRFTISRDNSKNTLYLQMNSLRPEDT |
| | | AVYYCTIGGSLSRSSQGTLVTVSS |
| Fc Fusion Molecule | Fc195-(G4S)3- | DKTHTCPPCPAPELLGGPSVFLFPPKPKDTLYITREPEVTCVVVDV |
| 38 | ABD1 | SHEDPEVKFNWYVDGVEVHNAKTKPREEQYNSTYRVVSVLTVLHQD |
| (SEQ ID NO: 53) | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVLHEALKFHYTQKSLSLSPGGGGG |
| | | SGGGGSGGGGSEVQLLESGGGLVQPGGSLRLSCAASGFTFRSFGMS |
| | | WVRQAPGKGPEWVSSISGSGSDTLYADSVKGRFTISRDNSKNTLYL |
| | | QMNSLRPEDTAVYYCTIGGSLSRSSQGTLVTVSS |
| Fc Fusion Molecule | ABD1- | EVQLLESGGGLVQPGGSLRLSCAASGFTFRSFGMSWVRQAPGKGPE |
| 39 | (G4S)3-Fc195 | WVSSISGSGSDTLYADSVKGRFTISRDNSKNTLYLQMNSLRPEDTA |
| (SEQ ID NO: 54) | | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVLHEALKFHYTQKSLSLSPG |
| Fc Fusion Molecule | ABD1- | EVQLLESGGGLVQPGGSLRLSCAASGFTFRSFGMSWVRQAPGKGPE |
| 40 | (G4S)3- | WVSSISGSGSDTLYADSVKGRFTISRDNSKNTLYLQMNSLRPEDTA |
| (SEQ ID NO: 55) | Fc195(KIH) | VYYCTIGGSLSRSSQGTLVTVSSGGGGSGGGGSGGGGSDKTHTCPP |
| | | CPAPELLGGPSVFLFPPKPKDTLYITREPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVCTLPPSRDELTKNQVSLS |
| | | CAVKGFYPSDIAVEWESNGQPENNYKTTPPVLDSDGSFFLVSKLTV |
| | | DKSRWQQGNVFSCSVLHEALKFHYTQKSLSLSPG |
| WT human IgG1 Fc | Human IGH1 | DKTHTCPPCPAPELLGGPSVFLFPPKPKDTLMISRTPEVTCVVVDV |
| (SEQ ID NO: 56) | (221-446) | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPG |
| Fc Fragment 10 | IgG1 | DKTHTCPPCPAPELLGGPSVFLFPPKPKDTLYITREPEVTCVVVDV |
| (SEQ ID NO: 57) | Fc_M252Y/ | SHEDPEVKFNWYVDGVEVHNAKTKPREEQYNSTYRVVSVLTVLHQD |
| | S254T/T256E/ | WLNGKEYKCKVSNKALPAPIEKTISKAKGQPREPQVYTLPPSRDEL |
| | M428L/H433K/ | TKNQVSLTCLVKGFYPSDIAVEWESNGQPENNYKTTPPVLDSDGSF |
| | N434Y | FLYSKLTVDKSRWQQGNVFSCSVLHEALKYHYTQKSLSLSPG |
| Fc Fragment 11 | IgG1 | DKTHTCPPCPAPELLGGPSVFLFPPKPKDTLYITREPEVTCVVVDV |
| (SEQ ID NO: 58) | Fc_M252Y/ | SHEDPEVKFNWYVDGVEVHNAKTKPREEQYNSTYRVVSVLTVLHQD |
| | S254T/T256E/ | WLNGKEYKCKVSNKALPAPIEKTISKAKGQPREPQVYTLPPSRDEL |
| | M428L/N434F | TKNQVSLTCLVKGFYPSDIAVEWESNGQPENNYKTTPPVLDSDGSF |
| | | FLYSKLTVDKSRWQQGNVFSCSVLHEALHFHYTQKSLSLSPG |
| Fc Fragment 12 | IgG1 | DKTHTCPPCPAPELLGGPSVFLFPPKPKDTLYITREPEVTCVVVDV |
| (SEQ ID NO: 59) | Fc_M252Y/ | SHEDPEVKFNWYVDGVEVHNAKTKPREEQYNSTYRVVSVLTVLHQD |
| | S254T/T256E/ | WLNGKEYKCKVSNKALPAPIEKTISKAKGQPREPQVYTLPPSRDEL |
| | H433R/N434Y | TKNQVSLTCLVKGFYPSDIAVEWESNGQPENNYKTTPPVLDSDGSF |
| | | FLYSKLTVDKSRWQQGNVFSCSVMHEALRYHYTQKSLSLSPG |
| Fc Fragment 13 | IgG1 | DKTHTCPPCPAPELLGGPSVFLFPPKPKDTLYITREPEVTCVVVDV |
| (SEQ ID NO: 60) | Fc_M252Y/ | SHEDPEVKFNWYVDGVEVHNAKTKPREEQYNSTYRVVSVLTVLHQD |
| | S254T/T256E/ | WLNGKEYKCKVSNKALPAPIEKTISKAKGQPREPQVYTLPPSRDEL |
| | H433R/N434F | TKNQVSLTCLVKGFYPSDIAVEWESNGQPENNYKTTPPVLDSDGSF |
| | | FLYSKLTVDKSRWQQGNVFSCSVMHEALRFHYTQKSLSLSPG |
| Fc Fragment 14 | IgG1 | DKTHTCPPCPAPELLGGPSVFLFPPKPKDTLYITREPEVTCVVVDV |
| (SEQ ID NO: 61) | Fc_M252Y/ | SHEDPEVKFNWYVDGVEVHNAKTKPREEQYNSTYRVVSVLIVLHQD |
| | S254T/T256E/ | WLNGKEYKCKVSNKALPAPIEKTISKAKGQPREPQVYTLPPSRDEL |
| | M428L/H433R/ | TKNQVSLTCLVKGFYPSDIAVEWESNGQPENNYKTTPPVLDSDGSF |
| | N434F | FLYSKLTVDKSRWQQGNVFSCSVLHEALRFHYTQKSLSLSPG |
| Fc Fragment 15 | IgG1 | DKTHTCPPCPAPELLGGPSVFLFPPKPKDTLYITREPEVTCVVVDV |
| (SEQ ID NO: 62) | Fc_M252Y/ | SHEDPEVKFNWYVDGVEVHNAKTKPREEQYNSTYRVVSVLTVDHQD |
| | S254T/T256E/ | WLNGKEYKCKVSNKALPAPIEKTISKAKGQPREPQVYTLPPSRDEL |
| | L309D/H433K/ | TKNQVSLTCLVKGFYPSDIAVEWESNGQPENNYKTTPPVLDSDGSF |
| | N434F | FLYSKLTVDKSRWQQGNVFSCSVMHEALKFHYTQKSLSLSPG |
| Fc Fragment 16 | IgG1 | DKTHTCPPCPAPELLGGPSVFLFPPKPKDTLYITREPEVTCVVVDV |
| (SEQ ID NO: 63) | Fc_M252Y/ | SHEDPEVKFNWYVDGVEVHNAKTKPREEQYNSTYRVVSVLTVDHQD |
| | 5S24T/T256E/ | WLNGKEYKCKVSNKALPAPIEKTISKAKGQPREPQVYTLPPSRDEL |
| | L309D/H433K/ | TKNQVSLTCLVKGFYPSDIAVEWESNGQPENNYKTTPPVLDSDGSF |
| | N434Y | FLYSKLTVDKSRWQQGNVFSCSVMHEALKYHYTQKSLSLSPG |
| Fc Fragment 17 | IgG1 | DKTHTCPPCPAPELLGGPSVFLFPPKPKDTLYITREPEVTCVVVDV |
| (SEQ ID NO: 64) | Fc_M252Y/ | SHEDPEVKFNWYVDGVEVHNAKTKPREEQYNSTYRVVSVLTVDHQD |
| | S254T/T256E/ | WLNGKEYKCKVSNKALPAPIEKTISKAKGQPREPQVYTLPPSRDEL |
| | L309D/M428L/ | TKNQVSLTCLVKGFYPSDIAVEWESNGQPENNYKTTPPVLDSDGSF |
| | H433K/N434F | FLYSKLTVDKSRWQQGNVFSCSVLHEALKFHYTQKSLSLSPG |
| Fc Fragment 18 | IgG1 | DKTHTCPPCPAPELLGGPSVFLFPPKPKDTLYITREPEVTCVVVDV |
| (SEQ ID NO: 65) | Fc_M252Y/ | SHEDPEVKFNWYVDGVEVHNAKTKPREEQYNSTYRVVSVLTVDHQD |
| | S254T/T256E/ | WLNGKEYKCKVSNKALPAPIEKTISKAKGQPREPQVYTLPPSRDEL |
| | L309D/M428L/ | TKNQVSLTCLVKGFYPSDIAVEWESNGQPENNYKTTPPVLDSDGSF |
| | H433K/N434Y | FLYSKLTVDKSRWQQGNVFSCSVLHEALKYHYTQKSLSLSPG |
| Fc Fragment 19 | IgG1 | DKTHTCPPCPAPELLGGPSVFLFPPKPKDTLYITREPEVTCVVVDV |
| (SEQ ID NO: 66) | Fc_M252Y/ | SHEDPEVKFNWYVDGVEVHNAKTKPREEQYNSTYRVVSVLTVDHKD |
| | 5S24T/T256E/ | WLNGKEYKCKVSNKALPAPIEKTISKAKGQPREPQVYTLPPSRDEL |
| | L309D/Q311K/ | TKNQVSLTCLVKGFYPSDIAVEWESNGQPENNYKTTPPVLDSDGSF |
| | H433K/N434F | FLYSKLTVDKSRWQQGNVFSCSVMHEALKFHYTQKSLSLSPG |
| Fc Fragment 20 | IgG1 | DKTHTCPPCPAPELLGGPSVFLFPPKPKDTLYITREPEVTCVVVDV |
| (SEQ ID NO: 67) | Fc_M252Y/ | SHEDPEVKFNWYVDGVEVHNAKTKPREEQYNSTYRVVSVLTVDHKD |
| | S254T/T256E/ | WLNGKEYKCKVSNKALPAPIEKTISKAKGQPREPQVYTLPPSRDEL |
| | L309D/Q311K/ | TKNQVSLTCLVKGFYPSDIAVEWESNGQPENNYKTTPPVLDSDGSF |
| | H433K/N434Y | FLYSKLTVDKSRWQQGNVFSCSVMHEALKYHYTQKSLSLSPG |
| Fc Fusion Molecule 41 | Fc197(KIH)- | DKTHTCPPCPAPELLGGPSVFLFPPKPKDTLYITREPEVTCVVVDVSHEDP |
| (SEQ ID NO: 68) | (G4S)6-ABD1 | EVKFNWYVDGVEVHNAKTKPREEQYNSTYRVVSVLIVLHQDWLNGKEYKCK |
| | | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSGGGGSGGGGSGGGG |
| | | SEVQLLESGGGLVQPGGSLRLSCAASGFTFRSFGMSWVRQAPGKGPEWVSS |
| | | ISGSGSDTLYADSVKGRFTISRDNSKNTLYLQMNSLRPEDTAVYYCTIGGS |
| | | LSRSSQGTLVTVSS |
| Fc Fusion Molecule 42 | Fc197(KIH)- | DKTHTCPPCPAPELLGGPSVFLFPPKPKDTLYITREPEVTCVVVDVSHEDP |
| (SEQ ID NO: 69) | (G4S)8-ABD1 | EVKFNWYVDGVEVHNAKTKPREEQYNSTYRVVSVLIVLHQDWLNGKEYKCK |
| | | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSGGGGSGGGGSGGGG |
| | | SGGGGSGGGGSEVQLLESGGGLVQPGGSLRLSCAASGFTFRSFGMSWVRQA |
| | | PGKGPEWVSSISGSGSDTLYADSVKGRFTISRDNSKNTLYLQMNSLRPEDT |
| | | AVYYCTIGGSLSRSSQGTLVTVSS |
| Fc Fusion Molecule 43 | Fc197(KIH)- | DKTHTCPPCPAPELLGGPSVFLFPPKPKDTLYITREPEVTCVVVDVSHEDP |
| (SEQ ID NO: 70) | (G4S)3-ABD1 | EVKFNWYVDGVEVHNAKTKPREEQYNSTYRVVSVLTVLHQDWLNGKEYKCK |
| | (F32A) | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSEVQLLESGGGLVQP |
| | | GGSLRLSCAASGFTFRSAGMSWVRQAPGKGPEWVSSISGSGSDTLYADSVK |
| | | GRFTISRDNSKNTLYLQMNSLRPEDTAVYYCTIGGSLSRSSQGTLVTVSS |
| Fc Fusion Molecule 44 | Fc197(KIH)- | DKTHTCPPCPAPELLGGPSVFLFPPKPKDTLYITREPEVTCVVVDVSHEDP |
| (SEQ ID NO: 71) | (G4S)6-ABD1 | EVKFNWYVDGVEVHNAKTKPREEQYNSTYRVVSVLTVLHQDWLNGKEYKCK |
| | (F32A) | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSGGGGSGGGGSGGGG |
| | | SEVQLLESGGGLVQPGGSLRLSCAASGFTERSAGMSWVRQAPGKGPEWVSS |
| | | ISGSGSDTLYADSVKGRFTISRDNSKNTLYLQMNSLRPEDTAVYYCTIGGS |
| | | LSRSSQGTLVTVSS |
| Fc Fusion Molecule 45 | Fc197(KIH)- | DKTHTCPPCPAPELLGGPSVFLFPPKPKDTLYITREPEVTCVVVDVSHEDP |
| (SEQ ID NO: 72) | (G4S)7-ABD1 | EVKFNWYVDGVEVHNAKTKPREEQYNSTYRVVSVLTVLHQDWLNGKEYKCK |
| | (F32A) | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSGGGGSGGGGSGGGG |
| | | SGGGGSEVQLLESGGGLVQPGGSLRLSCAASGFTFRSAGMSWVRQAPGKGP |
| | | EWVSSISGSGSDTLYADSVKGRFTISRDNSKNTLYLQMNSLRPEDTAVYYC |
| | | TIGGSLSRSSQGTLVTVSS |
| Fc Fusion Molecule 46 | Fc197(KIH)- | DKTHTCPPCPAPELLGGPSVFLFPPKPKDTLYITREPEVTCVVVDVSHEDP |
| (SEQ ID NO: 73) | (G4S)8-ABD1 | EVKFNWYVDGVEVHNAKTKPREEQYNSTYRVVSVLTVLHQDWLNGKEYKCK |
| | (F32A) | VSNKALPAPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVES |
| | | CSVMHEALKYHYTQKSLSLSPGGGGGSGGGGSGGGGSGGGGSGGGGSGGGG |
| | | SGGGGSGGGGSEVQLLESGGGLVQPGGSLRLSCAASGFTFRSAGMSWVRQA |
| | | PGKGPEWVSSISGSGSDTLYADSVKGRFTISRDNSKNTLYLQMNSLRPEDT |
| | | AVYYCTIGGSLSRSSQGTLVTVSS |
| (G4S)6 | Linker | GGGGSGGGGSGGGGSGGGGSGGGGSGGGGS |
| (SEQ ID NO: 74) | | |
| (G4S)8 | Linker | GGGGSGGGGSGGGGSGGGGSGGGGSGGGGSGGGGSGGGGS |
| (SEQ ID NO: 75) | | |
| ABD1 (F32A) | ABD1 with | EVQLLESGGGLVQPGGSLRLSCAASGFTFRSAGMSWVRQAPGKGPEWVSSI |
| (SEQ ID NO: 76) | F32A mutation | SGSGSDTLYADSVKGRFTISRDNSKNTLYLQMNSLRPEDTAVYYCTIGGSL |
| | | SRSSQGTLVTVSS |

### ENUMERATED EMBODIMENTS

Embodiment 1. A Fc fusion molecule that binds neonatal Fc receptor (FcRn) comprising from N-terminus to C-terminus or C-terminus to N-terminus:
a) a Fc fragment comprising a sequence having at least 80% sequence identity to the amino acid sequence of any one of SEQ ID NOs: 1-9;
b) a linker; and
c) an albumin or an albumin binding domain comprising a sequence having at least 80% sequence identity to the amino acid sequence of any one of SEQ ID NOs: 10-12.

Embodiment 2. The Fe fusion molecule of Embodiment 1, wherein the linker comprises a sequence according to any one of SEQ ID NOs: 13-15.

Embodiment 3. The Fc fusion molecule of any one of Embodiments 1-2, wherein the Fc fragment is derived from an IgG1, IgG2 or IgG4 immunoglobulin domain.

Embodiment 4. The Fc fusion molecule of any one of Embodiments 1-2, wherein the Fc fragment is derived from an IgG1 immunoglobulin domain.

Embodiment 5. The Fc fusion molecule of any one of Embodiments 1-2, wherein the Fc fragment is derived from an IgG2 immunoglobulin domain.

Embodiment 6. The Fc fusion molecule of any one of Embodiments 1-2, wherein the Fc fragment is derived from an IgG4 immunoglobulin domain.

Embodiment 7. The Fc fusion molecule of any one of Embodiments 1-6, wherein the Fc fragment comprises a sequence having at least 80% sequence identity to the amino acid sequence to SEQ ID NO: 1, the linker comprises a sequence according to SEQ ID NO: 13, and the albumin or albumin binding domain comprises a sequence having at least 80% sequence identity to the amino acid sequence according to SEQ ID NO: 10.

Embodiment 8. The Fc fusion molecule of any one of Embodiments 1-6, wherein the Fc fragment comprises a sequence having at least 80% sequence identity to the amino acid sequence to SEQ ID NO: 1, the linker comprises a sequence according to SEQ ID NO: 14, and the albumin or albumin binding domain comprises a sequence having at least 80% sequence identity to the amino acid sequence according to SEQ ID NO: 10.

Embodiment 9. The Fc fusion molecule of any one of Embodiments 1-6, wherein the Fc fragment comprises a sequence having at least 80% sequence identity to the amino acid sequence to SEQ ID NO: 1, the linker comprises a sequence according to SEQ ID NO: 13, and the albumin or albumin binding domain comprises a sequence having at least 80% sequence identity to the amino acid sequence according to SEQ ID NO: 11.

Embodiment 10. The Fc fusion molecule of any one of Embodiments 1-6, wherein the Fc fragment comprises a sequence having at least 80% sequence identity to the amino acid sequence to SEQ ID NO: 1, the linker comprises a sequence according to SEQ ID NO: 15, and the albumin or albumin binding domain comprises a sequence having at least 80% sequence identity to the amino acid sequence according to SEQ ID NO: 11.

Embodiment 11. The Fc fusion molecule of any one of Embodiments 1-6, wherein the Fc fragment comprises a sequence having at least 80% sequence identity to the amino acid sequence to SEQ ID NO: 4 or SEQ ID NO: 5, the linker comprises a sequence according to SEQ ID NO: 13, and the albumin or albumin binding domain comprises a sequence having at least 80% sequence identity to the amino acid sequence according to SEQ ID NO: 11.

Embodiment 12. The Fc fusion molecule of any one of Embodiments 1-6, wherein the Fc fragment comprises a sequence having at least 80% sequence identity to the amino acid sequence to SEQ ID NO: 4 or SEQ ID NO: 5, the linker comprises a sequence according to SEQ ID NO: 15, and the albumin or albumin binding domain comprises a sequence having at least 80% sequence identity to the amino acid sequence according to SEQ ID NO: 11.

Embodiment 13. The Fc fusion molecule of any one of Embodiments 1-6, wherein the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 10.

Embodiment 14. The Fc fusion molecule of any one of Embodiments 1-6, wherein the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 12.

Embodiment 15. The Fc fusion molecule of any one of Embodiments 1-6, wherein the Fc fragment comprises a sequence having at least 80% sequence identity to the amino acid sequence to SEQ ID NO: 3, the linker comprises a sequence according to SEQ ID NO: 13, and the albumin or albumin binding domain comprises a sequence having at least 80% sequence identity to the amino acid sequence according to SEQ ID NO: 11.

Embodiment 16. The Fc fusion molecule of any one of Embodiments 1-6, wherein the Fc fragment comprises a sequence having at least 80% sequence identity to the amino acid sequence to SEQ ID NO: 3, the linker comprises a sequence according to SEQ ID NO: 15, and the albumin or albumin binding domain comprises a sequence having at least 80% sequence identity to the amino acid sequence according to SEQ ID NO: 12.

Embodiment 17. The Fc fusion molecule of any one of Embodiments 1-6, wherein the Fc fragment comprises a sequence having at least 80% sequence identity to the amino acid sequence to SEQ ID NO: 3, the linker comprises a sequence according to SEQ ID NO: 15, and the albumin or albumin binding domain comprises a sequence having at least 80% sequence identity to the amino acid sequence according to SEQ ID NO: 11.

Embodiment 18. The Fc fusion molecule of any one of Embodiments 1-6, wherein the Fc fragment comprises a sequence having at least 80% sequence identity to the amino acid sequence to SEQ ID NO: 6 or SEQ ID NO: 7, the linker comprises a sequence according to SEQ ID NO: 13, and the albumin or albumin binding domain comprises a sequence having at least 80% sequence identity to the amino acid sequence according to SEQ ID NO: 12.

Embodiment 19. The Fc fusion molecule of any one of Embodiments 1-6, wherein the Fc fragment comprises a sequence having at least 80% sequence identity to the amino acid sequence to SEQ ID NO: 6 or SEQ ID NO: 7, the linker comprises a sequence according to SEQ ID NO: 13, and the albumin or albumin binding domain comprises a sequence having at least 80% sequence identity to the amino acid sequence according to SEQ ID NO: 11.

Embodiment 20. The Fc fusion molecule of any one of Embodiments 1-6, wherein the Fc fragment comprises a sequence having at least 80% sequence identity to the amino acid sequence to SEQ ID NO: 6 or SEQ ID NO: 7, the linker comprises a sequence according to SEQ ID NO: 15, and the albumin or albumin binding domain comprises a sequence having at least 80% sequence identity to the amino acid sequence according to SEQ ID NO: 12.

Embodiment 21. The Fc fusion molecule of any one of Embodiments 1-6, wherein the Fc fragment comprises a sequence having at least 80% sequence identity to the amino acid sequence to SEQ ID NO: 6 or SEQ ID NO: 7, the linker comprises a sequence according to SEQ ID NO: 15, and the albumin or albumin binding domain comprises a sequence having at least 80% sequence identity to the amino acid sequence according to SEQ ID NO: 11.

Embodiment 22. The Fc fusion molecule of any one of Embodiments 1-6, wherein the Fc fragment comprises a sequence having at least 80% sequence identity to the amino acid sequence to SEQ ID NO: 2, the linker comprises a sequence according to SEQ ID NO: 13, and the albumin or albumin binding domain comprises a sequence having at least 80% sequence identity to the amino acid sequence according to SEQ ID NO: 10.

Embodiment 23. The Fc fusion molecule of any one of Embodiments 1-6, wherein the Fc fragment comprises a sequence having at least 80% sequence identity to the amino acid sequence to SEQ ID NO: 8 or SEQ ID NO: 9, the linker comprises a sequence according to SEQ ID NO: 13, and the albumin or albumin binding domain comprises a sequence having at least 80% sequence identity to the amino acid sequence according to SEQ ID NO: 11.

Embodiment 24. The Fc fusion molecule of any one of Embodiments 1-6, wherein the Fc fragment comprises a sequence having at least 80% sequence identity to the amino acid sequence to SEQ ID NO: 8 or SEQ ID NO: 9, the linker comprises a sequence according to SEQ ID NO: 15, and the albumin or albumin binding domain comprises a sequence having at least 80% sequence identity to the amino acid sequence according to SEQ ID NO: 11.

Embodiment 25. The Fc fusion molecule of any one of Embodiments 1-6, wherein the Fc fragment comprises a sequence having at least 80% sequence identity to the amino acid sequence to SEQ ID NO: 2, the linker comprises a sequence according to SEQ ID NO: 13, and the albumin or albumin binding domain comprises a sequence having at least 80% sequence identity to the amino acid sequence according to SEQ ID NO: 11.

Embodiment 26. The Fc fusion molecule of any one of Embodiments 1-6, wherein the Fc fragment comprises a sequence having at least 80% sequence identity to the amino acid sequence to SEQ ID NO: 2, the linker comprises a sequence according to SEQ ID NO: 15, and the albumin or albumin binding domain comprises a sequence having at least 80% sequence identity to the amino acid sequence according to SEQ ID NO: 11.

Embodiment 27. The Fc fusion molecule of any one of Embodiments 1-26, further comprising a second Fc fragment comprising a sequence having at least 80% sequence identity to the amino acid sequence of any one of SEQ ID NOs: 1-9.

Embodiment 28. A Fc fusion molecule that binds neonatal Fc receptor (FcRn) comprising a sequence having at least 80% sequence identity to any one of SEQ ID NOs: 16-55.

Embodiment 29. The Fc fusion molecule of any one of Embodiments 1-28, wherein the Fc fusion molecule binds to FcRn with a KD of less than or equal to about 1×10-8 M, at pH 6.0 or pH 7.4 as measured by surface plasmon resonance (SPR).

Embodiment 30. The Fc fusion molecule of any one of Embodiments 1-29, wherein the Fc fusion molecule comprises a half-life at least 2 fold longer as compared to a Fc fusion molecule not comprising an albumin or an albumin binding domain.

Embodiment 31. The Fc fusion molecule of Embodiment 30, wherein the albumin or the albumin domain comprises a sequence having at least 80% sequence identity to the amino acid sequence of any one of SEQ ID NOs: 10-12 any one of SEQ ID NOs: 10-12.

Embodiment 32. A pharmaceutical composition, comprising the Fc fusion molecule of any one of Embodiments 1-31 and a pharmaceutically acceptable carrier.

Embodiment 33. A method of treating a disease or disorder in a patient in need thereof, the method comprising administering to the patient an effective amount of the Fc fusion molecule of any one of Embodiments 1-31 or a pharmaceutical composition of Embodiment 32.

Embodiment 34. The method of Embodiment 33, wherein the disease or disorder is an autoimmune disease.

Embodiment 35. The method of Embodiment 33 or 34, wherein the disease or disorder is generalized myasthenia gravis (gMG), chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenic purpura, primary Sjogren's Syndrome, systemic lupus erythematosus, rheumatoid arthritis, bullous pemphigoid, pemphigus foliaceus, pemphigus vulgaris, or cutaneous lupus erythematosus.

Embodiment 36. The method of Embodiment 33 or 34, wherein the method reduces disease severity in a patient and wherein disease severity is assessed by an gMG Disease Severity Outcome Measure.

Embodiment 37. A method for treating a pathology associated with elevated levels of an IgG in a patient in need thereof, the method comprising administering to the patient a therapeutically effective amount the isolated Fc fragment of any one of Embodiments 1-31 or a pharmaceutical composition of Embodiment 32.

Embodiment 38. A method of reducing biological activity of an IgG in a patient in need thereof, the method comprising administering to the patient a therapeutically effective amount of the Fc fusion molecule of any one of Embodiments 1-31 or a pharmaceutical composition of Embodiment 32.

Embodiment 39. The method of Embodiment 38, wherein the disease is an autoimmune disease.

Embodiment 40. A method of preventing a disorder in a patient in need thereof, the method comprising administering to the patient a therapeutically effective amount the Fc fusion molecule of any one of Embodiments 1-31 or a pharmaceutical composition of Embodiment 32; wherein the disorder is an unwanted side-effect of a therapeutic antibody.

### EQUIVALENTS AND SCOPE

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

1. An Fc fusion molecule that binds neonatal Fc receptor (FcRn) comprising from N-terminus to C-terminus or C-terminus to N-terminus:
a) an Fc fragment comprising amino acid substitutions (i) N434Y and (ii) H433R or H433K as compared to an amino acid sequence set forth in SEQ ID NO: 56;
b) a linker; and
c) an albumin or an albumin binding domain.

2. The Fc fusion molecule of claim 1, wherein the Fc fragment comprises amino acid substitutions N434Y and H433R.

3. The Fc fusion molecule of claim 1, wherein the Fc fragment comprises amino acid substitutions N434Y and H433K.

4. An Fc fusion molecule that binds neonatal Fc receptor (FcRn) comprising from N-terminus to C-terminus or C-terminus to N-terminus:
a) an Fc fragment comprising amino acid substitutions M428L and N434F as compared to an amino acid sequence set forth in SEQ ID NO: 56;
b) a linker; and
c) an albumin or an albumin binding domain.

5. The Fc fusion molecule of claim 4, wherein the Fc fragment further comprises amino acid substitution H433K.

6. An Fc fusion molecule that binds neonatal Fc receptor (FcRn) comprising from N-terminus to C-terminus or C-terminus to N-terminus:
a) an Fc fragment comprises amino acid substitutions H433R and N434F as compared to an amino acid sequence set forth in SEQ ID NO: 56.
b) a linker; and
c) an albumin or an albumin binding domain.

7. The Fc fusion molecule of claim 6, wherein the Fc fragment further comprises amino acid substitution M428L.

8. The Fc fusion molecule of any one of the preceding claims, wherein the Fc fragment further comprises amino acid substitutions M252Y, S254T, and T256E.

9. The Fc fusion molecule of any one of the preceding claims, wherein the Fc fragment comprises an amino acid sequence at least 80%, at least 85%, at least 90%, at least 95%, at least 96%, at least 97%, at least 98%, or at least 99% identical to any one of SEQ ID NOs: 1-9 or 57-67.

10. The Fc fusion molecule of any one of the preceding claims, wherein the Fc fragment comprises an amino acid sequence identical to any one of SEQ ID NOs: 1-9 or 57-67.

11. The Fc fusion molecule of any one of the preceding claims, wherein the Fc fusion molecule comprises an albumin.

12. The Fc fusion molecule of claim 11, wherein the albumin comprises an amino acid sequence at least 80%, at least 85%, at least 90%, at least 95%, at least 96%, at least 97%, at least 98%, or at least 99% identical to SEQ ID NO: 10.

13. The Fc fusion molecule of claim 11 or 12, wherein the albumin comprises an amino acid sequence identical to SEQ ID NO: 10.

14. The Fc fusion molecule of any one of claims 1-10, wherein the Fc fusion molecule comprises an albumin binding domain.

15. The Fc fusion molecule of claim 14, wherein the albumin binding domain comprises an amino acid sequence at least 80%, at least 85%, at least 90%, at least 95%, at least 96%, at least 97%, at least 98%, or at least 99% identical to SEQ ID NO: 11 or SEQ ID NO: 12.

16. The Fc fusion molecule of claim 14 or 15, wherein the albumin binding domain comprises an amino acid sequence identical to SEQ ID NO: 11 or SEQ ID NO: 12.

17. The Fc fusion molecule of any one of the preceding claims, wherein the linker comprises glycine and serine.

18. The Fc fusion molecule of claim 17, wherein the linker comprises one or more repeating units of GGGGS (SEQ ID NO: 13).

19. The Fc fusion molecule of claim 17 or 18, wherein the linker comprises at least 2, at least 3, at least 4, at least 5, at least 6, at least 7, or at least 8 repeating units of GGGGS (SEQ ID NO: 13).

20. The Fc fusion molecule of any one of claims 17-19, wherein the linker comprises 6 repeating units of GGGGS (SEQ ID NO: 13).

21. The Fc fusion molecule of any one of claims 17-19, wherein the linker comprises 8 repeating units of GGGGS (SEQ ID NO: 13).

22. The Fc fusion molecule of any one of the preceding claims, wherein the linker comprises a sequence according to any one of SEQ ID NOs: 13-15.

23. The Fc fusion molecule of any one of the preceding claims, wherein the Fc fragment is derived from an IgG1, IgG2 or IgG4 immunoglobulin domain.

24. The Fc fusion molecule of any one of the preceding claims, wherein the Fc fragment is derived from an IgG1 immunoglobulin domain.

25. The Fc fusion molecule of any one of claims 1-23, wherein the Fc fragment is derived from an IgG2 immunoglobulin domain.

26. The Fc fusion molecule of any one of claims 1-23, wherein the Fc fragment is derived from an IgG4 immunoglobulin domain.

27. The Fc fusion molecule of any one of the preceding claims, wherein
a) the Fc fragment comprises a sequence having at least 80% sequence identity to SEQ ID NO: 1,
b) the linker comprises a sequence of SEQ ID NO: 13, and
c) the albumin or albumin binding domain comprises a sequence having at least 80% sequence identity to SEQ ID NO: 10.

28. The Fc fusion molecule of any one of claims 1-26, wherein the
a) Fc fragment comprises a sequence having at least 80% sequence identity to SEQ ID NO: 1,
b) the linker comprises a sequence of SEQ ID NO: 14, and
c) the albumin or albumin binding domain comprises a sequence having at least 80% sequence identity to SEQ ID NO: 10.

29. The Fc fusion molecule of any one of claims 1-26,
a) wherein the Fc fragment comprises a sequence having at least 80% sequence identity to SEQ ID NO: 1,
b) the linker comprises a sequence of SEQ ID NO: 13,
c) and the albumin or albumin binding domain comprises a sequence having at least 80% sequence identity to SEQ ID NO: 11.

30. The Fc fusion molecule of any one of claims 1-26,
a) wherein the Fc fragment comprises a sequence having at least 80% sequence identity to SEQ ID NO: 1,
b) the linker comprises a sequence of SEQ ID NO: 15, and
c) the albumin or albumin binding domain comprises a sequence having at least 80% sequence identity to SEQ ID NO: 11.

31. The Fc fusion molecule of any one of claims 1-26,
a) wherein the Fc fragment comprises a sequence having at least 80% sequence identity to SEQ ID NO: 4 or SEQ ID NO: 5,
b) the linker comprises a sequence of SEQ ID NO: 13, and
c) the albumin or albumin binding domain comprises a sequence having at least 80% sequence identity to SEQ ID NO: 11.

32. The Fc fusion molecule of any one of claims 1-26, wherein the
a) Fc fragment comprises a sequence having at least 80% sequence identity to SEQ ID NO: 4 or SEQ ID NO: 5,
b) the linker comprises a sequence of SEQ ID NO: 15, and
c) the albumin or albumin binding domain comprises a sequence having at least 80% sequence identity to SEQ ID NO: 11.

33. The Fc fusion molecule of any one of claims 1-26, wherein the
a) Fc fragment comprises a sequence having at least 80% sequence identity to SEQ ID NO: 3,
b) the linker comprises a sequence of SEQ ID NO: 13, and
c) the albumin or albumin binding domain comprises a sequence having at least 80% sequence identity to SEQ ID NO: 10.

34. The Fc fusion molecule of any one of claims 1-26, wherein the
a) Fc fragment comprises a sequence having at least 80% sequence identity to SEQ ID NO: 3,
b) the linker comprises a sequence of SEQ ID NO: 13, and
c) the albumin or albumin binding domain comprises a sequence having at least 80% sequence identity to SEQ ID NO: 12.

35. The Fc fusion molecule of any one of claims 1-26, wherein the
a) Fc fragment comprises a sequence having at least 80% sequence identity to SEQ ID NO: 3,
b) the linker comprises a sequence of SEQ ID NO: 13, and
c) the albumin or albumin binding domain comprises a sequence having at least 80% sequence identity to SEQ ID NO: 11.

36. The Fc fusion molecule of any one of claims 1-26, wherein
a) the Fc fragment comprises a sequence having at least 80% sequence identity to SEQ ID NO: 3,
b) the linker comprises a sequence of SEQ ID NO: 15, and
c) the albumin or albumin binding domain comprises a sequence having at least 80% sequence identity to SEQ ID NO: 12.

37. The Fc fusion molecule of any one of claims 1-26, wherein
a) the Fc fragment comprises a sequence having at least 80% sequence identity to SEQ ID NO: 3,
b) the linker comprises a sequence of SEQ ID NO: 15, and
c) the albumin or albumin binding domain comprises a sequence having at least 80% sequence identity to SEQ ID NO: 11.

38. The Fc fusion molecule of any one of claims 1-26, wherein
a) the Fc fragment comprises a sequence having at least 80% sequence identity to SEQ ID NO: 6 or SEQ ID NO: 7,
b) the linker comprises a sequence of SEQ ID NO: 13, and
c) the albumin or albumin binding domain comprises a sequence having at least 80% sequence identity to SEQ ID NO: 12.

39. The Fc fusion molecule of any one of claims 1-26, wherein
a) the Fc fragment comprises a sequence having at least 80% sequence identity to SEQ ID NO: 6 or SEQ ID NO: 7,
b) the linker comprises a sequence of SEQ ID NO: 13, and
c) the albumin or albumin binding domain comprises a sequence having at least 80% sequence identity to SEQ ID NO: 11.

40. The Fc fusion molecule of any one of claims 1-26, wherein
a) the Fc fragment comprises a sequence having at least 80% sequence identity to SEQ ID NO: 6 or SEQ ID NO: 7,
b) the linker comprises a sequence of SEQ ID NO: 15, and
c) the albumin or albumin binding domain comprises a sequence having at least 80% sequence identity to SEQ ID NO: 12.

41. The Fc fusion molecule of any one of claims 1-26, wherein
a) the Fc fragment comprises a sequence having at least 80% sequence identity to SEQ ID NO: 6 or SEQ ID NO: 7,
b) the linker comprises a sequence of SEQ ID NO: 15, and
c) the albumin or albumin binding domain comprises a sequence having at least 80% sequence identity to SEQ ID NO: 11.

42. The Fc fusion molecule of any one of claims 1-26, wherein
a) the Fc fragment comprises a sequence having at least 80% sequence identity to SEQ ID NO: 2,
b) the linker comprises a sequence of SEQ ID NO: 13, and
c) the albumin or albumin binding domain comprises a sequence having at least 80% sequence identity to SEQ ID NO: 10.

43. The Fc fusion molecule of any one of claims 1-26, wherein
a) the Fc fragment comprises a sequence having at least 80% sequence identity to SEQ ID NO: 8 or SEQ ID NO: 9,
b) the linker comprises a sequence of SEQ ID NO: 13, and
c) the albumin or albumin binding domain comprises a sequence having at least 80% sequence identity to SEQ ID NO: 11.

44. The Fc fusion molecule of any one of claims 1-26, wherein
a) the Fc fragment comprises a sequence having at least 80% sequence identity to SEQ ID NO: 8 or SEQ ID NO: 9,
b) the linker comprises a sequence of SEQ ID NO: 15, and
c) the albumin or albumin binding domain comprises a sequence having at least 80% sequence identity to SEQ ID NO: 11.

45. The Fc fusion molecule of any one of claims 1-26, wherein
a) the Fc fragment comprises a sequence having at least 80% sequence identity to SEQ ID NO: 2,
b) the linker comprises a sequence of SEQ ID NO: 13, and
c) the albumin or albumin binding domain comprises a sequence having at least 80% sequence identity to SEQ ID NO: 11.

46. The Fc fusion molecule of any one of claims 1-26, wherein
a) the Fc fragment comprises a sequence having at least 80% sequence identity to SEQ ID NO: 2,
b) the linker comprises a sequence of SEQ ID NO: 15, and
c) the albumin or albumin binding domain comprises a sequence having at least 80% sequence identity to SEQ ID NO: 11.

47. The Fc fusion molecule of any one of the preceding claims, wherein the Fc fusion molecule comprises, from N-terminus to C-terminus, the Fc fragment, the linker, and the albumin or the albumin binding domain.

48. The Fc fusion molecule of any one of claims 1-46, wherein the Fc fusion molecule comprises, from C-terminus to N-terminus, the Fc fragment, the linker, and the albumin or the albumin binding domain.

49. An Fc fusion molecule that binds neonatal Fc receptor (FcRn) comprising a sequence having at least 80%, at least 85%, at least 90%, at least 95%, at least 96%, at least 97%, at least 98%, or at least 99% sequence identity to any one of SEQ ID NOs: 16-55 or 68-73.

50. The Fc fusion molecule of claim 42, wherein the Fc fusion molecule comprises a sequence identical to any one of SEQ ID NOs: 16-55 or 68-73.

51. The Fc fusion molecule of any one of the previous claims, wherein the Fc fusion molecule comprises an amino acid sequence having at least 80%, at least 85%, at least 90%, at least 95%, at least 96%, at least 97%, at least 98%, or at least 99% sequence identity to any one of SEQ ID NOs: 28-55 or 68-73.

52. The Fc fusion molecule of any one of the previous claims, wherein the Fc fusion molecule comprises an amino acid sequence identical to any one of SEQ ID NOs: 28-55 or 68-73.

53. The Fc fusion molecule of any one of the preceding claims, further comprising a second Fc fragment.

54. The Fc fusion molecule of claim 53, wherein the second Fc fragment comprises the same amino acid substitutions as the first Fc fragment.

55. The Fc fusion molecule of claim 54, wherein the first Fc fragment and the second Fc fragment are further modified to promote Fc heterodimerization.

56. The Fc fusion molecule of claim 54 or 55, wherein the first Fc fragment and the second Fc fragment further comprise one or more amino acid substitutions selected from Y349C, S354C, T366S, T366W, T366Y, L368A, Y407T, Y407V.

57. The Fc fusion molecule of any one of claims 54-56, wherein
a) the first Fc fragment further comprises amino acid substitutions Y349C, T366S, L368A, and Y407V, and the second Fc fragment further comprises amino acid substitutions S354C and T366W; or
b) the second Fc fragment further comprises amino acid substitutions Y349C, T366S, L368A, and Y407V, and the first Fc fragment further comprises amino acid substitutions S354C and T366W; or
c) the first Fc fragment further comprises amino acid substitutions T366S, L368A, and Y407V, and the second Fc fragment further comprises amino acid substitution T266W; or
d) the second Fc fragment further comprises amino acid substitutions T366S, L368A, and Y407V, and the first Fc fragment further comprises amino acid substitution T266W.

58. The Fc fusion molecule of any one of claims 53-57, wherein the second Fc fragment comprises an amino acid sequence at least 80%, at least 85%, at least 90%, at least 95%, at least 96%, at least 97%, at least 98%, or at least 99% identical to any one of SEQ ID NOs: 1-9 or 57-67.

59. The Fc fusion molecule of any one of claims 53-58, wherein the second Fc fragment comprises an amino acid sequence identical to any one of SEQ ID NOs: 1-9 or 57-67.

60. The Fc fusion molecule of any one of the preceding claims, wherein the Fc fusion molecule binds to FcRn with a K_{D} of less than or equal to about 1×10⁻⁸M, at pH 6.0 or pH 7.4 as measured by surface plasmon resonance (SPR).

61. The Fc fusion molecule of any one of the preceding claims, wherein the Fc fusion molecule comprises a half-life at least 2-fold longer as compared to an Fc fusion molecule not comprising an albumin or an albumin binding domain.

62. The Fc fusion molecule of any one of the preceding claims, wherein the Fc fusion molecule reduces IgG level to less than 50%, to less than 40%, to less than 30%, or to less than 25%.

63. A pharmaceutical composition, comprising the Fc fusion molecule of any one of claims 1-62, and a pharmaceutically acceptable carrier.

64. A method of treating a disease or disorder in a patient in need thereof, the method comprising administering to the patient an effective amount of the Fc fusion molecule of any one of claims 1-62 or a pharmaceutical composition of claim 63.

65. The method of claim 64, wherein the disease or disorder is an autoimmune disease.

66. The method of claim 64 or 65, wherein the disease or disorder is generalized myasthenia gravis (gMG), chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenic purpura, primary Sjogren's Syndrome, systemic lupus erythematosus, rheumatoid arthritis, bullous pemphigoid, pemphigus foliaceus, pemphigus vulgaris, or cutaneous lupus erythematosus.

67. The method of any one of claims 64-66, wherein the method reduces disease severity in a patient and wherein disease severity is assessed by an gMG Disease Severity Outcome Measure.

68. A method for treating a pathology associated with elevated levels of an IgG in a patient in need thereof, the method comprising administering to the patient a therapeutically effective amount the Fc fragment of any one of claims 1-62 or a pharmaceutical composition of claim 63.

69. A method of reducing biological activity of an IgG in a patient in need thereof, the method comprising administering to the patient a therapeutically effective amount of the Fc fusion molecule of any one of claims 1-62 or a pharmaceutical composition of claim 63.

70. A method of reducing IgG levels in a patient in need thereof, the method comprising administering to the patient a therapeutically effective amount of the Fc fusion molecule of any one of claims 1-62 or a pharmaceutical composition of claim 63.

71. The method of claim 69 or 70, wherein the Fc fusion molecule reduces IgG level by 50% within 2, days, 3 days, 5 days, or 7 days of the administration.

72. The method of claim 69 or 70, wherein the Fc fusion molecule reduces IgG level by 75% within 2, days, 3 days, 5 days, or 7 days of the administration.

73. The method of any one of the claims 68-72, wherein the administration of the Fc fusion molecule reduces IgG level to less than 50%, to less than 40%, to less than 30%, or to less than 25% for at least 5 days, 7 days, 10 days, 15 days, or 20 days post administration.

74. A method of preventing a disorder in a patient in need thereof, the method comprising administering to the patient a therapeutically effective amount of the Fc fusion molecule of any one of claims 1-62 or a pharmaceutical composition of claim 63; wherein the disorder is an unwanted side-effect of a therapeutic antibody.
